SEQUENCE LISTING (38 sequences)

SEQ ID NO: 1 - protein, 5 aa, synthetic construct
GGGGS

SEQ ID NO: 2 - DNA, 837 bp, synthetic construct
  source 1..837: mol_type = other DNA
atggaaactaccaccttggttttgttgttgccacatggtggtgctggtggtgttagaccagctgctgctgctactgctaaaagatcttatgttatgagaagatgctgctccactgttagagctgttatggctagacctcaagaagctccagcttctgctccagctaaaaaaactgaaactgctgctatgatgtctaccgttcaaacagaaactgcagctgccccaccagctactgtttacagagattcttggtttgataagttggccatcggttacttgtctaggaacttgcaagaagcttcaggtttgaagaacgaaaaggatggttacgaatccttgattgatgctgctttggccatctccagaattttctcattggataagcaatccgaaatcgttacccaagctttggaaagagctttgccatcttacattttgaccatgatcaaggttatgatgccaccatctagattctccagagaatactttgctgctttcaccactattttcttcccatggttggttggtccatgtgaagttatggaatctgaagtcgaaggcagaaaagaaaagaacgttgtttacatcccaaagtgcaggttcttggaatctactaattgtgttggtatgtgcaccaacttgtgtaagattccatgccaaaagttcatccaggattctttgggtatgaaggtttacatgtctccaaacttcgaagatatgtcctgcgaaatgattttcggtcaacaaccaccagaagatgatccagctttgaaacaaccatgtttcagaactaagtgcgttgccaaacaaaatcatggtgttaactgctccatctaa

SEQ ID NO: 3 - DNA, 1869 bp, synthetic construct
  source 1..1869: mol_type = other DNA
atgctgaccaaaatgtctttgccaattccaccaaagtttctgccaccattgaaatctccaccaatccatcatcatcaaactccaccaccattggctccaccaagagctgctatttctatttcaattccagataccggtttgggtagaaccggtactattttggatgaatctacttcctctgccttcagagattaccaatctttgttcgtgtctcagagatccgaaactattgaaccagttgttatcaagccaatcgaaggttctattccagttaattttccatctggcacttactatttggctggtccaggtttgtttactgatgatcatggttctactgttcacccattggatggtcatggttatttgagagctttccatatcgatggtaacaagagaaaggctactttcactgctaagtacgttaagaccgaagccaaaaaagaagaacacgatccagttactgatacttggagattcactcatagaggtccattctctgttttgaaaggtggtaagagattcggtaacaccaaggttatgaagaacgttgctaacacttccgttttgaaatgggctggtagattattgtgtttgtgggaaggtggtgaaccatacgaaattgaatctggttctttggataccgtcggtagattcaatgttgaaaacaacggttgcgaatcttgcgacgatgatgattcttctgatagagatttgtccggtcatgatatttgggatactgctgctgatttgttgaagccaattctacaaggtgttttcaagatgccaccaaagagattcttgtcccattacaaagttgacggtagaagaaagaggttgttgactgttacttgtaacgccgaagatatgttgttgccaagatctaacttcaccttctgcgaatacgattccgaattcaagttgattcagaccaaagagttcaagatcgacgatcacatgatgatccatgattgggcttttaccgatactcactacattttgtttgccaacagagtcaagttgaacccaattggttctattgctgctatgtgtggtatgtctccaatggtttctgctttgtctttgaacccatctaacgaatcttccccaatctatattttgccaaggttctccgataagtactctagaggtggcagagattggagagttccagttgaagtttcttctcaattgtggttgatccattctggtaacgcttacgaaactagagaagataacggtgacttgaagattcaaattcaagcttctgcttgctcctacagatggtttgattttcaaaagatgttcggttacgactggcagtctaacaaattggatccatctgttatgaacttgaacagaggtgatgacaagttgttaccacacttggttaaggtttctatgaccttggattctaccggtaactgtaactcttgtgatgttgaacctttgaacggttggaacaagccatctgattttccagttattaactcctcttggtccggcaaaaaaaacaagtatatgtactctgctgcctcctctggtactagatctgaattgccacattttccattcgatatggttgtcaagttcgacttggactctaacttggttagaacttggtctactggtgctagaagatttgttggtgaacctatgttcgtcccaaagaactctgttgaagagggtgaagaggaagatgacggttatatcgttgttgttgaatacgccgtttctgtcgaaagatgctacttggttattttggacgccaaaaagatcggtgaatctgatgctgttgtttccagattagaagtcccaagaaatctgactttcccaatgggttttcatggtttgtgggcttcagattga

SEQ ID NO: 4 - DNA, 1713 bp, synthetic construct
  source 1..1713: mol_type = other DNA
atggcttctttgatcacaaccaaagcaatgatgagtcatcatcatgttttgtcgtcaactagaatcactactctttattccgacaattccatcggcgatcaacaaataaaaacaaaacctcaagtccctcaccggttatttgctcggaggatcttcggtgtaaccagagctgtaattaattcagcggcaccgtctccgttgccggagaaagagaaggtggaaggtgagagacggtgtcatgttgcgtggacaagtgtacaacaagagaattgggagggtgaacttactgtccaaggaaagatacccacttggctgaatggtacgtacctaagaaacggtcctggtctatggaacattggagaccacgatttccggcatctcttcgacggctactccacactcgtcaagcttcaattcgatggcggtcgtatattcgccgcccaccgtctccttgaatccgacgcttacaaagccgccaagaaacacaataggctttgttaccgtgaattctccgagactccaaaatcggtgatcataaacaaaaaccctttctccgggatcggagaaatcgtcaggcttttctccggagagtctttaacggacaacgccaacaccggagtgatcaaactcggtgacgggcgggtcatgtgtctgacggagactcaaaaaggatcgattttagtcgaccatgagacgctagagacgatcgggaaatttgagtacgacgacgtattgtccgatcatatgatccaatcagcgcatccgatagtgacggagacggagatgtggacgttgataccggatttggttaaaccgggttatcgggtcgtgaggatggaagccgggtcgaataaaagagaggttgtggggcgggtgaggtgtcgaagtgggtcgtggggacccggttgggtccattcgtttgcggtgacggagaattatgttgtaataccggaaatgcccctgagatattcggtgaagaatcttcttagagctgagccgacgccactttacaagttcgagtggtgtccccaagacggagcttttattcatgtcatgtccaaactcaccggagaagtcgtggctagcgtggaggttccagcatacgtaacgtttcacttcataaacgcgtatgaagaagataaaaatggcgatggaaaagcgacggtcatcattgcagattgttgtgaacacaacgccgatactcggatactcgatatgctccgtctcgataccctacgttcttcccatggtcacgacgttttacccgatgctaggatcgggagattcaggataccattggacgggagcaaatacgggaaactagagacagccgtggaggcagagaagcatgggagagcgatggatatgtgcagcatcaatcctttgtatttgggtcaaaaataccgttacgtttatgcatgcggtgctcaacgaccttgtaacttccccaatgctctctccaaggttgatattgtggagaagaaagtgaagaactggcacgagcatggtatgataccatctgaaccattcttcgtgcctcgacccggtgcaacccatgaggatgatggagtggtgatatcgatagtaagtgaagaaaatggaggaagctttgcaatcttgcttgatgggagctcctttgaagaaatagcaagagccaagtttccctatggccttccttatggcttgcatggttgctggatccccaaagattaa

SEQ ID NO: 5 - DNA, 2079 bp, synthetic construct
  source 1..2079: mol_type = other DNA
atgacttctgccttgtatgcctctgatttgttcaagcaattgaagtccattatgggcaccgattctttgtctgatgatgttgttttggttatcgctactacctctttggctttggttgctggttttgttgttctgttgtggaaaaagactaccgctgatagatctggtgaattgaaaccattgatgatccccaaatctttgatggccaaagatgaagatgatgacttggacttaggttctggtaagactagagtttccattttcttcggtactcaaactggtactgctgaaggttttgctaaagctttgtccgaagaaatcaaggccagatacgaaaaagctgccgttaaggttattgatttggatgattatgctgccgatgacgaccaatacgaagaaaagttgaagaaagaaaccttggccttcttctgtgttgctacttatggtgatggtgaacctactgataatgctgctagattttacaagtggttcaccgaagagaacgaaagagatatcaagttgcaacaattggcctacggtgtttttgctttgggtaatagacaatacgagcacttcaacaagatcggtatcgttttggatgaagagttgtgtaaaaagggtgccaagagattgattgaagttggtttgggtgatgatgaccagtctatcgaagatgattttaacgcctggaaagaatccttgtggtctgaattggataagttgttgaaggacgaagatgacaaatctgttgctacaccatacactgctgttattccagagtatagagttgttactcacgatccaagattcacgactcaaaagtctatggaatctaacgttgctaacggtaacaccaccatcgatattcatcatccatgtagagttgatgtcgccgtccaaaaagaattgcatactcatgaatccgacagatcctgcattcatttggaattcgatatttccagaaccggtattacttacgaaaccggtgatcatgttggtgtttacgctgaaaatcacgttgaaatcgttgaagaagccggtaagttgttaggtcattcattggatttggtgttctccattcatgccgacaaagaagatggttctcctttggaatctgctgttccaccaccatttccaggtccatgtactttaggtactggtttggctagatatgctgacttgttgaatccaccaagaaagtctgctttagttgctttggctgcttatgctactgaaccatctgaagccgaaaaattgaaacatttgacttccccagatggtaaggacgaatattctcaatggatagttgcctctcagaggtctttgttggaagttatggctgcttttccatctgctaaaccaccattgggtgttttttttgctgctattgctccaagattgcaacctaggtattactccatttcttcatcaccaagattggccccatctagagttcatgttacatctgctttggtttatggtccaactccaactggtagaattcataagggtgtttgttctacctggatgaagaacgctgttccagctgaaaaatctcatgaatgttctggtgccccaattttcattagagcttctaatttcaagctgccaagcaatccatctactccaatagttatggttggtccaggtacaggtttagctccttttagaggtttcctacaagaaaggatggccttgaaagaggatggcgaagaattgggttcttccttgttgttttttggttgcagaaacagacagatggatttcatctatgaggacgagttgaacaacttcgttgatcaaggtgttatctccgaattgattatggccttttctagagaaggtgcccagaaagaatatgtccaacataagatgatggaaaaagccgctcaagtttgggacctaatcaaagaagaaggatacttgtacgtttgcggtgatgctaaaggtatggctagagatgttcatagaacattgcataccatcgtccaagaacaagaaggtgtttcatcttctgaagctgaagctatcgttaagaagttgcaaactgaaggtagatacttgagagatgtctggtga

SEQ ID NO: 6 - DNA, 1569 bp, synthetic construct
  source 1..1569: mol_type = other DNA
atgaagacccaacatcaatggtgggaagttttggatccattcttgactcaacatgaagctttgattgctttcttgactttcgctgctgttgttatcgttatctacttgtatagaccatcttggtctgtctgtaatgttccaggtccaactgctatgccattggttggtcatttgccattgatggctaaatatggtccagatgttttctccgttttggctaaacaatacggtccaatcttcagattccaaatgggtagacaacccttgattattatcgctgaagctgaattgtgtagagaggttggtattaagaagttcaaggacttgccaaacagatccattccatctccaatttctgcttctccattgcataagaagggtttgtttttcaccagagacaagagatggtcaaagatgagaaacaccatcctgtcattataccagccatctcatttgacctcattgattccaactatgcactcctttattacctctgctactcacaacttggattccaaacctagagatatcgtgttctccaacttgttcttgaagttgaccaccgatattattggtcaagctgcttttggtgttgactttggtttgtctggtaaaaagccaatcaaggatgttgaagttaccgacttcatcaaccagcatgtttactctactacccaattgaagatggacttgtctggttccttgtccattattttgggtttgttgatcccaatcctgcaagaaccttttagacaagtcttgaagagaatcccaggtactatggattggagagttgaaaagactaacgctagattgtctggtcagttgaacgaaatcgtttccaagagagctaaagaagctgaaaccgattctaaggacttcctgtctttgattttgaaggccagagaatctgatccattcgccaagaatattttcacctccgattacatttctgccgttacctatgaacatttgttggctggttctgctactaccgcttttactttatcttctgtcctgtacttggtttccggtcacttagatgtcgaaaagagactgttgcaagaaatcgatggtttcggtaacagagatttgattcctactgctcatgacttgcaacataagttcccatacttggaccaagttatcaaagaagccatgaggttctatatggtgtctccattagttgctagagaaaccgcaaaagaagttgaaattggcggttacttgttgccaaaaggtacttgggtttggttggctttgggtgttttggcaaaagatccaaagaatttcccagaaccagaaaagttcaagccagaaagatttgatccaaacggtgaagaagaaaagcacagacatccatacgcttttattccatttggtattggtcctagagcttgtgttggacaaagatttgctctacaagaaatcaagttgaccctgttgcacttgtacagaaactacatcttcagacacagcctggaaatggaaataccattgcaattggattacggcatcatcctgagttttaagaacggtgttaagttgagaaccatcaagaggttctga

SEQ ID NO: 7 - DNA, 1620 bp, synthetic construct
  source 1..1620: mol_type = other DNA
atggaaatctctactgttttgggtgctattttggctgaatacgctgttactttggttgctatggctgttggttttttggttgttggttacttgtatgagccatactggaaggttagacatgttccaggtccagttccattgccattgattggtcacttgcatttgttggctatgcatggtccagatgttttctctgttttgactagaaagtacggtccaatcttcagattccatatgggtagacaaccattggttatggttgctgatgctgaattgtgtaaagaagttggcgttaagaagttcaagaacttcccaaatagatccatgccatctccaattaccaattctccagttcatcaaaagggtctgtttttcacttctggttctagatggactaccatgagaaacatgatcttgtctatctaccaaccatctcatttggctaccttgattccttctatggaatcctgtattgaaagagctgctgaaaacttggaaggtcaagaagaaatcaacttctccaagctgtctttgtctttcactaccgatgttttaggtcaagctgcttttggtactgatttcggtttgtctaagaaattggcctcctctgatgatgatgaagataccagaaaaattgctgctgatacttgtgctgaagctaaagcttcttccgaattcatcaagatgcatgttcatgctaccacctcattgaagatggatatgtctggttccttgtccattatcgtcggtcaattattgccattcttgcacgaacctttcagacaagttttgaagagattgagatggactgccgatcacgaaattgatagagttaatttgactctgggcagacagttggatagaatagttgctgaaagaacagctgccatgaagagagatccagctgctttacaacaaagaaaggatttcttgtccgttatgttgaccgctagggaatctaacaaatcttctagagaattactgacgcccgattacatttctgctttgacttatgaacatctgttggctggttctgctactactgcttttactttgactactgccttgtacttggttgccaaacatccagaagttgaagaaaagttgttgagggaaattgatggtttcggtccaagagatagagttccaactgctgaagatctgcaaactaagtttccatacttggaccaggttttgaaagaagctatgaggtattacccatcctcacctttgattgccagagaattgaatcaacagttggaaatcggtggttacccattgccaaaaggtacttgggtttggatggctccaggtgtattgggtaaagatccaaagaattttccagagccagaagtgtttagaccagaaagatttgatccaaacggtgaagaagaaaagcgtagacatccatatgctttgttcccatttggtattggtcctagagcttgtattggtcaaaagttcgctatccaagaaatgaagttgtccgccattcatttctacagacactacgtttttagaccctctccatcaatggaatctccaccagaatttgtctactccatcgtttctaatttcaagaacggtgctaagttgcaggttatcaagagacatatctaa

SEQ ID NO: 8 - DNA, 1626 bp, synthetic construct
  source 1..1626: mol_type = other DNA
atggaaatcatctctaccgttttgggttctactgctgaatatgctgttactttggttgctatggctgttggtttgttgttgttgggttacttgtatgaaccatactggaaggttagacatgttccaggtccagttccattgccttttattggtcacttgcatttgttggctatgcatggtccagatgtttttactgttttggctagaaagtacggtccagttttcagatttcatatgggtagacaaccattggttatggttgctgatgctgaattgtgtaaagaagttggcgttaagaagttcaagtccattccaaatagatccatgccatccgctattgccaattccttgatcaatcaaaagggtttgtgtttcaccagaggttctagatggactgctttgagaaacatgatcatctcaatctaccaaccatctcatttggcctcattgattccaactatgcaatcttgcattgagtgcgtgtctaagaatttggatggtcaagaagatatcaccttctccgatttggctttgggttttgctactgatgttataggtcaagctgctttcggtactgattttggtttgtctaagatttccgcctcctctaacgatgatgatattgataagattgccaccgatacctctgctgaagctaaagcttcttctgaattcatcagaatgcatgttcatgccaccacatctttgaagatggatttgtctggttccctgtccattatcatcggtcaattattgccattcttacaagaaccattcaggcaggttttgaagagaattccatggacagctgatcacgaaatcgatcatgttaatttggcactaggtggtcaaatggataagatagttgctgaaagggctgctgctatggaaagagatcaagctgcaccacatgctcaacaaagaaaggattttttgtctgttgttttggccgccagagaatctaacaaatcttggagagaattactgaccccagattacatttctgctttgacctacgaacatttgttagctggttctgctactactgctttcactttgtctactgtcttgtacttggtttctaagcacccagaagttgaggaaaagttgttgagagaaattgatggtttcggtccacatgatcacgctccaactgctgaagatctacaaactaagtttccatacttggaccaggtcgtcaaagaatccatgaggttttactttctgtccccattgattgctagagaaacctgtgaacaagttgaaattggtggttacgctttgccaaaaggtacttgggtttggttggctccaggtgttttagcaaaagatccaaagaatttcccagagccagaagtctttagaccagaaagatttgatccaaacggtgaagaagaaaagcgtagacatccatacgcttttattccatttggtattggtccaagagcttgcattggtcaaaagttctctatccaagaaatcaagttgtccgtcatccacttgtacagaaactacgtttttagacactccccatctatggaatctccattggaatttcaatactccatcgtctgcaatttcaagtacggtgttaagttgagagtcatcaagagacatactgcctaa

SEQ ID NO: 9 - DNA, 1539 bp, synthetic construct
  source 1..1539: mol_type = other DNA
atggatgacatgcactctcaattgcaagctgctggtgctgcttgtcaacaatctaattctttgttgttgccaccaccagctgctgatagaccttgttcttcttcatcttcatcctcgttgtctttgttgggtacagctgctgctgcatgtttgtttttgtctgctgctatctactgcatcgtcgttattatcgttactacctcctctaagcagaacatcaacaacagattgatcaggcgtttgttgaagttcaagggtagaagatctaagaacgacagaagaagagactacaacaacaatgctgctccaccaccaccacctccaggtagaggttcttcttggtggtggtcagttgttgaaactttggcttttgtttccgctaacagatctggtagaggcttgtatcatttcgttgaagctagacatagaagatacggtccaccatgttttagaactgctttgttaggtgctacccacgtttttgtttcttcaccagatgctgctagaagtttgttggctgatgctggtggtttttctaagagatacgttagaaccgttgccgaattattgggtgaacattctttattgtgcgcttcccatgatgctcatagagctttgagaagggctgttgctcctttgtttaatgctcaagctactgcttctttggctgctaattttgatgctttggccagaagaattatcaccagagattgggctgctaaaactactgctgttgttgttttggatgctgctttggatgttaccttcgaagctatttgcgatatgttgattggtagaactaccaccttgaagcgtagaagattacaatctgatgttttggctgttaccagagctatgttggcttttccattgagattgccaggtactagatttcatgctggtttgagagccagaaaaagaatcatggatgtcttgagacaagaaatcgcttccagacaaagaaacatcatggatatggaagaaatggaagaggatgattccaagcacgacaatgatttcttgcagtccttgttgttattgaggcgtagaaagatgaagtcctctcaacagcaacaatctccatctaactctaacgatcatttgttcttgaccgacgatcaaatcttggataacatcctgaccttgattattgctggtcaagttactacagcttctgctattacttggatggttaagtacttggccgataacaaggatttccaagaaaccttgagatccgtgcaattggaaatggctttgaaacaccaacatggtgattctgatggtcctttgactctgcaacatttgaactctatggaattggcttacatgaccgtcaaagaaagtttgagaatggcctctatcgtttcctggtttccaagagttgctttggaagattgtcaagttgctggttttcatatcaacaaaggttggatcgttaacattgatgctagagccttgcattatgatgctaccttgtatgataacccaaccatgtttgatccatccagattcaaaatgggtgatggtagaaggtga

SEQ ID NO: 10 - DNA, 1557 bp, synthetic construct
  source 1..1557: mol_type = other DNA
atgaacatggaatctttggctgctggtgcttggtgggttgttgttttgttgttattggttttgaccatcgttgcctcttggtatagatcttggtggaaaactactgaagctggtggtccattattgccacctccagcagctggtgctggaccatggtgggtttgggtttggcaatggcgtgaaactgctgcttttttggcttctcatggttctggtagaggtttctaccattttgtccaagaaaggtacaagctgtacaaaggtgaaggtgagggtgaagctacatgttgttttagaactgctttgatgggtagagtccacgtttttgtttctgcttctcatccagctgcttcccaattattgactgctgaaccaccacatttgccaaaaagatatgctagaacagctgctgatttgttgggtccacattctattttgtgttctacctctcatgcccatcatagacatgctagaagggctttagctactactttgtttgctactccatctacagctgcttttgctgctgcatttgatagattggttattagacattggaccaccttgttgccaccacacaatcaaaatcaagttgttgttgtattggatgccgccttgcatatttcttacagagctatttgcgaaatgttgttaggtgctggtggtggtaagttaagaccattgcaatctgatgttttcgctgttactcaagctatgttggctttgccattgagatggttgccaggtactaggtttagaagaggtttacatgccagaaaaagaattatggctgccttgagagaagaaatggctgctagaaatcatcatcaccaccatcaccatcatcatcacgatttgttgtctgttttgatgcaaagaaggcaattgggtcatccagatgcattgactgaagatcaaattctggataacatgctgaccttgattattgctggtcaagttactactgctactgctattacttggatggtgaagtacttgtccgataacagattgatccaagataagttgagagctgaagccttcagattggaattgaaaggtgattactctttgaccatgcaacatttgaacgctatggattacgcttacaaggctgtcaaagaatcattgagaatggctactatcgttagctggtttccaagagttgctttgaaggattgtcaagttgctggttttcacatcaagaaggattggatcgttaacatcgatgccagatcattgcattacgatccagatgtttttgataacccaaccgttttcgatccatccagattcgatgaagaaggcgaaggcgacgacgctaaattgggtagagcacaaccacaaaagagaaggttgttggtttttggtgccggtggtagaacttgtttgggtatgaatcatgccaagatcatgatgctgattttcttgcataggctgttgactaacttcagatgggaaatggcagatgatgatccatctttggaaaagtgggctatgttcccaagattgaaaaatggttgcccaattttgttgaccccaatccataactcttaa

SEQ ID NO: 11 - DNA, 1476 bp, synthetic construct
  source 1..1476: mol_type = other DNA
atgctgaacttgtctgttgagggtttgactttggttgttcaaaaccattacggtatcttgatcgttgccgttttgtctattactatcacctccttgttgttgaaagcttggggttctactgttgatatcactgatgaagatggtatcccaggtagattgggtttgccattttttggtgaaaccttctctttcttctccgcatcttattctactaagggttgttacgatttcgtcaagcaaagaagaaagcagtacggtaaatggttcaagaccagaattttgggtaagacccatgttttcgttccatctgttgaaggtgctaagactattttggccaatgatttcgttcacttcaacaagtcctacgttaagtctatggctgatgctactggtgctatgtctgttttttctgttccacataagatccacaccagaatcagaagattattgtccgatccattctccatgtcctcattgtctaaattcgctgttaagttcgataagatggtctgcgaaagattggacaagttggaaaaatctggtaagtccttcagagtgatcgacttctctttgaaaattaccttcgatgccatcgtgtccatgttgatgtctgttactgaaaaccctttgttggaacagatcgaaaaggattgcactgatgtctctaactccatgttgtctattccattgatgattccaggtacgaggtactacaaaggtatgaaaggtagaggtaagctgaacgaaactttcggtaatatgattgccagaagaagaatcggcgaagaatacttcgatgatttcttgcaaaccgttgttgacagagattcttatcctgaagatgagaagttggacgaccaagagattattgataacctgatcaccttgattttggccggtcaaactactactgcttctgctatgatgtggtgcgttaagtttttgtccgaaaacaaggatgtcttggacagattgagagaagaacaattgtccatcgttagaaacaaagctgaaggtgcatctttgaccttggaagatttgactgaaaagagctacggtttcaaggttgtcaaagaaactttgagaatggccaacgttttgatctggttgccaagagttgctatggatgattgcattatcgatggtttcgaagtcaagaaaggttggttggttaatgttgatgctacctgcattcattacgatccaaacgtttacaaagacccaactagattcaacccatccagatttgatgattttcagaagccctactctttcttgccatttggtgctggtccaagaacttgtttgggtattaacatggctaaggttgccatgttggttttcgtccatagattgacatctggttacaagtggactttggatgatccagattctagcttggaaagaaaagaacacatcccaagattgagatccggttgtccaattactttgaaggctttgaacaagggcaagtaa

SEQ ID NO: 12 - DNA, 1455 bp, synthetic construct
  source 1..1455: mol_type = other DNA
atgctgaacttgtccagagaagaattggttatcgttgttgctttgttgtgcgttggtattacttacttggcttctaaggcttgtaaaagggcttcttctaacgaaagagaagatatcccaggtagattgggtttgccttttattggtgaaaccttctcctttttgtccgcttacaattctactagaggttcctacgatttcgttaccccaagaagattgagatttggtagatggtttaagaccaggttgttcggtaagatccatatctttgttccaaactctgaaggtgccagaattatcttggctaatgatttcgtcttgttcaacaagggttacgttaagtctttggctgaagctgctggtaagaactctttgttttgtgttccagttgaatcccacaagagaatgagaagattattgtccgaaccattctctatgacttctccatctgctttcattaccaagttcgataagaaaatgtgcgccaggttgcaaaaattggaagaaggtggtcaatccttcaaggttttggatttctgtatgaagatgtccttcgatggtatctgcgaaatgttgatgtctatcaccgaagattccttgttggaaaagatctggaaggattctattgctgctggtgaagccatgatttctattccagctatgattccaggttccaggtattacaaaggtatgaaggctagaagaaggctagttgaaactttcaccgaaattattgccagaagaagaagaggcgaagaatctgctgaagatttcttgcaatctatgttgcagagagatttgttcccagcttctgaaaagttggatgactctgaaatcatcgacaacatgctgacctttattttctctggtcaatctactactgctaccgctatgatgtggtctgttaagtttctacacgagaacaaagaagtccaagacatcttgagagaagaacagttgtctctgtctaagatgaagccagaaggtgctccattgacaaaagaggatattaacaatatgccatacggctggaaggtcttgaaagaaactttgagaatgtccaacatcgtcttgtggtatccaagagttgctttacaagattgcaccattgaaggtcgtgaaatcaaaaaaggttggcacgttaacattgatgctacctgtgttcattttgaccccgacttgtacaaagatccattgaagtttaacccacagagattcgacgaaactcaaaagccatactctttcattccatttggtgctggtccaagaacttgtttgggtatgtatatggctaagctgaagatgttgatcttcatccatagattggttggtggttacacttggactttggatgatttggataactccttgcaagccaaagagttggttccaaaattgagatctggttgcccaattaccttgaagtctttgtctaagtctagatccgaagcctga

SEQ ID NO: 13 - DNA, 1485 bp, synthetic construct
  source 1..1485: mol_type = other DNA
atgctgaacgtcttgatgagagaagaagttttgttggttgtccaaaactgctaccacattattttggttgccttgttgtctatcggtgttacttacttggcttctaaagcttggaaaagagctactaccaacaacagagaagaaatcccaggtagattgggtttgccatttgttggtgaaactttctctttcttgtctgctaccaattctaccagaggttgttacgatttcgtcagattgagaagattgtggaatggtagatggttcaagactaggttgttcggtaagatccatatcttcgttccaaatccagaaggtgctagaactattttcgccaatgatttcgtcttgttcaacaagggttacgttaagtctatggctgatgctgttggtaaaaagtctttgttgtgtgttccagtcgaatcccataagagaattagaaggttgttgtccgaacctttctctatgacttctttgtctgctttcgttaccaagttcgataagttgttgtgcgaaagattgcagaagttggaagaaagaggtaagtccttcaaggttttggatttctgtatgaagatgaccttcgatgctatgtgcgatatgttgatgtctatcaccgaagattctttgttgcagcaaattgaagaggattgcaacgctgtttctgatgccatgttatccattccaattatgattccaggtacgaggtactacaaaggtattaccgctagaaaaaggctgatggaaaccttcagagaaattatcggtagacgtagaagaggtgaagaaaccagagaagatttcttgcaatccatgttgcaaagggattctttgccaccatctgaaaagttggatgactccgaaattatggacaacttgctgaccttgattattgctggtcaaactactacagctgctgctatgatgtggtctgttaagttcttgcatgataacagggaagcccaagacatcttaagagaagaacaattgtccatcaccaacatcaaaccagatggtgcttctttgagtcacgaagatttgaacaacatcaggtacggtttgaaggttgtcaaagaaaccttgagaatgtccaacgtcttgttgtggtttccaagagttgctttacaagattgcaccattgaaggttacgacatcaaaaaaggttggcacgttaacattgatgccacctacattcatcatgactctgacttgtataacgaccccttgaagtttaacccaaagagattcgacgaacaccaaaagccatattcctttattccatttggttctggtccaagaacttgcttgggtattaacatggctaaggttaccatgttggttttcttgcatagattggctggtggttatacttggactttggatgatttggatacctgcttagaaaagaaggcccatattccaagattgagatctggttgtcctatcaccttgaagtccttgtctaaaactatgttggaagcctaa

SEQ ID NO: 14 - DNA, 1461 bp, synthetic construct
  source 1..1461: mol_type = other DNA
atgctgaccatgtccatggaagatatccttttgttgctgtccaagtactacgacatcttgttggtttccatcttggttatttctatcaccgccttgtacttgtctaagggtgctaaaaatgctaagtcttgcattccaggttctttgggtattccatttgttggtgaaactttcgctttgttgtctgctaccaattctgttaagggttgctacgaatttgtcaggttgagaagagaaagacacggtaaatggttcaagaccagaattttcggtaagatccatgttttcgttccatctgttgaaggtgctaaggctattttcactaatgatttcgccttgttcaacaaaggctacgttaagtctatggctgatgctgttggtaaaaagtctttgttgtgtgtcccacaagaatcccataagagaattagaaggttgttgtccgatcctttctccatgaattctttgtccaagttcgttcagagattcgacgaaatgttgtacgaaagattgaagaaggtccagaagcagagaaagtcttttaccgttttggacttcaacatgaagactacctttgatgctatgtgcgacatgttgatgtctatcaaggattcctctgttgtcgaacaaatcgaaaaagattgcaccgctatttctgatgccatgttgtcttttccagttatgattccaggtacgagatacttcaaaggtattaaggctagaggtaggctgatggaaactttcaagggtatgattgctgctagaagaaacggcaaagaatattacgaagatttcctgcagtccatgttggaaaaagatagttgtccagccaatcaaaagttggacgatgaagaaatcatggacaacttgctgaccttgattattgctggtcaaactactacagctgctgctatgatgtggtctgttaagtttttggatgaacatagagaagcccagaacagattgagagaagaacaattgtccatcttgagatctaaacctactggtgctttgttgaccatggatgatttgaactctatgtcctacgcttccaaggttgtcaaagaaactttgagaatgtccaacgtcttgttgtggtttccaagagttgctttgaacgattgctctattgaaggtttcgagatcaagaaaggttggcatgctaatattgatgctacctgcattcattacgatcccaccttgtacaaagatccaatgcaattcaatccatccaggttcgatgagattcaaaagccatattcctacattccattcggttctggtccaagaacttgtttgggtatcaatatggctaaggttaccatgttggttttcttgcatagattgactactggttacaagtggactgttgatgatccagatagatccttggaaagaaaggctcatattccaagattgagatctggttgtccaattactttgactgctttgccagatgagtga

SEQ ID NO: 15 - DNA, 1473 bp, synthetic construct
  source 1..1473: mol_type = other DNA
atgttggccttgtccatggaagatattctgttgttggtttccaagtactacgatatcttgttggcctccattttggttatttctgttaccgccatctacttgatcccaaaggtttctactagagccaaggattctaaatcttgtgttccaggtaatttgggtatcccatttgttggtgaaactttcgctttgttgtctgctaccaattctgttaagggttgctacgatttcgtcagattgagaagagaaagacatggtagatggttcaagaccagaattttcggtaagatccatgttttcgttccatctgttgaaggtgctaagaccattttcactaatgatttcgccttgttcaacaaaggctacgttaagtctatggctgatgctgttggtaaaaagtctttgttgtgtgtcccacaagaatcccataagagaattagaaggttgttgtccgatcctttctccatgaattctttgtccaagttcgttgaaaggttcgacaatatgttgtgcgagagattgaaaaaggtccagatggaagagaagtctttcaccgttttggatttcaacatgaagattaccttcgatgccatgtgcgatatgttgatgtctattaaggatgcctccttgttggaacaaatcgaaagagattgcaccgttgtttctgatgctatgttgtctttcccagttatgattccaggtactaggtactacaaaggtattaaggctagaaggcgtttgatggaaactttcaagaagatgattaccgccagaagatctggtaaggattatcacgaagatttcttgcagtccatgttggaaaaagattcttgtccagctgatcaaaagttggacgatgaagaaatcatggacaacctgctgactatgattattgctggtcaaactactacagctgctgcaatgatgtggtctgttaagtttttggatgaacatagagaagcccagaacagactaagagaagaacaattgtctgtcttgaggtctaaacctaatggtgctttgttggctttggatgacttgaactctatgtcttacgcttccaaggttgtcaaagaaaccttgagaatgtccaacgttttgttgtggttcccaagagttgctttgaacgattgctctattgaaggtttcgagatcaagaaaggttggcatgctaatattgatgctacctgcattcattacgaccccactatctacaaacatccaatgagattcaacccatccagattcgacgaaatgcaaaaaccatactcctacattccatttggttctggtccaagaacttgcttgggtattaacatggctaaggttaccatgctggttttcttgtacagattgacttctggttacaagtggaccgttgatgatttggattgctctttggaaagaaaggcccatattccaagattgagatctggttgtccaattactttgactgcattgcacgatgagtga

SEQ ID NO: 16 - DNA, 1479 bp, synthetic construct
  source 1..1479: mol_type = other DNA
atgtggaacttgttgagagaagaattggctttcgttggtcaaaactactacgatatcaccatcgtcttgttcatctctattggtttgacttacttcgcttctagagcttggaaaagagctaagactaacagagaagatatcccaggtagattgggtttgccaattattggtgaaaccttctctttcttgtctgctaccaattctactagaggttgctacgatttcgtcagattgagaagattgtggcatggtagatggtttaagactaggttgtttggtaaggtccatatcttcatcccaaatccagaaggtgctagaactattttcgctaacgatttcggtttgttcaacaagggttacgttaagtctatggctgatgctgttggtaaaaagtctttgttgtgtgttccagtcgaatcccataagagaattagaaggttgttgtccgaacctttctctatgacttctttgtctgctttcattaccaagttcgacaagttgttgtgcggtagattgcaaatcttggaagattctggtaagtccttcaaggttttggacttctcaatgaagatgaccttcgatgctatgtgtggtatgttgatgtctatcaccgaagattctttgttgaggcaaatcgaaaaggattgcaccgatgtttctaacgctatgttgtctttcccagttatgattccaggtactaggtactacaaaggtatcatggctagaaagcgtttgatggaaaccttcagagaaattattgccagaagaagaagaggcgaagaatctactgctgatttcttgcaatcaatgttgcagagagattcattgcctgctgatcaaaagttggatgactccgaaattatggacaacctgttgaccttgattattgctggtcaaactacaactgctgctgcaatgatgtggtctgttaagttcttgcatgataacagagatgcccaagacattttgagggaagaacaattgtctctgactaagatgaagcctgaaggtgcttctttgaatcaagaggatatcaacaacatgaggtacggtttgaaggttgtcaaagaaaccttgagaatgagcaacgtcttgttgtggtttccaagagttgctttgaacgattgcactattgaaggtcacgaaatcaagaaaggttggcacgttaatattgatgctacctgcattcactacgactccgatttgtttattgaccccttgaagtttaacccacagagatttgacgaaatgcagaagccatactctttcattccatttggttctggtccaagaacttgtttgggtatgaatatggctaaggttaccatgttggttttcttgcatagattgacctctggttacacttggactttggatgatttggatacctgtttggaaaaaaaggcccatattccaagactgagatctggttgtccaattaccttgaagtccatctctagatctatgccagaagcttga

SEQ ID NO: 17 - DNA, 1485 bp, synthetic construct
  source 1..1485: mol_type = other DNA
atgctgaacttgttgagagaagaagttttgttggtcgtccagaagtactactacgatttgattatggttgccttgttcaccattggtgttacttacttggcttctaaagcttggaaaagagctactaccaacaacagagaagaaatcccaggtagattgggtttgccttttattggtgaaaccttctctttcttgtctgctaccaattctactagaggttgctacgatttcgtcagattgagaagattgtggaatggtagatggttcaagactaggttgttcggtaagatccatatcttcattccatctccagaaggtgctagaactattttcgctaatgatttcgtcttgttcaacaagggttacgttaagtctatggctgatgctgttggtcaaaagtctttgttgtgtgttccagttgaatcccacaagagaattagaggtttgttgtctgaacctttctccatgacttctttgtctgctttcgttaccaagttcgataagatgttgtgtggtagattgcagaagttggaagaatctggtaagtccttcaaggttttggatttgtgtatgaagatgaccttcgatgctatgtgcgatatgttgatgtctatcaccgaagattctttgttgaggcaaatcgaagaagattgcactgctgtttctgatgccatgttgtctattccaattatgatcccaagaaccaggtactacaaaggtattaccgctagaaaacgtgtcatggaaactttcggtgaaattattgctagaagaagaagaggcgaagaaaccccagaagatttcttgcaatctatgttgcaaagggattctttgccagcttctgaaaagttggatgactccgaaattatggacaacctgttgaccttgattattgctggtcaaactactacagctgctgctatgatgtggtctgttaagttcttgcatgataacagagaaacccaggacattttgagggaagaacaattgtccattaccaagatgaagcctgaaggtgcttctatcaatcatgaggatttgaactctatgaggtacggtttgaaggttgtcaaagaaaccttgagaatgtccaacgtcttgttgtggtttccaagagttgctttggaggattgcactattgaaggttacgatatcaagaaaggttggcacgttaacattgatgctacccatattcatcacgactccgacttgtacaaagatccattgaagtttaacccacagaggttcgacgaaatgcaaaagccatattcctttattccattcggttctggtccaagaacttgcttgggtattaacatggctaaggttaccatgttggttttcttgcatagattgactggtggttacacttggactttggatgatttggatacctgtttggaaaaaaaggcccacattccaagattaagaaacggttgtccaatcaccttgaagtccttgtctaaatcaatgccagaagcctga

SEQ ID NO: 18 - DNA, 1476 bp, synthetic construct
  source 1..1476: mol_type = other DNA
atgttgtgcgccatctctaaagaagagtttttgttcttggtcaagaagcactacgacgttttgatagttgctctgttgttctctattggtgttgcctttttcgtttctaagttggcttggagaaaagctactgcttcttctagaggttatatcccaggtagattgggtttgccttttattggtgaaactgtcccatttttgtccgctgttaagtctactggtggttgctacgaattcatcagattgagaagaattagatacggcaagtggttcaagactagagttttcggtaagatccatgttttcgttccatctactgatggtgccagaatgattttgtctaacgatttcgtcaagttcaacaagggttacatgaaggctttgttggattgcattggtgacaagtctttgttcactgtttctcacgaaaaccacaagagaatcagagatttgttgtccgacttgttctcgatgaactctttgtcctccttcatcgaaaaattcgacaagatgttgtgccagaggttgaaaaaattggaacaaggtggtaagtccttctccgtttttgatttctctatgaagatggccttcgattctatgtgttgcatgttgatgtctattaccgatgaagccttgttgagacaattggaaaaggatattaccgctgtttccaacgctattttctctgccccaattatgattccaggtacgaaatattacaagggtattaaggccagacgtaggatcatggaaattttcaaggatatgattgccaggcgtagatctggtgaagaatggccagaaccagaagatttcttgcaatctttgttgagaagggactcttatcatccagacgaaaagttgcaagatccagaaatcatggataacctgatgaccttgttgatttcaggtcaagctactacatctgctactcaaatgtggtctgttaagttcttggacgaaaatcagcaagtccaagacatcttgagaaaagaggttttgtccattgctaggtctaaaccagaaggtgcttctgttactttggaagatattaacagaatgccctactgctggaaggttttgaaagaaactatgaggatctcctccgttgttatgtggtatccaagagttgctttggctgattgcactattgaaggtttcgaaatcaagaaaggttggcatgttaacgttgatgccaagtgcattcataatgatccagagttgtatcccgatccatctaagtttaatccatccagatttgacgagctgttgaagccatattcctttattccatttgcttccggtcctagaacttgcttgggtattaacatggctaagatgaccatgatggtgttcttgtacagattgacttctgcttattcttggaccgttgatgatttggatacttccttacaagaaaacgctcacttcccaagattgagatctggttgtccaattactttgaaccccttgtga

SEQ ID NO: 19 - DNA, 84 bp, synthetic construct
  source 1..84: mol_type = other DNA
atggaattatcacaagtttgtacaaaaaaggaggctggcgccggaaccaattcagtcgactggatccaagaaggagatataacc

SEQ ID NO: 20 - DNA, 144 bp, synthetic construct
  source 1..144: mol_type = other DNA
atggaattgttgtctgaatatggtttgtttttggcgaaaatcgttaccgttgtgctagcgattgcggcgattgccgccattattgtcaatgttgctcaacgtaataaacgccagcgtggcgagttacgggtcaacaatctcagc

SEQ ID NO: 21 - DNA, 666 bp, synthetic construct
  source 1..666: mol_type = other DNA
atggcagaaccctcagggaagcctgctcctatgggcaaaaagacacattacaaagattcctggcttgataacaccattctgtctatctgtatgcgtcgcctgggtaacgtgactggcgtcagcacaactaaaaagggatatgacggcttcgtcgaattgacgcgtaaagtcatggagacccgttcgccacttttgcagcgtgcgtcgtcaatgcgcgttcttcactccgcgatcccaccgtggttattgaaaattatccgccgttttcttcccaataatcaaaagacagccgagacatttgcagccgccactttatatgcagagtggctggttggaccatgcgaagttaaggaagtcgaggtgaacgggacgatgcagaaatcaggggtgttgattaagaaatgtcgttacctggagagcagcaattgcgtgggaatgtgcgtcaatttatgcaagattccgacacaggattttttcaccaactcgttgggtgttcccttgaccatgactccaaatttcgaagacatgtcttgtgagatgatctacggacaaaccccaccgtccatcgaagaagacccggcacttcaacaaccgtgtttcgccacgctgtgccccactgcccgtaaaaaagcattgacttgttcaaaaactccgttaccaccctga

SEQ ID NO: 22 - DNA, 786 bp, synthetic construct
  source 1..786: mol_type = other DNA
atgttgttgcaattggaagctttgagaggtggtgctactagaattccatttgcttctcatttggaattgaagcaacctagaaggcatagaccagatggtgttaatatggttagatgcagaatggctgaaccatcaggtaaaccagctccaatgggtaaaaaaacccattacaaagatagctggttggacaacaccattttgtctatctgtatgagaagattgggtaacgtcactggtgtttctactactaagaaaggttacgatggtttcgtcgaattgactagaaaagtcatggaaaccagatctcccttgttgcaaagggcttcttctatgagagttctgcattctgctattccaccatggttgttgaagatcatcagaagattcttgcccaacaatcaaaagactgctgaaacttttgctgctgctacattatatgctgaatggttggttggtccatgcgaagtaaaagaagttgaagttaacggcaccatgcaaaagtctggtgttttgattaagaagtgcaggtacttggaatcctctaactgtgttggtatgtgtgttaacttgtgcaagattccaactcaagacttcttcaccaattctttgggtgttccattgactatgactccaaacttcgaagatatgtcctgcgaaatgatctatggtcaaactccaccatccattgaagaagatccagctttacaacaaccttgtttcgctactttgtgtccaactgctagaaaaaaggctttgacttgttctaagactccattgccaccatag

SEQ ID NO: 23 - DNA, 789 bp, synthetic construct
  source 1..789: mol_type = other DNA
atggaagttgctgctgctacttgtactttgttggctgctgttgctttgccagtttcttcatctgctgctgcagctagatctgctacaaaacaatcttactctagaagaaagaccaccgctgttagagctgttatggctagaccacatcaagttccaccaccagctactgcaactgctacagctactacttatagagataactggttcgataagttggccatcggttatttgtctagaaacttgcaagaggcttctggtatgaagaatggtaaagatggttacgaaggtttgattgaagctgctttggctatttctgctttgttcagagttgaccaacaatgggatactgttgcttctgctttacaaagagctttcccatcttacatcttgaccatgatcaaagttatgatgccaccatccagattctccagagaatattttgctgctttcaccactgttttcttcccatggttggttggtccatgtgaagttagagaatctcaagttgatggtagagaagagagaaacgttgtttacattccaaagtgcaggttcttggaatctaccaattgtgttggtatgtgtaccaacttgtgtaagattccatgccaaagattcatccaggattctttgggtactgctgtttacatgtctccaaacttcgaagatatgtcctgcgaaatgattttcggtcaacaaccaccagaagatgatccagctttgaaacaaccatgtttcaggactaagtgcattgccaagcaaaaccatcaagttaactgctccatttag

SEQ ID NO: 24 - DNA, 840 bp, synthetic construct
  source 1..840: mol_type = other DNA
atgttgcatttgccattctgctcctacatcttccatccaatttctttgtccaagtctcagttgaccttgctgttgttctccttgtatcaaattccaatggatgccaagttggttacccaacatagatcacatgtttgggctggtaaaagaggtatgcataagccaagatcttctccaccaattttggctgttttggctagaccagctgataatttgactttggtcaaagaaaccccatccttgttgactgataattggttcgatagaatcgccatcaaccacttgtctcaatctgttcaagctactactggtttgaggaattctaagtctggttacgaatctttggttgaagctgctgctatggtttccagaaatttcgatccaattcaacagtgcgaattggttattgaagctctgaacaaagctttcccatctccaattttgtccttgatcagaactttgatgccacagtctaagttcaccagagaatactttgctgctttcactactttgttcttcgcttggttggttggtccatgcaaagttattgaatctgagatcaacggtaggcgtgaaaagaatgttgttcatatcaagaagtgcaggttcctggaagaatctaattgtgttggtatgtgcttgaacttgtgcaagaatccatctcagaagttcatcaaggattctttgggtatgccagttaacatggttccaaacttcgatgatatgtcctgccaaatgattttcggtcaagatccaccaggtgatgatccagttttgagacaaccttgttacaagttgtgtaaggccaagaagaaccattctgttaactgctccatttag

SEQ ID NO: 25 - DNA, 714 bp, synthetic construct
  source 1..714: mol_type = other DNA
atggccatgtcatctgtttcattgcactccagatctttgatcagatgcgaaattgctgaaccatctggtaaaccagctccaatgggtcaaaaaactagatacaaggattccatcttcgacagagctttcatgtctttgtttgccagaaagatggaaaacgctactggtagagcttctaaaaagactggttacgatggtttcgttgatgtttccagaggtgttttacaaggtagaaacccagttgaacaaagagccttggttagagaagttctgttgtctattatgccaccaggtgctccagaaacttttagaaagttgtttccaccaactaagtgggcttgtgaattcaatgctgctattactgttccattcttccaatggttggttggtccatgtgaaagattcgaagttgaagttaacggtgtcaagcaaaagtccggtgttaagattttgaagtgcaggtacttggaaaactctaactgtgttggtatgtgcgttaacatgtgtaagataccaactcaagacttcttcaccaacgattttggtttgccattgactatgactccaaacttcgaagatatgtcctgcgaaatgatctacggtttacaacctacttccttggaagaagatccagctttgaaacaaccatgcttgcaattgtgtccaacttctgctactactgctgtctgtacaaaattgcagcaagagtactag

SEQ ID NO: 26 - DNA, 879 bp, synthetic construct
  source 1..879: mol_type = other DNA
atggaagttgctgctacttgtatgccattggctcatgctcatggtgttggtgttttgccagcttggtcatctccatctactgctgctgctacagctagatctgttactagacaatcttacacctacaccagaagaaagagattggctactgctagaggtgttatggctagaccacaagaagttgttgttgctccagctccaccagctagaccaactccaccaccaccaactactacaaaagaaagaactgctttggctccacctcctactacaactacttatcatgattcctggttcgataagttggccattggttatttgtccagaaacttgcaagaggcttctggtatgaagaatggtaaagatggttacgaaggtttgattgaagctgccttggctatttctgctttgttcagagttgaccaacaattggaaactgttgctaaggctttggaacaagctttcccatcttacattttgaccatgatcaagatcatgatgccaccatctagattctccagagaatactttgctgctttcaccactattttcttcccatggttggttggtccatgtgaagttagagaatctgaagttgacggcagaaaagaaaagaacgttgtttacattccaaagtgcaggttcttggaatctaccaattgtgttggtatgtgtaccaacttgtgcaagattccatgccaaaagttcatccaagattctttgggtactgccgtttacatgtctccaaatttcgaagatatgtcctgcgaaatgatcttcggtcaacaaccacctgaagatgatccagctttgaaacaaccatgtttcaggactaagtgcattgccaagcaaaaccatcaagttaactgctccatttag

SEQ ID NO: 27 - DNA, 735 bp, synthetic construct
  source 1..735: mol_type = other DNA
atggaagccattatcttcccacaaaacagaggtccaattccatctcaaccattgccaagacaaactaacagattgaacaagtccagaatcttcgctgttttgactaagccaaccgaaaacatttctggtgtcaaagagaagaagtcctctgataatttgccaggcttgacctctaagatctctatctacagagattcctggttcgatcaattggccattaaccatttgtcccaatctgttcaagctgctactggtattggttgcatttctatttctaccaacttgcccattaagaccttgttgccacaatcaagattcaccagagaatactttgctgctttcactaccttgttcttcgtttggttgattggtccatgtcaagttagggaatctgaattcaacggccgtaaagaaaagaacgttgttcatatcaagaagtgcaggttcttggaagaaactaactgtgttggtatgtgcaccaacttgtgtaaagttccaactcaaaccttcatcaagcagtctttgggtatgccagttaatatggttccatctaagtacccaagatccaccttgttgaaacaagatcctccaattcctactgaagatccagcttttagacaaccatgttacaagttgtgtaacgctttgccacatccattttctccatctcatactcatatgcacatgcatgctcaaaacagaaatccacattctccaccaaacttggttgactag

SEQ ID NO: 28 - DNA, 828 bp, synthetic construct
  source 1..828: mol_type = other DNA
atgcaagttgctgctaattccttcggtaaggtttactgtcaaggtatcccaagacataactactcctcatcttacaagttccagagaaagaaccatagcttcggtttgagaagaaagatgattatcgaatgcggtattgctgaaccatctggtcaaccagctccaatgggtcaaaaaactagatacaacgataacctgttcgacaaggttttcatggctttgtttgcccgtaagatgaacaatattgctggtggtaaatccactggtagagaagaaggttacgaaagattcgttgaaacctccagatctgttatgttgggtagaactccaaagcaacaacaagaagctgttagacaagtcctgttgtctatgttgccaccaggtgctccagaaagattcagaaagttgtttccaccaacaaaatgggctgctgaattcaatgctgctgttactgctccatttttccattggttggttggtccatctgaagttgttgaagttgaagttaacggtgtcaagcaaaagtctggtgttcatattaagaagtgcaggtacttggaaaactctggttgtgttggtatgtgtgttaacatgtgcaaattgccaactcaagacttcttcactaacgaatttggtttgccattgactatgaccccaaacttcgaagatatgtcctgtgatatggtttacggtcaaccaccaccacctccagaagaagatccagcttttaaacaaccatgttacgctgctttttgctctatggctcaaccagattctgaagcttgtccaaaattgtccgtcagaaaaagattggacatgagcttctag

SEQ ID NO: 29 - DNA, 804 bp, synthetic construct
  source 1..804: mol_type = other DNA
atggatgctggtttcttgtgtcaaactagatctccagttccatctttgccaagacaaaaaagagcttgtaagctgaaacacagatcaccagttttggctgttttgactagaactccagataccaatatgaccggtgaagaaagaaaagcttctcatccaactgatatgttgttggcaggtttgactaagaaaaccaccgtttacaacgattcttggtttgctaagttggccatcaactacttgtctcaaagattccaagatgctaccggtatgagaaattccaagagagattacgaatccttgactcaaactgctagagatacttggagaaagttcaacccaactcaacaacatgaattggtcttgcagtctttgaacagagctattccagctactatttctaccttggctaaaatgatgttgccacaatgtactttcaccagagaatactttgctgctttcactaccttgttcttcgtttggttggttggtccatgtgaagttagagaatctgatttcaacggccgtaaagaaaagaacgttgttcatatcaagaagtgcaggttcttggaagaaactgattgcattggtatgtgtaccaacttgtgtaaggttccatctcagacctttatcaagcactcttttggtatgccagttaacatggttccaaacttcgaagatatgtcctgcgaaatgatctatggtcaagaaccaccagctattactgaagatccagcttttaaacaaccctgctacaagttgtgcaaagagaatagaaaacactccatgcagtgctcctcttag

SEQ ID NO: 30 - DNA, 759 bp, synthetic construct
  source 1..759: mol_type = other DNA
atggactccaagatgattgcccataacatgtctttgactccaactttggctcaatggaagaagttgagattgaagccaaaacacactttcgttgttggtgttttagctagaccaaccgatgatatttctgaggaaactttgagaaagaccaacgtctacaaggataactggtttgataagttggccatcaaccacttgtctaagtctgttcaagctgctactggtatctctaacaacaagtctggtttcgattctttggttgaagctgcaactgttgcttctcaaaagttcaacactactcaacaacagggcattattttggatgctttggatagagctttccccaagccaattttgtccgttattagaagagttatgccaccatctaagttggctagagaatactttgctgttttcaccaccattttcttcgcttggttgttgggtccatctgaagttagagaatctgaaatcaacggtaggcgtgaaaagaacatcgttcatattaagaagtgcaggttcctggaagaaactaactgtgttggtatgtgcattaacttgtgcaaaatgccatctcagctgttcatcaaggattcttttggtatgccagttaacatggttccaaacttcgatgatatgtcctgcgaaatgattttcggtcaagaaccaccagcttctactgatgatccagctttgaaacaaccatgctacaaattgtgcaaggctaagaaaaatcatgctacccaatgcctgtcttag

SEQ ID NO: 31 - DNA, 1638 bp, synthetic construct
  source 1..1638: mol_type = other DNA
atggaaatgggtactgttttgggtgctatggaagagtacacttttacttttttggctatggccgttggtttcttggttttggtttacttgtatgagccatactggaaggttagacatgttccaggtccagttccattgccattgattggtcacttgcatttgttggctaaacatggtccagatgtttttccagttttggccaagaaacacggtccaatttttagattccatgtcggtagacaaccattgattatagttgctgatgccgaattgtgcaaagaagtcggtattaagaaattcaagtccatgccaaacaggtctttgccatctccaattgctaattccccaattcatagaaagggtttgttcgctactagagactctagatggtctgctatgagaaacgttattgtctctatctaccaaccatctcatttggctggtttgatgccaactatggaatcttgtattgaaagagctgctaccaccaacttaggtgatggtgaagaagttgttttctccaagttggctttgtctttggccactgatattattggtcaagctgcttttggtactgactttggtttgtctggtaaaccagttgttccagatgatgatatgaagggtgttgatgttgttgttggtgatgctgctaaagctaaagcttcttcttccgaattcatcaacatgcatatccattccaccacctcattgaagatggatttgtcaggttctttgtctactatcgttggtgctttggttccattcttgcaaaatccattgagacaggttttgttgagagttccaggttctgctgatagagaaatcaatagagttaacggtgagttgagaagaatggttgatggtatcgttgctgctagagctgcagaaagagaaagagcaccagctgctactgctgctcaacaacataaggattttttgtccgttgttttggctgccagagaatctgatgcttctacaagagaattactgtccccagattatttgtctgctttgacctacgaacatttgattgctggtccagctactgcagcttttacattgtcatctgttgtttacttggttgctaagcacccagaagttgaagaaaagttgttaagagaaatggatgcctttggtccaagaggttctgttccaactgctgatgacttgcaaactaagtttccttacttggatcaggtcgtcaaagaatctatgaggttgtttatggtttccccattggttgcaagagaaacttctgaaagagttgaaattggcggttacgttttgccaaaaggtgcttgggtttggatggctccaggtgttttagcaaaagatgctcataattttcccgatccagagttgtttagaccagaaagatttgatccagctggtgacgaacaaaagaaaagacatccatacgctttcatcccatttggtattggtcctagagtatgcattggtcaaaagttcgctatccaagaaatcaagttggccattatccacttgtaccaacactacgtttttaggcattctccctcaatggaatcaccattggaatttcaattcggtatcgtcgttaatttcaagcacggtgttaagttgcacgttatcaaaagacacgttgagaacaactaa

SEQ ID NO: 32 - DNA, 1629 bp, synthetic construct
  source 1..1629: mol_type = other DNA
atgttggagttctccttgcaaagagttgttgaaattggtactaccttcatggcttctccaccagctttgtctactttggcttttactggtttggctattttgaccggtttggtgtcttacttgtatgctccatattggggtgttagaaaagttccaggtccaccagcatttccattggttggtcatttgccattgatggctaaatatggtccagacgttttctccattttggctaaaagatacggtccaatcttcagattccatatgggtagacaacccttgattattatcgctgatgctgaattgtgcaaagaggttggtattaagaagttcaaggacatcccaaacagatccattccatctccaattgctgcttctccattgcatcaaaagggtttgtttttcaccaaagatgctagatggttgaccatgagaaacaccattttgtccttgtaccaaccatctcatttggcttcattggttccaactatgcaagagtacattgaatctgctaccgaaaacctgcagtccttcaaagaagaagatattgccttctccaacctgtctttgaagttggctactgatgttattggtcaagctgcttttggtgttgatttcggtttgtctagaggtcaatctgaacaaggtcatggttcttcagctaacgaaaaaggtaatggtgatagagacaacgaagtctccgatttcatcaacaaacatatctactccaccacgcagttgaagatggatttgtctggttctttctccatcatcttgggtttgttagttccaatcctacaagaaccattcaggcagattttgaagagaattccaggtactatggacagaaaggttaaccaggctaacaaagaattggctggtagattgaatggcatcgtgtctaaaaggatgaaggaaaaagagcgttcctccaaggatttcttgtccttgattttgaacgccagagaatctgaaagagctgctaagaacgttttcactccagattatgtttctgccgttacctacgaacatttgttggctggttctgctactacctcttttactttgtcatccgttgtttacttggttgctggtcatccagaagttgaaaagaagttgttggaagaaatcgatggtttcggtccaagagatcaattgccaactgctcaagacttgcaaaaaaagttcccatacttggaccaggttatcaaagaagctatgaggttctatttggtgtcacctttggttgctagagaaacctctagagaagtagaaattggtggttacttgttgccaaaaggtacatgggtttggttggctccaggtgttttagcaaaagatccaaagaattttccagagccagaaaagtttaggccagaaagatttgatccaaactgcgaagaacaaaaatggcgtcatccatatgctcatattccatttggtattggtcctagagcttgcatcggtcaaaagttttcattgcaagaaatcaagctgtcgctgatccacttgtacagaaagtatatcttcaggcactccagctctatggaaaaaccattggaattggaattcggcatcgtcttgaatttcaaacacggtgttaagttgagagtcttggagagaaagtga

SEQ ID NO: 33 - DNA, 1560 bp, synthetic construct
  source 1..1560: mol_type = other DNA
atgatgttcttgtcctccgccattcaagaatctccaattgcttctaccattttcaccattttggctggtgttttggtctacttgtatagaccatattggagagttagaaaggttccaggtccaccagcttttccattggttggtcatttgccattgatggctaaatatggtccagatgttttctccgttttggctaaacaatacggtccaatctacagattccatatgggtagacaaccattggttatagttgctgatgctgaattgtgtagagaagtcggtattaagaagttcaaggacatcccaaacagatccattccatcaccaattgctgcttctccattgcatcaaaagggtttgtttttcaccagagactctagatggtctactatgagaaacaccatcttgtctgtttaccagccatcttacttggcaaagttggttccaattatgcagtcctacattgaatctgctaccaagaacttggattccgaaggtgatttgactttctccgatttgtctttgaagttggccactgatgttattggtcaagcttcttttggtgttgacttcggtttgtctaaacctatctctgataagatgtcccaccaccaagatgattctgaagtacaagaattcatcaagcagcacatctactctactacccaattgaagatggatttgtccggttccgtttccattattttgggtttgttagtcccaatcctgcaagaaccttttagacaagttttgaagagaattccaggtacgatggattggaaggttgaaagaactaacaagaacctgtcatccagattggacgaaatcgttgctaagagaatcgaagaaaaggacagatcctctaaggacttcttgtcattgattatgcaagccagggaatctgaaaagttggctaagaatgttttcacctccgattacatttctgccgttacttacgaacatttgttggctggttctgctactacttctttcactttgtcctccatcatctacttggttgcttgtcatcctgaagtcgaacaaaagttgttggcagaaattgatgcttttggtccagacgataatatgccaactgctaatgacttgcaacagaagtttccatacttggaccaagttatcaaagaagctatgcgttgctatatcgtgtctccattagttgctagagaaacctctgctgaagttgaaattggtggttacaaattgccaaaaggtacttgggtttggttggctttgggtgttttagcaaaagatccaaagaatttcccagagccagaaaagtttaagccagaaagatttgatccaaactgcgctgaagaaaagcaaagacatccatacgctaatatcccatttggtattggtcctagagcttgcattggtcaaaagttctctatccaagagatcaagctgtccttgattcacttgtacagaaagtacatcttccagcactcacctttgatggaatctccattggaattggaatacggtatcgtcttgaactacaaacacggtgttaaggttcatgccatcaagagaaagtga

SEQ ID NO: 34 - DNA, 1596 bp, synthetic construct
  source 1..1596: mol_type = other DNA
atgggcatggaattcttgattaccaacggttctttggtgtctaccatttttactgttttggcagttttggctggtgtcttgggttacttgtatgctccatattggggtgttagaagagttccaggtccaagaactattccatttttgggtcatttgcctttgttggctaaacatggtccagatttgttctccgttttggctaagcaatacggtccaattttcagattccatatgggtagacaaccattgattatcgttgctgatgctgaattgtgtagagaagtcggtattaagaagttcaaggacatcccaaacagatccattccatctccaatttctgcttctccattgcatcaaaagggtttgttcttcaccagagatattagatggtctaccatgagaaacaccatcttgtctgtttaccaaccatcttacttggcttcattggttccaactatgcagtcctatattgaatctgctacccaaaacttggacgactcctctaacaaagaagatatcaccttctccaacctgtctttgagattggctactgatgttattggtcaagcttctttcggtgttgatttcggtttgtctaagccacaatccatctctaactccatcaacaaggttgataacggtgaagataagaacgatgatgaagtctccgatttcatcaaccagcatatctactctactacccaattgaagatggacttgtctggttccttgtccattattttgggtttgttggtcccaatcatccaagaacctttcagacaaatcttgaagagaatcccaggtactatggattggaaagtcgacagaactaatcaaaatttggccggtagattggaccaaatcgttatgagaagaatgaaggattccgacagaggtactaagaacttcttgtccttgattatgaacgccagagaatctgaaggtgttgctaagtctgttttcaccccagattacatttctgctgttacctacgaacatttgttagctggttctgctactacctctttcactttgtcatctgccgtttacttgatttctggtcatccagaagttgagtctaagttgttggctgaaattgatggttttggtccacatgatcaaattccaactgctccagatctgcaacataagtttccatacttggaacaggttatcaaagaagccatgaggttctatttggtttcaccattggttgctagagaaacctctagagaagttgaagttggtggttacttattgccaaaaggtacttgggtttggttagctttgggtgttttagcaaaggatccaaagaatttcccagaaccagaaaagttcaagccagaaagatttgacccaaacggtaaagaagaaaagcaaagacatccatacgccttcattccatttggtattggtccaagagcttgcatcggtcaaaagttttctctgcaagagttgaagctgtcattgatccacttgtaccgtaaatacgttttcagacactctccaaatatggaagctccattggaattggaattcggtatcgttttgaacttcaagaagggtgttaagttgaccgtcattaagagaacctga

SEQ ID NO: 35 - DNA, 1602 bp, synthetic construct
  source 1..1602: mol_type = other DNA
atggaattcctgtccaccaacatccaacattctatcgttgataccgttgaagttttgactagaccaccaatgacttctaccatctgtactattttggctttgttggctaccgttttggtttacttttatggtccatattggagggttagaaaagttccaggtccaccaggttttccattggttggtcatttgccattgatggctagatatggtccagatgttttttccgttttggctaaacaatacggtccaatcttcagattccatatgggtagacaaccattggttatagttgctgatgctgaattgtgtagagaagtcggtattagaaagttcaaggacatcccaaacagatccattccatctccaattgctgcttctccattgcatcaaaagggtttgtttttcaccagagactctagatggtctactatgagaaacaccatcttgtctgtttaccagccatcttacttggctaagttggttccaattatgcagtccttcattgaatctgctaccaagaacttggattccgaaggtgatttgactttctccgatttgtctttgaagttggccactgatgttattggtcaagctgcttttggtgttgatttcggtttgtctaaacctatcaccgataagatgaaccaccaagaaaaggattctgaagtccaagaattcatcaaccagcataactactctactacccagttgaagatggatttgtctggttccgtttccattatcttgggtttgttagttccaatcctgcaagaaccattcagacagattttgaagagaatcccaggtactatcgattggaaggttgaaagaacgaacaagaacctgtctagaagattagacgaaatcgttgctaagaggatggaagaaaagtacggttcttctaaggacttcctgtctttgatattgcaagccagagaatctgaaaagttggctaagaacgttttcacctccgattacatttctgctgttacctacgaacatttgttagctggttctgctactacctctttcactttgtcatctatcatctacttggttgctggtcatccaaaggttgaacaaaagttgattgctgaaattgatgcctttggtccagacgatcatatgccaactgctaatgacttgcaacagaagttctcatacttggaccaagttatcaaagaagctatgcgttgttacactgtttcacctttggttgctagagaaacttctgccgaagttgaaattggtggttacaaattgccaaaaggtacatgggtttggttggctttgggtgttttggcaaaagatccaaagaattttccagagcctgagaagtttagaccagaaagatttgatccaaactgcaaagaagaaaagcagagacatccatacgctaacattccatttggtattggtcctagagcttgcattggtcaaaagttctctatccaagagatcaagttgtccttgatccacttgtacagaaagtacatcttcagacactcacccttgatggaaaaaccattggaattggaatacggcatcgtcttgaattacaaacacggtgttaaggtttgcgccatcaagagaaagtaa

SEQ ID NO: 36 - DNA, 1632 bp, synthetic construct
  source 1..1632: mol_type = other DNA
atgttggcttctgctgttttgagagctatggaagaatgtacttttacctctgctgctatggctgttggttttttgttggttgtttacttgtacgagccatactggaaggttagacatgttccaggtccagttccattgccatttgttggtcacttgcatttgttagctagacatggtcctgatgttttcttggttttggctaaaaagtacggtccaatcttcagattccatatgggtagacaaccattggttatcgttgctaatgctgaattgtgcaaagaagtcggcatcaaaaagttcaagtctatgccaaataggtccttgccatctgctattgctaattccccaattcatttgaagggtttgttctccactagagactctagatggtctgctttgagaaacatcatcgtgtctatctaccaaccatctcatttggctggtttgattccatctatgcaatcccatattgaaagagctgctaccaatttggatgatggtggtgaagctgaagttgctttttctaaattggctttgtctttggccaccgatgttattggtcaagctgcttttggtgctgattttggtttgactacaaaaccagctgctccaccaccacatcatggtccaccaagacaacatggtgaagaggatggtgatggttctcattctactagatcttccgaattcatcaagatgcatatccattctaccacctcattgaagatggatttgtctggttctttgtctaccatcgttggtactttgttgccagttttacaatggcctttgagacagttgttgttgagagttccaggtgctgctgatagagaaattcaacgtgttaatggtgctttgtgcagaatgatggatggtattgtcgcagatagagttgctgcaagagaaagagcaccacaagctcaaagacagaaggattttttgtcagttgttttggctgccagagattctgatgctgctgctagaaagttgttgactccagattatttgtccgctttgacctacgaacacttgttagctggttctgctactactgcttttactttgtcatctgtcttgtacttggttgcccaacatccaagagttgaagaaaagttgttaagagaagttgatgctttcggtccacctgatagagttccaactgctgaagatctacaatccagatttccatacaccgaccaagtcttgaaagaatctatgaggttcttcatggtttccccattggttgctagagaaacttctgaacaagttgatattgccggttacgttttgccaaaatctacttgggtttggatggctccaggtgttttagcaaaagatccagttaattttccagagccagagttgtttagaccagaaagatttgatccagctggtgatgaacaaaaaagaaggcatccatacgctttcattccatttggtattggtccaagaatctgcatcggtcaaagattctctatccaagaaatcaagttggccttgatccacttgtacagacaatacgtttttaggcactctccctctatggaatcaccattggaatttcaattcggtgtcgtcttgaacttcaaacacggtgttaagttgcagtccatcaagagacataagtgctga

SEQ ID NO: 37 - DNA, 1476 bp, synthetic construct
  source 1..1476: mol_type = other DNA
atgctgaagctgtctaaagaagagttgttgttcttggcccaaaacaagtacgatattgtcatggttgccttgttctccattgcagtttttgctgttttgaaggcttggagaaagaagattaccacctccaacaaagaagatattccaggcggtttgggtttgccatttgttggtgaaactttgtctttcttgtctgctaccaattctaccagaggttgttacgatttcgtcaggttgagaagaaaatggtacggtaaatggttcaagaccagaatcttcggtaagattcatgttttcgctccatctactgaagctgctagaaaggttttcactaatgacttcggtgaattcaacaagggctacattaagtctatggctactgttgtcggtgagaagtctgtttttgccgttccattggaatcccataagagaatcagacatattctgtccgctttgttctctatcccatccttgtctattttcgtccaaaacttcgaccaaatgttgtcccaaagactgaagaacttgcaagaaagaggtattaccttcgccgttttggatttcactatgaagttgaccttggactctatgtgcaacatgctgatgtctatcactgaagagtctttgttgaagcagatcttgagagattgtgctgctgtttctgatgctttgttgtctgttccattgatgattccaggtactacttactacaaaggtatgaaggcaagagaaaggctgatggaaatcttcaaagaaaagattgccagaagaagatctggcgaagagtacaaagatgacttcttgcaaagtttgttggaaagggattcttacccatcctctgaaagattgcaagactccgaaattatggacaacctgttgactttgttggtttccggtcaagtttcttctgctgctactatgatgtggtctgttaagtttctagacgaaaacaaagaggtcttggacaagttgagagaagaacaatctaacattgccaagaatatgcaaggtgcctctttgtctatggtcgatttgaacaaaatgtcctactgctacaaggtcgtcaaagaatcattgagaatgtccaacgctgttttgtggttgccaagagttgctcaaaaagactgtactgttgatggtttcgagatcaagaaaggttggaatgttaacgttgatgctacccatattcattacgatccagccttatacaaagacccattgagattcaatccatccagattcgacgaaatgcaaaagccatactctttcattccatttggtgctggtccaagaacttgtttgggtattgaaatggctaagctgtccatgttggttttcattcatagattgacctctgaatacgaatggcgtattgaagatccagatccatctttggaaagaactactcatgttccaagattgagaaccggtttgccaattactttgaagccattgaaatccgcttctcaggattga

SEQ ID NO: 38 - DNA, 1503 bp, synthetic construct
  source 1..1503: mol_type = other DNA
atgatcctgaagaacttcaagctgaagatgctgaagttgttcagagaacacgttttgttcttggtccaaaacaagtacgatattgtcttggctgccttcttgtccattgctatttttgcttttttcaaggccagacgtaaggtcaacactattaacgaagaagatgagatcccaggttctttgggtttgccatttgttggtgaaactttctctttcttgtctgctaccaattctaccagaggttgttacgatttcgtcaggttgagaagaaaatggtacggtaaatggttcaagtccagaatgttcggtaagatccatgttttcgttccatctactgaagctgctagaaaggttttcactaatgacttcggtgagttcaacaagtcctacattaagtctatggctactgttgtcggtgagaagtctgtttttgctgttccattggaaacccacaagagaatcagacatattttgtccgctttgttctccatgccatctttgtctaagttcgttgaaaagttcgaccagatgatctcccaaagattgaacaagttggaacaaaccggtaagtctttcgctgttttgccttttactatgaagttgaccttggattctgtctgcaacatgctgatgtctatcactgaagaatccttgttggaccaaatcttgtctgattgtgctgctgtttctgatgctttgttgtcagttccattgatgattccaggtacgatctactacaaaggtatgaaggctagacagaggctgatgaagattttcaaagaaatgatcgacagaaggcgttccggtaaagaacagaaggatgatttcttgcagtacttgttggaaagacatacctgtccatcctccgaaaaattggaagattccgaaatcatggacaacctgttgactttgttggtttctggtcaagtttcttctgctgctgctatgatgtggtctgttaagtttttggacgaaaactccgaagtcttggacaagttgagagaagaacaattggaaatcgctaagaacaagcaaggtggtacttctttgtcaatggaagatatcaacaagatgtcctacggtttgaaggtcgtcaaagaaactttgagaatgtccaacgttgttttgtggttgccaagagttgctcaaaatgattgcactttggatggtgtcgaaatgaagaaaggttgggttgttaatgttgatgctacctgcatccattttgatccagacttgtatgaagatcccatgagattcaatccatccagatttgacgaaatgcagaagccatattctttcttgccttttggtgctggtccaagaacttgtttgggtattgaaatggccaagttgtccatcttggttttcttgcatagattgactggtggttacgaatggcgtattgaaaacagagatccatctatggaaagaactacccatgttccaagattgagatctggtttgccaattactttgaaggctttggctaagaacggcaagtaa

(19) United States
(12) Patent Application Publication
(10) Pub. No.: US 2025/0354184 A1
(43) Pub. Date: Nov. 20, 2025

(54) STRIGOLACTONE-PRODUCING MICROBES AND METHODS OF MAKING AND USING THE SAME

(71) Applicant: The Regents of the University of California, Oakland, CA (US)

(72) Inventors: Yanran Li, Riverside, CA (US); Sheng Wu, Riverside, CA (US); Anqi Zhou, Riverside, CA (US)

(21) Appl. No.: 18/874,507

(22) PCT Filed: Jun. 16, 2023
(86) PCT No.: PCT/US2023/025568

§ 371 Date: Dec. 12, 2024

Related U.S. Application Data
(60) Provisional application No. 63/352,980, filed on Jun. 16, 2022.

(51) Int. Cl. C12P 17/04; C12N 1/16; C12N 1/20; C12N 9/02; C12P 39/00

(52) U.S. Cl. CPC C12P 17/04; C12N 1/16; C12N 1/20; C12N 9/0042; C12N 9/0069; C12P 39/00; C12Y 106/02004; C12Y 113/11068

(57) ABSTRACT

The present disclosure provides a bacterial and yeast co-culture system and methods of making and using such co-culture systems for producing strigolactones.

### CROSS-REFERENCE TO RELATED APPLICATIONS

This application is a U.S. National Phase Application Under Section 371 of PCT/US2023/025568, filed Jun. 16, 2023, which claims priority benefit of U.S. Provisional Application No. 63/352,980, filed Jun. 16, 2022, which is are incorporated by reference herein for all purposes in their entirety.

### SEQUENCE LISTING

The instant application contains a Sequence Listing which has been submitted electronically in XML format and is hereby incorporated by reference in its entirety. Said XML copy, created on Jul. 20, 2023, is named 081906-1390920-244410PC_SL.xml and is 80,727 bytes in size.

### BACKGROUND

Strigolactones (SLs) are a class of hormones that have diverse roles in plant growth and development. More than 30 natural SLs have been discovered, but the complete biosynthetic pathway for many of them remains unknown. SLs generally consist of a conserved butenolide ring (D ring) connected to a less conserved tricyclic lactone ring via an enol-ether bond (7) (FIG. 1). SLs can be classified into canonical and non-canonical SLs: the canonical SLs contained the tricyclic lactone-ring (ABC ring), while the non-canonical SLs lack of the tricyclic ring scaffold with one (C ring) or two rings (B ring and C ring) missing (8). The canonical SLs can be further subdivided into orobanchol (O)- and the strigol (S)-type SLs according to the stereochemistry in the C ring, which are represented by 4-Deoxyorobanchol (4DO) and 5-deoxystrigol (5DS), respectively (7). Some of the better-known non-canonical SLs include methyl carlactonoate (MeCLA) (9), heliolactone (10), avenaol (11), zealactone (10), and lotuslactone (12).

SLs exhibit extremely low abundance in nature (up to 70 pg of orobanchol/plant can be detected in the roots of red clover seedlings) (14, 15). Chemical synthesis has been useful in SL-related research through providing synthesized SL standards and analogues (4). However, the synthetic analogs are generally less active than natural SLs (16), partially because synthetic analogs are normally racemic mixtures, and different isomers play different roles in SL-signal transduction (17). In addition, due to the structural complexity and instability of SLs, the chemical synthesis of SLs is laborious and expensive to be an economic SL supply strategy for the SL-based agricultural applications (18).

Potential commercial applications for SLs include controlling crop traits, reducing parasitic weed infestations, or as anti-cancer therapeutics. Nevertheless, the extremely low abundance of SLs in nature, the highly challenging chemical synthesis, and the incomplete investigations on the structure-function correlation of SLs, are largely impeding the development of commercial agricultural applications of SLs. Synthesizing SLs from microbial hosts provides an alternative sourcing mechanism. However, de novo synthesis of canonical SLs in a microbial host has not yet been reported.

### BRIEF SUMMARY

The disclosure is based, in part, on the inventors' determination that the biosynthetic pathway for SL production can be dissected into two parts using a bacterial and yeast, e.g., E. coli-S. cerevisiae, co-culture strategy to generate a microbial SL platform for synthesizing both non-canonical and canonical SLs. Accordingly, in one aspect, the disclosure provides engineered E. coli-S. cerevisiae co-culture systems for the de novo biosynthesis of both noncanonical and canonical SLs, including but not limited to carlactone (CL), carlactonic acid (CLA), 5-deoxystrigol (5DS), 4-deoxyorobanchol (4DO) and orobanchol.

The inventors split the biosynthetic pathway of SLs at the position of carlactone (CL), a key intermediate in the pathway, and divided this pathway into two modules (FIG. 1): CL production module (1) and CL metabolic module (2). In one module, de novo synthesis of CL was achieved in the β-carotene-accumulating strains of E. coli by introducing an isomerase DWARF27 (D27) and two carotenoid cleavage dioxygenases (CCD7 and CCD8, respectively) (Module1). The biosynthetic pathway from CL to SLs (including 5DS, 4DO and orobanchol) was achieved by expressing various cytochrome P450s and the corresponding reductase in yeast strain (Module2).

In some embodiments, to synthesize CLA, the engineered yeast strain expresses a cytochrome P450 reductase gene (such as but not limited to the cytochrome P450 reductase from A. thaliana, AtATR1), a carlactonoic acid (CLA) synthetase gene (such as but not limited to MAX1 from A. thaliana, AtMAX1).

In some embodiments, to synthesize 5DS, the engineered yeast strain expresses a cytochrome P450 reductase gene (such as but not limited to the cytochrome P450 reductase from A. thaliana, AtATR1), a carlactonoic acid (CLA) synthetase gene (such as but not limited to MAX1 from A. thaliana, AtMAX1), and a 5DS synthase gene (such as CYP722C from Gossypium arboreum, GaCYP722c).

In some embodiments, to synthesize orobanchol, the engineered yeast strain expresses a cytochrome P450 reductase gene (such as but not limited to the cytochrome P450 reductase from A. thaliana, AtATR1), a carlactonoic acid (CLA) synthetase gene (such as but not limited to MAX1 from A. thaliana, AtMAX1), and an orobanchol synthase gene (such as CYP722C from Capsicum annuum, CaCYP722c).

In some embodiments, to synthesize 4DO, the engineered yeast strain can also express a cytochrome P450 reductase gene (such as but not limited to the cytochrome P450 reductase from A. thaliana, AtATR1), a 4DO synthase gene (such as but not limited to CYP711A2 from Oryza sativa, OsCYP711A2). Further addition of an orobanchol synthase gene (such as but not limited CYP711A3 from Oryza sativa, OsCYP711A3) led to the conversion from 4DO to orobanchol.

Engineered strains, e.g., the E. coli and S. cervisiase explained in the preceding paragraphs are then combined in a co-culture system for the production of various SLs. Accordingly, provide engineered bacterial and yeast strains, e.g., E. coli and S. cerevisiae strains expressing biosynthetic enzymes for producing SLs and precursors, such as CL, CLA and canonical SLs 4-DO, 5-DS, orobanchol.

In one embodiment, the method further comprises regulating various aspects of cell culture, including, for example, the components of the cell culture media, a two-stage fermentation process, temperature, strain ratio, and co-culture method. In some embodiments, a co-culture system described in the present disclosure increases the stability of intermediate CL and promote the production of SLs. In some embodiments, the titer of 4-DO, 5-DS and orobanchol is about 2, 12, and 15 μg/L, respectively.

In a further aspect, the disclosure provides a platform to identify new enzymes and maybe useful for producing SLs derivatives (non-naturally occurring SLs).

### BRIEF DESCRIPTION OF THE DRAWINGS

FIG. 1 Putative biosynthetic pathway of SLs. The part of the pathway localized natively in plastids or established in E. coli in this work is highlighted in green; and the part localized natively in cytosol or established in yeast in this work is highlighted in yellow. D27, DWARF27 from Oryza sativa; CCD7, carotenoid cleavage dioxygenase 7 from Arabidopsis thaliana; CCD8, carotenoid cleavage dioxygenase 8 from Arabidopsis thaliana; AtMAX1, MORE AXILLARY GROWTH 1 from Arabidopsis thaliana; CYP711A2, cytochrome P450 CYP711A2 from Oryza sativa; CYP711A3, cytochrome P450 CYP711A3 from Oryza sativa; VuCYP722C is from Vigna unguiculata; CaCYP722C is from Capsicum annuum; GaCYP722C is from Gossypium arboretum; CL is the division point. NADPH-cytochrome P450 reductase 1 from Arabidopsis thaliana (ATR1) is expressed in yeast for the functional reconstitution of the plant cytochrome P450s.

FIG. 2A-E Production of CL in E. coli. (A) HPLC analysis (λ=440 nm) of i) β-carotene standard, ii) 9-cis-carotene standard; cell extracts of E. coli harboring iii) β-carotene producing plasmid (pAC-BETAipi, Table 1) and empty plasmid (EV), iv) pAC-BETAipi and OsD27-expressing plasmid, iv) pAC-BETAipi and OsD27/CCD7-co-expressing plasmid. The samples were analyzed using Separation Method I (see Materials and Methods). (B) Using Separation Method II (see Materials and Methods), HPLC analysis (λ=390 nm) of E. coli harboring i) pAC-BETAipi and OsD27-expressing plasmid, ii) pAC-BETAipi and OsD27/CCD7-co-expressing plasmid. (C) Selected ion monitoring SIM (SIM) extracted ion chromatogram (EIC) at 9-cis-β-apo-10′-carotenol's characteristic m/z⁺=379.3 of E. coli harboring i) pAC-BETAipi and OsD27-expressing plasmid, ii) pAC-BETAipi and OsD27/CCD7-co-expressing plasmid. (D) Using Separation Method III (see Materials and Methods), LCMS analysis (λ=269 nm) of i) β-ionone standard, 9-cis-β-apo-10′-carotenol-producing E. coli harboring ii) empty plasmid (EV, cell pellet extracts), iii) CCD8-expressing plasmid (cell pellet extracts), iv) empty plasmid (EV, medium extracts), iv) CCD8-expressing plasmid (medium extracts). (E) Selected ion monitoring (SIM) extracted ion chromatogram (EIC) at CL's characteristic m/z⁺ signal (MW=302.19 Da, [C₁₉H₂₆O₃+H]⁺=[C₁₉H₂₇O₃]⁺=303.2) of 9-cis-β-apo-10′-carotenol-producing E. coli harboring i) empty plasmid (EV, cell pellet extracts), ii) CCD8-expressing plasmid (cell pellet extracts), iii) empty plasmid (EV, medium extracts), iv) CCD8-expressing plasmid (medium extracts). All traces are representative of at least three biological replicates for each engineered E. coli strain.

FIG. 3A-D Establishment of SL production using E. coli-S. cerevisiae co-cultures. (A) HPLC analysis (λ=269 nm) of CLA formed in CL-accumulating E. coli cocultured with ATR1-expressing S. cerevisiae harboring i) empty vector (EV, cell pellet extracts), ii) AtMAX1-expressing plasmid (cell pellet extracts), iii) empty vector (EV, medium extracts), iv) AtMAX1-expressing plasmid (medium extracts). (B) SIM EIC using CLA's characteristic m/z⁻ signal (MW=332.16 Da, [C₁₉H₂₄O₅—H]⁻=[C₁₉H₂₃O₄]⁻=331.1) of CL-accumulating E. coli cocultured with ATR1-expressing S. cerevisiae harboring i) empty vector (EV, cell pellet extracts), ii) AtMAX1-expressing plasmid (cell pellet extracts), iii) empty vector (EV, medium extracts), iv) AtMAX1-expressing plasmid (medium extracts). (C) SIM EIC using orobanchol's characteristic m/z⁺ signal (MW=346.14 Da, [C₁₉H₂₂O₆+H]⁺=[C₁₉H₂₃O₆]⁺=347.1) of i) orobanchol standard; CL-accumulating E. coli cocultured with ATR1-expressing S. cerevisiae harboring ii) AtMAX1-expressing plasmid (cell pellet extracts), iii) AtMAX1/CaCYP722C-expressing plasmid (cell pellet extracts), iv) CYP711A2-expressing plasmid (cell pellet extracts) v) CYP711A2/CYP711A3-expressing plasmid (cell pellet extracts), vi) AtMAX1-expressing plasmid (medium extracts), vii) AtMAX1/CaCYP722C-expressing plasmid (medium extracts), viii) CYP711A2-expressing plasmid (medium extracts), ix) CYP711A2/CYP711A3-expressing plasmid (medium extracts). (D) SIM EIC using 4DO or 5DS's characteristic m/z⁺ signal (MW=330.15 Da, [C₁₉H₂₂O₅+H]⁺=[C₁₉H₂₃O₅]⁺=331.1) of i) 4DO standard, ii) 5DS standard; CL-accumulating E. coli cocultured with ATR1-expressing S. cerevisiae harboring iii) MAX1-expressing plasmid (cell pellet extracts), iv) AtMAX1/GaCYP722C-expressing plasmids (cell pellet extracts), v) empty vector (EV, cell pellet extracts), vi) CYP711A2-expressing plasmid (cell pellet extracts), vii) AtMAX1-expressing plasmid (medium extracts), viii) AtMAX1/GaCYP722C-expressing plasmids (medium extracts), ix) empty vector (EV, medium extracts), x) CYP711A2-expressing plasmid (medium extracts). All traces are representative of at least three biological replicates for each engineered E. coli-S. cerevisiae consortium.

FIG. 4 Phylogenetic analysis of CYP722C and the functional mapping. Phylogenetic analyses were conducted in MEGA X. The asterisk means that these enzymes have been tested in this study, red indicates 5DS producing activity and blue indicates orobanchol-producing activity.

FIG. 5 Structures of natural SLs and synthetic SL analogues. Orobanchol-type SLs (I), strigol-type SLs (II), non-canonical SLs (III), synthetic SL analog rac-GR24 (IV).

FIG. 6A-D Failed construction of SL biosynthetic pathway in yeast. (A) HPLC analysis (λ=470 nm) of i) all-trans-β-carotene standard, ii) 9-cis-carotene standard; cell extracts of β-carotene producing yeast strains harboring plasmid with iii) DRE2-G₄S-OsD27, iv) DRE2-OsD27, v) mOsD27, vi) tOsD27, vii) OsD27, viii) tAtD27, ix) AtD27 and x) empty vector, respectively. The samples were analyzed using Separation Method 1 (see Materials and Methods). (B) HPLC analysis (λ=470 nm) of i) all-trans-β-carotene standard, ii) 9-cis-carotene standard; cell extracts of 0-carotene producing yeast harboring plasmids expressing iii) ISU1, ISU2, YFH1, tOsD27, iv) JAC1, YFH1, NFS1, tOsD27, v) CTT1, CTA1, tOsD27. The samples were analyzed using Separation Method 1 (see Materials and Methods). (C) HPLC analysis (λ=470 nm) of i) all-trans-β-carotene standard, ii) 9-cis-carotene standard; cell extracts of β-carotene producing yeast harboring plasmids expressing tOsD27 with the supplementation of iii) 500 μM FeSO₄, iv) 50 μM FeSO₄, v) no FeSO₄, and vi) empty vector. The samples were analyzed using Separation Method 1 (see Materials and Methods) (D) Using Separation Method 3 (see Materials and Methods), LCMS analysis (λ=269 nm) of i) β-ionone standard, cell extracts of yeast strains harboring ii) mtCCD7 (m: mitochondria targeted; t: truncated), iii) mCCD7, iv) tCCD7, v) CCD7, and vi) empty plasmid. Peak1, 2 and 3 refer to all-trans-β-carotene, 9-cis-carotene and β-ionone. respectively.

FIG. 7A-I Mass spectra and UV-Vis absorption spectra of compound 1-9. (A) 1 was eluted at 8.17 min, corresponding to all-trans-ß-carotene ([M]⁺=536.3); (B) 2 was eluted at 7.78 min, corresponding to 9-cis-β-carotene ([M]⁺=536.3); (C) 3′ was eluted at 12.64 min, corresponding to 9-cis-β-apo-10′-carotenol ([M+H]⁺=379.3); (D) 4 was eluted at 16.04 min, corresponding to 0-ionone ([M+H]⁺=193.2); (E) 5 was eluted at 20.69 min, corresponding to carlactone ([M+H]⁺=303.2); (F) 6 was eluted at 16.32 min, corresponding to carlactonic acid ([M−H]⁻=331.0); (G) 7 was eluted at 15.81 min, corresponding to 4-deoxyorobanchol ([M+H]⁺=331.1); (H) 8 was eluted at 15.97 min, corresponding to 5-deoxystrigol ([M+H]⁺=331.1); (I) 9 was eluted at 11.03 min, corresponding to orobanchol ([M+H]⁺=347.1).

FIG. 8 HPLC analysis of the activities of different D27s in E. coli. HPLC analysis (λ=440 nm) of i) β-carotene standard, ii) 9-cis-carotene standard; cell extracts of E. coli harboring iii) β-carotene producing plasmid (pAC-BETAipi, Table 1) and empty plasmid (EV) (resulting in strain CL-1), iv) pAC-BETAipi and truncated OsD27-expressing plasmid (CL-8), v) pAC-BETAipi and OsD27-expressing plasmid (CL-2), vi) pAC-BETAipi and AtD27-expressing plasmid (CL-9). The samples were analyzed using Separation Method I (see Materials and Methods).

FIG. 9A-B Detection of β-ionone upon the introduction of CCD7 gene in all-trans-β-carotene accumulating E. coli strain. (A) Extracted ion chromatogram (EIC) at m/z⁺=193.2 showed the formation of β-ionone (MW=192.15 Da, [C₁₃H₂₀O+H]⁺=[C₁₃H₂₁O]⁺=193.2) upon the introduction of CCD7 gene in all-trans-β-carotene accumulating E. coli strain. (B) duplicated panel The analysis is performed by LC-MS using separation method II.

FIG. 10A-G pH & medium for the detection of CL. (A) Detection of 9-cis-β-apo-10′-carotenol at wavelength 390 nm, the amount of 9-cis-β-apo-10′-carotenol was reduced upon the introduction of CCD8 in vivo. (B) EIC spectra at m/z⁺379.3 for 9-cis-β-apo-10′-carotenol in positive ion mode, the amount of 9-cis-β-apo-10′-carotenol was reduced upon the introduction CCD8 in vivo. (C) To improve the stability of carlactone, 1:2 (volume of buffer:volume of medium) 10 mM sodium citrate buffer (pH 6.0) was added to the growth medium. (D) 1:2 (volume of buffer:volume of medium) 200 mM HEPES buffer (pH 7.0) was added to the growth medium, the yield of carlactone was slightly increased by adjusting the pH of the growth medium. (E) Increased carlactone production in XY medium with CCD8 expressed from pET28b. (F) Further slightly enhanced carlactone production in XY medium with CCD8 expressed from pET21a. (G) EIC at carlactone's characteristic m/z⁺ signal (MW=302.19 Da, [C₁₉H₂₆O₃+H]⁺=[C₁₉H₂₇O₃]⁺=303.2) of 9-cis-β-apo-10′-carotenol-producing E. coli harboring i) empty plasmid (EV, cell pellet extracts), ii) CCD8-expressing plasmid (cell pellet extracts), iii) empty plasmid (EV, medium extracts), iv) CCD8-expressing plasmid (medium extracts). Carlactone can be detected both in pellet and medium by introducing CCD8 in vivo. The analysis is performed by LC-MS using Separation Method II, except that the organic phase was changed from methanol to acetonitrile.

FIG. 11A-B Failed synthesis of CLA in E. coli (A) CLA was not consumed as a substrate by introducing AtMAX1 into the CL-producing E. coli. (B) EIC using CLA's characteristic m/z⁻ signal (MW=332.16 Da, [C₁₉H₂₄O₅—H]⁻=[C₁₉H₂₃O₄]⁻=331.1) of the CL-producing E. coli expressing truncated AtMAX1 and ATR1. The analysis is performed by LC-MS using Separation Method II, except that the organic phase was changed from methanol to acetonitrile.

FIG. 12A-B To enhance CLA production through adjusting E. coli/yeast ratio. (A) The ratio between CLA to CL has risen from 0.4:1 to 1.6:1, calculated based on the peak areas at 269 nm, with E. coli-yeast volumetric ratio changed from 3:1 to 1:1. (B) EIC using CLA's characteristic m/z⁻ signal (MW=332.16 Da, [C₁₉H₂₄O₅—H]⁻=[C₁₉H₂₃O₄]⁻=331.1) of CLA-producing consortium with different E. coli-yeast ratio. The analysis is performed by LC-MS using Separation Method III.

FIG. 13A-C Production of orobanchol and detection of related biosynthetic intermediates based on introduction of VuCYP722C gene. (A) EIC using CLA's characteristic m/z⁻ signal (MW=332.16 Da, [C₁₉H₂₄O₅—H]⁻=[C₁₉H₂₃O₄]⁻=331.1) of CLA-producing consortium expressing VuCYP722C, and CLA was consumed as the substrate by introducing VuCYP722C in vivo. (B) EIC using 18-hydroxy-CLA's characteristic m/z⁻ signal (MW=348.16 Da, [C₁₉H₂₄O₆—H]⁻=[C₁₉H₂₃O₅]⁻=347.1) of CLA-producing consortium expressing VuCYP722C, and putative 18-hydroxy-CLA was mainly detected in the medium of strain expressing VuCYP722C. (C) EIC using orobanchol's characteristic m/z⁻ signal (MW=346.14 Da, [C₁₉H₂₂O₆+H]⁺=[C₁₉H₂₃O₆]⁺=347.1) of CLA-producing consortium expressing VuCYP722C, and orobanchol was mainly detected in the medium by introducing VuCYP722C in vivo. The analysis is performed by LC-MS using Separation Method III.

FIG. 14A-C Production of 5DS and detection of related biosynthetic intermediates based on introduction of GaCYP722C gene. (A) EIC using CLA's characteristic m/z⁻ signal (MW=332.16 Da, [C₁₉H₂₄O₅—H]⁻=[C₁₉H₂₃O₄]⁻=331.1) of CLA-producing consortium expressing GaCYP722C, and CLA was consumed as the substrate by introducing GaCYP722C in vivo. (B) EIC using 18-hydroxy-CLA's characteristic m/z⁻ signal (MW=348.16 Da, [C₁₉H₂₄O₆—H]⁻=[C₁₉H₂₃O₅]⁻=347.1) of CLA-producing consortium expressing GaCYP722C, and putative 18-hydroxy-CLA was mainly detected in the medium of strain expressing GaCYP722C. (C) EIC using 5DS's characteristic m/z⁻ signal (MW=330.15 Da, [C₁₉H₂₂O₅+H]⁺=[C₁₉H₂₃O₅]⁺=331.1) of CLA-producing consortium expressing GaCYP722C, and 5DS can be detected both in pellet and medium by introducing GaCYP722C in vivo. The analysis is performed by LC-MS using Separation Method III.

FIG. 15A-F Production of 4DO and detection of related biosynthetic intermediates upon introduction of OsCYP711A2 gene. (A) EIC using CLA's characteristic m/z⁻ signal (MW=332.16 Da, [C₁₉H₂₄O₅—H]⁻=[C₁₉H₂₃O₄]⁻=331.1) of CL-producing E. coli cocultured with yeast expressing CYP711A2. CLA was produced by introducing OsCYP711A2 in vivo, this suggest that OsCYP711A2 has the same ability as AtMAX1 to convert CL to CLA (B) EIC using 18-hydroxy-CLA's characteristic m/z⁻ signal (MW=348.16 Da, [C₁₉H₂₄O₆—H]⁻=[C₁₉H₂₃O₅]⁻=347.1) of CL-producing E. coli cocultured with yeast expressing CYP711A2. Putative 18-hydroxy-CLA was only detected in the medium of strain expressing OsCYP711A2. (C) EIC using 4DO's characteristic m/z⁻ signal (MW=330.15 Da, [C₁₉H₂₂O₅+H]⁺=[C₁₉H₂₃O₅]⁺=331.1) of CL-producing E. coli cocultured with yeast expressing CYP711A2. 4DO can be detected both in pellet and medium by introducing OsCYP711A2 in vivo. (D) EIC using 18-hydroxy-CLA's characteristic m/z⁻ signal (MW=348.16 Da, [C₁₉H₂₄O₆—H]⁻=[C₁₉H₂₃O₅]⁻=347.1) of CLA-producing consortium expressing OsCYP711A2, most of CLA was consumed as a substrate compared with the control without OsCYP722A2. (E) EIC using 18-hydroxy-CLA's characteristic m/z⁻ signal (MW=348.16 Da, [C₁₉H₂₄O₆—H]⁻=[C₁₉H₂₃O₅]⁻=347.1) of CLA-producing consortium expressing OsCYP711A2, and putative 18-hydroxy-CLA was only detected in the medium of strain expressing AtMAX1/OsCYP711A2. (F) EIC using 4DO's characteristic m/z⁻ signal (MW=330.15 Da, [C₁₉H₂₂O₅+H]⁺=[C₁₉H₂₃O₅]⁺=331.1) of CLA-producing consortium expressing OsCYP711A2, and 4DO can be detected both in pellet and medium. These results are consistent with the results of expressing OsCYP711A2 alone, indicating that additional introduction of AtMAX1 did not affect 4DO formation. The analysis is performed by LC-MS using Separation Method III.

FIG. 16A-C Production of orobanchol and detection of related biosynthetic intermediates upon introduction of OsCYP711A3 gene. (A) EIC using 4DO's characteristic m/z⁻ signal (MW=330.15 Da, [C₁₉H₂₂O₅+H]⁺=[C₁₉H₂₃O₅]⁺=331.1) of CL-producing E. coli cocultured with yeast expressing OsCYP711A2 and/or OsCYP711A3, and 4DO was consumed as the substrate by introducing OsCYP711A3. (B) EIC using 18-hydroxy-CLA's characteristic m/z⁻ signal (MW=348.16 Da, [C₁₉H₂₄O₆—H]⁻=[C₁₉H₂₃O₅]⁻=347.1) of CL-producing E. coli cocultured with yeast expressing OsCYP711A2 and/or OsCYP711A3, and putative 18-hydroxy-CLA was detected in the medium of strain expressing OsCYP711A2 or OsCYP711A2/OsCYP711A3 pair. (C) EIC using orobanchol's characteristic m/z⁻ signal (MW=346.14 Da, [C₁₉H₂₂O₆+H]⁺=[C₁₉H₂₃O₆]⁺=347.1) of CL-producing E. coli cocultured with yeast expressing OsCYP711A2 and/or OsCYP711A3, and orobanchol can only be detected in the medium of CL-producing E. coli cocultured with yeast coexpressing OsCYP711A2/OsCYP711A3, which suggests that OsCYP711A3 converted 4DO into orobanchol. The analysis is performed by LC-MS using Separation Method III.

FIG. 17A-C OsCYP722B and SbCYP722B are not involved in 4DO, 5DS or orobanchol biosynthesis. (A) EIC using CLA's characteristic m/z⁻ signal (MW=332.16 Da, [C₁₉H₂₄O₅—H]⁻=[C₁₉H₂₃O₄]⁻=331.1) of CL-producing E. coli cocultured with yeast expressing AtMAX1 and CYP722Bss, and CLA was not consumed as a substrate upon the introduction of OSCYP722B or SbCYP722B. (B) EIC using 18-hydroxy-CLA's characteristic m/z⁻ signal (MW=348.16 Da, [C₁₉H₂₄O₆—H]⁻=[C₁₉H₂₃O₅]⁻=347.1) of CL-producing E. coli cocultured with yeast expressing AtMAX1 and CYP722Bs, and putative 18-hydroxy-CLA was undetectable in the strain expressing OSCYP722B or SbCYP722B. (C) EIC using 4DO or 5DS's characteristic m/z⁻ signal (MW=330.15 Da, [C₁₉H₂₂O₅+H]⁺=[C₁₉H₂₃O₅]⁺=331.1) of CL-producing E. coli cocultured with yeast expressing AtMAX1 and CYP722Bs, and 4DO or 5DS was undetectable in the strain expressing OSCYP722B or SbCYP722B. The analysis is performed by LC-MS using Separation Method III

FIG. 18 Phylogenetic tree of CYP722C homologs from different plant species. The phylogenetic tree was conducted in MEGA X by using ClustalW for multiple sequence alignment and the neighbor-joining method. The parameters are set as follows, bootstrap value, 1000, p-distance mode, partial deletion (50%). The accession numbers of proteins are listed in Table 8.

FIG. 19A-B Detection of orobanchol and 5DS in CYP722C screening experiments. (A) Detection of Orobanchol from carlactone-accumulating E. coli co-cultured with yeast expression ATR1, AtMAX1 and CYP722Cs. (B) Detection of 5DS from carlactone-accumulating E. coli co-cultured with yeast expression ATR1, AtMAX1 and CYP722Cs. This indicate that conversion of CLA into 5DS or orobanchol is conserved among different CYP722C-encoding plant species.

FIG. 20. Characterization of different D27 analogs based on CLA production. Noted that the amount of CLA was presented as the peak area under the raw signal of LC-MS (EIC331.1 [−] m/z) obtained from 1 mL cell culture as no available standards.

FIG. 21A-B Characterization of different engineered enzymes based on CLA production in E. coli-S. cerevisiae consortium. (A) Comparison of the activity of each modified enzyme with the original one. NC represents the strains expressing all the original enzymes (PpD27, tAtCCD7, tAtCCD8). (B) Evaluation of the combined effect of 2 efficient engineered enzymes. Noted that the amount of CLA was presented as the peak area under the raw signal of LC-MS (EIC331.1 [−] m/z) obtained from 1 mL cell culture as no available standards.

FIG. 22A-B Rearrangement of the expression cassettes at plasmid level (A) The amount of CLA produced under different plasmid systems in the E. coli-S. cerevisiae co-culture. (B) The amount of CL remaining in the E. coli-S. cerevisiae co-culture. Noted that the amount of CL and CLA were presented as the peak area under the raw signal of the HPLC (UV detector at 269 nm) and LC-MS (EIC331.1 [−] m/z) obtained from 1 mL cell culture as no available standards.

FIG. 23 Characterization of different MAX1 analogs based on CLA production. Noted that the amount of CLA were presented as the peak area under the raw signal of LC-MS (EIC331.1 [−] m/z) obtained from 1 mL cell culture as no available standards.

FIG. 24A-B. Investigation of the effect of gene copy number on CLA production. (A)CLA production under different copy numbers of EgCYP711A. (B) Remaining CL. Noted that the amount of CL and CLA were presented as the peak area under the raw signal of the HPLC (UV detector at 269 nm) and LC-MS (EIC331.1 [−] m/z) obtained from 1 mL cell culture as no available standards.

FIG. 25A-B Characterization of different CYP722 analogs based on 5DS production. (A) 5DS titer in strains expressing different CYP722. (B) Remaining CLA. Noted that the amount of CLA was presented as the peak area under the raw signal of LC-MS (EIC331.1 [−] m/z) obtained from 1 mL cell culture as no available standards.

FIG. 26A-B Product profile. (A) 5DS titer in different strains. (B) Remaining CL. Noted that the amount of CL was presented as the peak area under the raw signal of the HPLC (UV detector at 269 nm) obtained from 1 mL cell culture as no available standards.

FIG. 27 Product profile. (A)CLA production under different IPTG concentration. (B) Remaining CL. Noted that the amount of CL and CLA were presented as the peak area under the raw signal of the HPLC (UV detector at 269 nm) and LC-MS (EIC331.1 [−] m/z) obtained from 1 mL cell culture as no available standards.

FIG. 28 Production of 6-OH-CLA in the microbial consortium. Selected ion monitoring (SIM) extracted ion chromatogram (EIC) at m/z⁻=331.1 (green), m/z⁻ 347.1 (purple) of CL-producing E. coli co-cultured with yeast expressing ATR1 and (i) an empty vector or (ii) PsCYP722A.

FIG. 29 Characterization of different CYP722A analogs in E. coli-yeast microbial consortia. EV represents the empty vector as negative control. Noted that the amount of 16-OH-CLA was presented as the peak area under the raw signal of LC-MS (EIC347.1 [−] m/z) obtained from 1 mL cell culture as no available standard.

FIG. 30A-B Functional characterization of PpMAX1c as a strigol synthase using SL-producing microbial consortium. (A) Phylogenetic analysis of MAX1 protein analogs. The phylogenetic tree was constructed by MEGA X using neighbor-joining method (90% partial deletion, 5000 bootstraps, p-distance mode, bootstrap values >60% are shown). A total of 29 MAX1 analogs from both monocotyledonous and dicotyledonous were selected for the analysis. The Genbank accession numbers can be found in Table 9. MAX1 with identified functions are marked with their functions. (B) Production of strigol, SL-1 and SL-2 in the microbial consortia. Selected ion monitoring (SIM) extracted ion chromatogram (EIC) at m/z⁻=331.1 (green), m/z⁻ 347.1 (purple), m/z⁺=331.1 (blue), and m/z⁺=347.1 (orange) of (i) strigol, sorgomol, OB, 4DO, and 5DS standard; CL-producing E. coli co-cultured with yeast expressing ATR1 and (ii) an empty vector, (iii) PpMAX1b, (iv) PpMAX1c. The characteristic m/z⁺ of strigol signal (MW=346.38) is [C₁₉H₂₂O₆+H]⁺=[C₁₉H₂₃O₆]⁺=347.1. The characteristic m/z⁺ signal of 4DO and 5DS (MW=330.38) is [C₁₉H₂₂O₅+H]⁺=[C₁₉H₂₃O₅]⁺=331.1.

FIG. 31A-B Media optimization for enhanced production of 16-OH-CLA in E. coli-yeast microbial consortia. (A) CLA and 16-OH-CLA production. (B) Remaining CL. CK represents the control group with no supplementation in SD media. Noted that the amount of CL, CLA, and 16-OHCLA were presented as the peak area under the raw signal of the HPLC (UV detector at 269 nm), LC-MS (EIC331.1 [−] m/z), and LC-MS (EIC347.1 [−] m/z) obtained from 1 mL cell culture as no available standards.

### DETAILED DESCRIPTION

The present disclosure provides methods and reagents for producing SLs using a bacterial and yeast co-culture expression system.

The invention employs various routine recombinant nucleic acid techniques. Generally, the nomenclature and the laboratory procedures in recombinant DNA technology described below are commonly employed in the art. Many manuals that provide direction for performing recombinant DNA manipulations are available, e.g., Sambrook & Russell, Molecular Cloning, A Laboratory Manual (3rd Ed, 2001); and Current Protocols in Molecular Biology (Ausubel, et al., John Wiley and Sons, New York, 2009-2014).

As used, herein, reference to “a polypeptide encoded by” a specified gene or other reference polynucleotide refers to a polypeptide that has the same amino acid sequence as the polypeptide encoded by the specified gene or reference polynucleotide gene, and thus includes polypeptides of the same sequence, but that may be encoded by a nucleic acid sequence that comprises a different codon, relative to the specified gene or reference polynucleotide, for the same amino acid.

### Bacterial Expression Systems for Co-Culture

As used, herein, reference to “a polypeptide encoded by” a specified gene or other reference polynucleotide refers to a polypeptide that has the same amino acid sequences as the polypeptide encoded by the specified gene or reference polynucleotide gene, and thus includes polypeptides of the same sequence, but may be encoded by a nucleic acid sequence that comprises a different codon, relative to the specified gene or reference polynucleotide, for the same amiono acid.

Bacterial cells, e.g., E. coli, are used to express the portion of the CL biosynthesis pathway that generates CL. Host cells are genetically modified to express DWARF27 (D27), and two carotenoid cleavage dioxygenases (CCD7 and CCD8) polypeptides encoded by D27, CCD7, and CCD8 polypeptides from plants. In some instances, these polypeptides are collectively referred to herein as “Module 1 polypeptides”.

As used here, the term “D27” with reference to a nucleic acid includes the gene represented by the accession numbers as well as orthology, homologs, and variants thereof. In typical embodiments, a “D27” polypeptide encoded by a D27 nucleic acid has at least 70%, at least 75%, at least 80%, or at least 85% identity to a naturally occurring D27 polypeptide sequence, e.g., a D27 encoded by a D27 gene from Oryza sativa, e.g., OsD27 (e.g., accession number Os11g37650); or to the region of the D27 polyepptide that lacks the chloroplast transit sequence. In some embodiments, the “D27” polypeptide has at least 90% identity or an least 95% identity to a naturally occurring OsD27 polypeptide; or to the region of the polypeptide that lackd the chloroplast transit sequence. In some embodiments, a D27 polypeptide is encoded by a DNA sequence as shown in Table 8, or is a variant of such a polypeptide that has at least 70%, at least 75%, at least 80%, or at least 85% identity to the polypeptide encoded by the DNA sequence shown in Table 8. In some embodiments, the variant polypeptide sequence hast at least 90%, often at least 95% identity to the polypeptide encoded by the DNA sequence shown in Table 8. In some embodiments, a D27 polypeptide has a sequence available under an accession number provided in Table 5 or is a variant of such a polypeptide that has at least 70%, at least 75%, at least 80%, or at least 85% identity to the polypeptide encoded by an accession number shown in Table 5. In some embodiments, the variant polypeptide sequence hast at least 90%, often at least 95% identity to the polypeptide encoded by an accession number shown in Table 5. In some embodiments, a D27 polypeptide is encoded by the nucleic acid sequence shown for tPpD27 or PpD27 in Table 8, or is a variant of such a polypeptide that has at least 90%, often at least 95% identity to the polypeptide encoded by the tPdD27 or PpD27 sequence provide in Table 8. In some embodiments, the DNA sequence encoding a D27 polypeptide has at least 70%, or at least 75% identity to a D27 DNA sequence shown in Table 8. In some embodiments, the DNA sequence encoding a D27 polypeptide has at least 80% or at least 85% identity to a D27 DNA sequence shown in Table 8. In some embodiments, the DNA sequence encoding a D27 polypeptide has at least 90% or at least 95% identity to a D27 DNA sequence shown in Table 8. In some embodiments, the nucleic acid sequence endoing a D27 polypeptide has at least 70%, or at least 75% identity to the DNA sequence tPpD27 or PpD27 shown in Table 8. In some embodiments, the nucleic acid sequence endoing a D27 polypeptide has at least 80%, or at least 85% identity to the DNA sequence tPpD27 or PpD27 shown in Table 8. In some embodiments, the nucleic acid sequence endoing a D27 polypeptide has at least 90%, or at least 95% identity to the DNA sequence tPpD27 or PpD27 shown in Table 8.

As used here, the term “CCD7” with reference to a nucleic acid includes the gene represented by the accession numbers as well as orthology, homologs, and variants thereof. In typical embodiments, a “CCD7” polypeptide has at least 70%, at least 75%, at least 80%, or at least 85% identity to a naturally occurring CCD7 polypeptide sequence, e.g., a CCD7 from Arabidopsis thaliana, that lacks the chloroplast transit peptide sequence (e.g., lacks the N-terminal 31 amino acids of A. thaliana CCD7 containing the chloroplast transit peptide sequence), e.g., the AtCCD7 polypeptide (e.g., accession number AT2G44990) encoded by an A. thaliana CCD7 gene. In some embodiments, the “CCD7” polypeptide has at least 90% identity or an least 95% identity to a naturally occurring CCD7 polypeptide that lacks the transit peptide, e.g., the AtCCD7 polypeptipde.

As used here, the term “CCD8” with reference to a nucleic acid includes the gene represented by the accession numbers as well as orthology, homologs, and variants thereof. In typical embodiments, a “CCD8” polypeptide has at least 70%, at least 75%, at least 80%, or at least 85% identity to a naturally occurring CCD8 polypeptide sequence, e.g., a CCD8 from Arabidopsis thaliana, that lacks the chloroplast transit peptide (e.g., the N-terminal 56 amino acids of the A. thaliana CCD8 containing the chloroplast transit peptide sequence), e.g., the AtCCD8 polypeptide (e.g., accession number AT4G323810) encoded by an A. thaliana CCD8 gene. In some embodiments, the “CCD8” polypeptide has at least 90% identity or an least 95% identity to a naturally occurring CCD8 polypeptide that lacks the transit peptide, e.g., the AtCCD8 polypeptipde.

The genes can be introduced into bacterial host cells using any number of known techniques. Gene can be expressed on separate expression vectors. or in some embodiments, two or more of the genes can be expressed on the same expression vector. In some embodiments, an expression vector that comprises an expression cassette that comprises the gene further comprises a promoter operably linked to the gene. In some embodiments, a promoter and/or other regulatory elements that direct transcription of the gene are endogenous to the microorganism and an expression cassette comprising the gene encoding the enzyme is introduced, e.g., by homologous recombination, such that the heterologous gene is operably linked to an endogenous promoter and is expression driven by the endogenous promoter.

As noted above, expression of the genes encoding Module 1 polpeptide e can be controlled by a number of regulatory sequences including promoters, which may be either constitutive or inducible; and, optionally, repressor sequences, if desired. For example, in one embodiment, the promoter is a T7 promoter. Additional examples of promoters include promoters such as the trp promoter, bla promoter bacteriophage lambda PL, and T5; inducible promoters such as promoters from the lac operon or other sugar-regulated genes in bacteria. In addition, synthetic promoters, such as the tac promoter can be used. Further examples of promoters include Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes. Suitable promoters are also described in Ausubel and Sambrook & Russell, both supra.

Although any suitable expression vector may be used to incorporate the desired sequences, readily available bacterial expression vectors include, without limitation: plasmids such as pSClOl, pBR322, pBBR1MCS-3, pUR, pET, pEX, pMR100, pCR4, pBAD24, p15a, pACYC, pCDF, pRSF, or pUC, or plasmids derived from these plasmids; and bacteriophages, such as Ml 3 phage and λ phage.

### Bacterial Host Cells

A number of bacterial host cells are suitable for genetic modification to express Module 1 polypeptides. These include, for example, E. coli; Bacillus sp., e.g., Bacillus subtilis; Lactococcus sp., e.g., Lactococcus lactis; and Pseudomonas sp. One of skill understand that genes for expression in the desired host cell can be codon-optimized for expression.

In some embodiments, a genetically modified bacterially host strain modified to express Module 1 polypeptides can comprises at least one additional genetic modification to enhance production of one or more components of the CL pathway.

### Yeast Expression Systems for Co-Culture

Yeast host cells, e.g., Saccharomyces cervisiae, are used to express the portion of the CL biosynthesis pathway that generates SLs from the bacterially produce CL. Host cells can be genetically modified to express a number of different SLs as described herein. Modifications include expression of a cytochrome P450 and a corresponding P450 reductase, and a synthetase to produce the desired SL in the yeast strain (Module 2). In some instances, the polypeptides expressed in yeast systems to generate SLs are collectively referred to herein as “Module 2 polypeptides”.

In some embodiments, the yeast strain for the co-culture system is genetically modfidied to express a cytochrome P450 reductase, e.g., such as ATR1, and additional polypeptides such as MORE AXILLARY GROWTH 1 (MAX1) (e.g., accession number AT4G24520), and Sl synthetase polypeptides. In some instances, the polypeptides expressed in yeast systems to generate SLs are collectively referred to herein as “Module 2 polypeptides”.

In some embodiments, the yeast strain for the co-culture system is genetically modfidied to synthesize CLA by engineering the strain to express a cytochrome P450 reductase gene e.g., the cytochrome P450 from A. thaliana, AtATR1; a carlactonoic acid (CLA) synthetase gene, e.g., MAX1 from A. thaliana, AtMAX1. In some embodiments, the yeast strain expressing the P450 reductase and MAX1 also comprises a genetic modification to express a 5DS synthase gene, e.g. CYP722C from Gossypium arboreum, GaCYP722C (e.g., accession number XP_016745621), in order to produce 5D2. In some embodiments, the yeast strain expressing the P450 reductase and MAX1 also comprises a genetic modification to express an orobanchol synthase gene, e.g., CYP722C from Capsicum annuum, CaCYP722C (e.g., accession number XP_016560669) to produce orobanchol.

In some embodiments, the yeast strain for the co-culture system is genetically modfidied to synthesize 4DO by engineering the strain to express a cytochrome P450 reductase gene e.g., the cytochrome P450 reductase from A. thaliana, AtATR1; a 4DO synthase gene, e.g., CYP711A2 from Oryza sativa, OsCYP711A2 (e.g., accession umber os01g0700900). In some embodiments, the yeast strain comprises a genetic modification to express an orobanchol synthase gene, e.g., CYP711A3 from Oryza sativa, OsCYP711A3 (e.g., accession number Os01g0701400), to convert 4DO to orobanchol.

In some embodiments, the yeast strain for the co-culture system is genetically modfidied to synthesize 16-OH-CLA by engineering the strain to express a cytochrome P450 reductase gene e.g., the cytochrome P450 reductase from A. thaliana, AtATR1; and a CYP722A gene, e.g., a CYP722a from Pisum sativum, or Aquilegia coerulea; or a CYP722A gene from a different plant species such as Cannabis sativa, Eucalyptus grandis, Fragaria vesca, Macadamia integrifolia, Nelumbo nucifera, Prunus mume, Prunus avium, Ricinus communis, or Prunus persica.

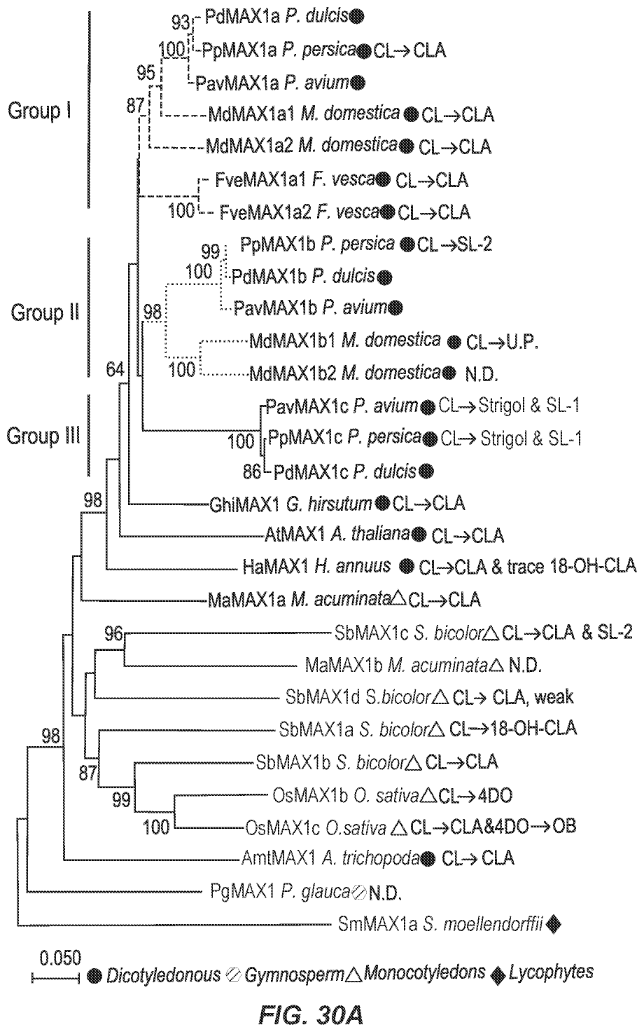

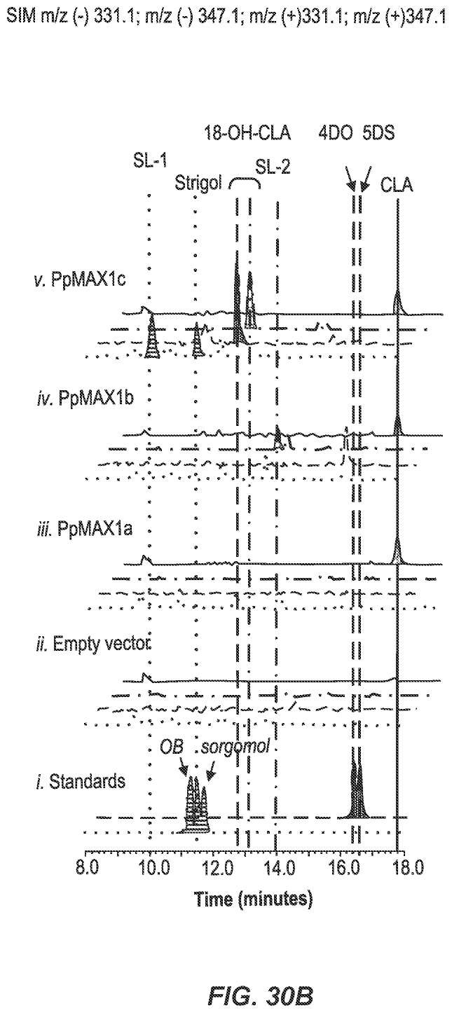

In some embodiments, the yeast strain for the co-culture system is genetically modified to synthesize strigol and an oxidized 5DS compound, referred to herein as SL-1, by engineering the strain to express a cytochrome P450 reductase gene e.g., the cytochrome P450 reductase gene from A. thaliana, AtATR1; and a MAX1 gene, such as PpMAX1c from peach, or a MAX1 gene from a different plant, e.g., for example selected from a gene listed in FIG. 30A that converts CL to CLA (see also, Table 9).

In some embodiments, the yeast strain for the co-culture system is genetically modified to synthesize a new hyudroxylated or oxidated CLA compound, referred to herein as SL-2, by engineering the strain to express a cytochrome P450 reductase gene e.g., the cytochrome P450 reductase from A. thaliana, AtATR1; and a MAX1 gene, such as PpMAX1b or SbMAX1c or from a different plant (see, Table 9).

The genes employed in the genetic modifications to yeast cells to convert CL to SLs specifically noted above are illustrative genes. One of skill understands that the corresponding genes from other plants can also be employed. Accordingly, orthologys, homologs and variants of the illustrative genes noted above may also be employed. In typical embodiments, a Module 2 polypeptide encoded by a Module 2 gene has at least 70%, at least 75%, at least 80%, or at least 85% identity to a naturally occurring Module 2 amino acid sequence. In some embodiments, the Module 2 polypeptide encoded by a Module 2 gene has at least 90% identity or at least 95% identity to a naturally occurring Module 2 amino acid sequence. For example, in some embodiments, a cytochrome P450 reductase gene employed for genetic modification of yeast cells encodes a P450 reductase having at least 70%, at least 75%, at least 80%, or at least 85% amino acid sequence identity to the polypeptide encoded by AtAR1. In some embodiments, the P450 reductase has at least 90% identity or at least 95% amino acid sequence identity to the polypeptide encoded by AtAR1. In some embodiments, a CLA synthetase gene employed for genetic modification of yeast cells encodes a CLA synthetase having at least 70%, at least 75%, at least 80%, or at least 85% amino acid sequence identity to the polypeptide encoded by MAX1. In some embodiments, the CLA synthetase gene has at least 90% identity or at least 95% amino acid sequence identity to the polypeptide encoded by MAX1. In some embodiments, the CLA synthetase gene has at least 90% identity or at least 95% identity to a polypeptide encoded by a MAX1 DNA sequence shown in Table 8. Polypeptide sequences are available under the accession number provided in Table 6. In some embodiments, a 5DS synthase gene employed for genetic modification of yeast cells encodes a 5DS synthase having at least 70%, at least 75%, at least 80%, or at least 85% amino acid sequence identity to the polypeptide encoded by GaCYP722C. In some embodiments, the 5DS synthase gene has at least 90% identity or at least 95% amino acid sequence identity to the polypeptide encoded by GaCYP722C. In some embodiments, a 5DS synthase gene employed for genetic modification of yeast cells encodes a 5DS synthase having at least 70%, at least 75%, at least 80%, or at least 85% amino acid sequence identity to the polypeptide encoded by the CYP722C synthase gene from Ricinus communis, RcCYP722C2 (e.g., accession number XP_002524333). In some embodiments, the 5DS polypeptide is an RcCYP722C2 polypeptide encoded by an RcCYP722C2 DNA sequence provided in Table 8 or a variant thereof having at least 90% or at least 95% identity to the RcCYP722C2 polypeptide encoded by the DNA sequence provided in Table 8. In some embodiments, the 5DS synthase gene has at least 90% identity or at least 95% amino acid sequence identity to the polypeptide encoded by RcCYP722C2. In some embodiments, an orobanchol synthase gene employed for genetic modification of yeast cells encodes an orobanchol synthase having at least 70%, at least 75%, at least 80%, or at least 85% amino acid sequence identity to the polypeptide encoded by CaCYP722C. In some embodiments, the orobanchol synthase gene has at least 90% identity or at least 95% amino acid sequence identity to the polypeptide encoded by CaCYP722C. In some embodiments, the 4DO gene employed for genetic modification of yeast cells encodes a 4DO synthase having at least 70%, at least 75%, at least 80%, or at least 85% amino acid sequence identity to the polypeptide encoded by OsCYP711A2. In some embodiments, the 4DO synthase gene has at least 90% identity or at least 95% amino acid sequence identity to the polypeptide encoded by OsCYP711A2. In some embodiments, the orobanchol synthase gene employed for genetic modification of yeast cells encodes an orobanchol synthase having at least 70%, at least 75%, at least 80%, or at least 85% amino acid sequence identity to the polypeptide encoded by OsCYP711A3. In some embodiments, the orobanchol synthase gene has at least 90% identity or at least 95% amino acid sequence identity to the polypeptide encoded by OsCYP711A3. In some embodiments, the orobanchol synthase gene employed for genetic modification of yeast cells encodes an orobanchol synthase having at least 70%, at least 75%, at least 80%, or at least 85% amino acid sequence identity to the polypeptide encoded by a cowpea VuCYP722C gene (e.g., accession number XP_027918387). In some embodiments, the orobanchol synthase gene has at least 90% identity or at least 95% amino acid sequence identity to the polypeptide encoded by the cowpea VuCYP722C. In some embodiments, the orobanchol synthase gene employed for genetic modification of yeast cells encodes an orobanchol synthase having at least 70%, at least 75%, at least 80%, or at least 85% amino acid sequence identity to the polypeptide encoded by a Trifolium pretense TPCYP722C gene (e.g., accession number Tp57577_TGAC_v2_mRNA22267); a Manihot esculenta, MeCYP722C1 gene (e.g., accession number XP_021622147); or a Vitis vinifera, VVCYP722c gene (e.g., accession number XP_002269279). In some embodiments, the orobanchol synthase gene has at least 90% identity or at least 95% amino acid sequence identity to the polypeptide encoded by the Trifolium pretense TPCYP722c gene; a Manihot esculenta, MeCYP722C1 gene; or a Vitis vinifera, VVCYP722C gene.

In some embodiments, a cytochrome P450 CYP722 gene employed for genetic modification of yeast cells encodes a polypeptide having at least 70%, at least 75%, at least 80%, or at least 85% amino acid sequence identity to a polypeptide encoded by a CYP gene listed in Table 4. In some embodiments, the orobanchol synthase gene has at least 90% identity or at least 95% amino acid sequence identity to the polypeptide encoded by the gene listed in Table 4.

The genes can be introduced into yeast host cells using any number of known techniques. Gene can be expressed on separate expression vectors. or in some embodiments, two or more of the genes can be expressed on the same expression vector. In some embodiments, an expression vector that comprises an expression cassette that comprises the gene further comprises a promoter operably linked to the gene. In some embodiments, a promoter and/or other regulatory elements that direct transcription of the gene are endogenous to the yeast cell and an expression cassette comprising the gene encoding the enzyme is introduced, e.g., by homologous recombination, such that the heterologous gene is operably linked to an endogenous promoter and is expression driven by the endogenous promoter.

Suitable promoters of use in a yeast host cell include promoters illustrated in the Examples section, e.g., PGK1 and TEF1 promoters, e.g., from Saccharomyces cervisiae. Additional illustrative promoters that can be employed include promoters obtained from the genes for Saccharomyces cerevisiae glyceraldehyde-3-phosphate dehydrogenase (TDH3), Saccharomyces cerevisiae translational elongation factor EF-1 alpha (TEF1), Saccharomyces cerevisiae pyruvate kinase (PYK1), Saccharomyces cerevisiae high-affinity glucose transporter (HXT7), Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), and Saccharomyces cerevisiae metallothionein (CUP1).

An expression vector may also comprise additional sequences that influence expression of the gene, including enhancer sequences or other sequences such as transcription termination sequences, and the like.

A vector expressing a nucleic acid for expression of an enzyme in yeast hybrid peroxidase may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host, is integrated into the genome and replicated together with the chromosome(s) into which it has been integrated.

### Host Cells

Any of a wide variety of yeast host cells may be used for expression of Module 2 polypeptides. In some embodiments, the host cell is a Candida, Hansenula, Kluyveromyces, Pichia, Saccharomyces, Schizosaccharomyces, or Yarrowia host cell. In some embodiments, the yeast host cell is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis cell. In some embodiments, the yeast host cell is a Kluyveromyces lactis cell. In another embodiment, the yeast host cell is a Yarrowia lipolytica cell. One of skill understand that genes for expression in the desired host cell can be codon-optimized for expression.

In some embodiments, a genetically modified yeast host strain modified to express Module 2 polypeptides can comprises at least one additional genetic modification to enhance production of one or more SLs.

### Co-Culture

The genetically modified bacterial and yeast strains to synthesize SLs are co-cultured in a co-culture system, e.g., as described in the technical section. In some embodiments, the pH of the growth media may be modulated, e.g., by increasing the pH from about pH 6.0 to about pH 7.0, e.g., plus or minus 5% of the designated pH, to support increased production of carlactone. One of skill understands that additional adjustments may be made to optimize producton of a desired SL. In some embodiments, a method of producing an SL as decribed herein further comprises purifying the SL from the co-culture system. Purificatoni can be performed using known techniques.

### Technical Section

The following techniques are provided to illustrate, but not limit the claimed invention.

### Introduction and Rationale:

Here, we dissected the biosynthetic pathway of SL into two modules and harness the previously developed E. coli-S. cerevisiae consortium strategy to establish a microbial SL platform for synthesizing both non-canonical and canonical SLs, including 5-deoxystrigol (5DS) at ˜6.65±1.71 μg/L, 4-deoxyorobanchol (4DO) at ˜3.46±0.28 μg/L, and orobanchol at 16.91±2.11 μg/L. The SL-producing platform enabled us to conduct functional screening of CYP722Cs from various plants, the function of which is consistent with the reported SL production profile from the corresponding plant. The functional validation of CYP722Cs will also enable the prediction on SL synthetic capacity from different plants. This work provides a unique platform for the elucidation of SL biosynthesis, and the supply of SLs that will meet the market demand for both fundamental SL-related research and agricultural applications.

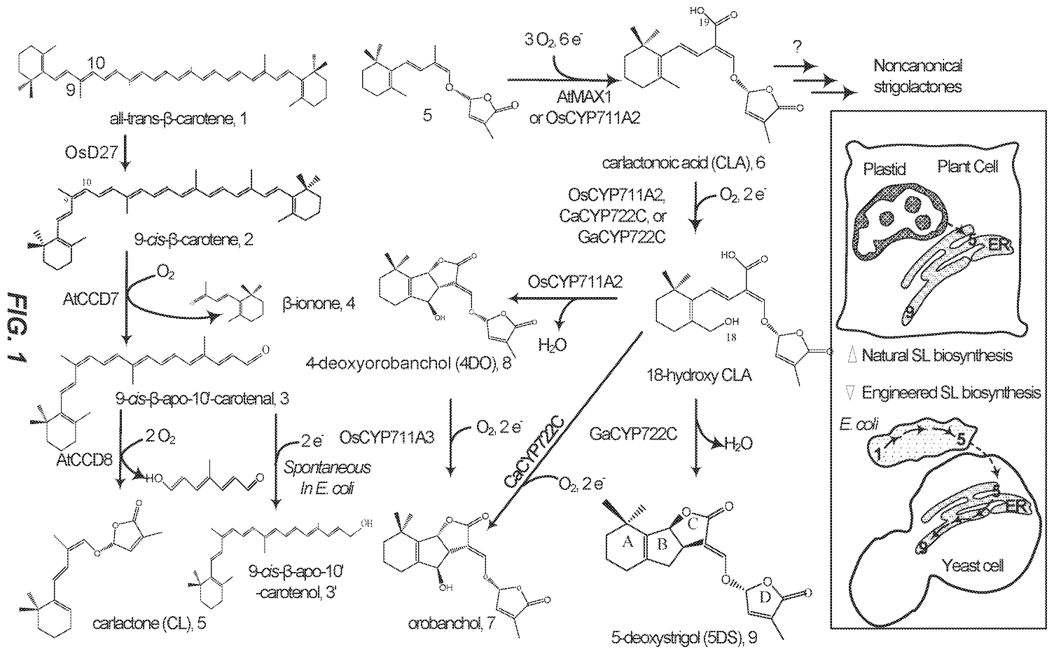

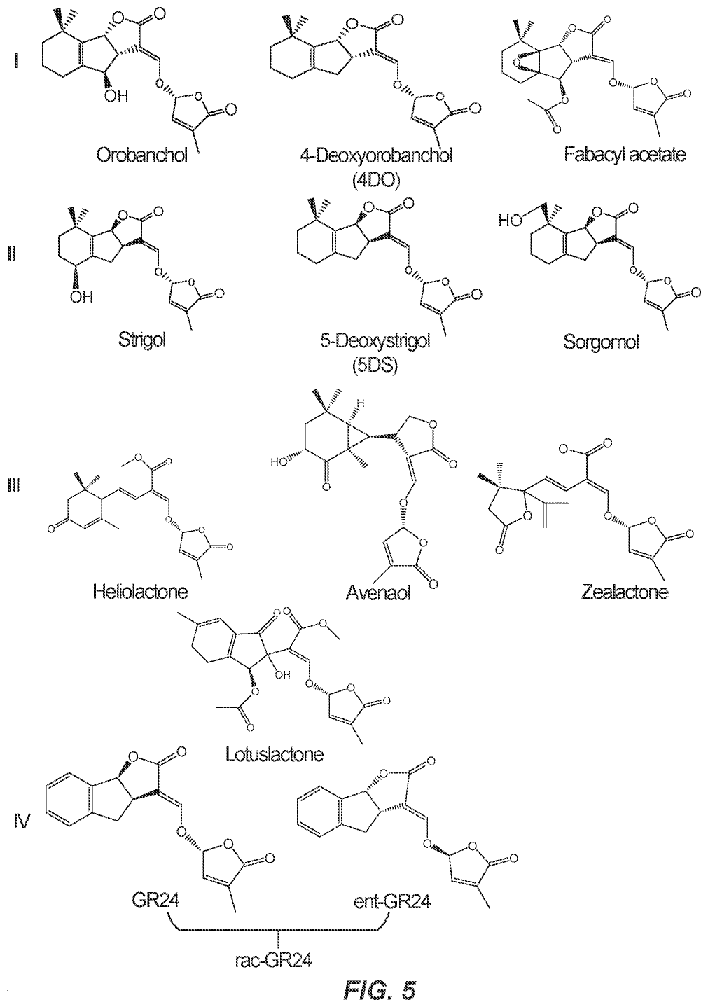

Strigolactones (SLs) were initially characterized as signaling molecules, which are released from plant roots, induce germination of root parasitic weed, regulate the hyphae branching of arbuscular mycorrhiza fungi (AMF), and promote the symbiotic relationship between plants and fungi (1, 2). Later, they were also identified as a novel class of plant hormones that control shoot branching, leaf growth and senescence, and promote the formation of lateral root and growth of primary root (3). SLs thus have been considered as promising agrochemicals, such as bio-stimulants that enhance the nutrient uptake efficiency through modulating plant-AMF symbiotic association (4, 5). To date, more than 30 natural SLs have been isolated (6) (FIG. 5). SLs generally consist of a conserved butenolide ring (D ring) connected to a less conserved tricyclic lactone ring via an enol-ether bond (7) (FIG. 1). SLs can be classified into canonical and non-canonical SLs: the canonical SLs contained the tricyclic lactone-ring (ABC ring), while the non-canonical SLs lack of the tricyclic ring scaffold with one (C ring) or two rings (B ring and C ring) missing (8). The canonical SLs can be further subdivided into orobanchol (O)- and the strigol (S)-type SLs according to the stereochemistry in the C ring, which are represented by 4-Deoxyorobanchol (4DO) and 5-deoxystrigol (5DS), respectively (7). Some of the better-known non-canonical SLs include methyl carlactonoate (MeCLA) (9), heliolactone (10), avenaol (11), zealactone (10), and lotuslactone (12).

SLs are derived from β-carotene, which is converted to carlactone (CL), the key branching point in SL biosynthesis (19), by the functions of three chloroplast enzymes: the isomerase DWARF27 (D27), carotenoid cleavage dioxygenase 7 and 8 (CCD7 and CCD8) (19) (FIG. 1). D27, a [2Fe-2S]-containing polypeptide, catalyzes the isomerization of all-trans-β-carotene to 9-cis-β-carotene in plastids (19), followed by CCD7, a non-heme iron-dependent enzyme that catalyzes the C9′-C10′ double bond cleavage of 9-cis-β-carotene to yield 9-cis-β-apo-10′-carotenal and β-ionone (19). Subsequently, CCD8, another non-heme iron-dependent enzyme, further catalyzes the oxidative cleavage of 9-cis-β-apo-10′-carotenal to synthesize CL, with the reaction mechanism remaining elusive (19, 20). CL is then exported into cytoplasm, and further oxidized by cytochrome P450s and other oxidases to afford various SL structures (21). The first oxidation step has been characterized to be the C19-oxidation of CL to synthesize carlactonic acid (CLA), which is catalyzed by the MORE AXILLARY GROWTH 1 (MAX1), a member of the CYP711A cytochrome P450 family (9), and conserved in a number of plant species (22). In addition to being the common precursor, CLA is generally found to be present in the root exudates, which suggests its role in plant-microbe communication (23).

The canonical SL scaffolds are generally believed to be synthesized from CL or CLA by the functions of cytochrome P450s that belong to either CYP711A (MAX1) (21) or CYP722C family. The distinct stereochemistry of C ring divides natural canonical SLs into two categories: O-type with α-oriented C ring and S-type with β-oriented C ring (24). Most plants only produce one type of SLs, with very few producing both types (24). Rice, known to produce O-type SL such as 4DO and orobanchol (25), encodes five MAX1 homologs. One MAX1 homolog, CYP711A2 encoded by Os900, was identified to catalyze the conversion of CL to the canonical 4-deoxyorobanchol (4DO), likely via CLA yet the exact enzymatic mechanism remains unclear (22, 26). 4DO can be further oxidized through C4-hydroxylation by MAX1 analogs (CYP711A3 encoded by Os1400 from rice, ZmMAX1b from maize) to afford orobanchol (22). However, 4DO is not produced in many orobanchol-producing plants (e.g. tomato, cowpea), which hints another synthetic route of orobanchol without passing through 4DO (27, 28). This direct conversion of orobanchol from CLA was later identified to be catalyzed by CYP722C in cowpea through a two-step C18-oxidation (29). On the other hand, previous ¹³C-tracing experiments in the 5DS-accumulating Lotus japonicus indicates that 5DS is also likely to be derived from CLA (30). Recently, CYP722C from Gossypium arboretum was characterized to catalyze the synthesis of 5DS from CLA (31). Moreover, one recent study found that inactivating LGS1 (LOW GERMINATION STIMULANT 1) in the S-type SL-producing sorghum led to an almost complete alteration from S-type to 0-type SLs (32). The study implies the possible existence of enzymatic mechanism to convert between S-type and O-type SLs, in addition to using different cytochrome P450s to determine the orientation of C ring downstream of the synthesis of CLA.

Despite the relatively well-elucidated SL biosynthesis, the de novo synthesis of SLs in microbial hosts have not been achieved yet. D27, CCD7, and CCD8 are localized in chloroplast, which is evolutionarily closer to E. coli, and not present in yeast. Specifically, functional expression of iron sulfur proteins in the cytosol of S. cerevisiae was found to be highly challenging (33). In contrast, E. coli can provide the cellular environment for the function reconstitution of iron sulfur proteins (34). In addition, the previous in vitro biochemical characterization for CL synthesis has demonstrated E. coli as an appropriate heterologous host for the functional expression of D27, CCD7, and CCD8 (19). On the other hand, although there are some successful examples, E. coli normally fails to provide the necessary membrane infrastructures for the functional reconstitution of plant cytochrome P450s (35). In contrast, S. cerevisiae has been demonstrated as a feasible platform for the functional reconstitution of plant cytochrome P450s (36). We hypothesized that the E. coli and S. cerevisiae can perform complementary roles in constructing the biotechnological production of SLs. Such E. coli-S. cerevisiae co-culture consortium strategy has been successfully developed and utilized in the bioproduction of oxygenated taxanes (37). In this study, we harnessed the E. coli-S. cerevisiae co-culture strategy to achieve an efficient and versatile production of different SLs. We engineered E. coli to produce CL, which was transformed in a modified S. cerevisiae to CLA, O- and S-type SLs. To further enhance the production of SLs, metabolic engineering and fermentation engineering were applied and led to enhanced levels of SLs. The bioproduction platform also enabled us to confirm the function of previously unknown CYP722Cs from various plants, functions of which is consistent with the reported SL production profile from the corresponding plant. This work highlights the strength of the E. coli-S. cerevisiae co-culture strategy, and provides a platform for elucidating the missing steps in SL biosynthesis, and producing natural SLs.

Attempts to Establish CL Production in S. cerevisiae.

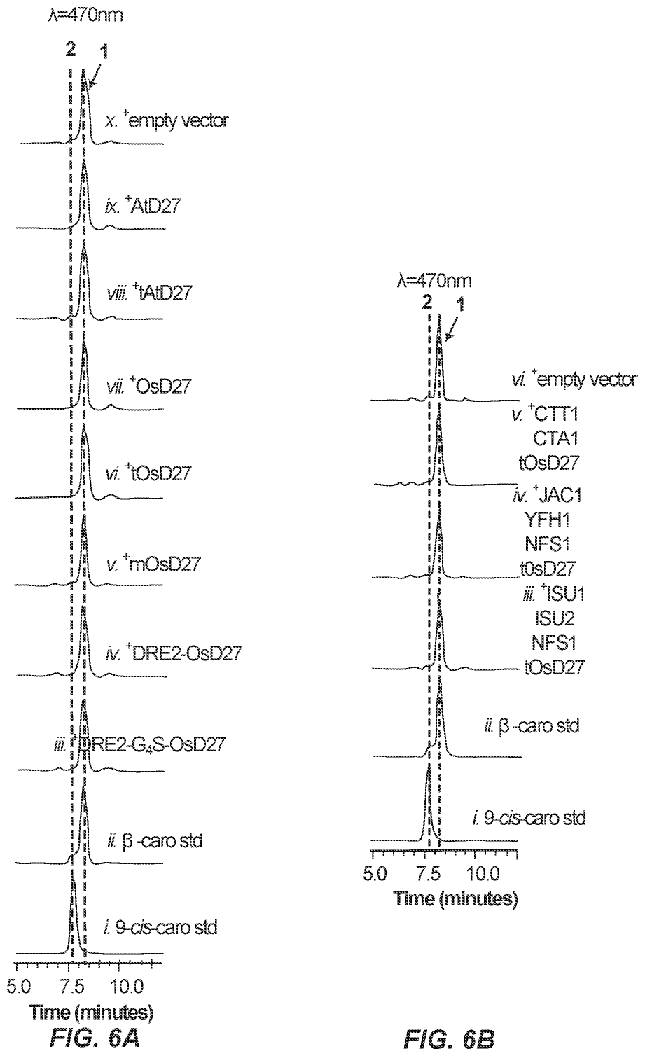

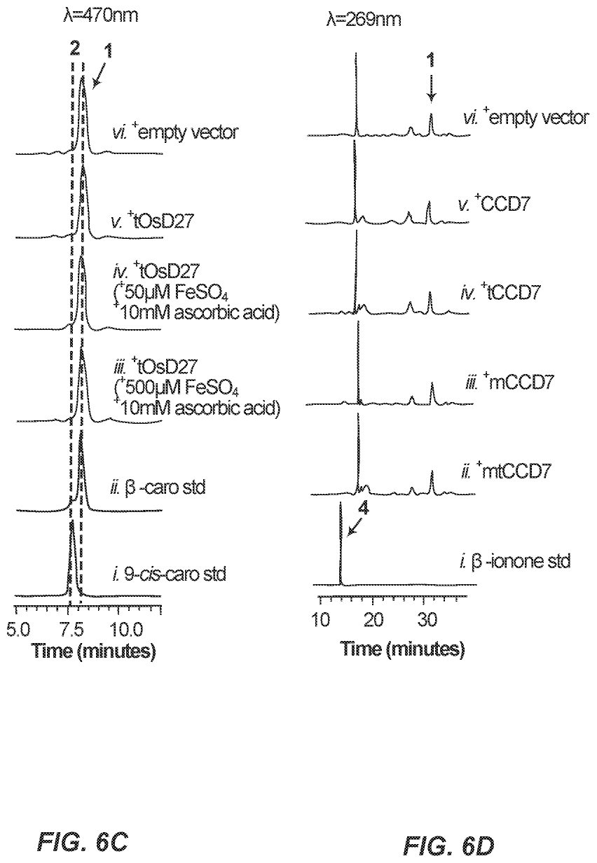

To synthesize CLA, we first attempted to functionally reconstitute D27, CCD7, and CCD8 in a β-carotene-producing S. cerevisiae strain. The biosynthesis of β-carotene in yeast has been well documented using the fungal β-carotene biosynthetic genes (38). Here, we reconstructed the β-carotene producing yeast strain as previously described, through genomic integration of crtE (GGPP synthase), crtYB (a bifunctional phytoene synthase and lycopene cyclase), and crtI (phytoene desaturase) from X. dendrorhous with an additional copy of yeast endogenous GGPP synthase BTS1 to afford YYL23. Trace amount of 9-cis-β-carotene naturally exists in the established 1-carotene-producing yeast strain with a ratio to the all-trans-β-carotene roughly ˜1:25 (FIG. 6A). D27, CCD7, and CCD8 are all plastid-localizing enzymes, which normally exhibit better activity in the microbial host when the N-terminus plastid-localizing peptide are truncated (39). However, introducing either full length or N-terminus truncated D27 from either A. thaliana or rice did not lead to any changes to the ratio between 9-cis-β-carotene and all-trans-β-carotene in YYL23 (FIG. 6A). D27 is a ferredoxin like 2Fe-2S protein while few plant 2Fe-2S proteins have been functionally expressed in yeast. The iron-sulfur cluster biogenesis takes place in mitochondria; and the microenvironment in plastid is evolutionary closer to the mitochondria, compared to the cytosol of the eukaryotic yeast. Thus, localizing to mitochondria could be a reasonable strategy for the functional reconstitution of D27. We attempted to localize D27 from Oryza sativa (OsD27) to mitochondria by fusing a well-documented 26-residue mitochondrial targeting signal to the N-terminus, which did not seem to help the activity of OsD27 in yeast (40) (FIG. 6A). We also fused D27 to the C-terminus of the cytosol Fe—S protein assembly (CIA) machinery component DRE2 with and without a flexible linker GGGGS (G4S) (SEQ ID NO: 1) (Table 1) yet no enhanced level of 9-cis-β-carotene was detected (FIG. 6A). Additionally, five proteins that are involved in the iron-sulfur biogenesis in mitochondria were overexpressed, including JAC1, ISU1, ISU2, YFH1, NFS1, yet no activity of D27 in converting all-trans-β-carotene to 9-cis-β-carotene was detected in YYL23 (FIG. 6B). Iron supplementation (50 μM and 500 μM FeSO₄) with reducing agent (10 Mm ascorbic acid) also did not lead to any observed D27 activity (FIG. 6C). Furthermore, the two endogenous catalases, CTT1 and CTA1, were overexpressed to mimic the previously reported in vitro CL synthesis yet with no detection of the activity of D27 (19) (FIG. 6B). In addition to repeated failures in the functional reconstitution of D27, we also tried to reconstitute the function of CCD7 from A. thaliana, due to the presence of the substrate of CCD7, 9-cis-β-carotene, though at low titer in the absence of a functional D27 in YYL23. Although CCD1 has been functionally expressed in yeast to cleave β-carotene to afford the synthesis of β-ionone (41), we did not detect the function of CCD7 possibly due to the fact the CCD1 is natively localized in cytosol while the other CCDs are in plastid (42) (FIG. 6D). Similarly, we also constructed N-terminus truncated CCD7, and mitochondria-targeting full length and truncated CCD7, and no cleavage activity of β-carotene was detected in YYL23 (FIG. 6D). The failure in establishing CL synthesis in yeast aligns with the challenge of functional reconstitution of plastid-localizing enzymes due to the distinct microenvironment.

Establishment of CL Production in E. coli

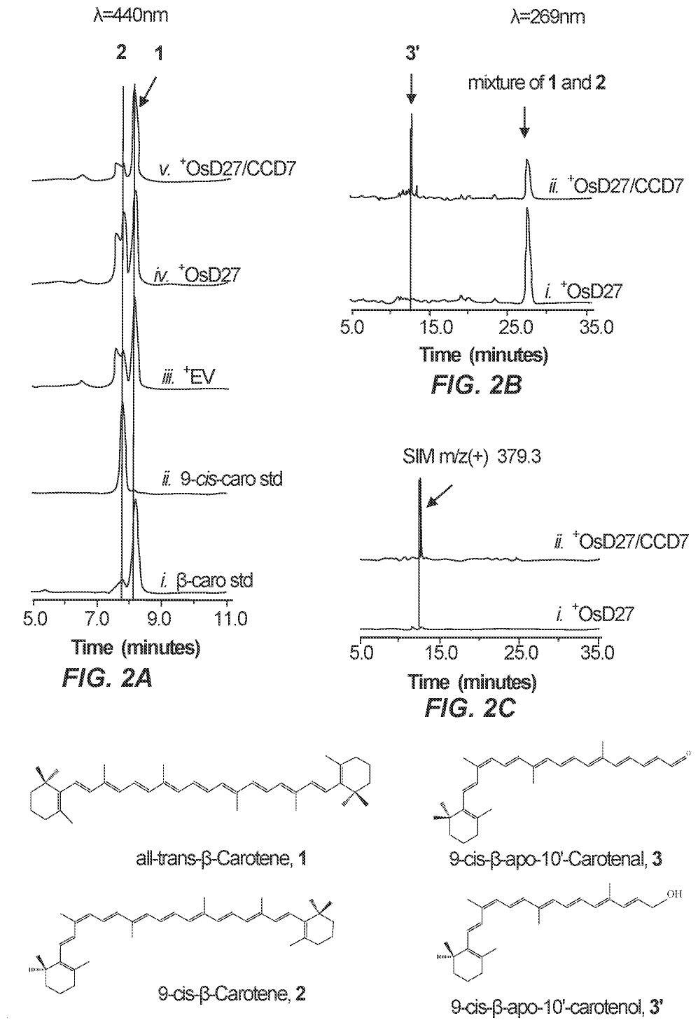

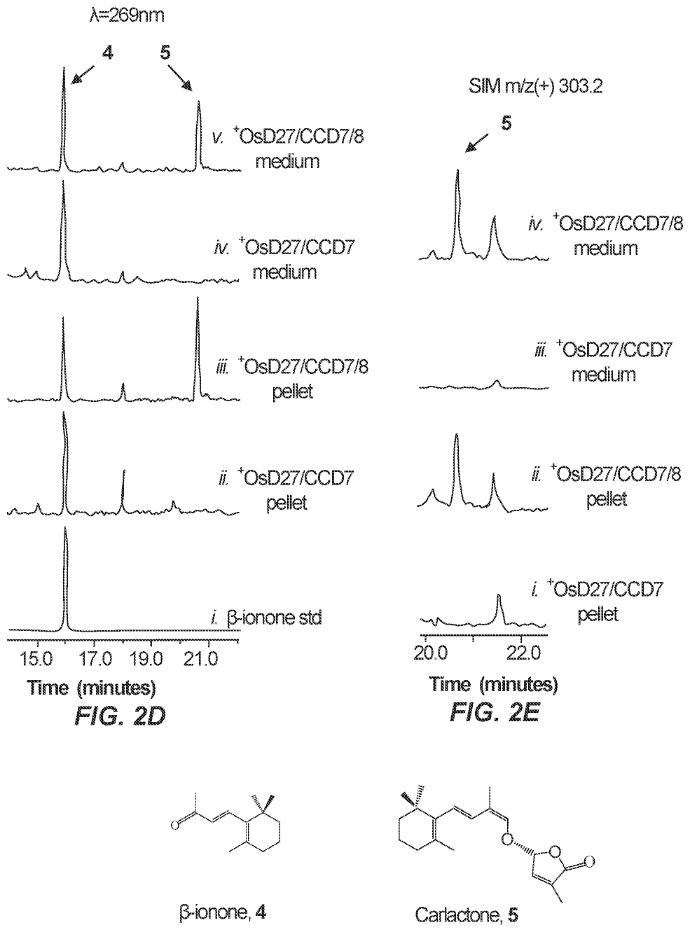

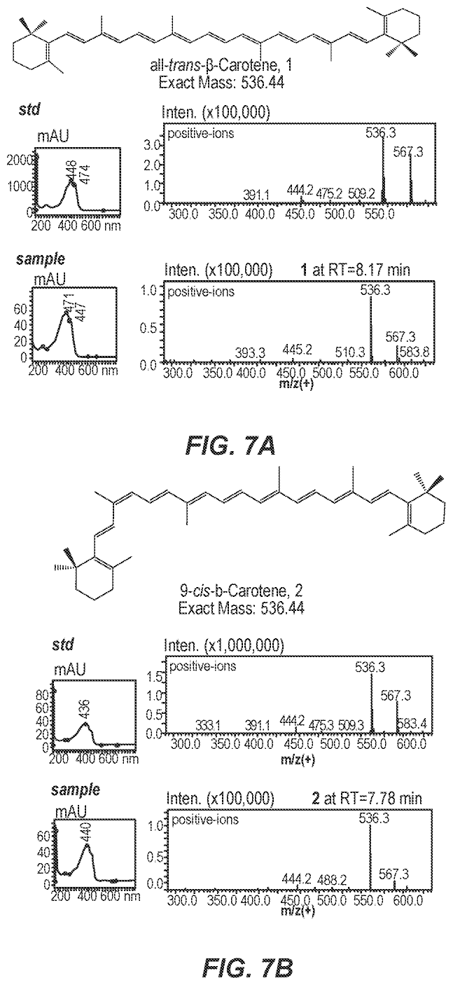

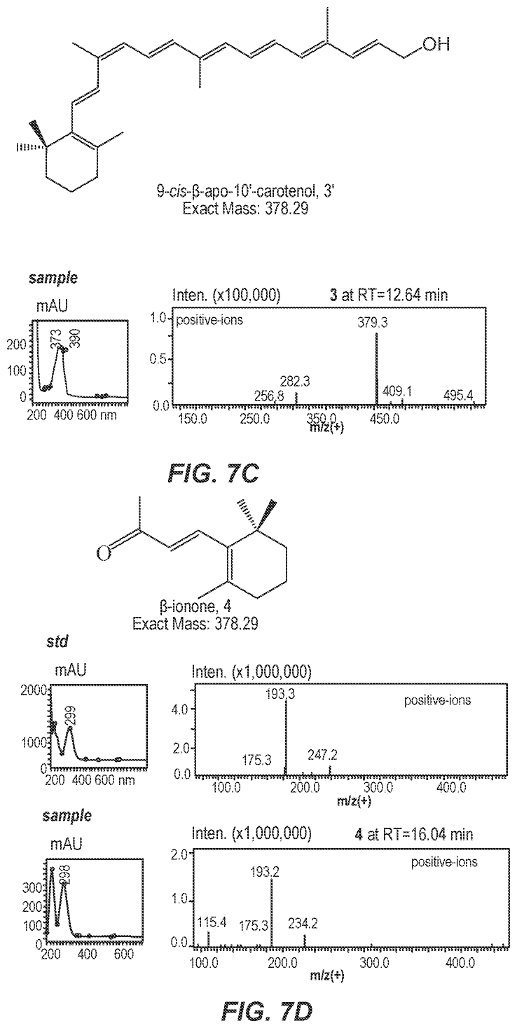

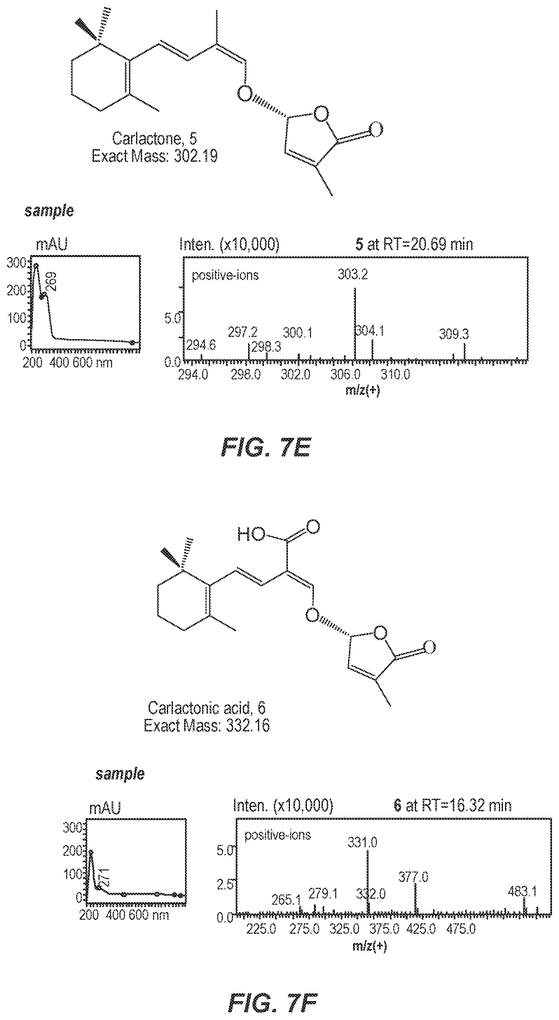

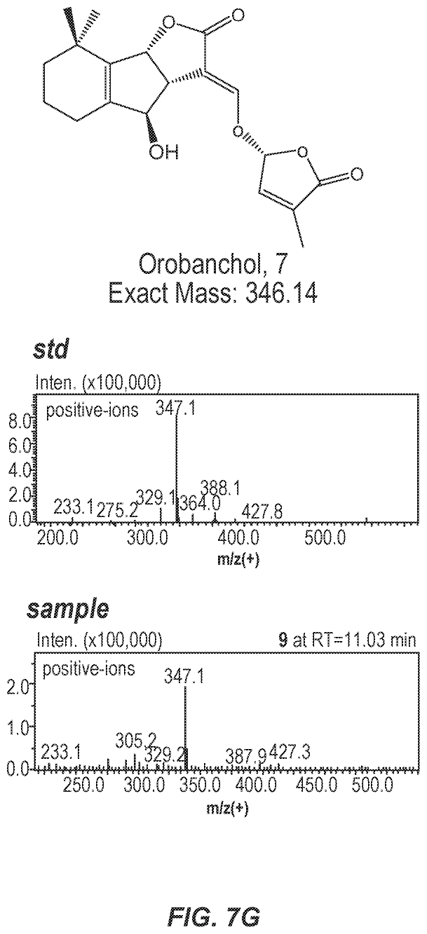

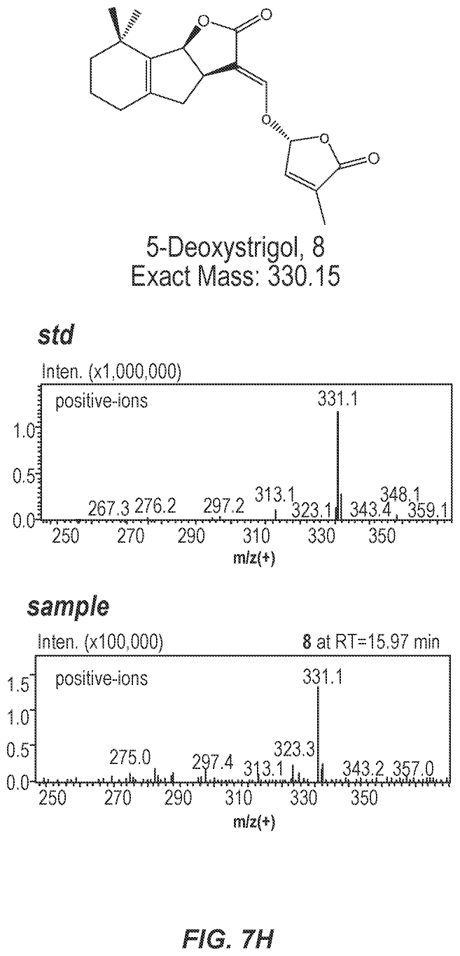

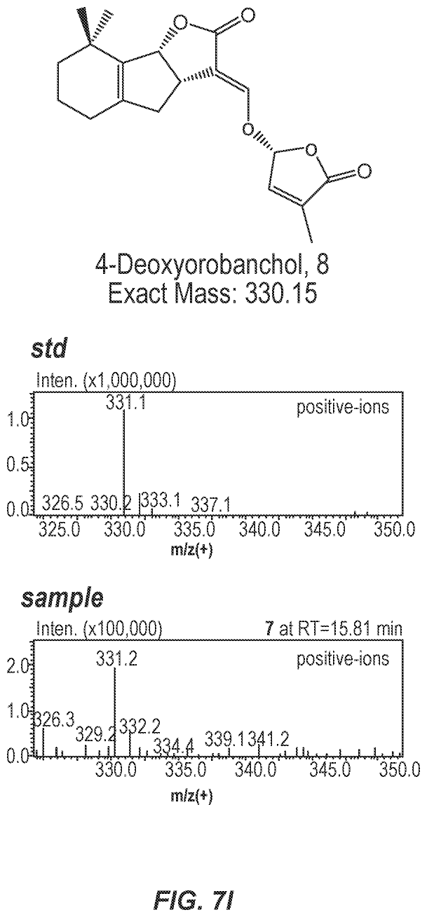

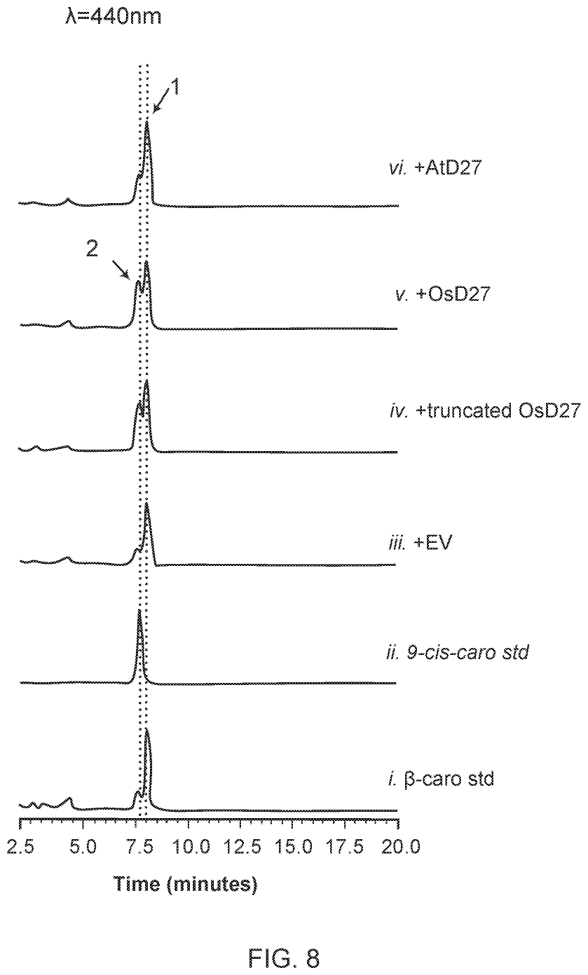

Previous investigations indicate that D27, CCD7, and CCD8 can be expressed and isolated in soluble form from E. coli for the in vitro biochemical investigations (34). Thus, we shifted the in vivo CL production from yeast to E. coli. First, OsD27 was expressed from a medium-copy number plasmid pCDFDuet (Table 1) in E. coli, under the control of T7 promoter, in the presence of the well-documented 0-carotene-producing plasmid pAC-BETAipi (Table 1). Upon the introduction of OsD27, the ratio between 9-cis- to all-trans-β-carotene was increased from ˜1:4.13 to ˜1:1.28, which indicates the functional reconstitution of OsD27 in E. coli (FIG. 2A, FIG. 7A, 7B). In the presence of OsD27, the titer of all-trans-carotene was estimated to be ˜1.80 mg/L and the titer of 9-cis-β-carotene ˜1.41 mg/L. The activity of D27 from A. thaliana was also examined in E. coli, which exhibited a similar level of activity (FIG. 8). As aforementioned, D27 is plastid-localizing enzyme, and the N-terminus plastid signal peptide may negatively affect the enzyme activity. Thus, we truncated the putative chloroplast transit peptide (first 40 amino acids) from OsD27 but did not detect obvious enhancement in the conversion towards 9-cis-β-carotene (FIG. 8). Previous investigation on D27 indicated that this β-carotene isomerase is a reversible enzyme that catalyze either directions between 9-cis- and all-trans-β-carotene (34), and thus the ratio between 9-cis- to all-trans-β-carotene is not likely to much more than 1:1.

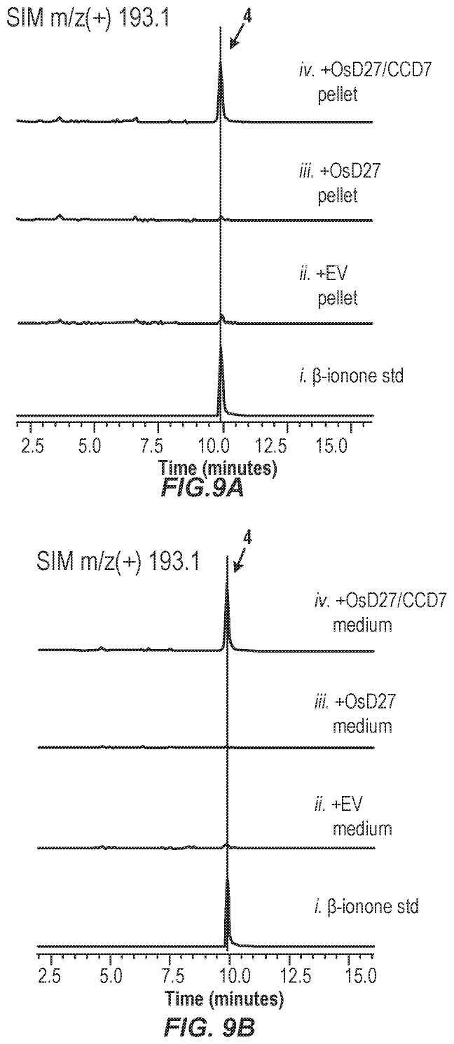

Subsequently, CCD7 from A. thaliana (AtCCD7) was introduced to the 9-cis-β-carotene-producing E. coli strain from the same medium-copy number plasmid pCDFDuet (Table 1), under the control of T7 promoter. According to previously reported heterologous expression efforts (34), the putative chloroplast transit peptide (first 31 amino acids) was truncated from AtCCD7 (tCCD7). Liquid chromatography-mass spectrometry (LC-MS) analysis indicated the synthesis of a new compound with m/z⁺=379.3 that agreed with 9-cis-β-apo-10′-carotenol, 3′ (FIG. 2B, C and FIG. 7C) (43, 44). Although the product of CCD7 in vitro is 9-cis-β-apo-10′-carotenal (19), the aldehyde can be reduced to the corresponding alcohol, 9-cis-β-apo-10′-carotenol, in E. coli (43, 44). The other product of CCD7, β-ionone, was confirmed by LC-MS analysis through comparison with the authentic standard (FIG. 7D, FIG. 9). No 9-cis-β-apo-10′-carotenol (FIG. 2C) but a trace amount of β-ionone was detected by LC-MS in the negative control of 9-cis-β-carotene-producing E. coli strain (FIG. 9), and the β-ionone is believed to be produced by the autoxidation of β-carotene (45).

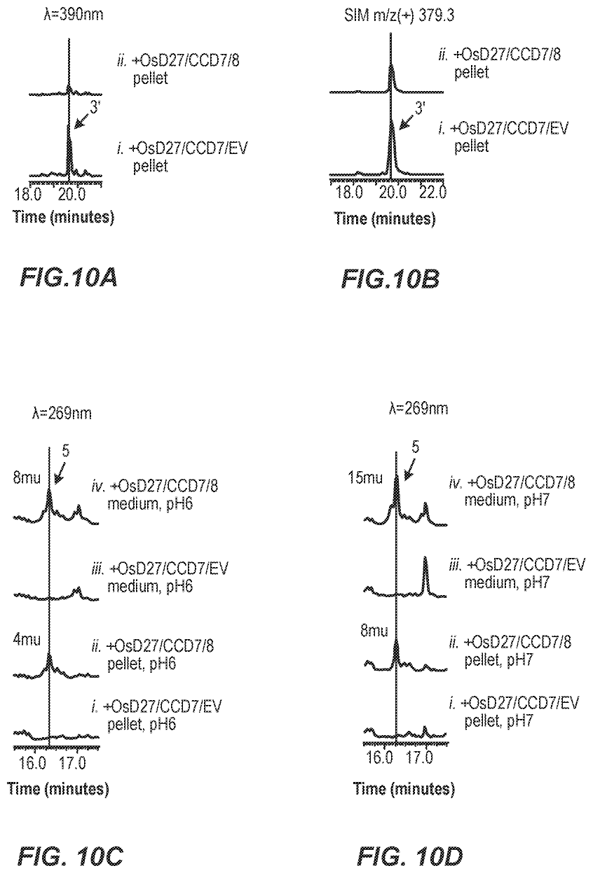

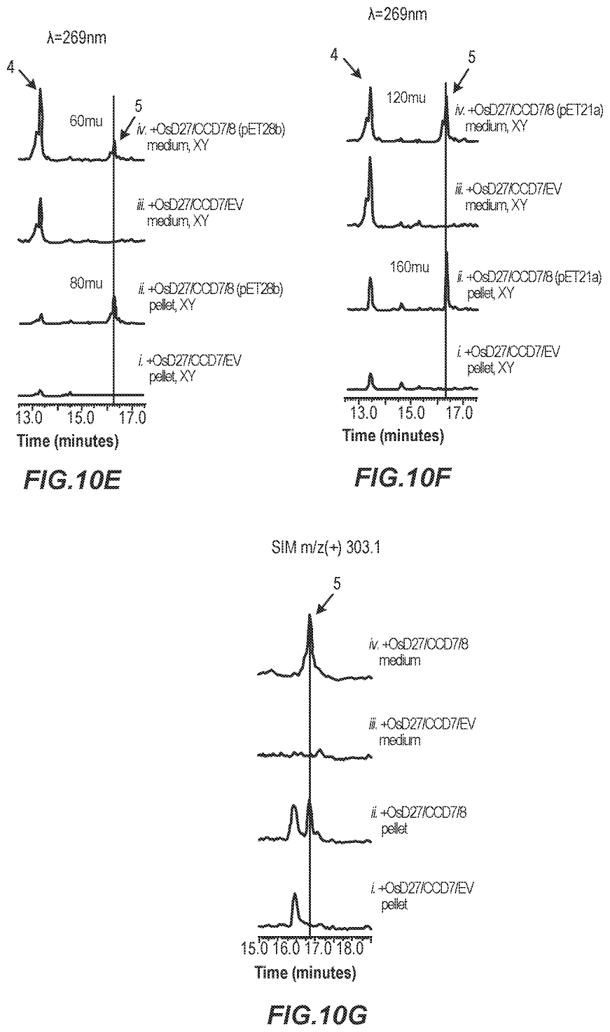

Similarly, the N-terminus 56 amino acids of AtCCD8 were also removed (tCCD8) and introduced into the 9-cis-β-apo-10′-carotenol-producing E. coli strain from a medium-copy number plasmid pET21a under the control of T7 promoter, as well. Although a drastic decrease in 9-cis-β-apo-10′-carotenol was detected upon the introduction of AtCCD8, we were unable to detect the synthesis of CL (FIG. 10A, 10B). Previous report implies that CL is unstable, and that pH, solvent composition, and temperature can affect its chemical stability (46). We added 1:2 (volume of buffer:volume of medium) 200 mM HEPES buffer (pH 7.0) to the growth medium upon IPTG induction, which led to the detection of a new tiny peak in both the cell pellets and the culture medium at RT=20.6 min with m/z⁺=303.2, which agrees with that of CL (FIGS. 7E, and 10C, 10D). However, the yield of CL was low, which is not proportional to the decrease of 9-cis-β-apo-10′-carotenol. Thus, we tried different medium conditions for the detection of CL. Under the optimum fermentation conditions (XY medium, FIG. 10E-10G), a distinguished peak with the same retention time as the putative CL was observed (FIG. 2D, E, and FIG. 7E), which showed an absorption at the maximum at 269 nm (FIG. 7E), consistent with the previously reported spectra data of CL (19, 34). High resolution MS indicates that the molecular formula of the compound C₁₉H₂₆O₃, which is consistent with CL.

Synthesis of CLA in E. coli-S. cerevisiae Co-Culture

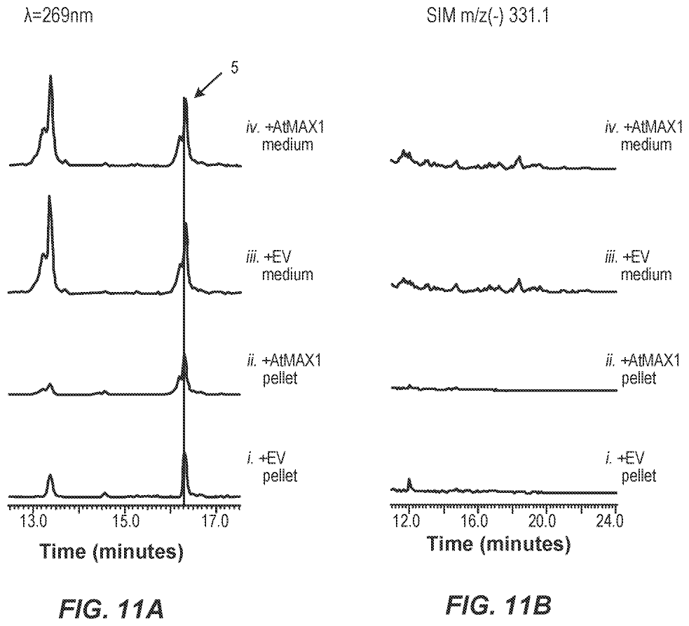

According to the pioneering in planta study, most of the canonical SLs are branched downstream of CLA, which is converted from CL with the function of MAX1 (22). However, further introduction of truncated MAX1 and cytochrome P450 reductase from A. thaliana (AtMAX1 and ATR1, respectively) on a signal polypeptide in the CL-producing E. coli did not convert CL towards CLA (35) (FIG. 11). While most of the cytochrome P450s that have been successfully reconstituted in E. coli are plastid-localizing P450s (47), AtMAX1 is an endoplasmic reticulum (ER)-localizing enzyme. The eukaryotic S. cerevisiae is thus likely a more suitable organism to reconstitute the activity of these ER-localized cytochrome P450s (48). Since CL can be detected in both cell pellets and culture medium, it is possible to establish the synthesis of downstream SLs using the E. coli-S. cerevisiae coculture strategy, with which CL is expected to be translocated from E. coli to the yeast for further functionalization.

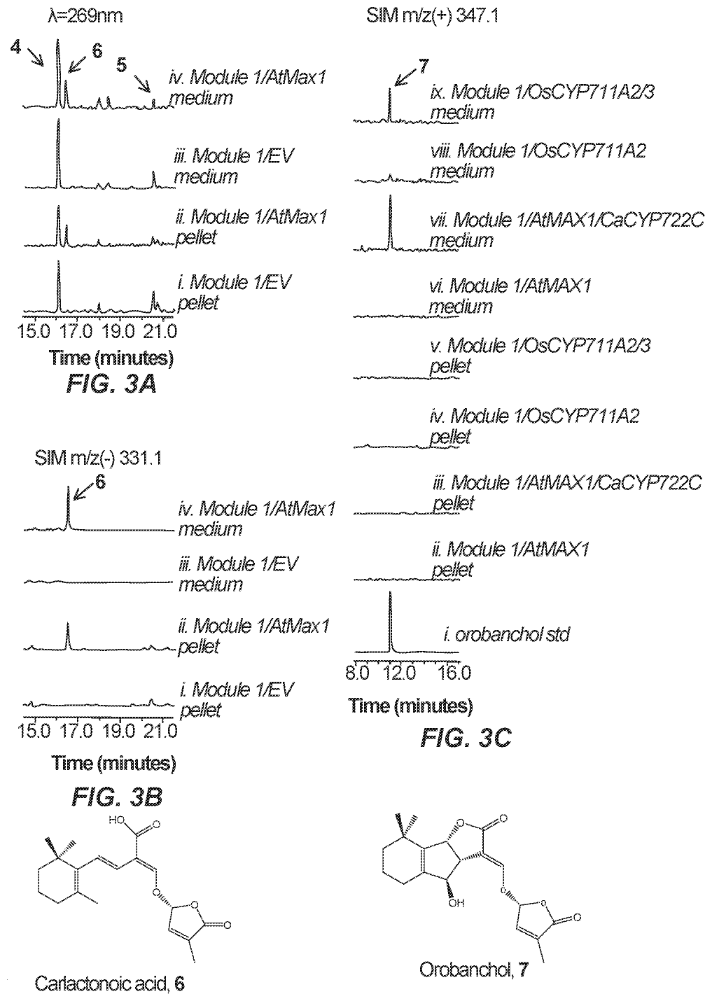

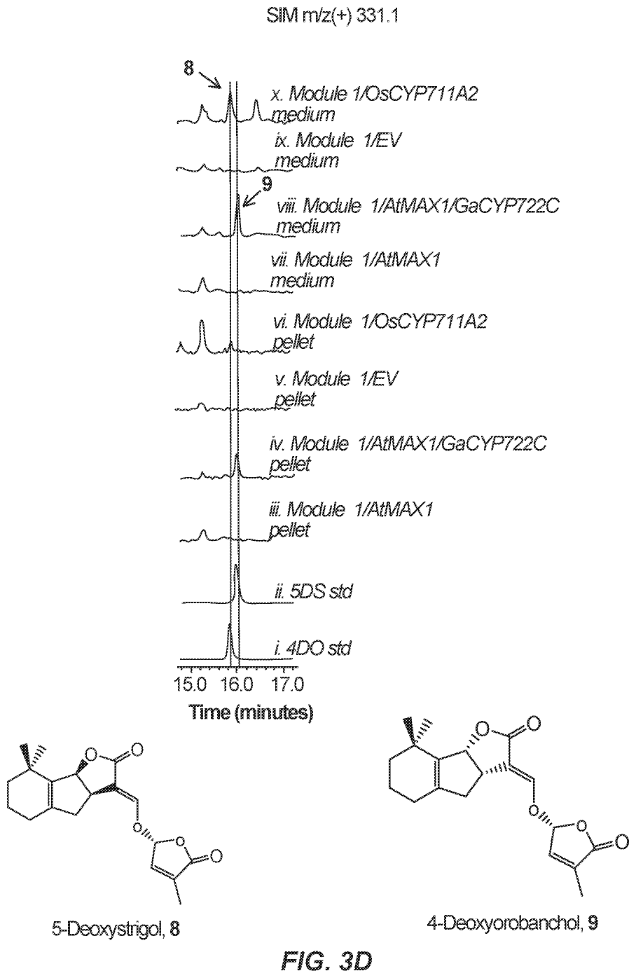

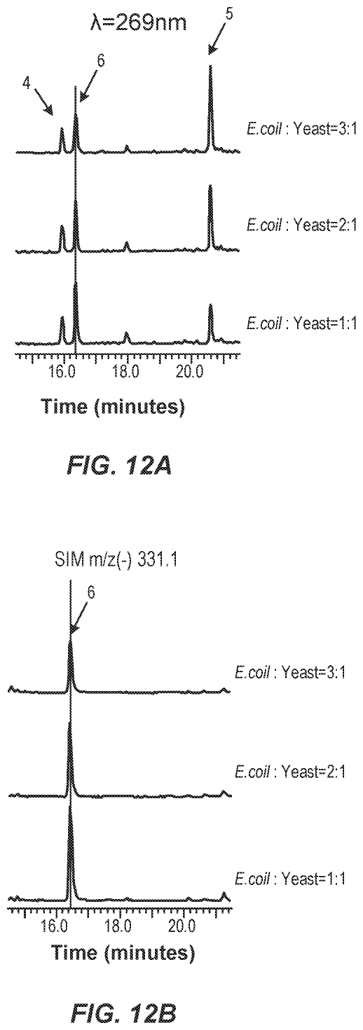

AtMAX1 and ATR1 were then introduced to S. cerevisiae on low-copy number plasmids and expressed downstream of PGK and TEF1 promoter, respectively. When CL-producing E. coli strain was co-cultured with yeast strain expressing ATR1 and AtMAX1, the peak of CL significantly decreased, and a new peak was detected in the organic extract of both cell pellets and medium under UV detection (FIG. 3A). The UV-VIS spectrum of the new peak has the maximum absorption at 271 nm, with [M−H]⁻=331.1 matching those of CLA (FIG. 7F, FIG. 3B). We then tried to improve the yield of CLA by adjusting the inoculum ratio between the CL-producing E. coli and AtMAX1/ATR1-expressing S. cerevisiae. We found that increasing the amount of yeast led to decreased titer of CL and enhanced titer of CLA, which indicates increased conversion of CL towards CLA (FIG. 12). The ratio between CLA to CL has risen from ˜0.4:1 to ˜1.6:1, calculated based on the peak areas at 269 nm, with E. coli-yeast inoculum ratio changed from 3:1 to 1:1. We thus conducted the co-culture assay with E. coli-yeast volumetric ratio at 1:1 in follow up study.

Synthesis of Orobanchol Through 4DO Using CYP711 as in E. coli-S. cerevisiae Coculture

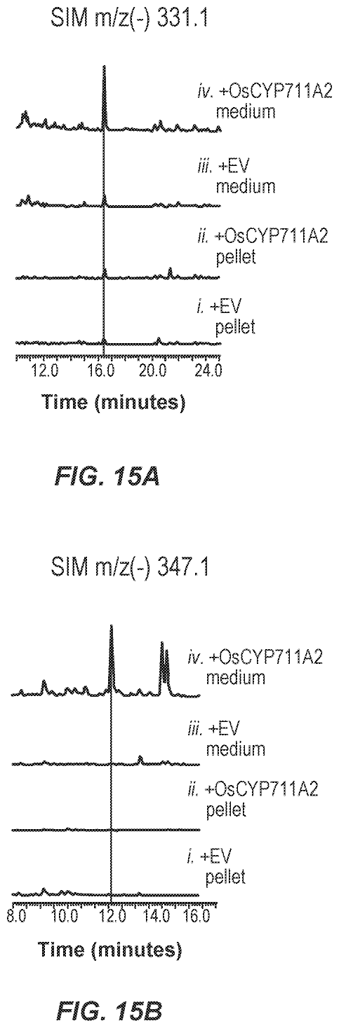

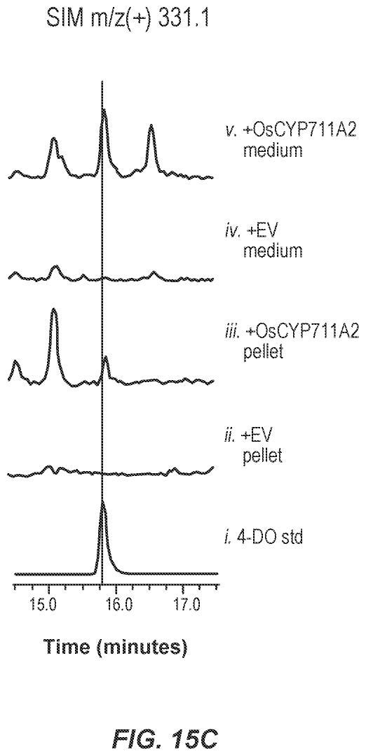

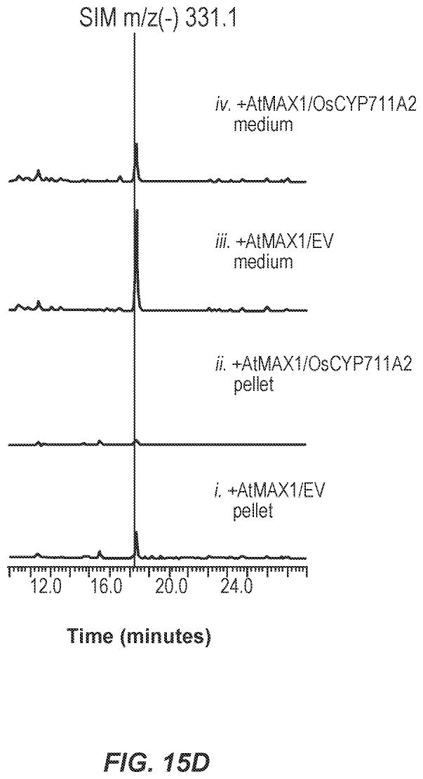

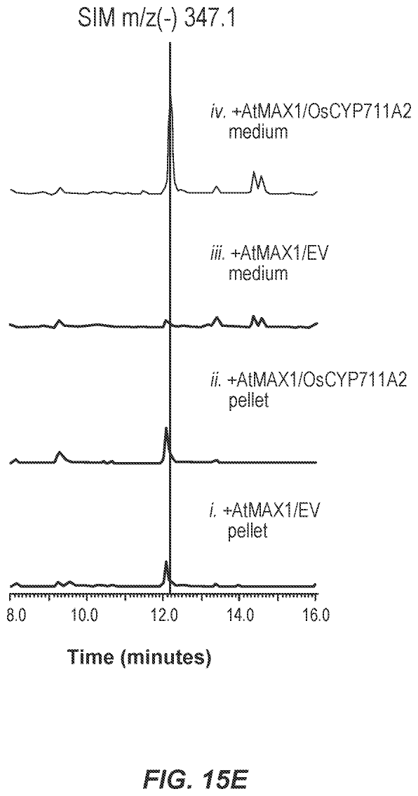

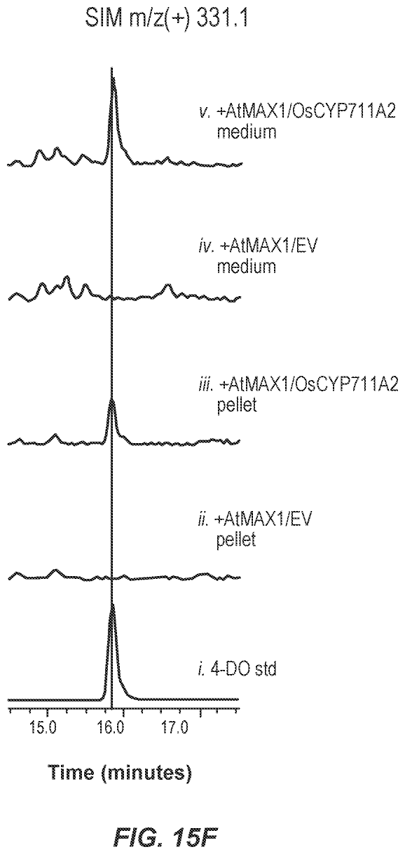

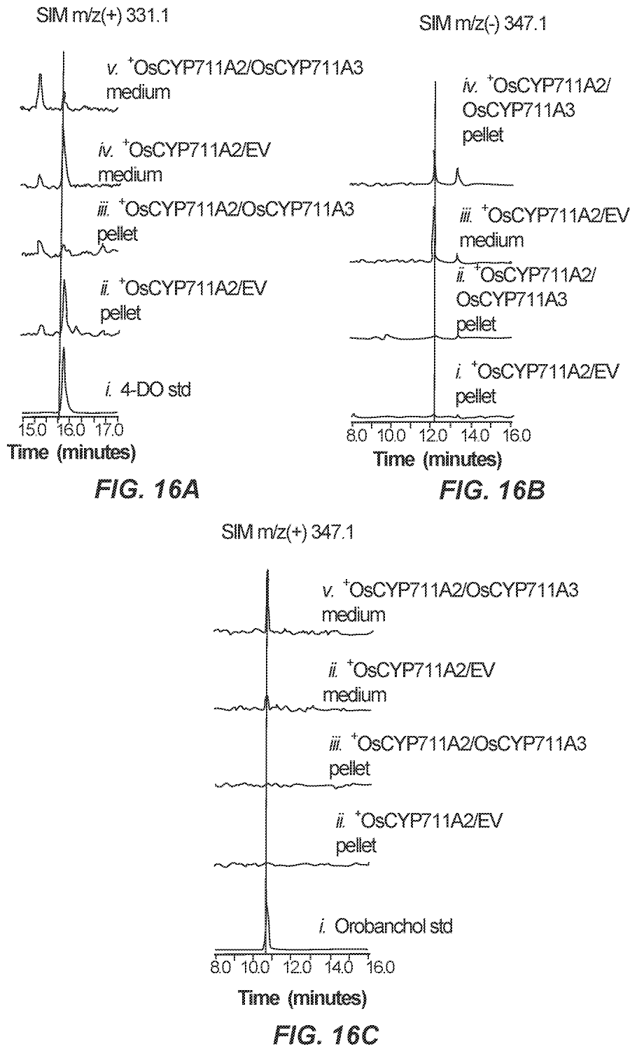

Different from the orobanchol only producing plants that directly synthesize orobanchol from CLA without converting through 4DO, rice (Oryza sativa L.) produce both 4DO and the hydroxylated product orobanchol (25), which indicates the catalytic capability of converting 4DO to orobanchol. The two MAX1 homologs, OsCYP711A2 encoded by Os900 and OsCYP711A3 encoded by Os1400, were identified to catalyze the conversion of CL to 4DO and 4DO to orobanchol, respectively (22, 26). According to the previous investigations, OsCYP711A2 alone can catalyze the formation of 4DO from CL (26). To synthesize 4DO in the microbial system, we codon-optimized and synthesized Os900 (OsCYP711A2 gene) and introduced the gene to the yeast strain expressing ATR1 using low-copy number plasmid downstream of TEF promoter. Trace amount of CLA and a new peak with m/z⁺ at 331.1 that matches with either 5DS or 4DO were detected (FIG. 3D). The retention time of the new peak is only 0.15 minute earlier than that of 5DS. The identity of the new peak was further confirmed to be 4DO through comparison of its retention time and mass spectrum with that of authentic 4DO and 5DS standards (FIG. 3D, FIG. 7I, FIG. 15A-15C). To explore the effect of adding AtMAX1 on 4DO producing, we introduced AtMAX1 to the above 4DO-producing yeast strain, i.e. co-culture the CL-producing E. coli with yeast strain expressing ATR1, AtMAX1 and OsCYP711A2, which led to the same results as in the absence of AtMAX1 (FIG. 15D-15F). Our results imply that OsCYP711A2 not only can catalyze the oxidation at two carbon positions (C18 and C19) of CL, but also can catalyze the BC-ring closure, which is consistent with previous studies (26). The titer of 4DO in the consortium using CYP711A2 was 3.46±0.28 μg/L.

Furthermore, we introduced codon-optimized Os1400 (OsCYP711A3 gene) to the 4DO-producing consortium on a low-copy plasmid downstream the GPD promoter. As expected, the addition of OsCYP711A3 significantly decreased the 4DO peak and synthesized a new peak that is consistent with the authentic orobanchol standard (FIG. 3D, and FIG. 7G). The successful reconstitution of orobanchol synthesis from CL in the microbial consortium using CYP711A2 and CYP711A3 confirmed the previously proposed synthetic pathway of orobanchol in rice. Interestingly and similarly, we were also able to detect the synthesis of the putative 18-hydroxy-CLA in the medium of the 4DO-producing consortium. This peak exists in the medium of all the canonical SL-producing consortiums, which supports the hypothesis that 18-hydroxy-CLA is a common intermediate for the biosynthesis of canonical SLs. Our results also confirmed that orobanchol can be generated through two different routes using different set of cytochrome P450s. The titer of orobanchol in the orobanchol-producing consortium harnessing CYP711A2/CYP711A3 mechanisms was 0.75±0.01 μg/L.

Synthesis of Orobanchol Using CYP722C in E. coli-S. cerevisiae c-oculture

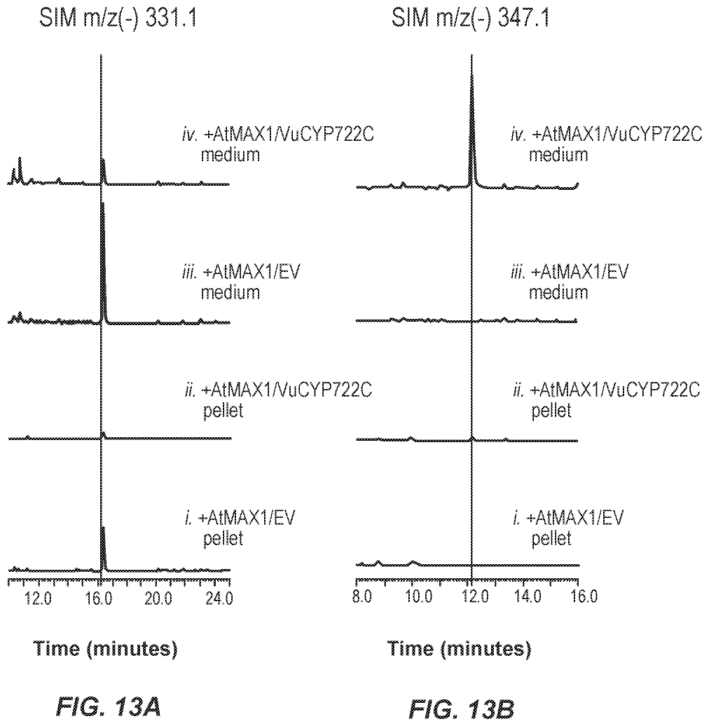

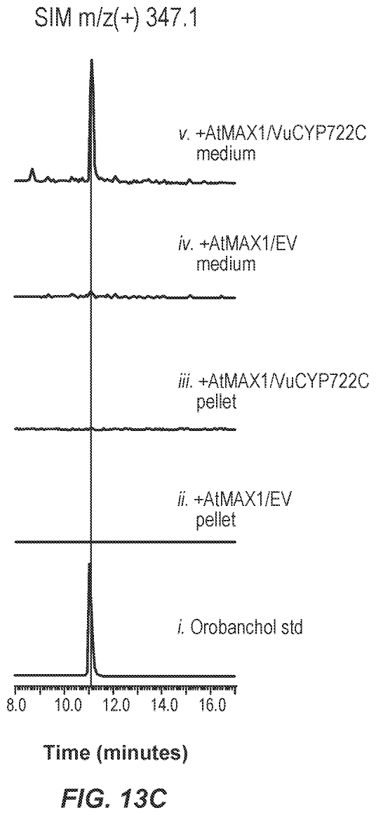

Previous investigations confirmed that the cowpea VuCYP722C can directly convert CLA to orobanchol and its diastereomer ent-2′-epi-orobanchol in vitro (29), which agrees with the fact that 4DO cannot be detected in many orobanchol-producing plants, such as red bell pepper (Capsicum annuum), red clover (Trifolium pratense L.), and cowpea (Vigna unguiculata (L.) Walp) (27). To produce orobanchol, we codon optimized and synthesized the gene encoding VuCYP722C (29), and introduced it to CLA-producing yeast strain on low-copy number plasmid downstream of the GPD promoter. The introduction of VuCYP722C to the CLA-producing yeast strain led to drastic decrease in CLA (FIG. 13A) and the synthesis of a new peak in the organic extract of the medium with m/z⁺=347.1 (FIG. 3C, and FIG. 7G), which agrees with that of orobanchol. The identity of the peak was further confirmed through comparison with the authentic orobanchol standard, which exhibits the identical retention time and mass spectrum (FIG. 3C, and FIG. 7G). Very few orobanchol was detected in the pellet which indicates that most orobanchol synthesized is exported into the medium by the engineered yeast strain. In addition to orobanchol, we were also able to detect another new peak in comparison to the negative control without the presence of VuCYP722C in the organic extract of the medium. The putative new peak showed m/z⁺ at 347.1(−) in the negative-ion mode FIG. 13B), according to chromatographic behavior, we speculate that this peak may putatively be 18-hydroxy-CLA (29). The titer of orobanchol in the orobanchol-producing consortium using VuCYP722C was 2.60±0.01 μg/L.

Synthesis of 5DS Using CYP722C in E. coli-S. cerevisiae Coculture

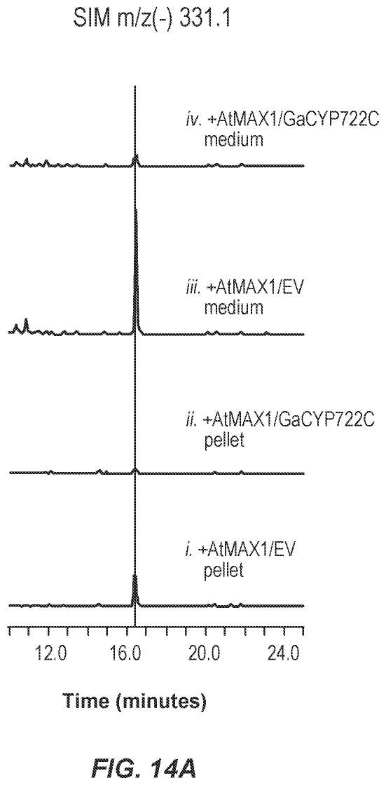

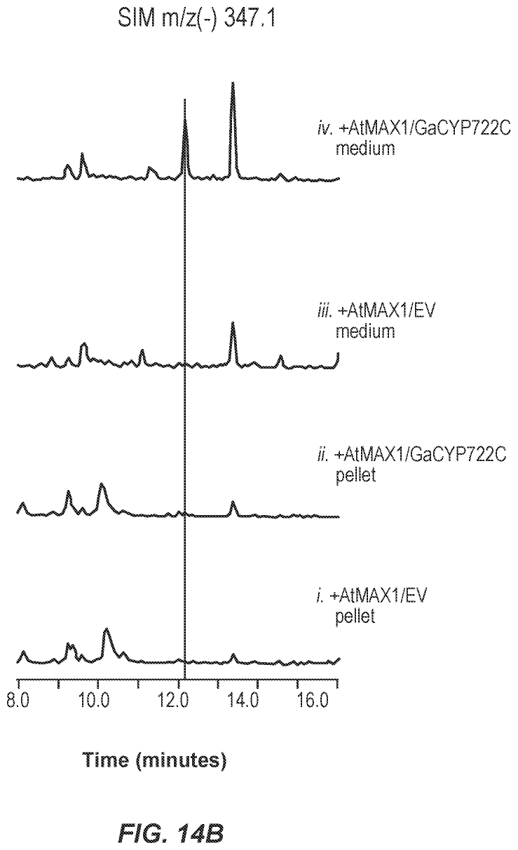

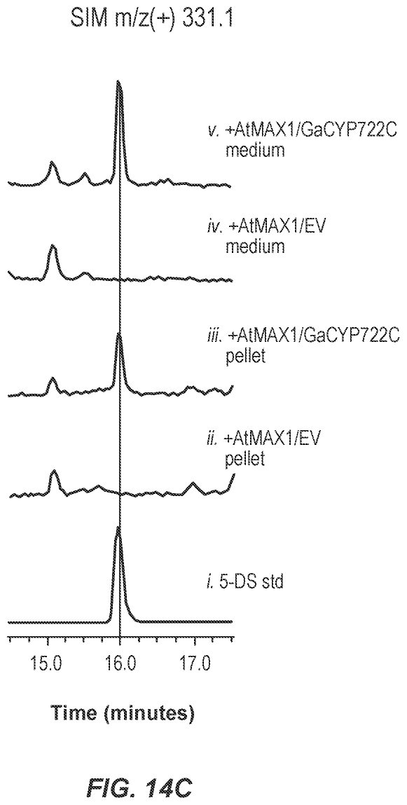

It is reported that cotton can produce both 5DS and the hydroxylated product, strigol (27). Later, GaCYP722C from cotton was reported to be responsible for the conversion of CLA to 5DS in vitro (31). To synthesize 5DS, we codon-optimized and synthesized GaCYP722C, and introduced it into yeast on a low-copy number plasmid downstream of GPD promoter. Upon the introduction of GaCYP722C to the CLA-producing yeast strain, we detected a drastic decrease of CLA (FIG. 14) with the synthesis of a new peak (RT=15.8 min) with a typical m/z⁺ at 331.1, which matches with that of 5DS. The identity of the peak was further confirmed by comparison of its retention times and mass spectrum with the authentic 5DS standard (FIG. 3D, and FIG. 7H). Different from orobanchol, 5DS was detected in the extract of both medium and pellet, which algins with the lower hydrophilicity of 5DS in comparison to orobanchol (FIG. 14). Similarly, the previously detected putative 18-hydroxy-CLA in the orobanchol-producing consortium was also detected in the medium of the 5DS-producing consortium. The titer of 5DS in the 5DS-producing consortium using GaCYP722C was about 6.65±1.71 μg/L.

### Functional Mapping of Various CYP722Cs

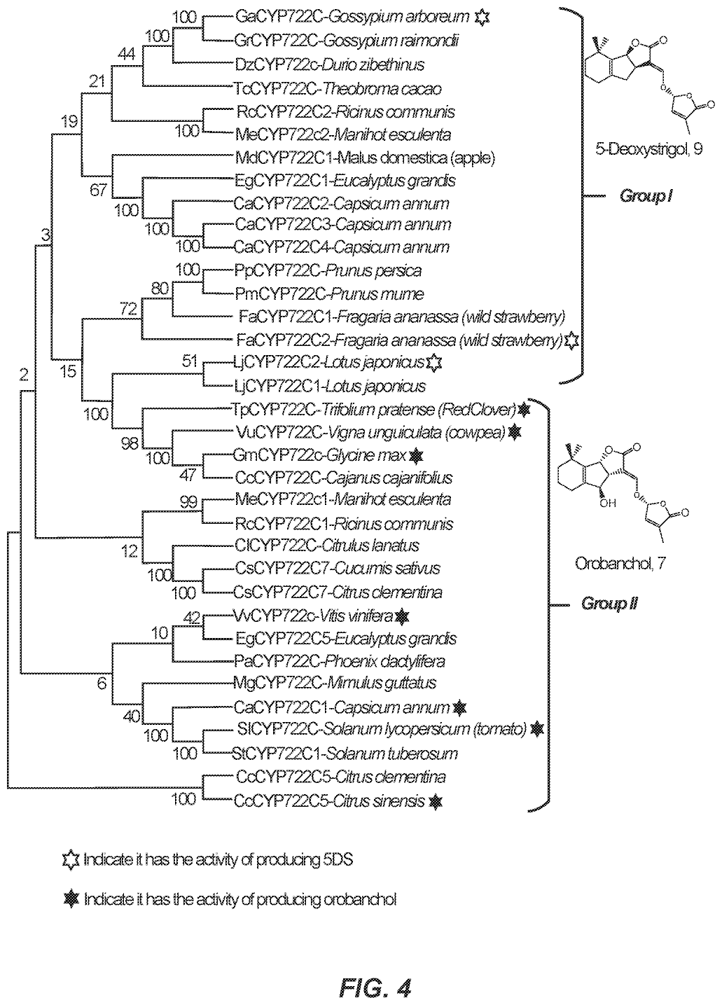

By using our co-culture expression system, we have confirmed that GaCYP772C and VuCYP722C are involved in the biosynthesis of 5DS and orobanchol, respectively. We next examined two other reported CYP722Cs (SlCYP722C and LjCYP722C) using our co-culture system. SlCYP722C is from tomato, which produce orobanchol and solanacol (orobanchol type SL) (49), CYP722C knockouts in tomato results in loss of these two canonical SLs but accumulation of CLA compared with wild-type (29). The recombinant SlCYP722C can catalyze the same reaction as VuCYP722C (29). LjCYP722C (also named DSD) is from L. japonicus, a good model plant that can produce canonical (5DS) and non-canonical SL (lotuslactone) respectively (50). It is identified that LjCYP722C is responsible for the formation of 5DS by mutant screening (50). But its function has not been characterized (50). It is intriguing to investigate whether there is a new function for CYP722Cs or whether the enzymatic function of homologous proteins is conserved across different plant species have not been investigated. CYP722C genes are widely distributed in flowering plants. GaCYP722C share 65% amino acid identity with VuCYP722C, yet they catalyze different reactions. The successful functional reconstitutions of GaCYP722C and VuCYP722C in the microbial consortium hints the potential of using this biosynthetic platform to propose and establish a sequence-function correlation of CYP722Cs, which will enable us to predict the function of unknown CYP722Cs. To establish a sequence-function correlation of CYP722C, we first used GaCYP722C protein sequence as a query and performed BLASTp search, we selected a total of 28 CYP722C sequences from different plant species including dicotyledon and monocotyledons. Some of the selected CYP722C genes are from plants that have been reported to produce specified SLs (Table 3). For example, birdsfoot trefoil (Lotus japonicus) (24, 50), and strawberry (Fragaria x ananassa) were reported to produce 5DS (24), while cowpea (Vigna unguiculata) (24), red bell pepper (Capsicum annuum) (27), and red clover (Trifolium pratense) were reported to produce 4DO (24). We also included CYP722A and CYP722B sequences as the outgroup (FIG. 4). It is noteworthy that CYP722C cannot be found in the genome of Arabidopsis, which only encode CYP722B and CYP722A, respectively (50). This is consistent with the result that known canonical SLs can't be detected in Arabidopsis (51).

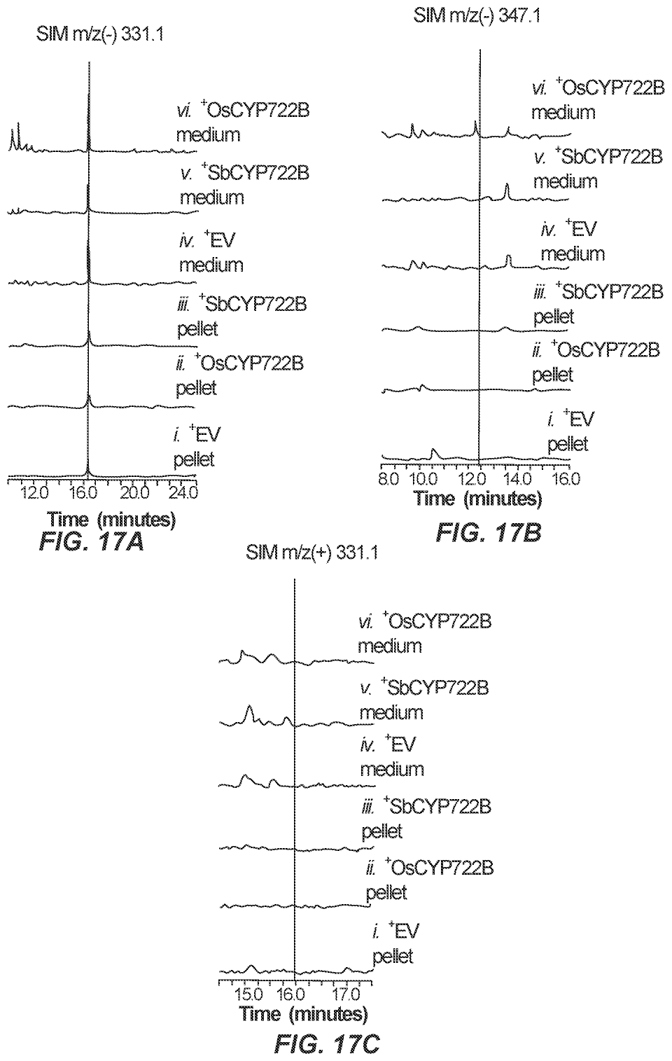

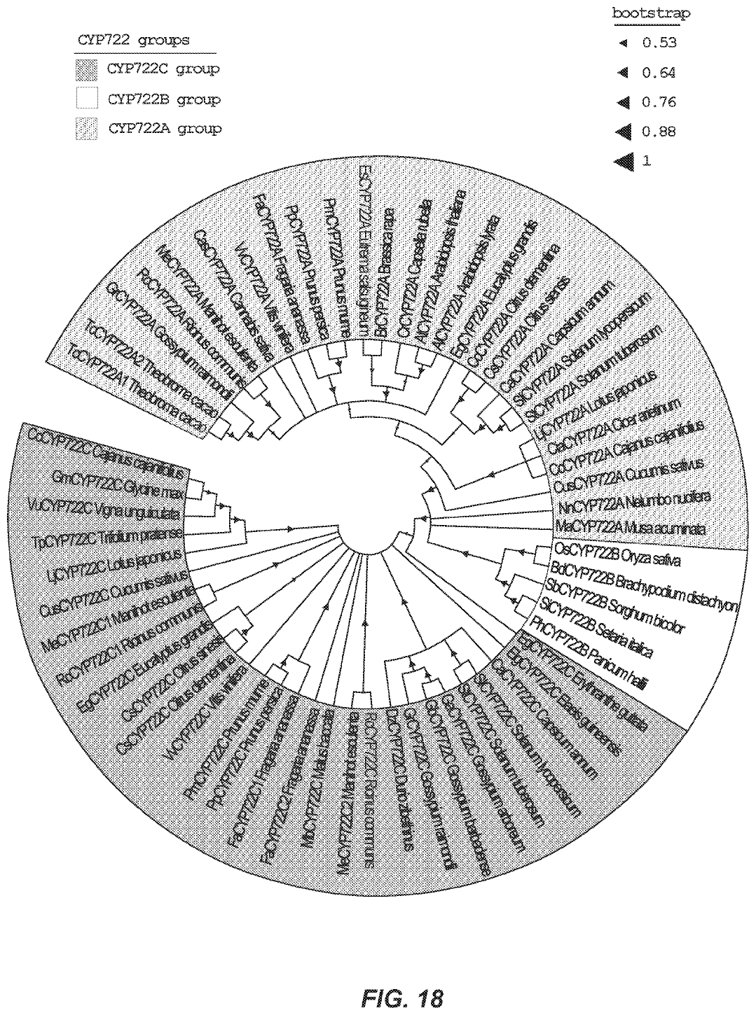

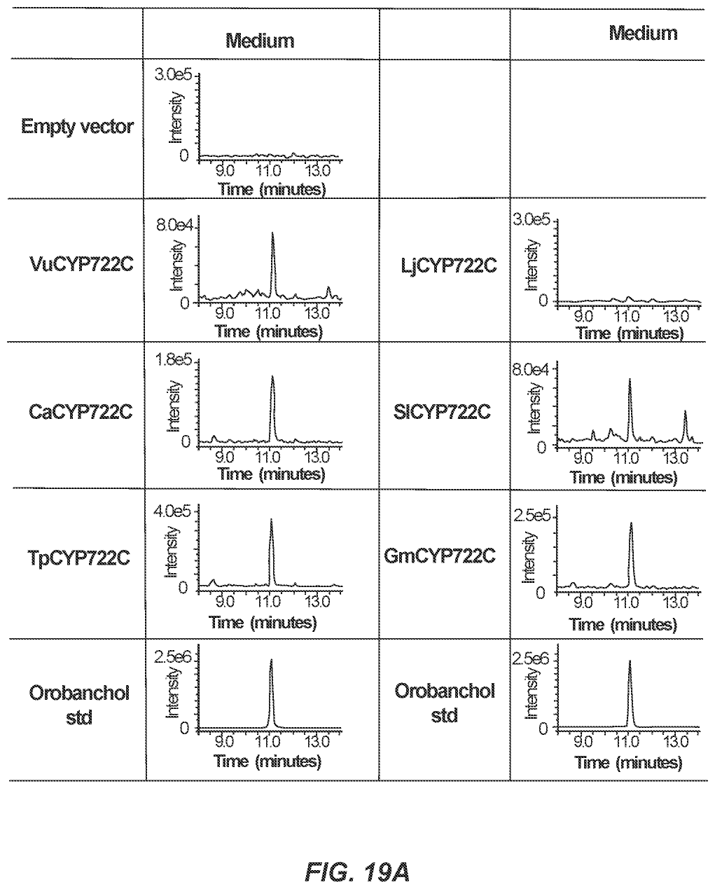

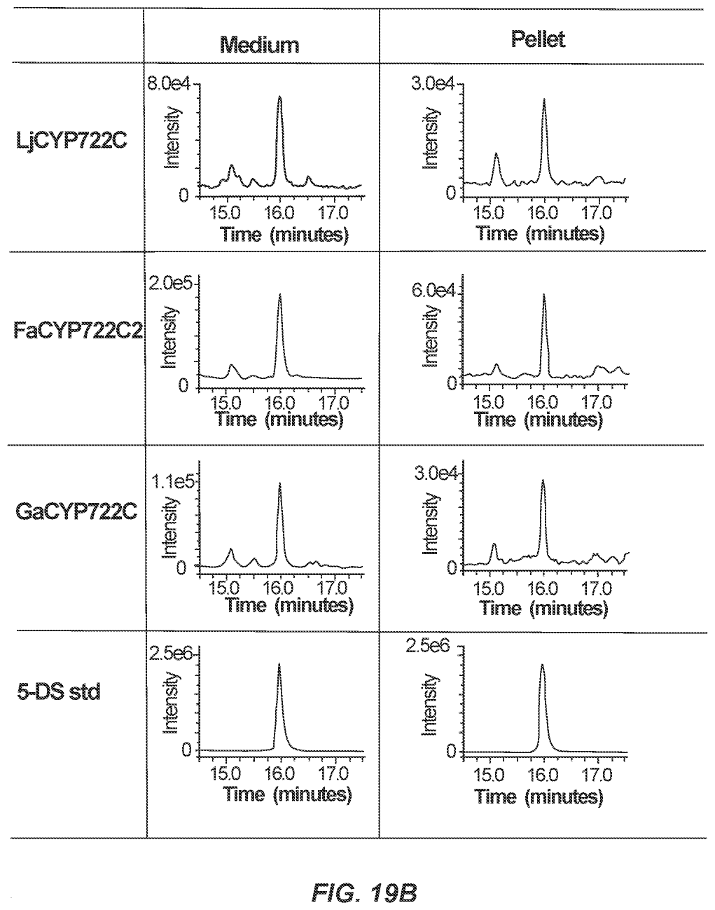

Phylogenetic analysis indicates that CYP722C subfamily can be divided into two groups (FIG. 4, FIG. 18): Group I and Group II. The speculative 5DS synthase LjCYP722C and characterized 5DS-producing enzymes GaCYP722C are members of Group I. The characterized orobanchol-producing enzymes VuCYP722C and SlCYP722C are members of Group II. To examine if we can simply predict the function based on the phylogenetic analysis, we synthesized 7 CYP722C genes from different branches and screened their functions by introducing each gene to the CLA-producing microbial consortium on a low-copy number plasmid regulated by GPD promoter. Among the 8 CYP722C genes we examined, CYP722C from Solanum lycopersicum, Capsicum annuum (Red bell pepper), Trifolium pratense (Red Clover), Glycine max, Citrus sinensis, and Vitis vinifera (SlCYP722C, CaCYP722C, TpCYP722C, GmCYP722C, CsCYP722C, VvCYP722C respectively) all belong to Group II converting CLA to orobanchol (FIG. 4); while CYP722C from Fragaria x ananassa (wild strawberry) and Lotus japonicus (FaCYP722C2 and LjCYP722C, respectively) belong to Group I synthesizing 5DS from CLA (FIG. 4, FIG. 19). Our work confirmed that LjCYP722C performs the same function with GaCYP722C as a 5DS synthase. OsCYP722B (Oryza sativa) and SbCYP722B (Sorghum bicolor) failed to convert CLA to either 5DS or orobanchol (FIG. 17). Our results on CaCYP722C, SlCYP722C, FaCYP722C₂, GmCYP722C, LjCYP722C, and TpCYP722C are consistent with the previously reported SL profiles from the corresponding plants (23, 24, 27), and indicate that Citrus sinensis and grape are capable to synthesize orobanchol though their SL profiles have not been reported.

The SL-profile of most plants are not reported. The phylogenic analyses and functional characterization of CYP722Cs from various plant species implies a sequence-function correlation, and provides an association between CYP722C sequences and SL synthetic capacity of the corresponding plants. If a plant encodes a group I CYP722C, it is able to produce 5DS-type SLs; while if a group II CYP722C is present, this plant can synthesize orobanchol-type SLs. Such sequence-function correlation may enable prediction on the SL synthetic capacity of a plant of interest.

Improving the Production of CL in E. coli

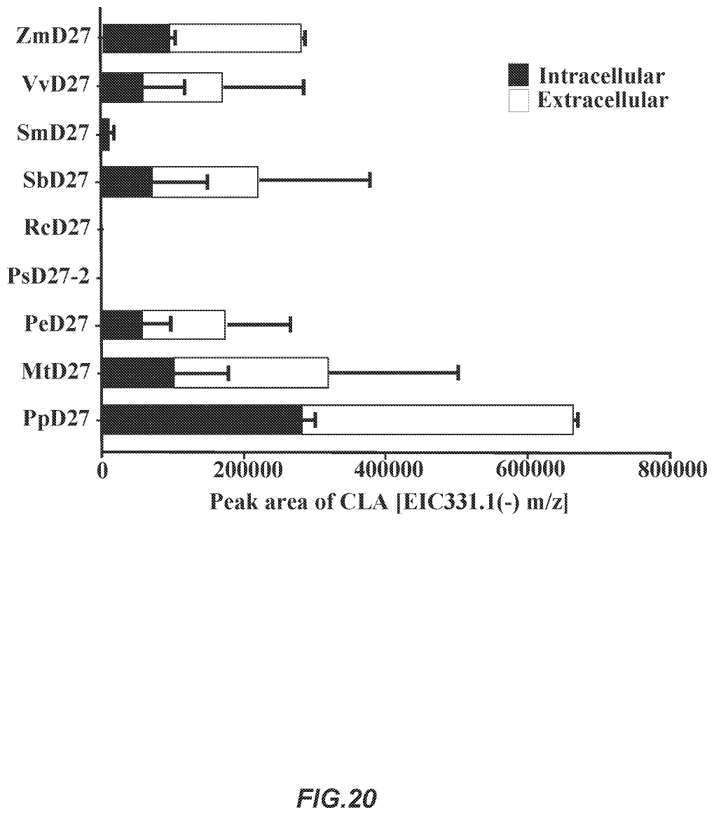

To improve the efficiency of D27, we screened several D27 homologs from different plant species in the CLA-producing E. coli-S. cerevisiae consortium (DNA sequence encoding protein are provided in Table 5. We identified a more efficient D27 variant (PpD27), resulting in higher CLA production (FIG. 20).

### Enhancing the Folding, Expression Level & Access to the Substrate of Each Enzyme

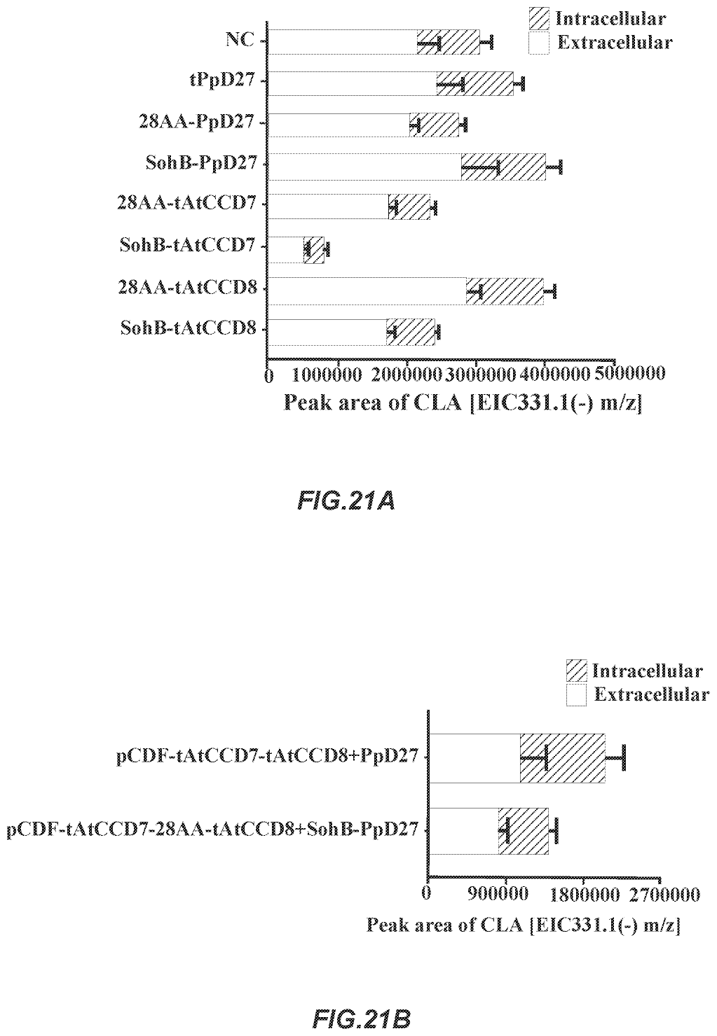

First, to enhance the activity of CL-producing enzymes (D27, CCD7, CCD8) in E. coli, we evaluated 2 different functional tags, 28aa tag and SohB, fused to the N-terminal of PpD27, tAtCCD7, and tAtCCD8; and truncated the chloroplast transit peptide of PpD27(tPpD27). SohB-PpD27 and 28AA-tAtCCD8 showed a significant increase in CLA production (FIG. 21A). DNA sequences encoding proteins are provided in Table 8. But when both D27 and CCD8 were replaced with SohB-PpD27 and 28AA-tAtCCD8, the CLA yield decreased (FIG. 21B).

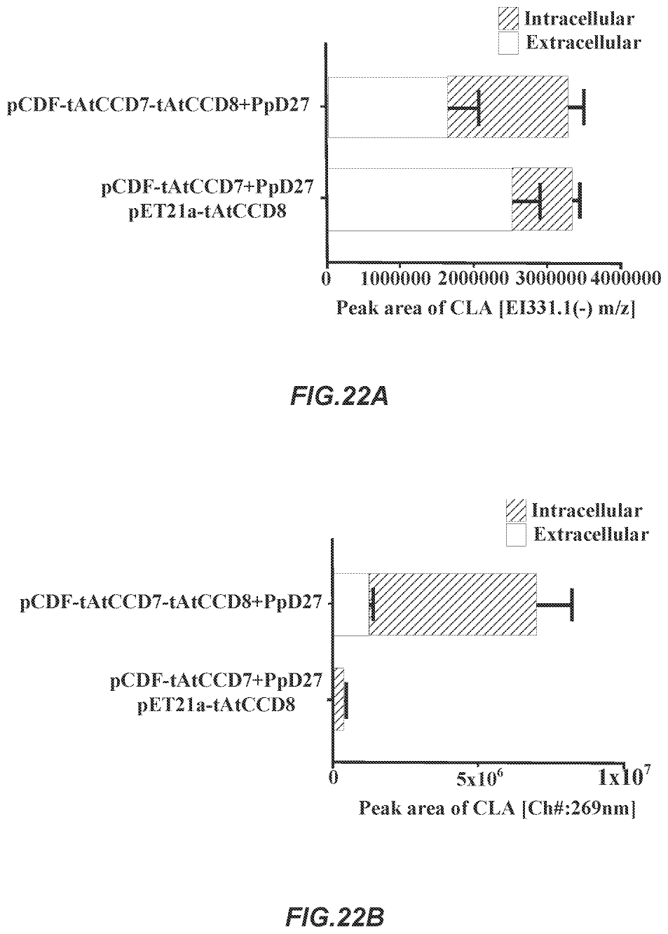

We also performed expierment to reduce the number of plasmids in E. coli. We assembled PpD27, tAtCCD7, tAtCCD8 into one single plasmid (pCDF-tAtCCD7-tAtCCD8+PpD27) by incorporating tAtCCD7 and tAtCCD8 into one operon. Using the resulting plasmid substantially improved CL production in E. coli, compared to using the two plasmid system (pCDF-tAtCCD7+PpD27 and pET21a-tAtCCD8) (FIG. 22).

### Enhancing the 5DS Titer to Mg/L Level in Yeast

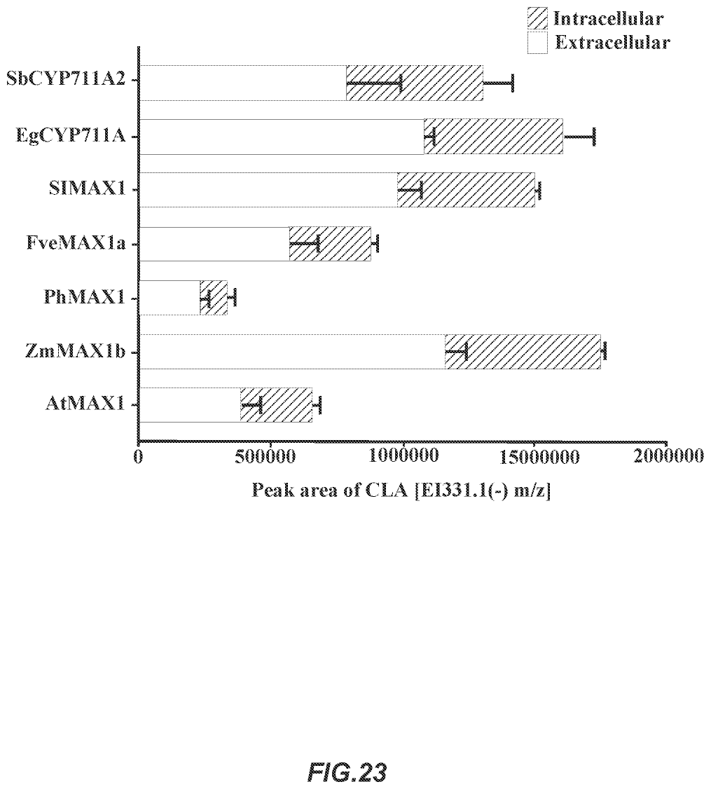

To identify MAX1 variant that performs well for an efficient CLA production, we compared the activity of 7 MAX1 analogs from different plant species in the CL-improved platform. DNA sequences encoding proteins are provided in Table 6. Among them, EgCYP711A and ZmMAX1b exhibited the highest activity towards CL production. FIG. 23.

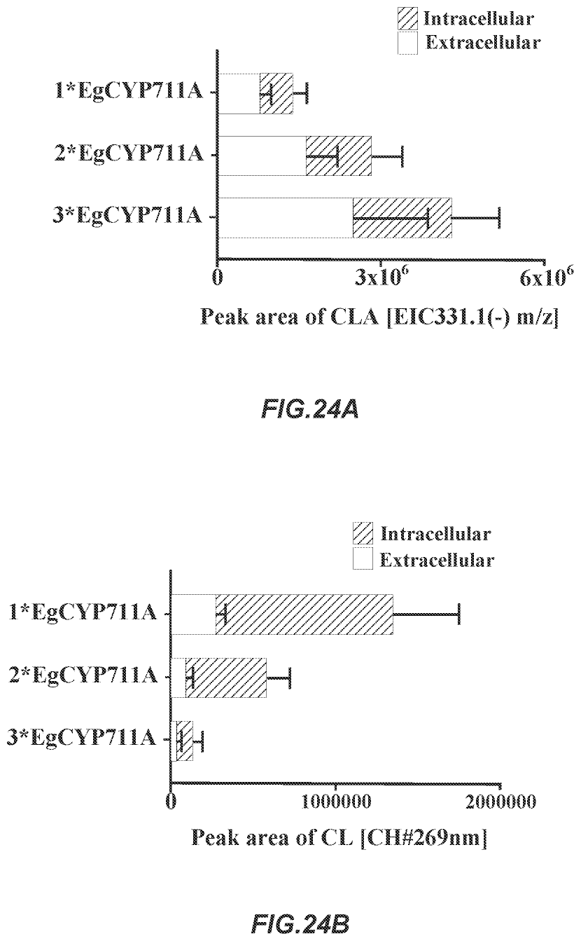

To further enhance the production of CLA, we also attempted to increase the copy number of EgCYP711A by introducing extra plasmids into the yeast strain. The yeast strain harboring three copies of EgCYP711A yielded highest CLA production in comparison to strains with two copies & single copy, suggesting that CLA production is directly proportional to the expression level of EgCYP711A in yeast. FIG. 24.

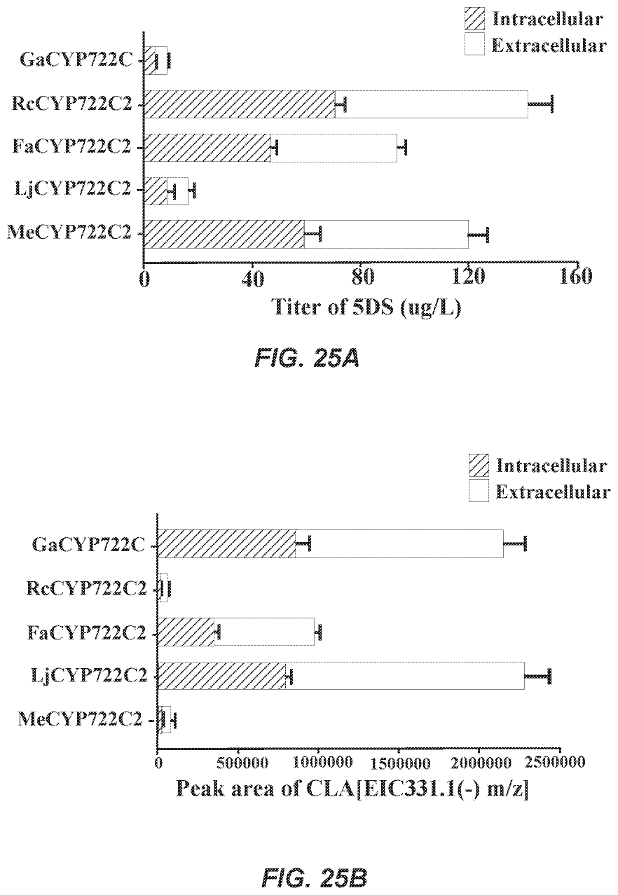

We also identified a high-performing CYP722 variant (RcCYP722C2, DNA sequences encoding proteins are provided in Table 8) by screening an array of analogs from different plant species for 5DS biosynthesis, which showed the best conversion efficiency with almost no CLA remaining. There was a 15-fold increase in 5DS titer compared to the original GaCYP722C. FIG. 25.

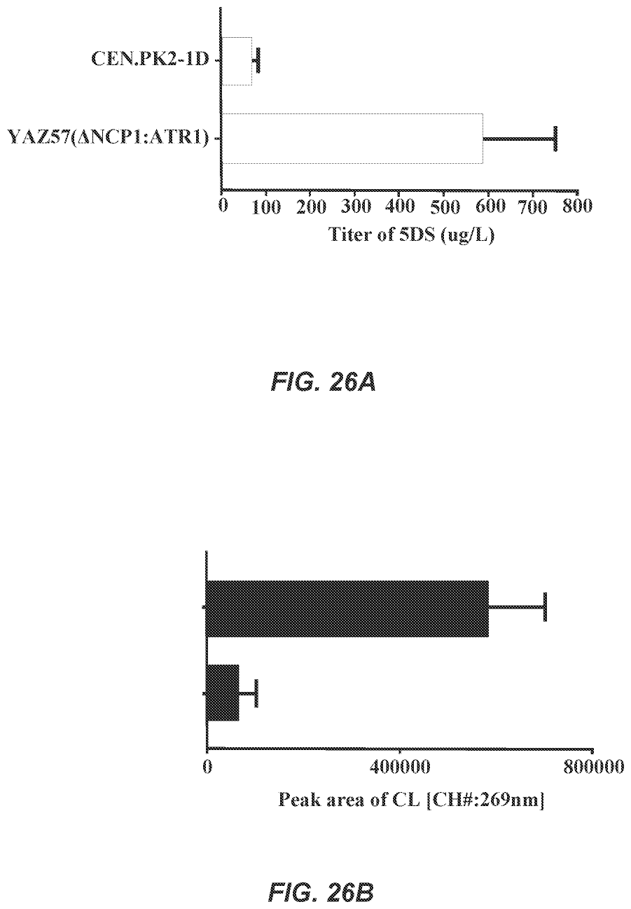

To further explore the CYP-CPR interaction to enhance catalytic activity of CYPs in yeast, we evaluated inactivation of the yeast endogenous CPR (ScCPR, DNA sequences encoding proteins are provided in Table 7) by replacing the NCP1 gene with AtCPR1 expression cassette. ScCPR has been found to possess low compatibility with plant CYPs and might interfere with the electron transfer between AtCPR1 and other plant-derived CYPs. The results showed that the ScCPR-inactivated & ATR1-integrated yeast strain (YAZ57) significantly enhanced the 5DS titer with a 7-fold increase than the wild type CEN.PK2-1D strain expressing the same CYP450s. FIG. 26.

### Improved Fermentation Conditions

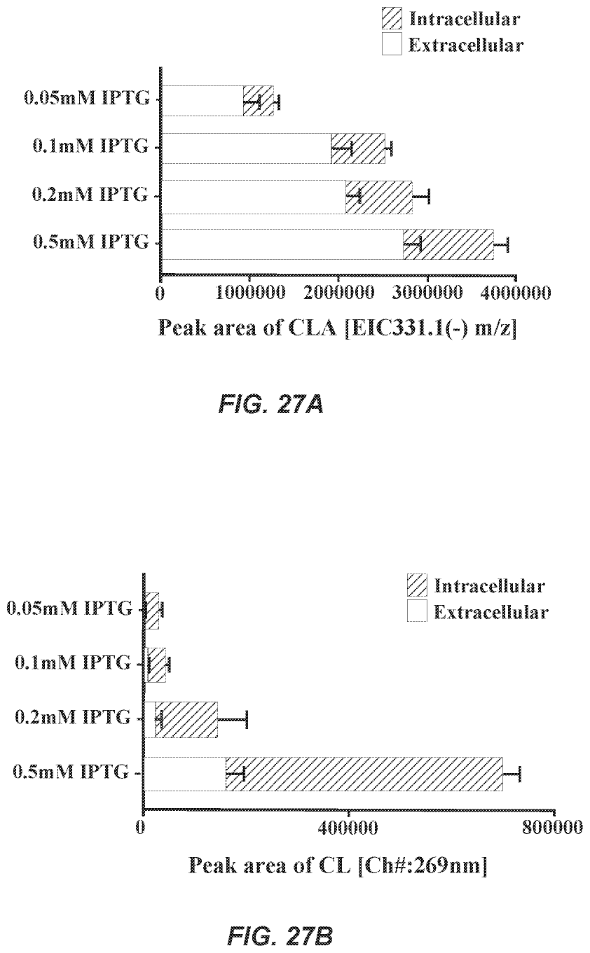

To optimize the culture condition, we determined an optimal IPTG concentration for inducible expression of enzymes in E. coli at the initial stage. The highest CLA production was achieved when 0.5 mM IPTG was used, which also led to a substantial accumulation of CL. FIG. 27.

In conclusion, in the experiments described above in the sections above, we have successfully reconstituted the biosynthesis of CL, CLA, and canonical SLs (4DO, 5DS, orobanchol) in an E. coli-yeast microbial consortium.

### Synthesis of 16-OH-CLA Using the Microbial Consortium

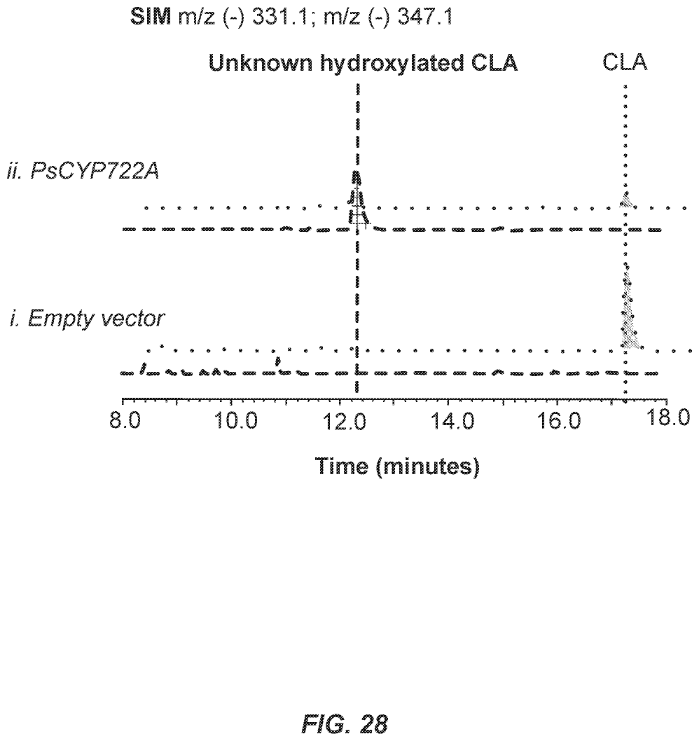

Based on the established E. coli-yeast co-culture platform, we successfully characterized the function of CYP722As, a CYP722 subfamily, which could produce an unknown SL derivative using CLA as substrate. According to LC-MS analysis, the mass and retention time of the unknown compound suggested that it may be a form of hydroxylated CLA that had not been identified previously. The unknown compound exhibits a negative mass/charge ratio (m/z⁻)=347.1, and can be synthesized by various plant derived CYP722As using CLA as substrate, such as PsCYP722A from Pisum sativum and AcCYP722A from Aquilegia coerulea. NMR analysis confirmed the identity of the compound as 16-OH-CLA. FIG. 28

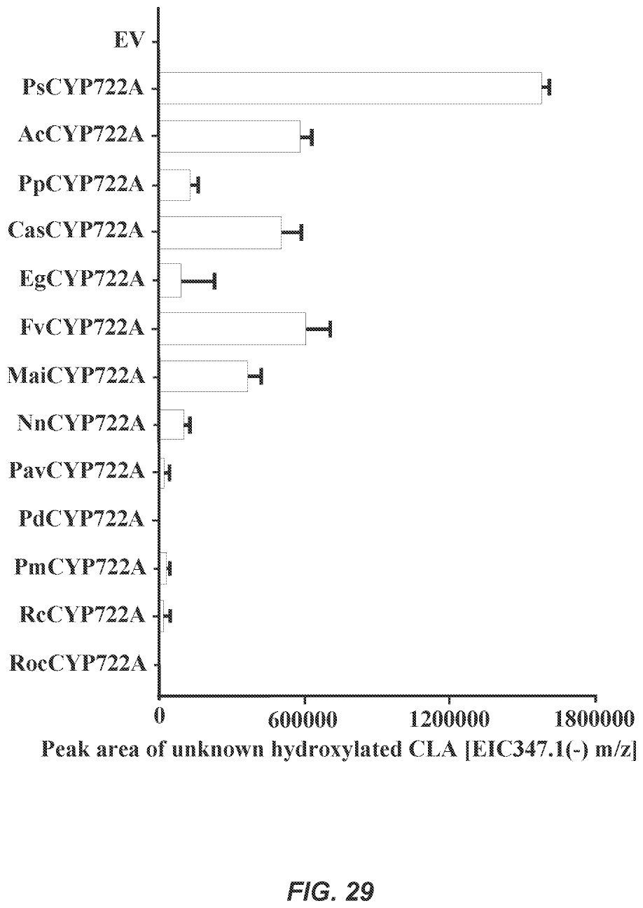

We tested various CYP722A proteins from different plant species (including but not limited to Cannabis sativa, Eucalyptus grandis, Fragaria vesca, Macadamia integrifolia, Nelumbo nucifera, Prunus mume, Prunus avium, Ricinus communis, Prunus persica, Pisum sativum, and Aquilegia coerulea), which all can convert CLA into 16-OH-CLA. Among them, PsCYP722A from Pisum sativum showed the highest enzymatic activity. FIG. 29

Synthesis of Strigol, SL-1 and SL-2 from the Microbial Consortia

Utilizing the established E. coli-yeast microbial consortium, we can also produce strigol, a previously uncharacterized oxidized 5DS (SL-1) and another previously unidentified hydroxylated CLA (SL-2). It was found that PpMAX1c from peach can produce CLA from CL and then convert CLA into strigol and an unknown compound SL-1 in the microbial consortium. Further investigation confirmed that SL-1 was an oxidation product of 5DS with a positive mass/charge ratio (m/z+)=347.1. PpMAX1b and SbMAX1c, on the other hand, can convert CLA into a new compound (SL-2) with m/z−=347.1 that agreed with the mass of a hydroxylated or oxidated CLA, and is different from t16-OH-CLA produced by CYP722As. FIGS. 30A,B

### Enhancement of Fermentation Conditions-16-OH-CLA

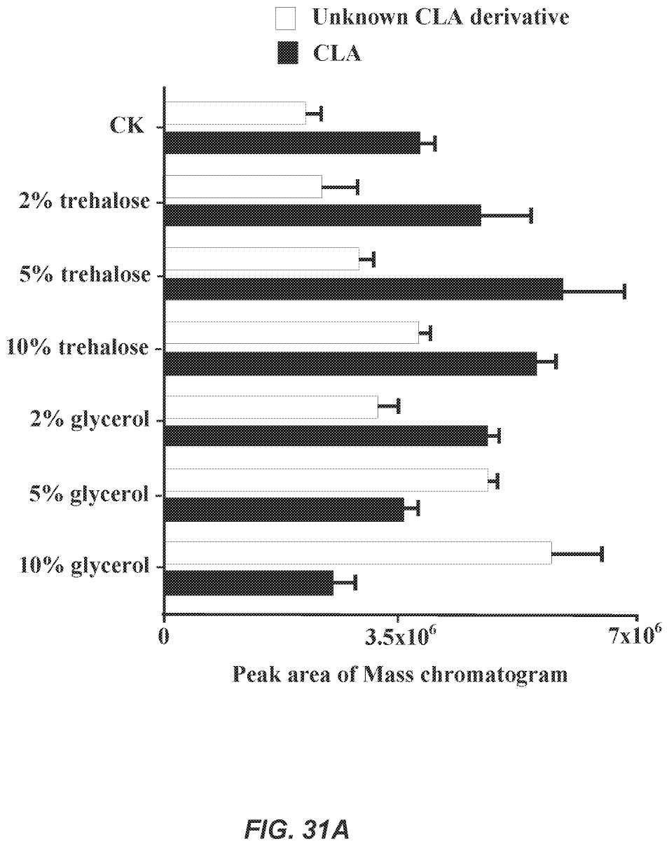

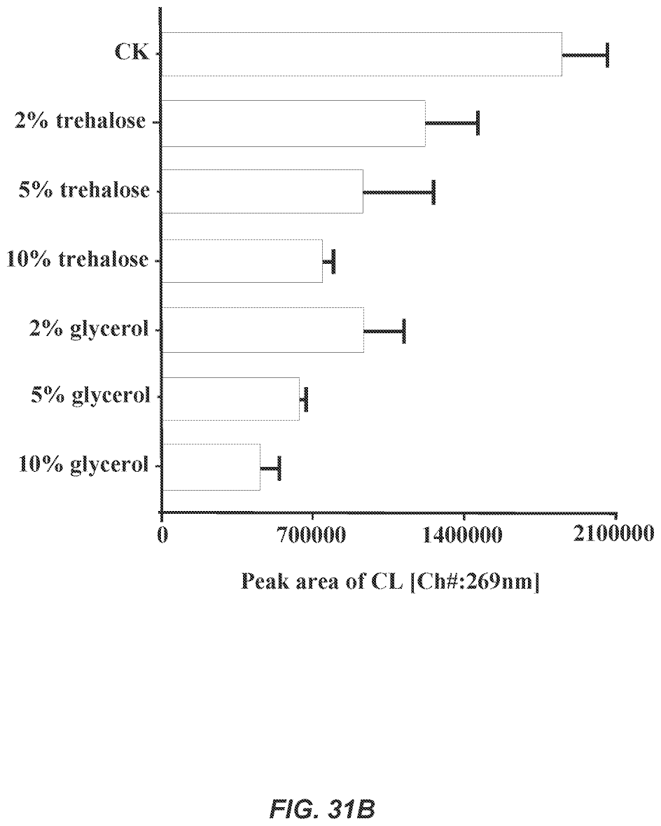

To investigate the effect of different carbon sources on SL production, we supplemented trehalose and glycerol with different concentrations (2%, 5% and 10%) into synthetic defined medium (SD) containing 2% dextrose respectively, which was used for yeast growth before coculturing with E. coli. The best performance in E. coli-yeast microbial consortia was observed when the engineered yeast strain was cultured in SD media supplemented with 10% glycerol, contributing to the best conversion efficiency from CL to 16-OH-CLA. FIG. 31

TABLE 1
Plasmids used in the study.

| | | Reference/ |
|---|---|---|
| Plasmids | Description | Source |
| pAC-BETAipi | Contains ctrE, crtB, crtI, crtY, and idi genes of Erwinia | Addgene |
| | herbicola (Pantoea agglomerans) Eho10 and thereby | #53277 |
| | produces beta-carotene in Escherichia coli; Derived | (1) |
| | from pACYCDuet-1, Replicon P15A (pACYC184); | |
| | Resistance, Chloramphenicol; | |
| pCDFDuet-1 | Replicon | Novagen |
| | CloDF13; Resistance, Spectinomycin | |
| pRSFDuet-1 | Replicon | Novagen |
| | RSF1030; Resistance, Kanamycin | |
| pET28b | Replicon | Novagen |
| | ColE1 (pBR322); Resistance, Kanamycin | |
| pET21a | Replicon | Novagen |
| | ColE1 (pBR322); Resistance, Ampicillin | |
| pYL725 (pCDFDuet-OsD27) | pCDFDuet-1 carrying D27 from Oryza sativa | This study |
| pYL726 (pCDFDuet-tCCD7- | pCDFDuet-1 carrying D27 from Oryza sativa and N- | This study |
| OsD27) | terminus 31 amino-acid truncated CCD7 from | |
| | Arabidopsis | |
| pYL735 (pET21a-tCCD8) | pET21a carrying N-terminus 56 amino-acid truncated | This study |
| | CCD8 from Arabidopsis | |
| pYL736 (pET28b-tCCD8) | pET28b carrying t N-terminus 56 amino-acid | This study |
| | truncated CCD8 from Arabidopsis | |
| pYL898 (pCDFDuet-tOsD27) | pCDFDuet-1 carrying N-terminus 40 amino-acid | This study |
| | truncated D27 from Oryza sativa | |
| pYL897 (pCDFDuet-AtD27) | pCDFDuet-1 carrying D27 from Arabidopsis | This study |
| pYL757 (pRSFtMAX1-tAtR1) | pRSFDuet-1 carrying N-terminus 46 amino-acid | This study |
| | truncated CPR1 from Arabidopsis and N-terminus 37 | |
| | amino-acid truncated MAX1 from Arabidopsis | |
| pAG414GPD-ccdB | Centromeric TRP, attR1-P_{GPD}-ccdB-T_{CYC1-}attR2 | (2) |
| pAG415GPD-ccdB | Centromeric LEU, attR1-P_{GPD}-ccdB-T_{CYC1-}attR2 | (2) |
| pAG416GPD-ccdB | Centromeric URA, attR1-P_{GPD}-ccdB-T_{CYC1-}attR2 | (2) |
| pYL6 | attL1-P_{TPI1}-ccdB-T_{Ste2}-attL2 | (3) |
| pYL7 | attL1-P_{TEF1}-ccdB-T_{CYC1}-attL2 | (3) |
| pYL8 | attL1-P_{PGK1}-ccdB-Tₚₕₒ₅-attL2 | (3) |
| pYL17 | Centromeric HIS, attR1-ccdB-attR2 | (3) |
| pYL19 | Centromeric LEU, attR1-ccdB-attR2 | (3) |
| pYL573 | Centromeric HIS, P_{TEF1-}AtATR1-T_{CYC1} | (4) |
| pYL759 | Centromeric LEU, P_{PGK1}-AtMAX1-Tₚₕₒ₅ | This study |
| pYL758 | Centromeric LEU, P_{TP1I}-AtMAX1-T_{STE2} | This study |
| pYL777 | Centromeric TRP, P_{GPD}-GaCYP722-T_{CYC1} | This study |
| pYL803 | Centromeric TRP, P_{GPD}-CaCYP722C-T_{CYC1} | This study |
| pYL795 | Centromeric TRP, P_{GPD}-LjCYP722C-T_{CYC1} | This study |
| pYL755 | Centromeric URA, P_{GPD}-VuCYP722C-T_{CYC1} | This study |
| pYL754 | Centromeric URA, P_{GPD}-SlCYP722C-T_{CYC1} | This study |
| pYL851 | Centromeric TRP, P_{GPD}-TpCYP722C-T_{CYC1} | This study |
| pYL1063 | Centromeric TRP, P_{GPD}-SbCYP722B-T_{CYC1} | This study |
| pYL889 | Centromeric TRP, P_{GPD}-OsCYP722B-T_{CYC1} | This study |
| pYL887 | Centromeric TRP, P_{GPD}-GmCYP722C-T_{CYC1} | This study |
| pYL878 | Centromeric TRP, P_{GPD}-FaCYP722C2-T_{CYC1} | This study |
| pYL761 | Centromeric TRP, P_{GPD}-OsCYP711A3-T_{CYC1} | This study |
| pYL770 | Centromeric URA, P_{PGK1}-OsCYP711A2-Tₚₕₒ₅ | This study |
| pYL387 | Centromeric TRP, P_{GPD}-AtD27-T_{CYC1} | This study |
| pYL928 | Centromeric TRP, P_{GPD}-tAtD27-T_{CYC1} | This study |
| pYL388 | Centromeric TRP, P_{GPD}-OsD27-T_{CYC1} | This study |
| pYL929 | Centromeric TRP, P_{GPD}-tOsD27-T_{CYC1} | This study |
| pYL407 | Centromeric TRP, P_{GPD}-mOsD27-T_{CYC1} | This study |
| pYL706 | Centromeric URA, P_{GPD}-DRE2-OsD27-T_{CYC1} | This study |
| pYL711 | Centromeric URA, P_{GPD}-DRE2-G4S-OsD27-T_{CYC1} | This study |
| pYL394 | Centromeric LEU, P_{GPD}-JAC1-T_{CYC1} | This study |
| pYL395 | Centromeric LEU, P_{GPD}-ISU1-T_{CYC1} | This study |
| pYL401 | Centromeric URA, P_{GPD}-YFH1-T_{CYC1} | This study |
| pYL396 | Centromeric URA, P_{GPD}-ISU2-T_{CYC1} | This study |
| pYL400 | Centromeric HIS, P_{GPD}-NFS1-T_{CYC1} | This study |
| pYL707 | Centromeric HIS, P_{GPD}-CTT1-T_{CYC1} | This study |
| pYL403 | Centromeric LEU, P_{GPD}-CTA1-T_{CYC1} | This study |
| pYL702 | Centromeric TRP, P_{GPD}-CCD7-T_{CYC1} | This study |
| pYL703 | Centromeric TRP, P_{GPD}-tCCD7-T_{CYC1} | This study |
| pYL704 | Centromeric TRP, P_{GPD}-mCCD7-T_{CYC1} | This study |
| pYL705 | Centromeric TRP, P_{GPD}-mtCCD7-T_{CYC1} | This study |

P, promoter;
T, terminator

TABLE 2
Strains used in the study.

| Yeast | | | |
|---|---|---|---|
| Strain | Description | Plasmid-based constructs | Reference |
| | CEN.PK2-1D (MATalpha; his3D1; | | (5) |
| | leu2-3_112; ura3-52; trp1-289; | | |
| | MAL2-8c; SUC2) | | |
| SWS1 | CEN.PK2-1D carrying pYL573 and | Centromeric HIS P_{TEF1-}AtATR1-T_{CYC1}, | This study |
| | pAG415GPD-ccdB | Centromeric LEU P_{GPD}-ccdB-T_{CYC1} | |
| SWS2 | CEN.PK2-1D carrying pYL573 and | Centromeric HIS P_{TEF1-}AtATR1-T_{CYC1}, | This study |
| | pYL758 | Centromeric LEU P_{TP1I}-AtMAX1-T_{STE2} | |
| SWS3 | CEN.PK2-1D carrying pYL573 and | Centromeric HIS P_{TEF1-}AtATR1-T_{CYC1}, | This study |
| | pYL759 | Centromeric LEU P_{PGK1}-AtMAX1-Tₚₕₒ₅ | |
| SWS4 | CEN.PK2-1D carrying pYL573 and | Centromeric HIS P_{TEF1-}AtATR1-T_{CYC1}, | This study |
| | pYL758 + pAG416GPD-ccdB | Centromeric LEU P_{TP1I}-AtMAX1-T_{STE2}, | |
| | | Centromeric URA P_{GPD}-ccdB-T_{CYC1} | |
| SWS5 | CEN.PK2-1D carrying pYL573 and | Centromeric HIS P_{TEF1-}AtATR1-T_{CYC1}, | This study |
| | pYL758 and pYL755 | Centromeric LEU P_{TP1I}-AtMAX1-T_{STE2}, | |
| | | Centromeric URA P_{GPD}-VuCYP722C-T_{CYC1} | |
| SWS6 | CEN.PK2-1D carrying pYL573 and | Centromeric HIS P_{TEF1-}AtATR1-T_{CYC1}, | This study |
| | pYL758 and pYL754 | Centromeric LEU P_{TP1I}-AtMAX1-T_{STE2}, | |
| | | Centromeric URA P_{GPD}-SlCYP722C-T_{CYC1} | |
| SWS7 | CEN.PK2-1D carrying pYL573 and | Centromeric HIS P_{TEF1-}AtATR1-T_{CYC1}, | This study |
| | pYL758 and pYL770 | Centromeric LEU P_{TP1I}-AtMAX1-T_{STE2}, | |
| | | Centromeric URA P_{PGK1}-OsCYP711A2-Tₚₕₒ₅, | |
| SWS8 | CEN.PK2-1D carrying pYL573 and | Centromeric HIS P_{TEF1-}AtATR1-T_{CYC1}, | This study |
| | pAG416GPD-ccdB | Centromeric URA P_{GPD}-ccdB-T_{CYC1} | |
| SWS9 | CEN.PK2-1D carrying pYL573 and | Centromeric HIS P_{TEF1-}AtATR1-T_{CYC1}, | This study |
| | pYL770 | Centromeric URA P_{PGK1}-OsCYP711A2-Tₚₕₒ₅, | |
| SWS10 | CEN.PK2-1D carrying pYL573 and | Centromeric HIS PT_{EF1-}AtATR1-T_{CYC1}, | This study |
| | pYL770 and pAG414GPD-ccdB | Centromeric URA P_{PGK1}-OsCYP711A2-Tₚₕₒ₅, | |
| | | Centromeric TRP P_{GPD}-ccdB-T_{CYC1} | |
| SWS11 | CEN.PK2-1D carrying pYL573 and | Centromeric HIS P_{TEF1-}AtATR1-T_{CYC1}, | This study |
| | pYL770 and pYL761 | Centromeric URA P_{PGK1}-OsCYP711A2-Tₚₕₒ₅, | |
| | | Centromeric TRP P_{GPD}- OsCYP711A3-T_{CYC1} | |
| SWS12 | CEN.PK2-1D carrying pYL573 and | Centromeric HIS P_{TEF1-}AtATR1-T_{CYC1}, | This study |
| | pYL759 and pAG414GPD-ccdB | Centromeric LEU P_{PGK1}-AtMAX1-Tₚₕₒ₅, | |
| | | Centromeric TRP P_{GPD}-ccdB-T_{CYC1} | |
| SWS13 | CEN.PK2-1D carrying pYL573 and | Centromeric HIS P_{TEF1-}AtATR1-T_{CYC1}, | This study |
| | pYL759 and pYL777 | Centromeric LEU P_{PGK1}-AtMAX1-Tₚₕₒ₅, | |
| | | Centromeric TRP P_{GPD}-GaCYP722C-T_{CYC1} | |
| SWS14 | CEN.PK2-1D carrying pYL573 and | Centromeric HIS P_{TEF1-}AtATR1-T_{CYC1}, | This study |
| | pYL759 and pYL803 | Centromeric LEU P_{PGK1}-AtMAX1-Tₚₕₒ₅, | |
| | | Centromeric TRP P_{GPD}-CaCYP722C-T_{CYC1} | |
| SWS15 | CEN.PK2-1D carrying pYL573 and | Centromeric HIS P_{TEF1-}AtATR1-T_{CYC1}, | This study |
| | pYL759 and pYL795 | Centromeric LEU P_{PGK1}-AtMAX1-Tₚₕₒ₅, | |
| | | Centromeric TRP P_{GPD}-LjCYP722C-T_{CYC1} | |
| SWS16 | CEN.PK2-1D carrying pYL573 and | Centromeric HIS P_{TEF1-}AtATR1-T_{CYC1}, | This study |
| | pYL759 and pYL851 | Centromeric LEU P_{PGK1}-AtMAX1-Tₚₕₒ₅, | |
| | | Centromeric TRP P_{GPD}-TpCYP722C-T_{CYC1} | |
| SWS17 | CEN.PK2-1D carrying pYL573 and | Centromeric HIS P_{TEF1-}AtATR1-T_{CYC1}, | This study |
| | pYL759 and pYL878 | Centromeric LEU P_{PGK1}-AtMAX1-Tₚₕₒ₅, | |
| | | Centromeric TRP P_{GPD}-FaCYP722C2-T_{CYC1} | |
| SWS18 | CEN.PK2-1D carrying pYL573 and | Centromeric HIS P_{TEF1-}AtATR1-T_{CYC1}, | This study |
| | pYL759 and pYL887 | Centromeric LEU P_{PGK1}-AtMAX1-Tₚₕₒ₅, | |
| | | Centromeric TRP P_{GPD}-GmCYP722C-T_{CYC1} | |
| SWS19 | CEN.PK2-1D carrying pYL573 and | Centromeric HIS P_{TEF1-}AtATR1-T_{CYC1}, | This study |
| | pYL759 and pYL889 | Centromeric LEU P_{PGK1}-AtMAX1-Tₚₕₒ₅, | |
| | | Centromeric TRP P_{GPD}-OsCYP722B-T_{CYC1} | |
| SWS20 | CEN.PK2-1D carrying pYL573 and | Centromeric HIS P_{TEF1-}AtATR1-T_{CYC1}, | This study |
| | pYL759 and pYL1063 | Centromeric LEU P_{PGK1}-AtMAX1-Tₚₕₒ₅, | |
| | | Centromeric TRP P_{GPD}-SbCYP722B-T_{CYC1} | |

| E. coil | | | |
|---|---|---|---|
| Strain | Genotype | Usage | |
| CL-1 | BL21(DE3) carrying pAC-BETAipi | all-trans-β-carotene production | This study |
| | and pCDFDuet-1 | | |
| CL-2 | BL21(DE3) carrying pAC-BETAipi | 9-cis-β-carotene production | This study |
| | and pYL725 | | |
| CL-3 | BL21(DE3) carrying pAC-BETAipi | 9-cis-β-apo-10′-carotenol | This study |
| | and pYL726 | production | |
| CL-4 | BL21(DE3) carrying pAC-BETAipi, | Negative control | This study |
| | pYL726 and pET21a | | |
| CL-5 | BL21(DE3) carrying pAC-BETAipi, | Carlactone production | This study |
| | pYL726 and pYL735 | | |
| CL-6 | BL21(DE3) carrying pAC-BETAipi, | Negative control | This study |
| | pYL726 and pET28b | | |
| CL-7 | BL21(DE3) carrying pAC-BETAipi, | Carlactone production | This study |
| | pYL726 and pYL736 | | |
| CL-8 | BL21(DE3) carrying pAC-BETAipi | 9-cis-β-carotene production | This study |
| | and pYL898 | | |
| CL-9 | BL21(DE3) carrying pAC-BETAipi | 9-cis-β-carotene production | This study |
| | and pYL897 | | |
| CL-10 | BL21(DE3) carrying pAC-BETAipi, | Negative control | This study |
| | pYL726, pYL735 and pRSFDuet-1 | | |
| CL-11 | BL21(DE3) carrying pAC-BETAipi, | Failed CLA production | This study |
| | pYL726, pYL735 and pYL757 | | |

TABLE 3
CYPs used in this study and summary of results.

| | | | Experimental | |
|---|---|---|---|---|
| Gene | Species | Notation | results/product | Reference |
| AtMAX1 (CYP711A1) | A. thaliana | CL → CLA | CLA | (6, 7) |
| OsCYP711A2 | Oryza sativa (rice) | CL → CLA → 4DO | Trace CLA & 4DO | (6, 8) |
| OsCYP711A3 | Oryza sativa (rice) | CL → CLA & | orobanchol | (6, 8) |
| | | 4DO → orobanchol | | |
| GaCYP722C | Gossypium | CLA → 5DS | 5DS | (9) |
| | arboreum (cotton) | | | |
| LjCYP722C | Lotus japonicus | CLA → ? | 5DS | (10) |
| VuCYP722C | Vigna unguiculata | CLA → orobanchol | orobanchol | (11) |
| | (cowpea) | | | |
| SlCYP722C | Solanum | CLA → orobanchol | orobanchol | (11) |
| | lycopersicum | | | |
| | (tomato) | | | |
| CaCYP722C | Capsicum annuum | | orobanchol | (12) |
| | (Red bell pepper) | | | |
| TpCYP722C | Trifolium pratense | | orobanchol | (13) |
| | (Red Clover) | | | |
| SbCYP722B | Sorghum bicolor | | None | |
| OsCYP722B | Oryza sativa (rice) | | None | |
| GmCYP722C | Glycine max | | orobanchol | (12) |
| AcCYP722 | Aquilegia coerulea | | 16-OH-CLA | |
| PsCYP722A | Pisum sativum | | 16-OH-CLA | |
| FaCYP722C2 | Fragaria ananassa | | 5DS | (13) |
| | (wild strawberry) | | | |

TABLE 4
Accession numbers of CYP722Cs used for the phylogenetic tree construction
in FIG. 4. The amino acid sequences are downloadable from NCBI, except for
TpCYP722C, which is downloaded from https://plants.ensembl.org/index.html.

| | | Size | |
|---|---|---|---|
| Gene | Species | (a.a.) | Accession numbers |
| GaCYP722C | Gossypium arboreum | 491 | XP_016745621 |
| VuCYP722C | Vigna unguiculata (cowpea) | 494 | XP_027918387 |
| SlCYP722C | Solanum lycopersicum (tomato) | 486 | XP_004232430 |
| GmCYP722C | Glycine max | 494 | XP_003548874 |
| TpCYP722C | Trifolium pratense (Red Clover) | 492 | Tp57577_TGAC_v2_mRNA22267 |
| GbCYP722C | Gossypium barbadense | 491 | KAB2051573 |
| DzCYP722C | Durio zibethinus | 490 | XP_022741804 |
| StCYP722C | Solanum tuberosum | 495 | XP_006340657 |
| CaCYP722C | Capsicum annum | 490 | XP_016560669 |
| EgCYP722C | Erythranthe guttata | 483 | EYU24120 |
| GrCYP722C | Gossypium raimondii | 491 | XP_012436859 |
| CcCYP722C | Citrus clementina | 485 | ESR38866 |
| CsCYP722C | Citrus sinensis | 497 | XP_006467149 |
| EgCYP722C | Eucalyptus grandis | 486 | XP_010029758 |
| CusCYP722C | Cucumis sativus | 484 | XP_004150006 |
| PpCYP722C | Prunus persica | 491 | XP_020424098 |
| PmCYP722C | Prunus mume | 491 | XP_008241894 |
| MbCYP722C | Malus baccata | 497 | TQE02546 |
| FaCYP722C1 | Fragaria ananassa (wild strawberry) | 481 | XP_004287873 |
| FaCYP722C2 | Fragaria ananassa (wild strawberry) | 491 | XP_004303111 |
| LjCYP722C2 | Lotus japonicus | 484 | BBO93647 |
| CcCYP722C | Cajanus cajanifolius | 493 | XP_020238265 |
| RcCYP722C1 | Ricinus communis | 492 | XP_002521537 |
| RcCYP722C2 | Ricinus communis | 491 | XP_002524333 |
| MeCYP722C1 | Manihot esculenta | 506 | XP_021622147 |
| MeCYP722C2 | Manihot esculenta | 500 | XP_021600819 |
| VvCYP722C | Vitis vinifera | 491 | XP_002269279 |
| ElgCYP722C | Elaeis guineensis | 564 | XP_029117801 |
| OsCYP722B | Oryza sativa (rice) | 518 | XP_015639511 |
| BdCYP722B | Brachypodium distachyon | 510 | XP_010230530 |
| SiCYP722B | Setaria italica | 542 | XP_004960948 |
| PhCYP722B | Panicum hallii | 551 | XP_025809022 |
| SbCYP722B | Sorghum bicolor | 512 | OQU77193 |
| SlCYP722A | Solanum lycopersicum | 481 | XP_004238286 |
| AtCYP722A | Arabidopsis thaliana | 476 | NP_173393 |
| StCYP722A | Solanum tuberosum | 481 | XP_006342223 |
| CaCYP722A | Capsicum annum | 490 | XP_016568530 |
| AlCYP722A | Arabidopsis lyrata | 477 | XP_020869333 |
| EsCYP722A | Eutrema salsugineum | 489 | XP_006416485 |
| CrCYP722A | Capsella rubella | 481 | XP_006307378 |
| BrCYP722A | Brassica rapa | 489 | RID51716 |
| TcCYP722A1 | Theobroma cacao | 488 | EOY14821 |
| TcCYP722A2 | Theobroma cacao | 488 | EOY14823 |
| GrCYP722A | Gossypium raimondii | 498 | XP_012452669 |
| CcCYP722A | Citrus clementina | 523 | ESR38694 |
| CsCYP722A | Citrus sinensis | 498 | XP_006467169 |
| EgCYP722A | Eucalyptus grandis | 500 | XP_010060975 |
| CusCYP722A | Cucumis sativus | 483 | XP_011649632 |
| PpCYP722A | Prunus persica | 496 | XP_007226877 |
| PmCYP722A | Prunus mume | 525 | XP_008220838 |
| FaCYP722A | Fragaria ananassa | 493 | XP_011459540 |
| | (wild strawberry) | | |
| CasCYP722A | Cannabis sativa | 533 | XP_030484637 |
| LjCYP722A | Lotus japonicus | 499 | AFK39117 |
| CiaCYP722A | Cicer arietinum | 488 | XP_004501123 |
| CcCYP722A | Cajanus cajanifolius | 485 | XP_020219964 |
| RcCYP722A | Ricinus communis | 489 | XP_002510423 |
| MeCYP722A | Manihot esculenta | 508 | XP_021600571 |
| VvCYP722A | Vitis vinifera | 496 | CAN64558 |
| NnCYP722A | Nelumbo nucifera (sacred lotus) | 481 | XP_010263599 |
| MaCYP722A | Musa acuminata (banana) | 491 | XP_009390176 |

TABLE 5
Accession numbers for D27 enzymes used for the
efficient D27 variant screening in FIG. 20.

| | | Size | |
|---|---|---|---|
| Gene | Species | (a.a.) | Accession numbers |
| ZmD27 | Zea mays | 262 | NP_001144840.1 |
| VvD27 | Vitis vinifera | 279 | XP_003634993.2 |
| SmD27 | Selaginella moellendorffii | 237 | XP_024534389.1 |
| SbD27 | Sorghum bicolor | 292 | XP_021316884.1 |
| RcD27 | Ricinus communis | 244 | EEF41385.1 |
| PsD27-2 | Picea sitchensis | 275 | ABK23534.1 |
| PeD27 | Populus euphratica | 267 | XP_011026332.1 |
| MtD27 | Medicago truncatula | 252 | R4HZ96.1 |
| PpD27 | Physcomitrium patens | 261 | XP_024384448.1 |

TABLE 6
Accession numbers for MAX1 enzymes used for the
efficient MAX1 variant screening in FIG. 23

| | | Size | |
|---|---|---|---|
| Gene | Species | (a.a.) | Accession numbers |
| SbCYP711A2 | Sorghum bicolor | 545 | XP_002456213 |
| EgCYP711A | Eucalyptus grandis | 542 | XP_010047358 |
| SlMAX1 | Solanum lycopersicum | 519 | XP_004245085 |
| FveMAX1a | Fragaria vesca subsp. | 531 | XP_004291053 |
| | vesca | | |
| PhMAX1 | Petunia × hybrida | 533 | AEB97383 |
| ZmMAX1b | Zea mays | 543 | ONM29770 |
| AtMAX1 | Arabidopsis thaliana | 522 | NP_565617 |

TABLE 7
Accession numbers of CPR enzymes involved
in the YAZ57 construction in FIG. 26.

| | | Size | |
|---|---|---|---|
| Gene | Species | (a.a.) | Accession numbers |
| NCP1(ScCPR) | Saccharomyces cerevisiae | 691 | NP_011908.1 |
| AtR1(AtCPR1) | Arabidopsis thaliana | 692 | NP_194183.1 |

TABLE 8
Synthetic gene sequences used in this study

| Gene | Sequence (5′-3′) |
|---|---|
| OsD27 | ATGGAAACTACCACCTTGGTTTTGTTGTTGCCACATGGTGGTGCTGGTGGTGTTA |
| | GACCAGCTGCTGCTGCTACTGCTAAAAGATCTTATGTTATGAGAAGATGCTGCTC |
| | CACTGTTAGAGCTGTTATGGCTAGACCTCAAGAAGCTCCAGCTTCTGCTCCAGCT |
| | AAAAAAACTGAAACTGCTGCTATGATGTCTACCGTTCAAACAGAAACTGCAGCTG |
| | CCCCACCAGCTACTGTTTACAGAGATTCTTGGTTTGATAAGTTGGCCATCGGTTAC |
| | TTGTCTAGGAACTTGCAAGAAGCTTCAGGTTTGAAGAACGAAAAGGATGGTTACG |
| | AATCCTTGATTGATGCTGCTTTGGCCATCTCCAGAATTTTCTCATTGGATAAGCAA |
| | TCCGAAATCGTTACCCAAGCTTTGGAAAGAGCTTTGCCATCTTACATTTTGACCAT |
| | GATCAAGGTTATGATGCCACCATCTAGATTCTCCAGAGAATACTTTGCTGCTTTCA |
| | CCACTATTTTCTTCCCATGGTTGGTTGGTCCATGTGAAGTTATGGAATCTGAAGTC |
| | GAAGGCAGAAAAGAAAAGAACGTTGTTTACATCCCAAAGTGCAGGTTCTTGGAAT |
| | CTACTAATTGTGTTGGTATGTGCACCAACTTGTGTAAGATTCCATGCCAAAAGTTC |
| | ATCCAGGATTCTTTGGGTATGAAGGTTTACATGTCTCCAAACTTCGAAGATATGTC |
| | CTGCGAAATGATTTTCGGTCAACAACCACCAGAAGATGATCCAGCTTTGAAACAA |
| | CCATGTTTCAGAACTAAGTGCGTTGCCAAACAAAATCATGGTGTTAACTGCTCCAT |
| | CTAA (SEQ ID NO: 2) |
| CCD7 | ATGCTGACCAAAATGTCTTTGCCAATTCCACCAAAGTTTCTGCCACCATTGAAATC |
| | TCCACCAATCCATCATCATCAAACTCCACCACCATTGGCTCCACCAAGAGCTGCT |
| | ATTTCTATTTCAATTCCAGATACCGGTTTGGGTAGAACCGGTACTATTTTGGATGA |
| | ATCTACTTCCTCTGCCTTCAGAGATTACCAATCTTTGTTCGTGTCTCAGAGATCCG |
| | AAACTATTGAACCAGTTGTTATCAAGCCAATCGAAGGTTCTATTCCAGTTAATTTTC |
| | CATCTGGCACTTACTATTTGGCTGGTCCAGGTTTGTTTACTGATGATCATGGTTCT |
| | ACTGTTCACCCATTGGATGGTCATGGTTATTTGAGAGCTTTCCATATCGATGGTAA |
| | CAAGAGAAAGGCTACTTTCACTGCTAAGTACGTTAAGACCGAAGCCAAAAAAGAA |
| | GAACACGATCCAGTTACTGATACTTGGAGATTCACTCATAGAGGTCCATTCTCTGT |
| | TTTGAAAGGTGGTAAGAGATTCGGTAACACCAAGGTTATGAAGAACGTTGCTAAC |
| | ACTTCCGTTTTGAAATGGGCTGGTAGATTATTGTGTTTGTGGGAAGGTGGTGAAC |
| | CATACGAAATTGAATCTGGTTCTTTGGATACCGTCGGTAGATTCAATGTTGAAAAC |
| | AACGGTTGCGAATCTTGCGACGATGATGATTCTTCTGATAGAGATTTGTCCGGTC |
| | ATGATATTTGGGATACTGCTGCTGATTTGTTGAAGCCAATTCTACAAGGTGTTTTC |
| | AAGATGCCACCAAAGAGATTCTTGTCCCATTACAAAGTTGACGGTAGAAGAAAGA |
| | GGTTGTTGACTGTTACTTGTAACGCCGAAGATATGTTGTTGCCAAGATCTAACTTC |
| | ACCTTCTGCGAATACGATTCCGAATTCAAGTTGATTCAGACCAAAGAGTTCAAGAT |
| | CGACGATCACATGATGATCCATGATTGGGCTTTTACCGATACTCACTACATTTTGT |
| | TTGCCAACAGAGTCAAGTTGAACCCAATTGGTTCTATTGCTGCTATGTGTGGTATG |
| | TCTCCAATGGTTTCTGCTTTGTCTTTGAACCCATCTAACGAATCTTCCCCAATCTAT |
| | ATTTTGCCAAGGTTCTCCGATAAGTACTCTAGAGGTGGCAGAGATTGGAGAGTTC |
| | CAGTTGAAGTTTCTTCTCAATTGTGGTTGATCCATTCTGGTAACGCTTACGAAACT |
| | AGAGAAGATAACGGTGACTTGAAGATTCAAATTCAAGCTTCTGCTTGCTCCTACAG |
| | ATGGTTTGATTTTCAAAAGATGTTCGGTTACGACTGGCAGTCTAACAAATTGGATC |
| | CATCTGTTATGAACTTGAACAGAGGTGATGACAAGTTGTTACCACACTTGGTTAAG |
| | GTTTCTATGACCTTGGATTCTACCGGTAACTGTAACTCTTGTGATGTTGAACCTTT |
| | GAACGGTTGGAACAAGCCATCTGATTTTCCAGTTATTAACTCCTCTTGGTCCGGC |
| | AAAAAAAACAAGTATATGTACTCTGCTGCCTCCTCTGGTACTAGATCTGAATTGCC |
| | ACATTTTCCATTCGATATGGTTGTCAAGTTCGACTTGGACTCTAACTTGGTTAGAA |
| | CTTGGTCTACTGGTGCTAGAAGATTTGTTGGTGAACCTATGTTCGTCCCAAAGAA |
| | CTCTGTTGAAGAGGGTGAAGAGGAAGATGACGGTTATATCGTTGTTGTTGAATAC |
| | GCCGTTTCTGTCGAAAGATGCTACTTGGTTATTTTGGACGCCAAAAAGATCGGTG |
| | AATCTGATGCTGTTGTTTCCAGATTAGAAGTCCCAAGAAATCTGACTTTCCCAATG |
| | GGTTTTCATGGTTTGTGGGCTTCAGATTGA (SEQ ID NO: 3) |
| CCD8 | ATGGCTTCTTTGATCACAACCAAAGCAATGATGAGTCATCATCATGTTTTGTCGTC |
| | AACTAGAATCACTACTCTTTATTCCGACAATTCCATCGGCGATCAACAAATAAAAA |
| | CAAAACCTCAAGTCCCTCACCGGTTATTTGCTCGGAGGATCTTCGGTGTAACCAG |
| | AGCTGTAATTAATTCAGCGGCACCGTCTCCGTTGCCGGAGAAAGAGAAGGTGGA |
| | AGGTGAGAGACGGTGTCATGTTGCGTGGACAAGTGTACAACAAGAGAATTGGGA |
| | GGGTGAACTTACTGTCCAAGGAAAGATACCCACTTGGCTGAATGGTACGTACCTA |
| | AGAAACGGTCCTGGTCTATGGAACATTGGAGACCACGATTTCCGGCATCTCTTCG |
| | ACGGCTACTCCACACTCGTCAAGCTTCAATTCGATGGCGGTCGTATATTCGCCGC |
| | CCACCGTCTCCTTGAATCCGACGCTTACAAAGCCGCCAAGAAACACAATAGGCTT |
| | TGTTACCGTGAATTCTCCGAGACTCCAAAATCGGTGATCATAAACAAAAACCCTTT |
| | CTCCGGGATCGGAGAAATCGTCAGGCTTTTCTCCGGAGAGTCTTTAACGGACAAC |
| | GCCAACACCGGAGTGATCAAACTCGGTGACGGGCGGGTCATGTGTCTGACGGAG |
| | ACTCAAAAAGGATCGATTTTAGTCGACCATGAGACGCTAGAGACGATCGGGAAAT |
| | TTGAGTACGACGACGTATTGTCCGATCATATGATCCAATCAGCGCATCCGATAGT |
| | GACGGAGACGGAGATGTGGACGTTGATACCGGATTTGGTTAAACCGGGTTATCG |
| | GGTCGTGAGGATGGAAGCCGGGTCGAATAAAAGAGAGGTTGTGGGGGGGGTGA |
| | GGTGTCGAAGTGGGTCGTGGGGACCCGGTTGGGTCCATTCGTTTGCGGTGACG |
| | GAGAATTATGTTGTAATACCGGAAATGCCCCTGAGATATTCGGTGAAGAATCTTCT |
| | TAGAGCTGAGCCGACGCCACTTTACAAGTTCGAGTGGTGTCCCCAAGACGGAGC |
| | TTTTATTCATGTCATGTCCAAACTCACCGGAGAAGTCGTGGCTAGCGTGGAGGTT |
| | CCAGCATACGTAACGTTTCACTTCATAAACGCGTATGAAGAAGATAAAAATGGCG |
| | ATGGAAAAGCGACGGTCATCATTGCAGATTGTTGTGAACACAACGCCGATACTCG |
| | GATACTCGATATGCTCCGTCTCGATACCCTACGTTCTTCCCATGGTCACGACGTTT |
| | TACCCGATGCTAGGATCGGGAGATTCAGGATACCATTGGACGGGAGCAAATACG |
| | GGAAACTAGAGACAGCCGTGGAGGCAGAGAAGCATGGGAGAGCGATGGATATGT |
| | GCAGCATCAATCCTTTGTATTTGGGTCAAAAATACCGTTACGTTTATGCATGCGGT |
| | GCTCAACGACCTTGTAACTTCCCCAATGCTCTCTCCAAGGTTGATATTGTGGAGA |
| | AGAAAGTGAAGAACTGGCACGAGCATGGTATGATACCATCTGAACCATTCTTCGT |
| | GCCTCGACCCGGTGCAACCCATGAGGATGATGGAGTGGTGATATCGATAGTAAG |
| | TGAAGAAAATGGAGGAAGCTTTGCAATCTTGCTTGATGGGAGCTCCTTTGAAGAA |
| | ATAGCAAGAGCCAAGTTTCCCTATGGCCTTCCTTATGGCTTGCATGGTTGCTGGA |
| | TCCCCAAAGATTAA (SEQ ID NO: 4) |
| AtR1 | ATGACTTCTGCCTTGTATGCCTCTGATTTGTTCAAGCAATTGAAGTCCATTATGGG |
| | CACCGATTCTTTGTCTGATGATGTTGTTTTGGTTATCGCTACTACCTCTTTGGCTTT |
| | GGTTGCTGGTTTTGTTGTTCTGTTGTGGAAAAAGACTACCGCTGATAGATCTGGT |
| | GAATTGAAACCATTGATGATCCCCAAATCTTTGATGGCCAAAGATGAAGATGATGA |
| | CTTGGACTTAGGTTCTGGTAAGACTAGAGTTTCCATTTTCTTCGGTACTCAAACTG |
| | GTACTGCTGAAGGTTTTGCTAAAGCTTTGTCCGAAGAAATCAAGGCCAGATACGA |
| | AAAAGCTGCCGTTAAGGTTATTGATTTGGATGATTATGCTGCCGATGACGACCAAT |
| | ACGAAGAAAAGTTGAAGAAAGAAACCTTGGCCTTCTTCTGTGTTGCTACTTATGGT |
| | GATGGTGAACCTACTGATAATGCTGCTAGATTTTACAAGTGGTTCACCGAAGAGA |
| | ACGAAAGAGATATCAAGTTGCAACAATTGGCCTACGGTGTTTTTGCTTTGGGTAAT |
| | AGACAATACGAGCACTTCAACAAGATCGGTATCGTTTTGGATGAAGAGTTGTGTA |
| | AAAAGGGTGCCAAGAGATTGATTGAAGTTGGTTTGGGTGATGATGACCAGTCTAT |
| | CGAAGATGATTTTAACGCCTGGAAAGAATCCTTGTGGTCTGAATTGGATAAGTTGT |
| | TGAAGGACGAAGATGACAAATCTGTTGCTACACCATACACTGCTGTTATTCCAGA |
| | GTATAGAGTTGTTACTCACGATCCAAGATTCACGACTCAAAAGTCTATGGAATCTA |
| | ACGTTGCTAACGGTAACACCACCATCGATATTCATCATCCATGTAGAGTTGATGTC |
| | GCCGTCCAAAAAGAATTGCATACTCATGAATCCGACAGATCCTGCATTCATTTGGA |
| | ATTCGATATTTCCAGAACCGGTATTACTTACGAAACCGGTGATCATGTTGGTGTTT |
| | ACGCTGAAAATCACGTTGAAATCGTTGAAGAAGCCGGTAAGTTGTTAGGTCATTC |
| | ATTGGATTTGGTGTTCTCCATTCATGCCGACAAAGAAGATGGTTCTCCTTTGGAAT |
| | CTGCTGTTCCACCACCATTTCCAGGTCCATGTACTTTAGGTACTGGTTTGGCTAGA |
| | TATGCTGACTTGTTGAATCCACCAAGAAAGTCTGCTTTAGTTGCTTTGGCTGCTTA |
| | TGCTACTGAACCATCTGAAGCCGAAAAATTGAAACATTTGACTTCCCCAGATGGTA |
| | AGGACGAATATTCTCAATGGATAGTTGCCTCTCAGAGGTCTTTGTTGGAAGTTATG |
| | GCTGCTTTTCCATCTGCTAAACCACCATTGGGTGTTTTTTTTGCTGCTATTGCTCC |
| | AAGATTGCAACCTAGGTATTACTCCATTTCTTCATCACCAAGATTGGCCCCATCTA |
| | GAGTTCATGTTACATCTGCTTTGGTTTATGGTCCAACTCCAACTGGTAGAATTCAT |
| | AAGGGTGTTTGTTCTACCTGGATGAAGAACGCTGTTCCAGCTGAAAAATCTCATG |
| | AATGTTCTGGTGCCCCAATTTTCATTAGAGCTTCTAATTTCAAGCTGCCAAGCAAT |
| | CCATCTACTCCAATAGTTATGGTTGGTCCAGGTACAGGTTTAGCTCCTTTTAGAGG |
| | TTTCCTACAAGAAAGGATGGCCTTGAAAGAGGATGGCGAAGAATTGGGTTCTTCC |
| | TTGTTGTTTTTTGGTTGCAGAAACAGACAGATGGATTTCATCTATGAGGACGAGTT |
| | GAACAACTTCGTTGATCAAGGTGTTATCTCCGAATTGATTATGGCCTTTTCTAGAG |
| | AAGGTGCCCAGAAAGAATATGTCCAACATAAGATGATGGAAAAAGCCGCTCAAGT |
| | TTGGGACCTAATCAAAGAAGAAGGATACTTGTACGTTTGCGGTGATGCTAAAGGT |
| | ATGGCTAGAGATGTTCATAGAACATTGCATACCATCGTCCAAGAACAAGAAGGTG |
| | TTTCATCTTCTGAAGCTGAAGCTATCGTTAAGAAGTTGCAAACTGAAGGTAGATAC |
| | TTGAGAGATGTCTGGTGA (SEQ ID NO: 5) |
| AtMAX1 | ATGAAGACCCAACATCAATGGTGGGAAGTTTTGGATCCATTCTTGACTCAACATGA |
| | AGCTTTGATTGCTTTCTTGACTTTCGCTGCTGTTGTTATCGTTATCTACTTGTATAG |
| | ACCATCTTGGTCTGTCTGTAATGTTCCAGGTCCAACTGCTATGCCATTGGTTGGTC |
| | ATTTGCCATTGATGGCTAAATATGGTCCAGATGTTTTCTCCGTTTTGGCTAAACAA |
| | TACGGTCCAATCTTCAGATTCCAAATGGGTAGACAACCCTTGATTATTATCGCTGA |
| | AGCTGAATTGTGTAGAGAGGTTGGTATTAAGAAGTTCAAGGACTTGCCAAACAGA |
| | TCCATTCCATCTCCAATTTCTGCTTCTCCATTGCATAAGAAGGGTTTGTTTTTCACC |
| | AGAGACAAGAGATGGTCAAAGATGAGAAACACCATCCTGTCATTATACCAGCCAT |
| | CTCATTTGACCTCATTGATTCCAACTATGCACTCCTTTATTACCTCTGCTACTCACA |
| | ACTTGGATTCCAAACCTAGAGATATCGTGTTCTCCAACTTGTTCTTGAAGTTGACC |
| | ACCGATATTATTGGTCAAGCTGCTTTTGGTGTTGACTTTGGTTTGTCTGGTAAAAA |
| | GCCAATCAAGGATGTTGAAGTTACCGACTTCATCAACCAGCATGTTTACTCTACTA |
| | CCCAATTGAAGATGGACTTGTCTGGTTCCTTGTCCATTATTTTGGGTTTGTTGATC |
| | CCAATCCTGCAAGAACCTTTTAGACAAGTCTTGAAGAGAATCCCAGGTACTATGG |
| | ATTGGAGAGTTGAAAAGACTAACGCTAGATTGTCTGGTCAGTTGAACGAAATCGT |
| | TTCCAAGAGAGCTAAAGAAGCTGAAACCGATTCTAAGGACTTCCTGTCTTTGATTT |
| | TGAAGGCCAGAGAATCTGATCCATTCGCCAAGAATATTTTCACCTCCGATTACATT |
| | TCTGCCGTTACCTATGAACATTTGTTGGCTGGTTCTGCTACTACCGCTTTTACTTT |
| | ATCTTCTGTCCTGTACTTGGTTTCCGGTCACTTAGATGTCGAAAAGAGACTGTTGC |
| | AAGAAATCGATGGTTTCGGTAACAGAGATTTGATTCCTACTGCTCATGACTTGCAA |
| | CATAAGTTCCCATACTTGGACCAAGTTATCAAAGAAGCCATGAGGTTCTATATGGT |
| | GTCTCCATTAGTTGCTAGAGAAACCGCAAAAGAAGTTGAAATTGGCGGTTACTTG |
| | TTGCCAAAAGGTACTTGGGTTTGGTTGGCTTTGGGTGTTTTGGCAAAAGATCCAA |
| | AGAATTTCCCAGAACCAGAAAAGTTCAAGCCAGAAAGATTTGATCCAAACGGTGA |
| | AGAAGAAAAGCACAGACATCCATACGCTTTTATTCCATTTGGTATTGGTCCTAGAG |
| | CTTGTGTTGGACAAAGATTTGCTCTACAAGAAATCAAGTTGACCCTGTTGCACTTG |
| | TACAGAAACTACATCTTCAGACACAGCCTGGAAATGGAAATACCATTGCAATTGGA |
| | TTACGGCATCATCCTGAGTTTTAAGAACGGTGTTAAGTTGAGAACCATCAAGAGG |
| | TTCTGA (SEQ ID NO: 6) |
| OsCYP711A2 | ATGGAAATCTCTACTGTTTTGGGTGCTATTTTGGCTGAATACGCTGTTACTTTGGT |
| | TGCTATGGCTGTTGGTTTTTTGGTTGTTGGTTACTTGTATGAGCCATACTGGAAGG |
| | TTAGACATGTTCCAGGTCCAGTTCCATTGCCATTGATTGGTCACTTGCATTTGTTG |
| | GCTATGCATGGTCCAGATGTTTTCTCTGTTTTGACTAGAAAGTACGGTCCAATCTT |
| | CAGATTCCATATGGGTAGACAACCATTGGTTATGGTTGCTGATGCTGAATTGTGTA |
| | AAGAAGTTGGCGTTAAGAAGTTCAAGAACTTCCCAAATAGATCCATGCCATCTCCA |
| | ATTACCAATTCTCCAGTTCATCAAAAGGGTCTGTTTTTCACTTCTGGTTCTAGATG |
| | GACTACCATGAGAAACATGATCTTGTCTATCTACCAACCATCTCATTTGGCTACCT |
| | TGATTCCTTCTATGGAATCCTGTATTGAAAGAGCTGCTGAAAACTTGGAAGGTCAA |
| | GAAGAAATCAACTTCTCCAAGCTGTCTTTGTCTTTCACTACCGATGTTTTAGGTCA |
| | AGCTGCTTTTGGTACTGATTTCGGTTTGTCTAAGAAATTGGCCTCCTCTGATGATG |
| | ATGAAGATACCAGAAAAATTGCTGCTGATACTTGTGCTGAAGCTAAAGCTTCTTCC |
| | GAATTCATCAAGATGCATGTTCATGCTACCACCTCATTGAAGATGGATATGTCTGG |
| | TTCCTTGTCCATTATCGTCGGTCAATTATTGCCATTCTTGCACGAACCTTTCAGAC |
| | AAGTTTTGAAGAGATTGAGATGGACTGCCGATCACGAAATTGATAGAGTTAATTTG |
| | ACTCTGGGCAGACAGTTGGATAGAATAGTTGCTGAAAGAACAGCTGCCATGAAGA |
| | GAGATCCAGCTGCTTTACAACAAAGAAAGGATTTCTTGTCCGTTATGTTGACCGCT |
| | AGGGAATCTAACAAATCTTCTAGAGAATTACTGACGCCCGATTACATTTCTGCTTT |
| | GACTTATGAACATCTGTTGGCTGGTTCTGCTACTACTGCTTTTACTTTGACTACTG |
| | CCTTGTACTTGGTTGCCAAACATCCAGAAGTTGAAGAAAAGTTGTTGAGGGAAATT |
| | GATGGTTTCGGTCCAAGAGATAGAGTTCCAACTGCTGAAGATCTGCAAACTAAGT |
| | TTCCATACTTGGACCAGGTTTTGAAAGAAGCTATGAGGTATTACCCATCCTCACCT |
| | TTGATTGCCAGAGAATTGAATCAACAGTTGGAAATCGGTGGTTACCCATTGCCAA |
| | AAGGTACTTGGGTTTGGATGGCTCCAGGTGTATTGGGTAAAGATCCAAAGAATTT |
| | TCCAGAGCCAGAAGTGTTTAGACCAGAAAGATTTGATCCAAACGGTGAAGAAGAA |
| | AAGCGTAGACATCCATATGCTTTGTTCCCATTTGGTATTGGTCCTAGAGCTTGTAT |
| | TGGTCAAAAGTTCGCTATCCAAGAAATGAAGTTGTCCGCCATTCATTTCTACAGAC |
| | ACTACGTTTTTAGACCCTCTCCATCAATGGAATCTCCACCAGAATTTGTCTACTCC |
| | ATCGTTTCTAATTTCAAGAACGGTGCTAAGTTGCAGGTTATCAAGAGACATATCTA |
| | A (SEQ ID NO: 7) |
| OsCYP711A3 | ATGGAAATCATCTCTACCGTTTTGGGTTCTACTGCTGAATATGCTGTTACTTTGGT |
| | TGCTATGGCTGTTGGTTTGTTGTTGTTGGGTTACTTGTATGAACCATACTGGAAGG |
| | TTAGACATGTTCCAGGTCCAGTTCCATTGCCTTTTATTGGTCACTTGCATTTGTTG |
| | GCTATGCATGGTCCAGATGTTTTTACTGTTTTGGCTAGAAAGTACGGTCCAGTTTT |
| | CAGATTTCATATGGGTAGACAACCATTGGTTATGGTTGCTGATGCTGAATTGTGTA |
| | AAGAAGTTGGCGTTAAGAAGTTCAAGTCCATTCCAAATAGATCCATGCCATCCGC |
| | TATTGCCAATTCCTTGATCAATCAAAAGGGTTTGTGTTTCACCAGAGGTTCTAGAT |
| | GGACTGCTTTGAGAAACATGATCATCTCAATCTACCAACCATCTCATTTGGCCTCA |
| | TTGATTCCAACTATGCAATCTTGCATTGAGTGCGTGTCTAAGAATTTGGATGGTCA |
| | AGAAGATATCACCTTCTCCGATTTGGCTTTGGGTTTTGCTACTGATGTTATAGGTC |
| | AAGCTGCTTTCGGTACTGATTTTGGTTTGTCTAAGATTTCCGCCTCCTCTAACGAT |
| | GATGATATTGATAAGATTGCCACCGATACCTCTGCTGAAGCTAAAGCTTCTTCTGA |
| | ATTCATCAGAATGCATGTTCATGCCACCACATCTTTGAAGATGGATTTGTCTGGTT |
| | CCCTGTCCATTATCATCGGTCAATTATTGCCATTCTTACAAGAACCATTCAGGCAG |
| | GTTTTGAAGAGAATTCCATGGACAGCTGATCACGAAATCGATCATGTTAATTTGGC |
| | ACTAGGTGGTCAAATGGATAAGATAGTTGCTGAAAGGGCTGCTGCTATGGAAAGA |
| | GATCAAGCTGCACCACATGCTCAACAAAGAAAGGATTTTTTGTCTGTTGTTTTGGC |
| | CGCCAGAGAATCTAACAAATCTTGGAGAGAATTACTGACCCCAGATTACATTTCTG |
| | CTTTGACCTACGAACATTTGTTAGCTGGTTCTGCTACTACTGCTTTCACTTTGTCTA |
| | CTGTCTTGTACTTGGTTTCTAAGCACCCAGAAGTTGAGGAAAAGTTGTTGAGAGA |
| | AATTGATGGTTTCGGTCCACATGATCACGCTCCAACTGCTGAAGATCTACAAACTA |
| | AGTTTCCATACTTGGACCAGGTCGTCAAAGAATCCATGAGGTTTTACTTTCTGTCC |
| | CCATTGATTGCTAGAGAAACCTGTGAACAAGTTGAAATTGGTGGTTACGCTTTGC |
| | CAAAAGGTACTTGGGTTTGGTTGGCTCCAGGTGTTTTAGCAAAAGATCCAAAGAA |
| | TTTCCCAGAGCCAGAAGTCTTTAGACCAGAAAGATTTGATCCAAACGGTGAAGAA |
| | GAAAAGCGTAGACATCCATACGCTTTTATTCCATTTGGTATTGGTCCAAGAGCTTG |
| | CATTGGTCAAAAGTTCTCTATCCAAGAAATCAAGTTGTCCGTCATCCACTTGTACA |
| | GAAACTACGTTTTTAGACACTCCCCATCTATGGAATCTCCATTGGAATTTCAATAC |
| | TCCATCGTCTGCAATTTCAAGTACGGTGTTAAGTTGAGAGTCATCAAGAGACATAC |
| | TGCCTAA (SEQ ID NO: 8) |
| SbCYP722B | ATGGATGACATGCACTCTCAATTGCAAGCTGCTGGTGCTGCTTGTCAACAATCTA |
| | ATTCTTTGTTGTTGCCACCACCAGCTGCTGATAGACCTTGTTCTTCTTCATCTTCAT |
| | CCTCGTTGTCTTTGTTGGGTACAGCTGCTGCTGCATGTTTGTTTTTGTCTGCTGCT |
| | ATCTACTGCATCGTCGTTATTATCGTTACTACCTCCTCTAAGCAGAACATCAACAA |
| | CAGATTGATCAGGCGTTTGTTGAAGTTCAAGGGTAGAAGATCTAAGAACGACAGA |
| | AGAAGAGACTACAACAACAATGCTGCTCCACCACCACCACCTCCAGGTAGAGGTT |
| | CTTCTTGGTGGTGGTCAGTTGTTGAAACTTTGGCTTTTGTTTCCGCTAACAGATCT |
| | GGTAGAGGCTTGTATCATTTCGTTGAAGCTAGACATAGAAGATACGGTCCACCAT |
| | GTTTTAGAACTGCTTTGTTAGGTGCTACCCACGTTTTTGTTTCTTCACCAGATGCT |
| | GCTAGAAGTTTGTTGGCTGATGCTGGTGGTTTTTCTAAGAGATACGTTAGAACCG |
| | TTGCCGAATTATTGGGTGAACATTCTTTATTGTGCGCTTCCCATGATGCTCATAGA |
| | GCTTTGAGAAGGGCTGTTGCTCCTTTGTTTAATGCTCAAGCTACTGCTTCTTTGGC |
| | TGCTAATTTTGATGCTTTGGCCAGAAGAATTATCACCAGAGATTGGGCTGCTAAAA |
| | CTACTGCTGTTGTTGTTTTGGATGCTGCTTTGGATGTTACCTTCGAAGCTATTTGC |
| | GATATGTTGATTGGTAGAACTACCACCTTGAAGCGTAGAAGATTACAATCTGATGT |
| | TTTGGCTGTTACCAGAGCTATGTTGGCTTTTCCATTGAGATTGCCAGGTACTAGAT |
| | TTCATGCTGGTTTGAGAGCCAGAAAAAGAATCATGGATGTCTTGAGACAAGAAAT |
| | CGCTTCCAGACAAAGAAACATCATGGATATGGAAGAAATGGAAGAGGATGATTCC |
| | AAGCACGACAATGATTTCTTGCAGTCCTTGTTGTTATTGAGGCGTAGAAAGATGAA |
| | GTCCTCTCAACAGCAACAATCTCCATCTAACTCTAACGATCATTTGTTCTTGACCG |
| | ACGATCAAATCTTGGATAACATCCTGACCTTGATTATTGCTGGTCAAGTTACTACA |
| | GCTTCTGCTATTACTTGGATGGTTAAGTACTTGGCCGATAACAAGGATTTCCAAGA |
| | AACCTTGAGATCCGTGCAATTGGAAATGGCTTTGAAACACCAACATGGTGATTCT |
| | GATGGTCCTTTGACTCTGCAACATTTGAACTCTATGGAATTGGCTTACATGACCGT |
| | CAAAGAAAGTTTGAGAATGGCCTCTATCGTTTCCTGGTTTCCAAGAGTTGCTTTGG |
| | AAGATTGTCAAGTTGCTGGTTTTCATATCAACAAAGGTTGGATCGTTAACATTGAT |
| | GCTAGAGCCTTGCATTATGATGCTACCTTGTATGATAACCCAACCATGTTTGATCC |
| | ATCCAGATTCAAAATGGGTGATGGTAGAAGGTGA (SEQ ID NO: 9) |
| OsCYP722B | ATGAACATGGAATCTTTGGCTGCTGGTGCTTGGTGGGTTGTTGTTTTGTTGTTATT |
| | GGTTTTGACCATCGTTGCCTCTTGGTATAGATCTTGGTGGAAAACTACTGAAGCT |
| | GGTGGTCCATTATTGCCACCTCCAGCAGCTGGTGCTGGACCATGGTGGGTTTGG |
| | GTTTGGCAATGGCGTGAAACTGCTGCTTTTTTGGCTTCTCATGGTTCTGGTAGAG |
| | GTTTCTACCATTTTGTCCAAGAAAGGTACAAGCTGTACAAAGGTGAAGGTGAGGG |
| | TGAAGCTACATGTTGTTTTAGAACTGCTTTGATGGGTAGAGTCCACGTTTTTGTTT |
| | CTGCTTCTCATCCAGCTGCTTCCCAATTATTGACTGCTGAACCACCACATTTGCCA |
| | AAAAGATATGCTAGAACAGCTGCTGATTTGTTGGGTCCACATTCTATTTTGTGTTC |
| | TACCTCTCATGCCCATCATAGACATGCTAGAAGGGCTTTAGCTACTACTTTGTTTG |
| | CTACTCCATCTACAGCTGCTTTTGCTGCTGCATTTGATAGATTGGTTATTAGACAT |
| | TGGACCACCTTGTTGCCACCACACAATCAAAATCAAGTTGTTGTTGTATTGGATGC |
| | CGCCTTGCATATTTCTTACAGAGCTATTTGCGAAATGTTGTTAGGTGCTGGTGGTG |
| | GTAAGTTAAGACCATTGCAATCTGATGTTTTCGCTGTTACTCAAGCTATGTTGGCT |
| | TTGCCATTGAGATGGTTGCCAGGTACTAGGTTTAGAAGAGGTTTACATGCCAGAA |
| | AAAGAATTATGGCTGCCTTGAGAGAAGAAATGGCTGCTAGAAATCATCATCACCA |
| | CCATCACCATCATCATCACGATTTGTTGTCTGTTTTGATGCAAAGAAGGCAATTGG |
| | GTCATCCAGATGCATTGACTGAAGATCAAATTCTGGATAACATGCTGACCTTGATT |
| | ATTGCTGGTCAAGTTACTACTGCTACTGCTATTACTTGGATGGTGAAGTACTTGTC |
| | CGATAACAGATTGATCCAAGATAAGTTGAGAGCTGAAGCCTTCAGATTGGAATTG |
| | AAAGGTGATTACTCTTTGACCATGCAACATTTGAACGCTATGGATTACGCTTACAA |
| | GGCTGTCAAAGAATCATTGAGAATGGCTACTATCGTTAGCTGGTTTCCAAGAGTT |
| | GCTTTGAAGGATTGTCAAGTTGCTGGTTTTCACATCAAGAAGGATTGGATCGTTAA |
| | CATCGATGCCAGATCATTGCATTACGATCCAGATGTTTTTGATAACCCAACCGTTT |
| | TCGATCCATCCAGATTCGATGAAGAAGGCGAAGGCGACGACGCTAAATTGGGTA |
| | GAGCACAACCACAAAAGAGAAGGTTGTTGGTTTTTGGTGCCGGTGGTAGAACTTG |
| | TTTGGGTATGAATCATGCCAAGATCATGATGCTGATTTTCTTGCATAGGCTGTTGA |
| | CTAACTTCAGATGGGAAATGGCAGATGATGATCCATCTTTGGAAAAGTGGGCTAT |
| | GTTCCCAAGATTGAAAAATGGTTGCCCAATTTTGTTGACCCCAATCCATAACTCTT |
| | AA (SEQ ID NO: 10) |
| GaCYP722C | ATGCTGAACTTGTCTGTTGAGGGTTTGACTTTGGTTGTTCAAAACCATTACGGTAT |
| | CTTGATCGTTGCCGTTTTGTCTATTACTATCACCTCCTTGTTGTTGAAAGCTTGGG |
| | GTTCTACTGTTGATATCACTGATGAAGATGGTATCCCAGGTAGATTGGGTTTGCCA |
| | TTTTTTGGTGAAACCTTCTCTTTCTTCTCCGCATCTTATTCTACTAAGGGTTGTTAC |
| | GATTTCGTCAAGCAAAGAAGAAAGCAGTACGGTAAATGGTTCAAGACCAGAATTT |
| | TGGGTAAGACCCATGTTTTCGTTCCATCTGTTGAAGGTGCTAAGACTATTTTGGCC |
| | AATGATTTCGTTCACTTCAACAAGTCCTACGTTAAGTCTATGGCTGATGCTACTGG |
| | TGCTATGTCTGTTTTTTCTGTTCCACATAAGATCCACACCAGAATCAGAAGATTATT |
| | GTCCGATCCATTCTCCATGTCCTCATTGTCTAAATTCGCTGTTAAGTTCGATAAGA |
| | TGGTCTGCGAAAGATTGGACAAGTTGGAAAAATCTGGTAAGTCCTTCAGAGTGAT |
| | CGACTTCTCTTTGAAAATTACCTTCGATGCCATCGTGTCCATGTTGATGTCTGTTA |
| | CTGAAAACCCTTTGTTGGAACAGATCGAAAAGGATTGCACTGATGTCTCTAACTCC |
| | ATGTTGTCTATTCCATTGATGATTCCAGGTACGAGGTACTACAAAGGTATGAAAGG |
| | TAGAGGTAAGCTGAACGAAACTTTCGGTAATATGATTGCCAGAAGAAGAATCGGC |
| | GAAGAATACTTCGATGATTTCTTGCAAACCGTTGTTGACAGAGATTCTTATCCTGA |
| | AGATGAGAAGTTGGACGACCAAGAGATTATTGATAACCTGATCACCTTGATTTTGG |
| | CCGGTCAAACTACTACTGCTTCTGCTATGATGTGGTGCGTTAAGTTTTTGTCCGAA |
| | AACAAGGATGTCTTGGACAGATTGAGAGAAGAACAATTGTCCATCGTTAGAAACA |
| | AAGCTGAAGGTGCATCTTTGACCTTGGAAGATTTGACTGAAAAGAGCTACGGTTT |
| | CAAGGTTGTCAAAGAAACTTTGAGAATGGCCAACGTTTTGATCTGGTTGCCAAGA |
| | GTTGCTATGGATGATTGCATTATCGATGGTTTCGAAGTCAAGAAAGGTTGGTTGG |
| | TTAATGTTGATGCTACCTGCATTCATTACGATCCAAACGTTTACAAAGACCCAACT |
| | AGATTCAACCCATCCAGATTTGATGATTTTCAGAAGCCCTACTCTTTCTTGCCATTT |
| | GGTGCTGGTCCAAGAACTTGTTTGGGTATTAACATGGCTAAGGTTGCCATGTTGG |
| | TTTTCGTCCATAGATTGACATCTGGTTACAAGTGGACTTTGGATGATCCAGATTCT |
| | AGCTTGGAAAGAAAAGAACACATCCCAAGATTGAGATCCGGTTGTCCAATTACTTT |
| | GAAGGCTTTGAACAAGGGCAAGTAA (SEQ ID NO: 11) |
| LjCYP722C | ATGCTGAACTTGTCCAGAGAAGAATTGGTTATCGTTGTTGCTTTGTTGTGCGTTGG |
| | TATTACTTACTTGGCTTCTAAGGCTTGTAAAAGGGCTTCTTCTAACGAAAGAGAAG |
| | ATATCCCAGGTAGATTGGGTTTGCCTTTTATTGGTGAAACCTTCTCCTTTTTGTCC |
| | GCTTACAATTCTACTAGAGGTTCCTACGATTTCGTTACCCCAAGAAGATTGAGATT |
| | TGGTAGATGGTTTAAGACCAGGTTGTTCGGTAAGATCCATATCTTTGTTCCAAACT |
| | CTGAAGGTGCCAGAATTATCTTGGCTAATGATTTCGTCTTGTTCAACAAGGGTTAC |
| | GTTAAGTCTTTGGCTGAAGCTGCTGGTAAGAACTCTTTGTTTTGTGTTCCAGTTGA |
| | ATCCCACAAGAGAATGAGAAGATTATTGTCCGAACCATTCTCTATGACTTCTCCAT |
| | CTGCTTTCATTACCAAGTTCGATAAGAAAATGTGCGCCAGGTTGCAAAAATTGGAA |
| | GAAGGTGGTCAATCCTTCAAGGTTTTGGATTTCTGTATGAAGATGTCCTTCGATGG |
| | TATCTGCGAAATGTTGATGTCTATCACCGAAGATTCCTTGTTGGAAAAGATCTGGA |
| | AGGATTCTATTGCTGCTGGTGAAGCCATGATTTCTATTCCAGCTATGATTCCAGGT |
| | TCCAGGTATTACAAAGGTATGAAGGCTAGAAGAAGGCTAGTTGAAACTTTCACCG |
| | AAATTATTGCCAGAAGAAGAAGAGGCGAAGAATCTGCTGAAGATTTCTTGCAATCT |
| | ATGTTGCAGAGAGATTTGTTCCCAGCTTCTGAAAAGTTGGATGACTCTGAAATCAT |
| | CGACAACATGCTGACCTTTATTTTCTCTGGTCAATCTACTACTGCTACCGCTATGA |
| | TGTGGTCTGTTAAGTTTCTACACGAGAACAAAGAAGTCCAAGACATCTTGAGAGA |
| | AGAACAGTTGTCTCTGTCTAAGATGAAGCCAGAAGGTGCTCCATTGACAAAAGAG |
| | GATATTAACAATATGCCATACGGCTGGAAGGTCTTGAAAGAAACTTTGAGAATGTC |
| | CAACATCGTCTTGTGGTATCCAAGAGTTGCTTTACAAGATTGCACCATTGAAGGTC |
| | GTGAAATCAAAAAAGGTTGGCACGTTAACATTGATGCTACCTGTGTTCATTTTGAC |
| | CCCGACTTGTACAAAGATCCATTGAAGTTTAACCCACAGAGATTCGACGAAACTC |
| | AAAAGCCATACTCTTTCATTCCATTTGGTGCTGGTCCAAGAACTTGTTTGGGTATG |
| | TATATGGCTAAGCTGAAGATGTTGATCTTCATCCATAGATTGGTTGGTGGTTACAC |
| | TTGGACTTTGGATGATTTGGATAACTCCTTGCAAGCCAAAGAGTTGGTTCCAAAAT |
| | TGAGATCTGGTTGCCCAATTACCTTGAAGTCTTTGTCTAAGTCTAGATCCGAAGCC |
| | TGA (SEQ ID NO: 12) |
| VuCYP722C | ATGCTGAACGTCTTGATGAGAGAAGAAGTTTTGTTGGTTGTCCAAAACTGCTACCA |
| | CATTATTTTGGTTGCCTTGTTGTCTATCGGTGTTACTTACTTGGCTTCTAAAGCTTG |
| | GAAAAGAGCTACTACCAACAACAGAGAAGAAATCCCAGGTAGATTGGGTTTGCCA |
| | TTTGTTGGTGAAACTTTCTCTTTCTTGTCTGCTACCAATTCTACCAGAGGTTGTTAC |
| | GATTTCGTCAGATTGAGAAGATTGTGGAATGGTAGATGGTTCAAGACTAGGTTGT |
| | TCGGTAAGATCCATATCTTCGTTCCAAATCCAGAAGGTGCTAGAACTATTTTCGCC |
| | AATGATTTCGTCTTGTTCAACAAGGGTTACGTTAAGTCTATGGCTGATGCTGTTGG |
| | TAAAAAGTCTTTGTTGTGTGTTCCAGTCGAATCCCATAAGAGAATTAGAAGGTTGT |
| | TGTCCGAACCTTTCTCTATGACTTCTTTGTCTGCTTTCGTTACCAAGTTCGATAAGT |
| | TGTTGTGCGAAAGATTGCAGAAGTTGGAAGAAAGAGGTAAGTCCTTCAAGGTTTT |
| | GGATTTCTGTATGAAGATGACCTTCGATGCTATGTGCGATATGTTGATGTCTATCA |
| | CCGAAGATTCTTTGTTGCAGCAAATTGAAGAGGATTGCAACGCTGTTTCTGATGC |
| | CATGTTATCCATTCCAATTATGATTCCAGGTACGAGGTACTACAAAGGTATTACCG |
| | CTAGAAAAAGGCTGATGGAAACCTTCAGAGAAATTATCGGTAGACGTAGAAGAGG |
| | TGAAGAAACCAGAGAAGATTTCTTGCAATCCATGTTGCAAAGGGATTCTTTGCCAC |
| | CATCTGAAAAGTTGGATGACTCCGAAATTATGGACAACTTGCTGACCTTGATTATT |
| | GCTGGTCAAACTACTACAGCTGCTGCTATGATGTGGTCTGTTAAGTTCTTGCATGA |
| | TAACAGGGAAGCCCAAGACATCTTAAGAGAAGAACAATTGTCCATCACCAACATC |
| | AAACCAGATGGTGCTTCTTTGAGTCACGAAGATTTGAACAACATCAGGTACGGTTT |
| | GAAGGTTGTCAAAGAAACCTTGAGAATGTCCAACGTCTTGTTGTGGTTTCCAAGA |
| | GTTGCTTTACAAGATTGCACCATTGAAGGTTACGACATCAAAAAAGGTTGGCACG |
| | TTAACATTGATGCCACCTACATTCATCATGACTCTGACTTGTATAACGACCCCTTG |
| | AAGTTTAACCCAAAGAGATTCGACGAACACCAAAAGCCATATTCCTTTATTCCATT |
| | TGGTTCTGGTCCAAGAACTTGCTTGGGTATTAACATGGCTAAGGTTACCATGTTG |
| | GTTTTCTTGCATAGATTGGCTGGTGGTTATACTTGGACTTTGGATGATTTGGATAC |
| | CTGCTTAGAAAAGAAGGCCCATATTCCAAGATTGAGATCTGGTTGTCCTATCACCT |
| | TGAAGTCCTTGTCTAAAACTATGTTGGAAGCCTAA (SEQ ID NO: 13) |
| SlCYP722C | ATGCTGACCATGTCCATGGAAGATATCCTTTTGTTGCTGTCCAAGTACTACGACAT |
| | CTTGTTGGTTTCCATCTTGGTTATTTCTATCACCGCCTTGTACTTGTCTAAGGGTG |
| | CTAAAAATGCTAAGTCTTGCATTCCAGGTTCTTTGGGTATTCCATTTGTTGGTGAA |
| | ACTTTCGCTTTGTTGTCTGCTACCAATTCTGTTAAGGGTTGCTACGAATTTGTCAG |
| | GTTGAGAAGAGAAAGACACGGTAAATGGTTCAAGACCAGAATTTTCGGTAAGATC |
| | CATGTTTTCGTTCCATCTGTTGAAGGTGCTAAGGCTATTTTCACTAATGATTTCGC |
| | CTTGTTCAACAAAGGCTACGTTAAGTCTATGGCTGATGCTGTTGGTAAAAAGTCTT |
| | TGTTGTGTGTCCCACAAGAATCCCATAAGAGAATTAGAAGGTTGTTGTCCGATCCT |
| | TTCTCCATGAATTCTTTGTCCAAGTTCGTTCAGAGATTCGACGAAATGTTGTACGA |
| | AAGATTGAAGAAGGTCCAGAAGCAGAGAAAGTCTTTTACCGTTTTGGACTTCAAC |
| | ATGAAGACTACCTTTGATGCTATGTGCGACATGTTGATGTCTATCAAGGATTCCTC |
| | TGTTGTCGAACAAATCGAAAAAGATTGCACCGCTATTTCTGATGCCATGTTGTCTT |
| | TTCCAGTTATGATTCCAGGTACGAGATACTTCAAAGGTATTAAGGCTAGAGGTAG |
| | GCTGATGGAAACTTTCAAGGGTATGATTGCTGCTAGAAGAAACGGCAAAGAATAT |
| | TACGAAGATTTCCTGCAGTCCATGTTGGAAAAAGATAGTTGTCCAGCCAATCAAAA |
| | GTTGGACGATGAAGAAATCATGGACAACTTGCTGACCTTGATTATTGCTGGTCAA |
| | ACTACTACAGCTGCTGCTATGATGTGGTCTGTTAAGTTTTTGGATGAACATAGAGA |
| | AGCCCAGAACAGATTGAGAGAAGAACAATTGTCCATCTTGAGATCTAAACCTACT |
| | GGTGCTTTGTTGACCATGGATGATTTGAACTCTATGTCCTACGCTTCCAAGGTTGT |
| | CAAAGAAACTTTGAGAATGTCCAACGTCTTGTTGTGGTTTCCAAGAGTTGCTTTGA |
| | ACGATTGCTCTATTGAAGGTTTCGAGATCAAGAAAGGTTGGCATGCTAATATTGAT |
| | GCTACCTGCATTCATTACGATCCCACCTTGTACAAAGATCCAATGCAATTCAATCC |
| | ATCCAGGTTCGATGAGATTCAAAAGCCATATTCCTACATTCCATTCGGTTCTGGTC |
| | CAAGAACTTGTTTGGGTATCAATATGGCTAAGGTTACCATGTTGGTTTTCTTGCAT |
| | AGATTGACTACTGGTTACAAGTGGACTGTTGATGATCCAGATAGATCCTTGGAAA |
| | GAAAGGCTCATATTCCAAGATTGAGATCTGGTTGTCCAATTACTTTGACTGCTTTG |
| | CCAGATGAGTGA (SEQ ID NO: 14) |
| CaCYP722C | ATGTTGGCCTTGTCCATGGAAGATATTCTGTTGTTGGTTTCCAAGTACTACGATAT |
| | CTTGTTGGCCTCCATTTTGGTTATTTCTGTTACCGCCATCTACTTGATCCCAAAGG |
| | TTTCTACTAGAGCCAAGGATTCTAAATCTTGTGTTCCAGGTAATTTGGGTATCCCA |
| | TTTGTTGGTGAAACTTTCGCTTTGTTGTCTGCTACCAATTCTGTTAAGGGTTGCTA |
| | CGATTTCGTCAGATTGAGAAGAGAAAGACATGGTAGATGGTTCAAGACCAGAATT |
| | TTCGGTAAGATCCATGTTTTCGTTCCATCTGTTGAAGGTGCTAAGACCATTTTCAC |
| | TAATGATTTCGCCTTGTTCAACAAAGGCTACGTTAAGTCTATGGCTGATGCTGTTG |
| | GTAAAAAGTCTTTGTTGTGTGTCCCACAAGAATCCCATAAGAGAATTAGAAGGTTG |
| | TTGTCCGATCCTTTCTCCATGAATTCTTTGTCCAAGTTCGTTGAAAGGTTCGACAA |
| | TATGTTGTGCGAGAGATTGAAAAAGGTCCAGATGGAAGAGAAGTCTTTCACCGTT |
| | TTGGATTTCAACATGAAGATTACCTTCGATGCCATGTGCGATATGTTGATGTCTAT |
| | TAAGGATGCCTCCTTGTTGGAACAAATCGAAAGAGATTGCACCGTTGTTTCTGAT |
| | GCTATGTTGTCTTTCCCAGTTATGATTCCAGGTACTAGGTACTACAAAGGTATTAA |
| | GGCTAGAAGGCGTTTGATGGAAACTTTCAAGAAGATGATTACCGCCAGAAGATCT |
| | GGTAAGGATTATCACGAAGATTTCTTGCAGTCCATGTTGGAAAAAGATTCTTGTCC |
| | AGCTGATCAAAAGTTGGACGATGAAGAAATCATGGACAACCTGCTGACTATGATT |
| | ATTGCTGGTCAAACTACTACAGCTGCTGCAATGATGTGGTCTGTTAAGTTTTTGGA |
| | TGAACATAGAGAAGCCCAGAACAGACTAAGAGAAGAACAATTGTCTGTCTTGAGG |
| | TCTAAACCTAATGGTGCTTTGTTGGCTTTGGATGACTTGAACTCTATGTCTTACGC |
| | TTCCAAGGTTGTCAAAGAAACCTTGAGAATGTCCAACGTTTTGTTGTGGTTCCCAA |
| | GAGTTGCTTTGAACGATTGCTCTATTGAAGGTTTCGAGATCAAGAAAGGTTGGCA |
| | TGCTAATATTGATGCTACCTGCATTCATTACGACCCCACTATCTACAAACATCCAA |
| | TGAGATTCAACCCATCCAGATTCGACGAAATGCAAAAACCATACTCCTACATTCCA |
| | TTTGGTTCTGGTCCAAGAACTTGCTTGGGTATTAACATGGCTAAGGTTACCATGCT |
| | GGTTTTCTTGTACAGATTGACTTCTGGTTACAAGTGGACCGTTGATGATTTGGATT |
| | GCTCTTTGGAAAGAAAGGCCCATATTCCAAGATTGAGATCTGGTTGTCCAATTACT |
| | TTGACTGCATTGCACGATGAGTGA (SEQ ID NO: 15) |
| TpCYP722C | ATGTGGAACTTGTTGAGAGAAGAATTGGCTTTCGTTGGTCAAAACTACTACGATAT |
| | CACCATCGTCTTGTTCATCTCTATTGGTTTGACTTACTTCGCTTCTAGAGCTTGGA |
| | AAAGAGCTAAGACTAACAGAGAAGATATCCCAGGTAGATTGGGTTTGCCAATTATT |
| | GGTGAAACCTTCTCTTTCTTGTCTGCTACCAATTCTACTAGAGGTTGCTACGATTT |
| | CGTCAGATTGAGAAGATTGTGGCATGGTAGATGGTTTAAGACTAGGTTGTTTGGT |
| | AAGGTCCATATCTTCATCCCAAATCCAGAAGGTGCTAGAACTATTTTCGCTAACGA |
| | TTTCGGTTTGTTCAACAAGGGTTACGTTAAGTCTATGGCTGATGCTGTTGGTAAAA |
| | AGTCTTTGTTGTGTGTTCCAGTCGAATCCCATAAGAGAATTAGAAGGTTGTTGTCC |
| | GAACCTTTCTCTATGACTTCTTTGTCTGCTTTCATTACCAAGTTCGACAAGTTGTTG |
| | TGCGGTAGATTGCAAATCTTGGAAGATTCTGGTAAGTCCTTCAAGGTTTTGGACTT |
| | CTCAATGAAGATGACCTTCGATGCTATGTGTGGTATGTTGATGTCTATCACCGAAG |
| | ATTCTTTGTTGAGGCAAATCGAAAAGGATTGCACCGATGTTTCTAACGCTATGTTG |
| | TCTTTCCCAGTTATGATTCCAGGTACTAGGTACTACAAAGGTATCATGGCTAGAAA |
| | GCGTTTGATGGAAACCTTCAGAGAAATTATTGCCAGAAGAAGAAGAGGCGAAGAA |
| | TCTACTGCTGATTTCTTGCAATCAATGTTGCAGAGAGATTCATTGCCTGCTGATCA |
| | AAAGTTGGATGACTCCGAAATTATGGACAACCTGTTGACCTTGATTATTGCTGGTC |
| | AAACTACAACTGCTGCTGCAATGATGTGGTCTGTTAAGTTCTTGCATGATAACAGA |
| | GATGCCCAAGACATTTTGAGGGAAGAACAATTGTCTCTGACTAAGATGAAGCCTG |
| | AAGGTGCTTCTTTGAATCAAGAGGATATCAACAACATGAGGTACGGTTTGAAGGT |
| | TGTCAAAGAAACCTTGAGAATGAGCAACGTCTTGTTGTGGTTTCCAAGAGTTGCTT |
| | TGAACGATTGCACTATTGAAGGTCACGAAATCAAGAAAGGTTGGCACGTTAATATT |
| | GATGCTACCTGCATTCACTACGACTCCGATTTGTTTATTGACCCCTTGAAGTTTAA |
| | CCCACAGAGATTTGACGAAATGCAGAAGCCATACTCTTTCATTCCATTTGGTTCTG |
| | GTCCAAGAACTTGTTTGGGTATGAATATGGCTAAGGTTACCATGTTGGTTTTCTTG |
| | CATAGATTGACCTCTGGTTACACTTGGACTTTGGATGATTTGGATACCTGTTTGGA |
| | AAAAAAGGCCCATATTCCAAGACTGAGATCTGGTTGTCCAATTACCTTGAAGTCCA |
| | TCTCTAGATCTATGCCAGAAGCTTGA (SEQ ID NO: 16) |
| GmCYP722C | ATGCTGAACTTGTTGAGAGAAGAAGTTTTGTTGGTCGTCCAGAAGTACTACTACG |
| | ATTTGATTATGGTTGCCTTGTTCACCATTGGTGTTACTTACTTGGCTTCTAAAGCTT |
| | GGAAAAGAGCTACTACCAACAACAGAGAAGAAATCCCAGGTAGATTGGGTTTGCC |
| | TTTTATTGGTGAAACCTTCTCTTTCTTGTCTGCTACCAATTCTACTAGAGGTTGCTA |
| | CGATTTCGTCAGATTGAGAAGATTGTGGAATGGTAGATGGTTCAAGACTAGGTTG |
| | TTCGGTAAGATCCATATCTTCATTCCATCTCCAGAAGGTGCTAGAACTATTTTCGC |
| | TAATGATTTCGTCTTGTTCAACAAGGGTTACGTTAAGTCTATGGCTGATGCTGTTG |
| | GTCAAAAGTCTTTGTTGTGTGTTCCAGTTGAATCCCACAAGAGAATTAGAGGTTTG |
| | TTGTCTGAACCTTTCTCCATGACTTCTTTGTCTGCTTTCGTTACCAAGTTCGATAAG |
| | ATGTTGTGTGGTAGATTGCAGAAGTTGGAAGAATCTGGTAAGTCCTTCAAGGTTTT |
| | GGATTTGTGTATGAAGATGACCTTCGATGCTATGTGCGATATGTTGATGTCTATCA |
| | CCGAAGATTCTTTGTTGAGGCAAATCGAAGAAGATTGCACTGCTGTTTCTGATGC |
| | CATGTTGTCTATTCCAATTATGATCCCAAGAACCAGGTACTACAAAGGTATTACCG |
| | CTAGAAAACGTGTCATGGAAACTTTCGGTGAAATTATTGCTAGAAGAAGAAGAGG |
| | CGAAGAAACCCCAGAAGATTTCTTGCAATCTATGTTGCAAAGGGATTCTTTGCCA |
| | GCTTCTGAAAAGTTGGATGACTCCGAAATTATGGACAACCTGTTGACCTTGATTAT |
| | TGCTGGTCAAACTACTACAGCTGCTGCTATGATGTGGTCTGTTAAGTTCTTGCATG |
| | ATAACAGAGAAACCCAGGACATTTTGAGGGAAGAACAATTGTCCATTACCAAGAT |
| | GAAGCCTGAAGGTGCTTCTATCAATCATGAGGATTTGAACTCTATGAGGTACGGT |
| | TTGAAGGTTGTCAAAGAAACCTTGAGAATGTCCAACGTCTTGTTGTGGTTTCCAAG |
| | AGTTGCTTTGGAGGATTGCACTATTGAAGGTTACGATATCAAGAAAGGTTGGCAC |
| | GTTAACATTGATGCTACCCATATTCATCACGACTCCGACTTGTACAAAGATCCATT |
| | GAAGTTTAACCCACAGAGGTTCGACGAAATGCAAAAGCCATATTCCTTTATTCCAT |
| | TCGGTTCTGGTCCAAGAACTTGCTTGGGTATTAACATGGCTAAGGTTACCATGTT |
| | GGTTTTCTTGCATAGATTGACTGGTGGTTACACTTGGACTTTGGATGATTTGGATA |
| | CCTGTTTGGAAAAAAAGGCCCACATTCCAAGATTAAGAAACGGTTGTCCAATCAC |
| | CTTGAAGTCCTTGTCTAAATCAATGCCAGAAGCCTGA (SEQ ID NO: 17) |
| FaCYP722C2 | ATGTTGTGCGCCATCTCTAAAGAAGAGTTTTTGTTCTTGGTCAAGAAGCACTACGA |
| | CGTTTTGATAGTTGCTCTGTTGTTCTCTATTGGTGTTGCCTTTTTCGTTTCTAAGTT |
| | GGCTTGGAGAAAAGCTACTGCTTCTTCTAGAGGTTATATCCCAGGTAGATTGGGT |
| | TTGCCTTTTATTGGTGAAACTGTCCCATTTTTGTCCGCTGTTAAGTCTACTGGTGG |
| | TTGCTACGAATTCATCAGATTGAGAAGAATTAGATACGGCAAGTGGTTCAAGACTA |
| | GAGTTTTCGGTAAGATCCATGTTTTCGTTCCATCTACTGATGGTGCCAGAATGATT |
| | TTGTCTAACGATTTCGTCAAGTTCAACAAGGGTTACATGAAGGCTTTGTTGGATTG |
| | CATTGGTGACAAGTCTTTGTTCACTGTTTCTCACGAAAACCACAAGAGAATCAGAG |
| | ATTTGTTGTCCGACTTGTTCTCGATGAACTCTTTGTCCTCCTTCATCGAAAAATTC |
| | GACAAGATGTTGTGCCAGAGGTTGAAAAAATTGGAACAAGGTGGTAAGTCCTTCT |
| | CCGTTTTTGATTTCTCTATGAAGATGGCCTTCGATTCTATGTGTTGCATGTTGATG |
| | TCTATTACCGATGAAGCCTTGTTGAGACAATTGGAAAAGGATATTACCGCTGTTTC |
| | CAACGCTATTTTCTCTGCCCCAATTATGATTCCAGGTACGAAATATTACAAGGGTA |
| | TTAAGGCCAGACGTAGGATCATGGAAATTTTCAAGGATATGATTGCCAGGCGTAG |
| | ATCTGGTGAAGAATGGCCAGAACCAGAAGATTTCTTGCAATCTTTGTTGAGAAGG |
| | GACTCTTATCATCCAGACGAAAAGTTGCAAGATCCAGAAATCATGGATAACCTGAT |
| | GACCTTGTTGATTTCAGGTCAAGCTACTACATCTGCTACTCAAATGTGGTCTGTTA |
| | AGTTCTTGGACGAAAATCAGCAAGTCCAAGACATCTTGAGAAAAGAGGTTTTGTC |
| | CATTGCTAGGTCTAAACCAGAAGGTGCTTCTGTTACTTTGGAAGATATTAACAGAA |
| | TGCCCTACTGCTGGAAGGTTTTGAAAGAAACTATGAGGATCTCCTCCGTTGTTAT |
| | GTGGTATCCAAGAGTTGCTTTGGCTGATTGCACTATTGAAGGTTTCGAAATCAAGA |
| | AAGGTTGGCATGTTAACGTTGATGCCAAGTGCATTCATAATGATCCAGAGTTGTAT |
| | CCCGATCCATCTAAGTTTAATCCATCCAGATTTGACGAGCTGTTGAAGCCATATTC |
| | CTTTATTCCATTTGCTTCCGGTCCTAGAACTTGCTTGGGTATTAACATGGCTAAGA |
| | TGACCATGATGGTGTTCTTGTACAGATTGACTTCTGCTTATTCTTGGACCGTTGAT |
| | GATTTGGATACTTCCTTACAAGAAAACGCTCACTTCCCAAGATTGAGATCTGGTTG |
| | TCCAATTACTTTGAACCCCTTGTGA (SEQ ID NO: 18) |
| 28 aa tag | ATGGAATTATCACAAGTTTGTACAAAAAAGGAGGCTGGCGCCGGAACCAATTCAG |
| | TCGACTGGATCCAAGAAGGAGATATAACC (SEQ ID NO: 19) |
| SohB tag | ATGGAATTGTTGTCTGAATATGGTTTGTTTTTGGCGAAAATCGTTACCGTTGTGCT |
| | AGCGATTGCGGCGATTGCCGCCATTATTGTCAATGTTGCTCAACGTAATAAACGC |
| | CAGCGTGGCGAGTTACGGGTCAACAATCTCAGC (SEQ ID NO: 20) |
| tPpD27 | ATGGCAGAACCCTCAGGGAAGCCTGCTCCTATGGGCAAAAAGACACATTACAAAG |
| | ATTCCTGGCTTGATAACACCATTCTGTCTATCTGTATGCGTCGCCTGGGTAACGT |
| | GACTGGCGTCAGCACAACTAAAAAGGGATATGACGGCTTCGTCGAATTGACGCG |
| | TAAAGTCATGGAGACCCGTTCGCCACTTTTGCAGCGTGCGTCGTCAATGCGCGTT |
| | CTTCACTCCGCGATCCCACCgTGGTTATTGAAAATTATCCGCCGTTTTCTTCCCAA |
| | TAATCAAAAGACAGCCGAGACATTTGCAGCCGCCACTTTATATGCAGAGTGGCTG |
| | GTTGGACCATGCGAAGTTAAGGAAGTCGAGGTGAACGGGACGATGCAGAAATCA |
| | GGGGTGTTGATTAAGAAATGTCGTTACCTGGAGAGCAGCAATTGCGTGGGAATGT |
| | GCGTCAATTTATGCAAGATTCCGACACAGGATTTTTTCACCAACTCGTTGGGTGTT |
| | CCCTTGACCATGACTCCAAATTTCGAAGACATGTCTTGTGAGATGATCTACGGAC |
| | AAACCCCACCGTCCATCGAAGAAGACCCGGCACTTCAACAACCGTGTTTCGCCAC |
| | GCTGTGCCCCACTGCCCGTAAAAAAGCATTGACTTGTTCAAAAACTCCGTTACCA |
| | CCCTGA (SEQ ID NO: 21) |
| PpD27 | ATGTTGTTGCAATTGGAAGCTTTGAGAGGTGGTGCTACTAGAATTCCATTTGCTTC |
| | TCATTTGGAATTGAAGCAACCTAGAAGGCATAGACCAGATGGTGTTAATATGGTTA |
| | GATGCAGAATGGCTGAACCATCAGGTAAACCAGCTCCAATGGGTAAAAAAACCCA |
| | TTACAAAGATAGCTGGTTGGACAACACCATTTTGTCTATCTGTATGAGAAGATTGG |
| | GTAACGTCACTGGTGTTTCTACTACTAAGAAAGGTTACGATGGTTTCGTCGAATTG |
| | ACTAGAAAAGTCATGGAAACCAGATCTCCCTTGTTGCAAAGGGCTTCTTCTATGA |
| | GAGTTCTGCATTCTGCTATTCCACCATGGTTGTTGAAGATCATCAGAAGATTCTTG |
| | CCCAACAATCAAAAGACTGCTGAAACTTTTGCTGCTGCTACATTATATGCTGAATG |
| | GTTGGTTGGTCCATGCGAAGTAAAAGAAGTTGAAGTTAACGGCACCATGCAAAAG |
| | TCTGGTGTTTTGATTAAGAAGTGCAGGTACTTGGAATCCTCTAACTGTGTTGGTAT |
| | GTGTGTTAACTTGTGCAAGATTCCAACTCAAGACTTCTTCACCAATTCTTTGGGTG |
| | TTCCATTGACTATGACTCCAAACTTCGAAGATATGTCCTGCGAAATGATCTATGGT |
| | CAAACTCCACCATCCATTGAAGAAGATCCAGCTTTACAACAACCTTGTTTCGCTAC |
| | TTTGTGTCCAACTGCTAGAAAAAAGGCTTTGACTTGTTCTAAGACTCCATTGCCAC |
| | CATAG (SEQ ID NO: 22) |
| ZmD27 | ATGGAAGTTGCTGCTGCTACTTGTACTTTGTTGGCTGCTGTTGCTTTGCCAGTTTC |
| | TTCATCTGCTGCTGCAGCTAGATCTGCTACAAAACAATCTTACTCTAGAAGAAAGA |
| | CCACCGCTGTTAGAGCTGTTATGGCTAGACCACATCAAGTTCCACCACCAGCTAC |
| | TGCAACTGCTACAGCTACTACTTATAGAGATAACTGGTTCGATAAGTTGGCCATCG |
| | GTTATTTGTCTAGAAACTTGCAAGAGGCTTCTGGTATGAAGAATGGTAAAGATGGT |
| | TACGAAGGTTTGATTGAAGCTGCTTTGGCTATTTCTGCTTTGTTCAGAGTTGACCA |
| | ACAATGGGATACTGTTGCTTCTGCTTTACAAAGAGCTTTCCCATCTTACATCTTGA |
| | CCATGATCAAAGTTATGATGCCACCATCCAGATTCTCCAGAGAATATTTTGCTGCT |
| | TTCACCACTGTTTTCTTCCCATGGTTGGTTGGTCCATGTGAAGTTAGAGAATCTCA |
| | AGTTGATGGTAGAGAAGAGAGAAACGTTGTTTACATTCCAAAGTGCAGGTTCTTG |
| | GAATCTACCAATTGTGTTGGTATGTGTACCAACTTGTGTAAGATTCCATGCCAAAG |
| | ATTCATCCAGGATTCTTTGGGTACTGCTGTTTACATGTCTCCAAACTTCGAAGATA |
| | TGTCCTGCGAAATGATTTTCGGTCAACAACCACCAGAAGATGATCCAGCTTTGAA |
| | ACAACCATGTTTCAGGACTAAGTGCATTGCCAAGCAAAACCATCAAGTTAACTGCT |
| | CCATTTAG (SEQ ID NO: 23) |
| VvD27 | ATGTTGCATTTGCCATTCTGCTCCTACATCTTCCATCCAATTTCTTTGTCCAAGTCT |
| | CAGTTGACCTTGCTGTTGTTCTCCTTGTATCAAATTCCAATGGATGCCAAGTTGGT |
| | TACCCAACATAGATCACATGTTTGGGCTGGTAAAAGAGGTATGCATAAGCCAAGA |
| | TCTTCTCCACCAATTTTGGCTGTTTTGGCTAGACCAGCTGATAATTTGACTTTGGT |
| | CAAAGAAACCCCATCCTTGTTGACTGATAATTGGTTCGATAGAATCGCCATCAACC |
| | ACTTGTCTCAATCTGTTCAAGCTACTACTGGTTTGAGGAATTCTAAGTCTGGTTAC |
| | GAATCTTTGGTTGAAGCTGCTGCTATGGTTTCCAGAAATTTCGATCCAATTCAACA |
| | GTGCGAATTGGTTATTGAAGCTCTGAACAAAGCTTTCCCATCTCCAATTTTGTCCT |
| | TGATCAGAACTTTGATGCCACAGTCTAAGTTCACCAGAGAATACTTTGCTGCTTTC |
| | ACTACTTTGTTCTTCGCTTGGTTGGTTGGTCCATGCAAAGTTATTGAATCTGAGAT |
| | CAACGGTAGGCGTGAAAAGAATGTTGTTCATATCAAGAAGTGCAGGTTCCTGGAA |
| | GAATCTAATTGTGTTGGTATGTGCTTGAACTTGTGCAAGAATCCATCTCAGAAGTT |
| | CATCAAGGATTCTTTGGGTATGCCAGTTAACATGGTTCCAAACTTCGATGATATGT |
| | CCTGCCAAATGATTTTCGGTCAAGATCCACCAGGTGATGATCCAGTTTTGAGACA |
| | ACCTTGTTACAAGTTGTGTAAGGCCAAGAAGAACCATTCTGTTAACTGCTCCATTT |
| | AG (SEQ ID NO: 24) |
| SmD27 | ATGGCCATGTCATCTGTTTCATTGCACTCCAGATCTTTGATCAGATGCGAAATTGC |
| | TGAACCATCTGGTAAACCAGCTCCAATGGGTCAAAAAACTAGATACAAGGATTCC |
| | ATCTTCGACAGAGCTTTCATGTCTTTGTTTGCCAGAAAGATGGAAAACGCTACTGG |
| | TAGAGCTTCTAAAAAGACTGGTTACGATGGTTTCGTTGATGTTTCCAGAGGTGTTT |
| | TACAAGGTAGAAACCCAGTTGAACAAAGAGCCTTGGTTAGAGAAGTTCTGTTGTC |
| | TATTATGCCACCAGGTGCTCCAGAAACTTTTAGAAAGTTGTTTCCACCAACTAAGT |
| | GGGCTTGTGAATTCAATGCTGCTATTACTGTTCCATTCTTCCAATGGTTGGTTGGT |
| | CCATGTGAAAGATTCGAAGTTGAAGTTAACGGTGTCAAGCAAAAGTCCGGTGTTA |
| | AGATTTTGAAGTGCAGGTACTTGGAAAACTCTAACTGTGTTGGTATGTGCGTTAAC |
| | ATGTGTAAGATACCAACTCAAGACTTCTTCACCAACGATTTTGGTTTGCCATTGAC |
| | TATGACTCCAAACTTCGAAGATATGTCCTGCGAAATGATCTACGGTTTACAACCTA |
| | CTTCCTTGGAAGAAGATCCAGCTTTGAAACAACCATGCTTGCAATTGTGTCCAACT |
| | TCTGCTACTACTGCTGTCTGTACAAAATTGCAGCAAGAGTACTAG (SEQ ID NO: |
| | 25) |
| SbD27 | ATGGAAGTTGCTGCTACTTGTATGCCATTGGCTCATGCTCATGGTGTTGGTGTTTT |
| | GCCAGCTTGGTCATCTCCATCTACTGCTGCTGCTACAGCTAGATCTGTTACTAGA |
| | CAATCTTACACCTACACCAGAAGAAAGAGATTGGCTACTGCTAGAGGTGTTATGG |
| | CTAGACCACAAGAAGTTGTTGTTGCTCCAGCTCCACCAGCTAGACCAACTCCACC |
| | ACCACCAACTACTACAAAAGAAAGAACTGCTTTGGCTCCACCTCCTACTACAACTA |
| | CTTATCATGATTCCTGGTTCGATAAGTTGGCCATTGGTTATTTGTCCAGAAACTTG |
| | CAAGAGGCTTCTGGTATGAAGAATGGTAAAGATGGTTACGAAGGTTTGATTGAAG |
| | CTGCCTTGGCTATTTCTGCTTTGTTCAGAGTTGACCAACAATTGGAAACTGTTGCT |
| | AAGGCTTTGGAACAAGCTTTCCCATCTTACATTTTGACCATGATCAAGATCATGAT |
| | GCCACCATCTAGATTCTCCAGAGAATACTTTGCTGCTTTCACCACTATTTTCTTCC |
| | CATGGTTGGTTGGTCCATGTGAAGTTAGAGAATCTGAAGTTGACGGCAGAAAAGA |
| | AAAGAACGTTGTTTACATTCCAAAGTGCAGGTTCTTGGAATCTACCAATTGTGTTG |
| | GTATGTGTACCAACTTGTGCAAGATTCCATGCCAAAAGTTCATCCAAGATTCTTTG |
| | GGTACTGCCGTTTACATGTCTCCAAATTTCGAAGATATGTCCTGCGAAATGATCTT |
| | CGGTCAACAACCACCTGAAGATGATCCAGCTTTGAAACAACCATGTTTCAGGACT |
| | AAGTGCATTGCCAAGCAAAACCATCAAGTTAACTGCTCCATTTAG (SEQ ID NO: |
| | 26) |
| RcD27 | ATGGAAGCCATTATCTTCCCACAAAACAGAGGTCCAATTCCATCTCAACCATTGCC |
| | AAGACAAACTAACAGATTGAACAAGTCCAGAATCTTCGCTGTTTTGACTAAGCCAA |
| | CCGAAAACATTTCTGGTGTCAAAGAGAAGAAGTCCTCTGATAATTTGCCAGGCTT |
| | GACCTCTAAGATCTCTATCTACAGAGATTCCTGGTTCGATCAATTGGCCATTAACC |
| | ATTTGTCCCAATCTGTTCAAGCTGCTACTGGTATTGGTTGCATTTCTATTTCTACCA |
| | ACTTGCCCATTAAGACCTTGTTGCCACAATCAAGATTCACCAGAGAATACTTTGCT |
| | GCTTTCACTACCTTGTTCTTCGTTTGGTTGATTGGTCCATGTCAAGTTAGGGAATC |
| | TGAATTCAACGGCCGTAAAGAAAAGAACGTTGTTCATATCAAGAAGTGCAGGTTC |
| | TTGGAAGAAACTAACTGTGTTGGTATGTGCACCAACTTGTGTAAAGTTCCAACTCA |
| | AACCTTCATCAAGCAGTCTTTGGGTATGCCAGTTAATATGGTTCCATCTAAGTACC |
| | CAAGATCCACCTTGTTGAAACAAGATCCTCCAATTCCTACTGAAGATCCAGCTTTT |
| | AGACAACCATGTTACAAGTTGTGTAACGCTTTGCCACATCCATTTTCTCCATCTCA |
| | TACTCATATGCACATGCATGCTCAAAACAGAAATCCACATTCTCCACCAAACTTGG |
| | TTGACTAG (SEQ ID NO: 27) |
| PsD27-2 | ATGCAAGTTGCTGCTAATTCCTTCGGTAAGGTTTACTGTCAAGGTATCCCAAGACA |
| | TAACTACTCCTCATCTTACAAGTTCCAGAGAAAGAACCATAGCTTCGGTTTGAGAA |
| | GAAAGATGATTATCGAATGCGGTATTGCTGAACCATCTGGTCAACCAGCTCCAAT |
| | GGGTCAAAAAACTAGATACAACGATAACCTGTTCGACAAGGTTTTCATGGCTTTGT |
| | TTGCCCGTAAGATGAACAATATTGCTGGTGGTAAATCCACTGGTAGAGAAGAAGG |
| | TTACGAAAGATTCGTTGAAACCTCCAGATCTGTTATGTTGGGTAGAACTCCAAAGC |
| | AACAACAAGAAGCTGTTAGACAAGTCCTGTTGTCTATGTTGCCACCAGGTGCTCC |
| | AGAAAGATTCAGAAAGTTGTTTCCACCAACAAAATGGGCTGCTGAATTCAATGCTG |
| | CTGTTACTGCTCCATTTTTCCATTGGTTGGTTGGTCCATCTGAAGTTGTTGAAGTT |
| | GAAGTTAACGGTGTCAAGCAAAAGTCTGGTGTTCATATTAAGAAGTGCAGGTACT |
| | TGGAAAACTCTGGTTGTGTTGGTATGTGTGTTAACATGTGCAAATTGCCAACTCAA |
| | GACTTCTTCACTAACGAATTTGGTTTGCCATTGACTATGACCCCAAACTTCGAAGA |
| | TATGTCCTGTGATATGGTTTACGGTCAACCACCACCACCTCCAGAAGAAGATCCA |
| | GCTTTTAAACAACCATGTTACGCTGCTTTTTGCTCTATGGCTCAACCAGATTCTGA |
| | AGCTTGTCCAAAATTGTCCGTCAGAAAAAGATTGGACATGAGCTTCTAG (SEQ ID |
| | NO: 28) |
| PeD27 | ATGGATGCTGGTTTCTTGTGTCAAACTAGATCTCCAGTTCCATCTTTGCCAAGACA |
| | AAAAAGAGCTTGTAAGCTGAAACACAGATCACCAGTTTTGGCTGTTTTGACTAGAA |
| | CTCCAGATACCAATATGACCGGTGAAGAAAGAAAAGCTTCTCATCCAACTGATAT |
| | GTTGTTGGCAGGTTTGACTAAGAAAACCACCGTTTACAACGATTCTTGGTTTGCTA |
| | AGTTGGCCATCAACTACTTGTCTCAAAGATTCCAAGATGCTACCGGTATGAGAAAT |
| | TCCAAGAGAGATTACGAATCCTTGACTCAAACTGCTAGAGATACTTGGAGAAAGTT |
| | CAACCCAACTCAACAACATGAATTGGTCTTGCAGTCTTTGAACAGAGCTATTCCAG |
| | CTACTATTTCTACCTTGGCTAAAATGATGTTGCCACAATGTACTTTCACCAGAGAA |
| | TACTTTGCTGCTTTCACTACCTTGTTCTTCGTTTGGTTGGTTGGTCCATGTGAAGT |
| | TAGAGAATCTGATTTCAACGGCCGTAAAGAAAAGAACGTTGTTCATATCAAGAAGT |
| | GCAGGTTCTTGGAAGAAACTGATTGCATTGGTATGTGTACCAACTTGTGTAAGGTT |
| | CCATCTCAGACCTTTATCAAGCACTCTTTTGGTATGCCAGTTAACATGGTTCCAAA |
| | CTTCGAAGATATGTCCTGCGAAATGATCTATGGTCAAGAACCACCAGCTATTACTG |
| | AAGATCCAGCTTTTAAACAACCCTGCTACAAGTTGTGCAAAGAGAATAGAAAACAC |
| | TCCATGCAGTGCTCCTCTTAG (SEQ ID NO: 29) |
| MtD27 | ATGGACTCCAAGATGATTGCCCATAACATGTCTTTGACTCCAACTTTGGCTCAATG |
| | GAAGAAGTTGAGATTGAAGCCAAAACACACTTTCGTTGTTGGTGTTTTAGCTAGAC |
| | CAACCGATGATATTTCTGAGGAAACTTTGAGAAAGACCAACGTCTACAAGGATAA |
| | CTGGTTTGATAAGTTGGCCATCAACCACTTGTCTAAGTCTGTTCAAGCTGCTACTG |
| | GTATCTCTAACAACAAGTCTGGTTTCGATTCTTTGGTTGAAGCTGCAACTGTTGCT |
| | TCTCAAAAGTTCAACACTACTCAACAACAGGGCATTATTTTGGATGCTTTGGATAG |
| | AGCTTTCCCCAAGCCAATTTTGTCCGTTATTAGAAGAGTTATGCCACCATCTAAGT |
| | TGGCTAGAGAATACTTTGCTGTTTTCACCACCATTTTCTTCGCTTGGTTGTTGGGT |
| | CCATCTGAAGTTAGAGAATCTGAAATCAACGGTAGGCGTGAAAAGAACATCGTTC |
| | ATATTAAGAAGTGCAGGTTCCTGGAAGAAACTAACTGTGTTGGTATGTGCATTAAC |
| | TTGTGCAAAATGCCATCTCAGCTGTTCATCAAGGATTCTTTTGGTATGCCAGTTAA |
| | CATGGTTCCAAACTTCGATGATATGTCCTGCGAAATGATTTTCGGTCAAGAACCAC |
| | CAGCTTCTACTGATGATCCAGCTTTGAAACAACCATGCTACAAATTGTGCAAGGCT |
| | AAGAAAAATCATGCTACCCAATGCCTGTCTTAG (SEQ ID NO: 30) |
| SbCYP711A2 | ATGGAAATGGGTACTGTTTTGGGTGCTATGGAAGAGTACACTTTTACTTTTTTGGC |
| | TATGGCCGTTGGTTTCTTGGTTTTGGTTTACTTGTATGAGCCATACTGGAAGGTTA |
| | GACATGTTCCAGGTCCAGTTCCATTGCCATTGATTGGTCACTTGCATTTGTTGGCT |
| | AAACATGGTCCAGATGTTTTTCCAGTTTTGGCCAAGAAACACGGTCCAATTTTTAG |
| | ATTCCATGTCGGTAGACAACCATTGATTATAGTTGCTGATGCCGAATTGTGCAAAG |
| | AAGTCGGTATTAAGAAATTCAAGTCCATGCCAAACAGGTCTTTGCCATCTCCAATT |
| | GCTAATTCCCCAATTCATAGAAAGGGTTTGTTCGCTACTAGAGACTCTAGATGGTC |
| | TGCTATGAGAAACGTTATTGTCTCTATCTACCAACCATCTCATTTGGCTGGTTTGA |
| | TGCCAACTATGGAATCTTGTATTGAAAGAGCTGCTACCACCAACTTAGGTGATGG |
| | TGAAGAAGTTGTTTTCTCCAAGTTGGCTTTGTCTTTGGCCACTGATATTATTGGTC |
| | AAGCTGCTTTTGGTACTGACTTTGGTTTGTCTGGTAAACCAGTTGTTCCAGATGAT |
| | GATATGAAGGGTGTTGATGTTGTTGTTGGTGATGCTGCTAAAGCTAAAGCTTCTTC |
| | TTCCGAATTCATCAACATGCATATCCATTCCACCACCTCATTGAAGATGGATTTGT |
| | CAGGTTCTTTGTCTACTATCGTTGGTGCTTTGGTTCCATTCTTGCAAAATCCATTG |
| | AGACAGGTTTTGTTGAGAGTTCCAGGTTCTGCTGATAGAGAAATCAATAGAGTTAA |
| | CGGTGAGTTGAGAAGAATGGTTGATGGTATCGTTGCTGCTAGAGCTGCAGAAAGA |
| | GAAAGAGCACCAGCTGCTACTGCTGCTCAACAACATAAGGATTTTTTGTCCGTTG |
| | TTTTGGCTGCCAGAGAATCTGATGCTTCTACAAGAGAATTACTGTCCCCAGATTAT |
| | TTGTCTGCTTTGACCTACGAACATTTGATTGCTGGTCCAGCTACTGCAGCTTTTAC |
| | ATTGTCATCTGTTGTTTACTTGGTTGCTAAGCACCCAGAAGTTGAAGAAAAGTTGT |
| | TAAGAGAAATGGATGCCTTTGGTCCAAGAGGTTCTGTTCCAACTGCTGATGACTT |
| | GCAAACTAAGTTTCCTTACTTGGATCAGGTCGTCAAAGAATCTATGAGGTTGTTTA |
| | TGGTTTCCCCATTGGTTGCAAGAGAAACTTCTGAAAGAGTTGAAATTGGCGGTTA |
| | CGTTTTGCCAAAAGGTGCTTGGGTTTGGATGGCTCCAGGTGTTTTAGCAAAAGAT |
| | GCTCATAATTTTCCCGATCCAGAGTTGTTTAGACCAGAAAGATTTGATCCAGCTGG |
| | TGACGAACAAAAGAAAAGACATCCATACGCTTTCATCCCATTTGGTATTGGTCCTA |
| | GAGTATGCATTGGTCAAAAGTTCGCTATCCAAGAAATCAAGTTGGCCATTATCCAC |
| | TTGTACCAACACTACGTTTTTAGGCATTCTCCCTCAATGGAATCACCATTGGAATT |
| | TCAATTCGGTATCGTCGTTAATTTCAAGCACGGTGTTAAGTTGCACGTTATCAAAA |
| | GACACGTTGAGAACAACTAA (SEQ ID NO: 31) |
| EgCYP711A | ATGTTGGAGTTCTCCTTGCAAAGAGTTGTTGAAATTGGTACTACCTTCATGGCTTC |
| | TCCACCAGCTTTGTCTACTTTGGCTTTTACTGGTTTGGCTATTTTGACCGGTTTGG |
| | TGTCTTACTTGTATGCTCCATATTGGGGTGTTAGAAAAGTTCCAGGTCCACCAGCA |
| | TTTCCATTGGTTGGTCATTTGCCATTGATGGCTAAATATGGTCCAGACGTTTTCTC |
| | CATTTTGGCTAAAAGATACGGTCCAATCTTCAGATTCCATATGGGTAGACAACCCT |
| | TGATTATTATCGCTGATGCTGAATTGTGCAAAGAGGTTGGTATTAAGAAGTTCAAG |
| | GACATCCCAAACAGATCCATTCCATCTCCAATTGCTGCTTCTCCATTGCATCAAAA |
| | GGGTTTGTTTTTCACCAAAGATGCTAGATGGTTGACCATGAGAAACACCATTTTGT |
| | CCTTGTACCAACCATCTCATTTGGCTTCATTGGTTCCAACTATGCAAGAGTACATT |
| | GAATCTGCTACCGAAAACCTGCAGTCCTTCAAAGAAGAAGATATTGCCTTCTCCAA |
| | CCTGTCTTTGAAGTTGGCTACTGATGTTATTGGTCAAGCTGCTTTTGGTGTTGATT |
| | TCGGTTTGTCTAGAGGTCAATCTGAACAAGGTCATGGTTCTTCAGCTAACGAAAAA |
| | GGTAATGGTGATAGAGACAACGAAGTCTCCGATTTCATCAACAAACATATCTACTC |
| | CACCACGCAGTTGAAGATGGATTTGTCTGGTTCTTTCTCCATCATCTTGGGTTTGT |
| | TAGTTCCAATCCTACAAGAACCATTCAGGCAGATTTTGAAGAGAATTCCAGGTACT |
| | ATGGACAGAAAGGTTAACCAGGCTAACAAAGAATTGGCTGGTAGATTGAATGGCA |
| | TCGTGTCTAAAAGGATGAAGGAAAAAGAGCGTTCCTCCAAGGATTTCTTGTCCTT |
| | GATTTTGAACGCCAGAGAATCTGAAAGAGCTGCTAAGAACGTTTTCACTCCAGATT |
| | ATGTTTCTGCCGTTACCTACGAACATTTGTTGGCTGGTTCTGCTACTACCTCTTTT |
| | ACTTTGTCATCCGTTGTTTACTTGGTTGCTGGTCATCCAGAAGTTGAAAAGAAGTT |
| | GTTGGAAGAAATCGATGGTTTCGGTCCAAGAGATCAATTGCCAACTGCTCAAGAC |
| | TTGCAAAAAAAGTTCCCATACTTGGACCAGGTTATCAAAGAAGCTATGAGGTTCTA |
| | TTTGGTGTCACCTTTGGTTGCTAGAGAAACCTCTAGAGAAGTAGAAATTGGTGGTT |
| | ACTTGTTGCCAAAAGGTACATGGGTTTGGTTGGCTCCAGGTGTTTTAGCAAAAGA |
| | TCCAAAGAATTTTCCAGAGCCAGAAAAGTTTAGGCCAGAAAGATTTGATCCAAACT |
| | GCGAAGAACAAAAATGGCGTCATCCATATGCTCATATTCCATTTGGTATTGGTCCT |
| | AGAGCTTGCATCGGTCAAAAGTTTTCATTGCAAGAAATCAAGCTGTCGCTGATCC |
| | ACTTGTACAGAAAGTATATCTTCAGGCACTCCAGCTCTATGGAAAAACCATTGGAA |
| | TTGGAATTCGGCATCGTCTTGAATTTCAAACACGGTGTTAAGTTGAGAGTCTTGGA |
| | GAGAAAGTGA (SEQ ID NO: 32) |
| SlMAX1 | ATGATGTTCTTGTCCTCCGCCATTCAAGAATCTCCAATTGCTTCTACCATTTTCAC |
| | CATTTTGGCTGGTGTTTTGGTCTACTTGTATAGACCATATTGGAGAGTTAGAAAGG |
| | TTCCAGGTCCACCAGCTTTTCCATTGGTTGGTCATTTGCCATTGATGGCTAAATAT |
| | GGTCCAGATGTTTTCTCCGTTTTGGCTAAACAATACGGTCCAATCTACAGATTCCA |
| | TATGGGTAGACAACCATTGGTTATAGTTGCTGATGCTGAATTGTGTAGAGAAGTC |
| | GGTATTAAGAAGTTCAAGGACATCCCAAACAGATCCATTCCATCACCAATTGCTGC |
| | TTCTCCATTGCATCAAAAGGGTTTGTTTTTCACCAGAGACTCTAGATGGTCTACTA |
| | TGAGAAACACCATCTTGTCTGTTTACCAGCCATCTTACTTGGCAAAGTTGGTTCCA |
| | ATTATGCAGTCCTACATTGAATCTGCTACCAAGAACTTGGATTCCGAAGGTGATTT |
| | GACTTTCTCCGATTTGTCTTTGAAGTTGGCCACTGATGTTATTGGTCAAGCTTCTT |
| | TTGGTGTTGACTTCGGTTTGTCTAAACCTATCTCTGATAAGATGTCCCACCACCAA |
| | GATGATTCTGAAGTACAAGAATTCATCAAGCAGCACATCTACTCTACTACCCAATT |
| | GAAGATGGATTTGTCCGGTTCCGTTTCCATTATTTTGGGTTTGTTAGTCCCAATCC |
| | TGCAAGAACCTTTTAGACAAGTTTTGAAGAGAATTCCAGGTACGATGGATTGGAA |
| | GGTTGAAAGAACTAACAAGAACCTGTCATCCAGATTGGACGAAATCGTTGCTAAG |
| | AGAATCGAAGAAAAGGACAGATCCTCTAAGGACTTCTTGTCATTGATTATGCAAGC |
| | CAGGGAATCTGAAAAGTTGGCTAAGAATGTTTTCACCTCCGATTACATTTCTGCCG |
| | TTACTTACGAACATTTGTTGGCTGGTTCTGCTACTACTTCTTTCACTTTGTCCTCCA |
| | TCATCTACTTGGTTGCTTGTCATCCTGAAGTCGAACAAAAGTTGTTGGCAGAAATT |
| | GATGCTTTTGGTCCAGACGATAATATGCCAACTGCTAATGACTTGCAACAGAAGTT |
| | TCCATACTTGGACCAAGTTATCAAAGAAGCTATGCGTTGCTATATCGTGTCTCCAT |
| | TAGTTGCTAGAGAAACCTCTGCTGAAGTTGAAATTGGTGGTTACAAATTGCCAAAA |
| | GGTACTTGGGTTTGGTTGGCTTTGGGTGTTTTAGCAAAAGATCCAAAGAATTTCCC |
| | AGAGCCAGAAAAGTTTAAGCCAGAAAGATTTGATCCAAACTGCGCTGAAGAAAAG |
| | CAAAGACATCCATACGCTAATATCCCATTTGGTATTGGTCCTAGAGCTTGCATTGG |
| | TCAAAAGTTCTCTATCCAAGAGATCAAGCTGTCCTTGATTCACTTGTACAGAAAGT |
| | ACATCTTCCAGCACTCACCTTTGATGGAATCTCCATTGGAATTGGAATACGGTATC |
| | GTCTTGAACTACAAACACGGTGTTAAGGTTCATGCCATCAAGAGAAAGTGA (SEQ |
| | ID NO: 33) |
| FveMAX1a | ATGGGCATGGAATTCTTGATTACCAACGGTTCTTTGGTGTCTACCATTTTTACTGT |
| | TTTGGCAGTTTTGGCTGGTGTCTTGGGTTACTTGTATGCTCCATATTGGGGTGTTA |
| | GAAGAGTTCCAGGTCCAAGAACTATTCCATTTTTGGGTCATTTGCCTTTGTTGGCT |
| | AAACATGGTCCAGATTTGTTCTCCGTTTTGGCTAAGCAATACGGTCCAATTTTCAG |
| | ATTCCATATGGGTAGACAACCATTGATTATCGTTGCTGATGCTGAATTGTGTAGAG |
| | AAGTCGGTATTAAGAAGTTCAAGGACATCCCAAACAGATCCATTCCATCTCCAATT |
| | TCTGCTTCTCCATTGCATCAAAAGGGTTTGTTCTTCACCAGAGATATTAGATGGTC |
| | TACCATGAGAAACACCATCTTGTCTGTTTACCAACCATCTTACTTGGCTTCATTGG |
| | TTCCAACTATGCAGTCCTATATTGAATCTGCTACCCAAAACTTGGACGACTCCTCT |
| | AACAAAGAAGATATCACCTTCTCCAACCTGTCTTTGAGATTGGCTACTGATGTTAT |
| | TGGTCAAGCTTCTTTCGGTGTTGATTTCGGTTTGTCTAAGCCACAATCCATCTCTA |
| | ACTCCATCAACAAGGTTGATAACGGTGAAGATAAGAACGATGATGAAGTCTCCGA |
| | TTTCATCAACCAGCATATCTACTCTACTACCCAATTGAAGATGGACTTGTCTGGTT |
| | CCTTGTCCATTATTTTGGGTTTGTTGGTCCCAATCATCCAAGAACCTTTCAGACAA |
| | ATCTTGAAGAGAATCCCAGGTACTATGGATTGGAAAGTCGACAGAACTAATCAAA |
| | ATTTGGCCGGTAGATTGGACCAAATCGTTATGAGAAGAATGAAGGATTCCGACAG |
| | AGGTACTAAGAACTTCTTGTCCTTGATTATGAACGCCAGAGAATCTGAAGGTGTTG |
| | CTAAGTCTGTTTTCACCCCAGATTACATTTCTGCTGTTACCTACGAACATTTGTTAG |
| | CTGGTTCTGCTACTACCTCTTTCACTTTGTCATCTGCCGTTTACTTGATTTCTGGTC |
| | ATCCAGAAGTTGAGTCTAAGTTGTTGGCTGAAATTGATGGTTTTGGTCCACATGAT |
| | CAAATTCCAACTGCTCCAGATCTGCAACATAAGTTTCCATACTTGGAACAGGTTAT |
| | CAAAGAAGCCATGAGGTTCTATTTGGTTTCACCATTGGTTGCTAGAGAAACCTCTA |
| | GAGAAGTTGAAGTTGGTGGTTACTTATTGCCAAAAGGTACTTGGGTTTGGTTAGC |
| | TTTGGGTGTTTTAGCAAAGGATCCAAAGAATTTCCCAGAACCAGAAAAGTTCAAGC |
| | CAGAAAGATTTGACCCAAACGGTAAAGAAGAAAAGCAAAGACATCCATACGCCTT |
| | CATTCCATTTGGTATTGGTCCAAGAGCTTGCATCGGTCAAAAGTTTTCTCTGCAAG |
| | AGTTGAAGCTGTCATTGATCCACTTGTACCGTAAATACGTTTTCAGACACTCTCCA |
| | AATATGGAAGCTCCATTGGAATTGGAATTCGGTATCGTTTTGAACTTCAAGAAGGG |
| | TGTTAAGTTGACCGTCATTAAGAGAACCTGA (SEQ ID NO: 34) |
| PhMAX1 | ATGGAATTCCTGTCCACCAACATCCAACATTCTATCGTTGATACCGTTGAAGTTTT |
| | GACTAGACCACCAATGACTTCTACCATCTGTACTATTTTGGCTTTGTTGGCTACCG |
| | TTTTGGTTTACTTTTATGGTCCATATTGGAGGGTTAGAAAAGTTCCAGGTCCACCA |
| | GGTTTTCCATTGGTTGGTCATTTGCCATTGATGGCTAGATATGGTCCAGATGTTTT |
| | TTCCGTTTTGGCTAAACAATACGGTCCAATCTTCAGATTCCATATGGGTAGACAAC |
| | CATTGGTTATAGTTGCTGATGCTGAATTGTGTAGAGAAGTCGGTATTAGAAAGTTC |
| | AAGGACATCCCAAACAGATCCATTCCATCTCCAATTGCTGCTTCTCCATTGCATCA |
| | AAAGGGTTTGTTTTTCACCAGAGACTCTAGATGGTCTACTATGAGAAACACCATCT |
| | TGTCTGTTTACCAGCCATCTTACTTGGCTAAGTTGGTTCCAATTATGCAGTCCTTC |
| | ATTGAATCTGCTACCAAGAACTTGGATTCCGAAGGTGATTTGACTTTCTCCGATTT |
| | GTCTTTGAAGTTGGCCACTGATGTTATTGGTCAAGCTGCTTTTGGTGTTGATTTCG |
| | GTTTGTCTAAACCTATCACCGATAAGATGAACCACCAAGAAAAGGATTCTGAAGTC |
| | CAAGAATTCATCAACCAGCATAACTACTCTACTACCCAGTTGAAGATGGATTTGTC |
| | TGGTTCCGTTTCCATTATCTTGGGTTTGTTAGTTCCAATCCTGCAAGAACCATTCA |
| | GACAGATTTTGAAGAGAATCCCAGGTACTATCGATTGGAAGGTTGAAAGAACGAA |
| | CAAGAACCTGTCTAGAAGATTAGACGAAATCGTTGCTAAGAGGATGGAAGAAAAG |
| | TACGGTTCTTCTAAGGACTTCCTGTCTTTGATATTGCAAGCCAGAGAATCTGAAAA |
| | GTTGGCTAAGAACGTTTTCACCTCCGATTACATTTCTGCTGTTACCTACGAACATT |
| | TGTTAGCTGGTTCTGCTACTACCTCTTTCACTTTGTCATCTATCATCTACTTGGTTG |
| | CTGGTCATCCAAAGGTTGAACAAAAGTTGATTGCTGAAATTGATGCCTTTGGTCCA |
| | GACGATCATATGCCAACTGCTAATGACTTGCAACAGAAGTTCTCATACTTGGACCA |
| | AGTTATCAAAGAAGCTATGCGTTGTTACACTGTTTCACCTTTGGTTGCTAGAGAAA |
| | CTTCTGCCGAAGTTGAAATTGGTGGTTACAAATTGCCAAAAGGTACATGGGTTTG |
| | GTTGGCTTTGGGTGTTTTGGCAAAAGATCCAAAGAATTTTCCAGAGCCTGAGAAG |
| | TTTAGACCAGAAAGATTTGATCCAAACTGCAAAGAAGAAAAGCAGAGACATCCATA |
| | CGCTAACATTCCATTTGGTATTGGTCCTAGAGCTTGCATTGGTCAAAAGTTCTCTA |
| | TCCAAGAGATCAAGTTGTCCTTGATCCACTTGTACAGAAAGTACATCTTCAGACAC |
| | TCACCCTTGATGGAAAAACCATTGGAATTGGAATACGGCATCGTCTTGAATTACAA |
| | ACACGGTGTTAAGGTTTGCGCCATCAAGAGAAAGTAA (SEQ ID NO: 35) |
| ZmMAX1b | ATGTTGGCTTCTGCTGTTTTGAGAGCTATGGAAGAATGTACTTTTACCTCTGCTGC |
| | TATGGCTGTTGGTTTTTTGTTGGTTGTTTACTTGTACGAGCCATACTGGAAGGTTA |
| | GACATGTTCCAGGTCCAGTTCCATTGCCATTTGTTGGTCACTTGCATTTGTTAGCT |
| | AGACATGGTCCTGATGTTTTCTTGGTTTTGGCTAAAAAGTACGGTCCAATCTTCAG |
| | ATTCCATATGGGTAGACAACCATTGGTTATCGTTGCTAATGCTGAATTGTGCAAAG |
| | AAGTCGGCATCAAAAAGTTCAAGTCTATGCCAAATAGGTCCTTGCCATCTGCTATT |
| | GCTAATTCCCCAATTCATTTGAAGGGTTTGTTCTCCACTAGAGACTCTAGATGGTC |
| | TGCTTTGAGAAACATCATCGTGTCTATCTACCAACCATCTCATTTGGCTGGTTTGA |
| | TTCCATCTATGCAATCCCATATTGAAAGAGCTGCTACCAATTTGGATGATGGTGGT |
| | GAAGCTGAAGTTGCTTTTTCTAAATTGGCTTTGTCTTTGGCCACCGATGTTATTGG |
| | TCAAGCTGCTTTTGGTGCTGATTTTGGTTTGACTACAAAACCAGCTGCTCCACCAC |
| | CACATCATGGTCCACCAAGACAACATGGTGAAGAGGATGGTGATGGTTCTCATTC |
| | TACTAGATCTTCCGAATTCATCAAGATGCATATCCATTCTACCACCTCATTGAAGA |
| | TGGATTTGTCTGGTTCTTTGTCTACCATCGTTGGTACTTTGTTGCCAGTTTTACAAT |
| | GGCCTTTGAGACAGTTGTTGTTGAGAGTTCCAGGTGCTGCTGATAGAGAAATTCA |
| | ACGTGTTAATGGTGCTTTGTGCAGAATGATGGATGGTATTGTCGCAGATAGAGTT |
| | GCTGCAAGAGAAAGAGCACCACAAGCTCAAAGACAGAAGGATTTTTTGTCAGTTG |
| | TTTTGGCTGCCAGAGATTCTGATGCTGCTGCTAGAAAGTTGTTGACTCCAGATTAT |
| | TTGTCCGCTTTGACCTACGAACACTTGTTAGCTGGTTCTGCTACTACTGCTTTTAC |
| | TTTGTCATCTGTCTTGTACTTGGTTGCCCAACATCCAAGAGTTGAAGAAAAGTTGT |
| | TAAGAGAAGTTGATGCTTTCGGTCCACCTGATAGAGTTCCAACTGCTGAAGATCT |
| | ACAATCCAGATTTCCATACACCGACCAAGTCTTGAAAGAATCTATGAGGTTCTTCA |
| | TGGTTTCCCCATTGGTTGCTAGAGAAACTTCTGAACAAGTTGATATTGCCGGTTAC |
| | GTTTTGCCAAAATCTACTTGGGTTTGGATGGCTCCAGGTGTTTTAGCAAAAGATCC |
| | AGTTAATTTTCCAGAGCCAGAGTTGTTTAGACCAGAAAGATTTGATCCAGCTGGTG |
| | ATGAACAAAAAAGAAGGCATCCATACGCTTTCATTCCATTTGGTATTGGTCCAAGA |
| | ATCTGCATCGGTCAAAGATTCTCTATCCAAGAAATCAAGTTGGCCTTGATCCACTT |
| | GTACAGACAATACGTTTTTAGGCACTCTCCCTCTATGGAATCACCATTGGAATTTC |
| | AATTCGGTGTCGTCTTGAACTTCAAACACGGTGTTAAGTTGCAGTCCATCAAGAG |
| | ACATAAGTGCTGA (SEQ ID NO: 36) |
| RcCYP722C2 | ATGCTGAAGCTGTCTAAAGAAGAGTTGTTGTTCTTGGCCCAAAACAAGTACGATAT |
| | TGTCATGGTTGCCTTGTTCTCCATTGCAGTTTTTGCTGTTTTGAAGGCTTGGAGAA |
| | AGAAGATTACCACCTCCAACAAAGAAGATATTCCAGGCGGTTTGGGTTTGCCATT |
| | TGTTGGTGAAACTTTGTCTTTCTTGTCTGCTACCAATTCTACCAGAGGTTGTTACG |
| | ATTTCGTCAGGTTGAGAAGAAAATGGTACGGTAAATGGTTCAAGACCAGAATCTT |
| | CGGTAAGATTCATGTTTTCGCTCCATCTACTGAAGCTGCTAGAAAGGTTTTCACTA |
| | ATGACTTCGGTGAATTCAACAAGGGCTACATTAAGTCTATGGCTACTGTTGTCGGT |
| | GAGAAGTCTGTTTTTGCCGTTCCATTGGAATCCCATAAGAGAATCAGACATATTCT |
| | GTCCGCTTTGTTCTCTATCCCATCCTTGTCTATTTTCGTCCAAAACTTCGACCAAAT |
| | GTTGTCCCAAAGACTGAAGAACTTGCAAGAAAGAGGTATTACCTTCGCCGTTTTG |
| | GATTTCACTATGAAGTTGACCTTGGACTCTATGTGCAACATGCTGATGTCTATCAC |
| | TGAAGAGTCTTTGTTGAAGCAGATCTTGAGAGATTGTGCTGCTGTTTCTGATGCTT |
| | TGTTGTCTGTTCCATTGATGATTCCAGGTACTACTTACTACAAAGGTATGAAGGCA |
| | AGAGAAAGGCTGATGGAAATCTTCAAAGAAAAGATTGCCAGAAGAAGATCTGGCG |
| | AAGAGTACAAAGATGACTTCTTGCAAAGTTTGTTGGAAAGGGATTCTTACCCATCC |
| | TCTGAAAGATTGCAAGACTCCGAAATTATGGACAACCTGTTGACTTTGTTGGTTTC |
| | CGGTCAAGTTTCTTCTGCTGCTACTATGATGTGGTCTGTTAAGTTTCTAGACGAAA |
| | ACAAAGAGGTCTTGGACAAGTTGAGAGAAGAACAATCTAACATTGCCAAGAATAT |
| | GCAAGGTGCCTCTTTGTCTATGGTCGATTTGAACAAAATGTCCTACTGCTACAAG |
| | GTCGTCAAAGAATCATTGAGAATGTCCAACGCTGTTTTGTGGTTGCCAAGAGTTG |
| | CTCAAAAAGACTGTACTGTTGATGGTTTCGAGATCAAGAAAGGTTGGAATGTTAAC |
| | GTTGATGCTACCCATATTCATTACGATCCAGCCTTATACAAAGACCCATTGAGATT |
| | CAATCCATCCAGATTCGACGAAATGCAAAAGCCATACTCTTTCATTCCATTTGGTG |
| | CTGGTCCAAGAACTTGTTTGGGTATTGAAATGGCTAAGCTGTCCATGTTGGTTTTC |
| | ATTCATAGATTGACCTCTGAATACGAATGGCGTATTGAAGATCCAGATCCATCTTT |
| | GGAAAGAACTACTCATGTTCCAAGATTGAGAACCGGTTTGCCAATTACTTTGAAGC |
| | CATTGAAATCCGCTTCTCAGGATTGA (SEQ ID NO: 37) |
| MeCYP722C2 | ATGATCCTGAAGAACTTCAAGCTGAAGATGCTGAAGTTGTTCAGAGAACACGTTTT |
| | GTTCTTGGTCCAAAACAAGTACGATATTGTCTTGGCTGCCTTCTTGTCCATTGCTA |
| | TTTTTGCTTTTTTCAAGGCCAGACGTAAGGTCAACACTATTAACGAAGAAGATGAG |
| | ATCCCAGGTTCTTTGGGTTTGCCATTTGTTGGTGAAACTTTCTCTTTCTTGTCTGCT |
| | ACCAATTCTACCAGAGGTTGTTACGATTTCGTCAGGTTGAGAAGAAAATGGTACG |
| | GTAAATGGTTCAAGTCCAGAATGTTCGGTAAGATCCATGTTTTCGTTCCATCTACT |
| | GAAGCTGCTAGAAAGGTTTTCACTAATGACTTCGGTGAGTTCAACAAGTCCTACAT |
| | TAAGTCTATGGCTACTGTTGTCGGTGAGAAGTCTGTTTTTGCTGTTCCATTGGAAA |
| | CCCACAAGAGAATCAGACATATTTTGTCCGCTTTGTTCTCCATGCCATCTTTGTCT |
| | AAGTTCGTTGAAAAGTTCGACCAGATGATCTCCCAAAGATTGAACAAGTTGGAAC |
| | AAACCGGTAAGTCTTTCGCTGTTTTGCCTTTTACTATGAAGTTGACCTTGGATTCT |
| | GTCTGCAACATGCTGATGTCTATCACTGAAGAATCCTTGTTGGACCAAATCTTGTC |
| | TGATTGTGCTGCTGTTTCTGATGCTTTGTTGTCAGTTCCATTGATGATTCCAGGTA |
| | CGATCTACTACAAAGGTATGAAGGCTAGACAGAGGCTGATGAAGATTTTCAAAGA |
| | AATGATCGACAGAAGGCGTTCCGGTAAAGAACAGAAGGATGATTTCTTGCAGTAC |
| | TTGTTGGAAAGACATACCTGTCCATCCTCCGAAAAATTGGAAGATTCCGAAATCAT |
| | GGACAACCTGTTGACTTTGTTGGTTTCTGGTCAAGTTTCTTCTGCTGCTGCTATGA |
| | TGTGGTCTGTTAAGTTTTTGGACGAAAACTCCGAAGTCTTGGACAAGTTGAGAGA |
| | AGAACAATTGGAAATCGCTAAGAACAAGCAAGGTGGTACTTCTTTGTCAATGGAA |
| | GATATCAACAAGATGTCCTACGGTTTGAAGGTCGTCAAAGAAACTTTGAGAATGTC |
| | CAACGTTGTTTTGTGGTTGCCAAGAGTTGCTCAAAATGATTGCACTTTGGATGGTG |
| | TCGAAATGAAGAAAGGTTGGGTTGTTAATGTTGATGCTACCTGCATCCATTTTGAT |
| | CCAGACTTGTATGAAGATCCCATGAGATTCAATCCATCCAGATTTGACGAAATGCA |
| | GAAGCCATATTCTTTCTTGCCTTTTGGTGCTGGTCCAAGAACTTGTTTGGGTATTG |
| | AAATGGCCAAGTTGTCCATCTTGGTTTTCTTGCATAGATTGACTGGTGGTTACGAA |
| | TGGCGTATTGAAAACAGAGATCCATCTATGGAAAGAACTACCCATGTTCCAAGATT |
| | GAGATCTGGTTTGCCAATTACTTTGAAGGCTTTGGCTAAGAACGGCAAGTAA |
| | (SEQ ID NO: 38) |

TABLE 9
Accession numbers of MAX1 analogs used for the phylogenetic tree analysis
in FIG. 30A. The amino acid sequences are downloadable from NCBI
or Phytozom (website phytozome.jgi.doe.gov/pz/portal.html).

| | | Size | |
|---|---|---|---|
| Gene | Species | (a.a.) | Accession numbers |
| AtMAX1 | Arabidopsis thaliana | 522 | NP_565617 |
| SmMAX1a | Selaginella moellendorffii | 512 | AGI65366 |
| OsMAX1b | Oryza sativa (Rice) | 539 | XP_015633367 |
| (Os01g0700900) | | | |
| OsMAX1c | Oryza sativa (Rice) | 541 | XP_015644699 |
| (Os01g0701400) | | | |
| OsMAX1d | Oryza sativa (Rice) | 516 | XP_015642272 |
| (Os01g0701500) | | | |
| OsMAX1e | Oryza sativa (Rice) | 548 | XP_015626073 |
| (Os02g0221900) | | | |
| OsMAX1f | Oryza sativa (Rice) | 540 | XP_015644019 |
| (Os06g0565100) | | | |
| SlMAX1 | Solanum lycopersicum (Tomato) | 519 | XP_004245085 |
| ZmMAX1a | Zea mays (Corn) | 537 | PWZ07057 |
| ZmMAX1b | Zea mays (Corn) | 543 | ONM29770 |
| ZmMAX1c | Zea mays (Corn) | 560 | XP_020407074 |
| AmtMAX1 | Amborella trichopoda | 555 | XP_011626843 |
| PpMAX1a | Prunus persica (Peach) | 538 | XP_007222310 |
| (Prupe.1G410300) | | | |
| PpMAX1b | Prunus persica (Peach) | 533 | XP_007224581 |
| PpMAX1c | Prunus persica (Peach) | 536 | XP_007225050 |
| (Prupe.1G410100) | | | |
| MdMAX1a1 | Malus domestica (Apple) | 540 | XP_008393629 |
| (MDP0000130133) | | | |
| MdMAX1a2 | Malus domestica (Apple) | 532 | XP_028955031 |
| (MDP0000148030) | | | |
| MdMAX1b1 | Malus domestica (Peach) | 526 | XP_008357300 |
| (MDP0000215198) | | | |
| MdMAX1b2 | Malus domestica (Apple) | 542 | RXH72971 |
| (MDP0000231714) | | | |
| FveMAX1a1 | Fragaria vesca (Woodland | 531 | XP_004291053 |
| (FvH4_2g31680) | strawberry) | | |
| FveMAX1a2 | Fragaria vesca (Woodland | 531 | FvH4_2g31660.t1 |
| (FvH4_2g31660) | strawberry) | | |
| AcMAX1 | Actinidia chinensis (Chinese | 537 | PSR91738 |
| | kiwifruit) | | |
| ClMAX1 | Citrullus lanatus (Watermelon) | 526 | Cla97C05G099260.1 |
| CcMAX1a | Citrus clementina (Clementine) | 538 | ESR64106 |
| CcMAX1b | Citrus clementina (Clementine) | 547 | ESR38800 |
| HaMAX1 | Helianthus annuus (Common | 521 | OTG21718 |
| | sunflower) | | |
| MeMAX1 | Manihot esculenta (Cassava) | 527 | OAY26871 |
| MaMAX1a | Musa acuminata (Banana) | 529 | XP_009380454 |
| MaMAX1b | Musa acuminata (Banana) | 526 | XP_009408870 |
| PdMAX1a | Prunus dulcis (Almond) | 538 | XP_034198777 |
| PdMAX1b | Prunus dulcis (Almond) | 533 | XP_034200540 |
| PdMAX1c | Prunus dulcis (Almond) | 535 | XP_034203783 |
| PavMAX1a | Prunus avium (Sweet Cherry) | 533 | XP_021823617 |
| PavMAX1b | Prunus avium (Sweet Cherry) | 535 | XP_021823330 |
| PavMAX1c | Prunus avium (Sweet Cherry) | 541 | XP_021823488 |
| ParMAX1a | Prunus armeniaca (Apricot) | 538 | CAB4266066 |
| ParMAX1b | Prunus armeniaca (Apricot) | 533 | CAB4296645 |
| ParMAX1c | Prunus armeniaca (Apricot) | 544 | CAB4296646 |
| GhiMAX1 | Gossypium hirsutum (Cotton) | 539 | XP_016689695 |
| AhMAX1a | Arachis hypogaea (Peanut) | 539 | XP_025606701 |
| AhMAX1b | Arachis hypogaea (Peanut) | 539 | XP_025660007 |
| AhMAX1c | Arachis hypogaea (Peanut) | 528 | XP_025613410 |
| GmMAX1a | Glycine max (Soybean) | 551 | AQY54419 |
| GmMAX1b | Glycine max (Soybean) | 548 | AQY54420 |
| GmMAX1c | Glycine max (Soybean) | 532 | XP_003549345 |
| GmMAX1d | Glycine max (Soybean) | 538 | XP_003544542 |
| PgMAX1 | Picea glauca (White Spruce) | 544 | AGI65359 |
| SbMAX1a | Sorghum bicolor (Sorghum) | 547 | XP_002458367 |
| SbMAX1b | Sorghum bicolor (Sorghum) | 545 | XP_002456213 |
| SbMAX1c | Sorghum bicolor (Sorghum) | 545 | XP_002453551 |
| SbMAX1d | Sorghum bicolor (Sorghum) | 540 | XP_002438586 |
| DcMAX1 | Daucus carota subsp. sativus | 526 | KZN06895 |
| | (Carrot) | | |
| CmMAX1 | Cucumis melo (Muskmelon) | 531 | XP_008452735 |
| AoMAX1a | Asparagus officinalis (Asparagus) | 552 | XP_020251236 |
| AoMAX1b | Asparagus officinalis (Asparagus) | 549 | XP_020251248 |

### Materials and Methods

### Chemicals and General Culture Conditions

Standards of (±)5-Deoxy-strigol (purity >98%) and (±)-Orobanchol were purchased from Strigolab (Italy), standards of (±)4-deoxyorobanchol (also named as (±)-2′-epi-5-deoxystrigol) were acquired from Chempep Inc. (USA), β-ionone is purchased from Fisher Scientific (USA), 9-cis-ß-carotene and all-trans-ß-carotene were purchased from Sigma-Aldrich Co. (USA). The chemically competent E. coli strain TOP10 (Life Technologies) was used for DNA manipulation and amplification, and was grown at 37° C. in lysogeny broth (LB) medium (Fisher Scientific) supplemented with appropriate amount of antibiotics (100 μg ml⁻¹ ampicillin (Fisher Scientific), 50 μg ml⁻¹ kanamycin (Fisher Scientific), 25 μg ml⁻¹ chloramphenicol (Fisher Scientific), 50 μg ml⁻¹ spectinomycin (Sigma-Aldrich) for plasmid maintenance. For protein expression and CL-production, we used chemically competent E. coli strain BL21(DE3) (Novagen). LB, M9 (Fisher Scientific), and XY medium were used for the fermentation of E. coli strains. XY medium contains 13.3 g/L KH₂PO₄, 4 g/L (NH₄)₂HPO₄, 1.7 g/L citric acid, 0.0025 g/L CoCl₂, 0.015 g/L MnCl₂, 0.0015 g/L CuCl₂, 0.003 g/L H₃BO₃, 0.0025 g/L Na₂MoO₄, 0.008 g/L Zn(CH₃COO)₂), 0.06 g/L Fe(III) citrate, 0.0045 g/L thiamine, 1.3 g/L MgSO₄, 5 g/L yeast extract and 40 g/L xylose, pH 7.0. For the first stage of the co-culture fermentation, yeast strains were cultured at 28° C. in complex yeast extract peptone dextrose (YPD, all components from BD Diagnostics) medium, or SDM containing yeast nitrogen base (YNB) without amino acids (BD Diagnostics), ammonium sulfate (Fisher Scientific), 2% dextrose, and synesthetic complete or the appropriate dropout solution (Clontech) for plasmid maintenance. XY medium was used in the second stage of the co-culture fermentation. Unless specified, all the chemicals used in this study were purchase from Fisher Scientific or Sigma-Aldrich Co.

### General Techniques for DNA Manipulation

Plasmid DNA was prepared using the Econospin columns (Epoch Life Science) according to manufacturer's protocols. PCR reactions were performed using Q5 DNA polymerase (NEB) and Expand High Fidelity PCR System (Roche Life Science) according to manufacturer's protocols. PCR products were purified by Zymoclean Gel DNA Recovery Kit (Zymo Research). All DNA constructs were confirmed through DNA sequencing by Source Bioscience (L A, USA). Restriction enzymes (NEB) and T4 ligase (NEB) were used to digest and ligate the DNA fragments, respectively. BP Clonase 11 Enzyme Mix, Gateway pDONR221 Vector and LR Clonase II Enzyme Mix (Life Technologies) and the S. cerevisiae Advanced Gateway Destination Vector Kit (Addgene) were used to perform Gateway Cloning (57). Using this method, the yeast expression cassette vectors were constructed, then the vectors were transformed into yeast cells using Frozen-EZ Yeast Transformation II Kit (Zymo Research). Gibson one-pot, isothermal DNA assembly was conducted at 10 μl scale by incubating T5 exonuclease (NEB), Phusion polymerase (NEB), Taq ligase (NEB) and 50 ng of each DNA fragment at 50° C. for 1 h to assemble multiple DNA fragments into one circular plasmid (58). Integrated yeast strains are constructed through homologous recombination and DNA assembly (59). Plasmids and E. coli or yeast strains utilized in this study are listed in Tables 1 and 2. Custom oligonucleotides were synthesized by Integrated DNA Technologies (IDT) and Life Technologies. The plant gene sequences were codon-optimized for expression in S. cerevisiae using the GeneArt GeneOptimizer program (Life Technologies) and synthesized by IDT and Twist Bioscience (San Francisco, CA). DNA sequences of genes involved in this work are listed in Table 5.

For the construction of E. coli expression vectors, the CCD7 gene was amplified by PCR and cloned into the pCDFDuet-1 plasmid (Novagen) using NcoI and NotI to yield the plasmid pCDFDuet-tCCD7. The OsD27 gene was amplified by PCR, digested by NdeI and AvrII and ligated into accordingly digested pCDFDuet-tCCD7, yielding the plasmid pCDFDuet-tCCD7-OsD27. The CCD8 gene was amplified by PCR and cloned into pET21a using Gibson assembly. For the construction of yeast expression cassettes, NADPH-P450 reductase and each individual p450 gene were constructed using Gateway Cloning as described previously.

Culture Conditions for E. coli-Based CL Related Intermediates Production

For the in vivo production 9-cis-β-carotene, E. coli BL21(DE3) was transformed with pAC-BETAipi (Addgene) and pCDFDuet-OsD27, generating strains CL-2. For 9-cis-β-apo-10′-carotenol production, E. coli BL21(DE3) was transformed with pAC-BETAipi (Addgene) and pCDFDuet-OsD27-tCCD7 to generate strains CL-3, then the yellow colonies were picked up and grown in LB with the appropriate antibiotics at 37° C., overnight. 500 μL of the overnight culture was then used to inoculate 5 ml fresh LB with the corresponding antibiotics with a starting OD₆₀₀ at ˜0.05 and cultured at 37° C. and 200 rpm in the 50 ml Erlenmeyer flask. When OD₆₀₀ reached ˜0.6, the isopropyl β-D-1-thiogalactopyranoside (IPTG) was added to make the final concentration at 0.2 mM, with ferrous sulfate supplemented at the same time (final concentration at 10 mg/L). Then the system was cooled and the cells were cultivated at 22° C. for 72 hours.

Culture Conditions for E. coli-Yeast Consortium-Based SL Production

For the in vivo production of SLs, E. coli BL21(DE3) was co-transformed with the plasmids pAC-BETAipi (Addgene), pCDFDuet-OsD27-tCCD7, pET21a-tCCD8, generating strains CL-5. Single yellow colony was then picked and grown overnight at 37° C. in 1 ml LB supplemented with 100 μg ml⁻¹ ampicillin, 25 μg ml⁻¹ chloramphenicol, and 50 μg ml-1 spectinomycin. 500 μL of the overnight culture was then used to inoculate 5 ml fresh LB with the corresponding antibiotics with a starting OD₆₀₀ at ˜0.05 and cultured at 37° C. and 200 rpm in the 50 ml Erlenmeyer flask. When OD₆₀₀ reached ˜0.6, IPTG was added with the final concentration at 0.2 mM, with ferrous sulfate supplemented at the same time (final concentration at 10 mg/L). Then the system was cooled and the cells were induced at 22° C., for 15 hours.

At the same time, single colonies of each yeast strains harboring the corresponding cytochrome P450-expression constructs were precultured overnight at 28° C. in YNB media supplemented with 0.2% (w/v) glucose and the appropriate dropout solution (YNB-DO). 100 μL of the overnight seed culture was used to inoculate 5 mL of the corresponding YNB-DO media in a 50 ml Erlenmeyer flask and grown for 15 hours. The next day, the E. coli and yeast cells were harvested by centrifugation at 3,500 rpm for 5 min, respectively. Then the E. coli and S. cerevisiae cells were mixed and resuspended in 5 ml TY media (OD₆₀₀˜8.0), and t cultured at 22° C. and 200 rpm for an additional 60 hours (final OD₆₀₀˜40). In the case of CL production, wildtype strain S. cerevisiae CEN.PK2-1D was mixed with the CL-producing E. coli cells, and CEN.PK2-1D is precultivated in YPD.

### Isolation and Characterization of SLs and Related Pathway Intermediates

Unless specified, 5 ml culture was used for compound extraction. The cell pellets were resuspended in 150 μl dimethylformamide (DMF) and shaken vigorously, followed by the addition of 800 μl acetone and vigorous shaking for 15 minutes; and the medium was extracted using 4 ml ethyl acetate. The organic phase was collected upon centrifugation, evaporated to dryness, and dissolved in 120 μl of acetone, which is centrifuged at 12,000 rpm for 10 min before applied to LC-MS analysis. SLs and other pathway intermediates were identified and quantified by reverse phase high-performance liquid chromatography mass spectrometry (HPLC-MS) on Shimadzu LC-MS 2020 (Kyoto, Japan).

The synthesis of 9-cis-β-carotene was analyzed by Separation Method Ion C₁₈ column, Kinetex® C18 (100 mm×2.1 mm, 100 Å, particle size 2.6 μm; Phenomex, Torrance, CA, USA) at 40° C.: metabolites were separated with an isocratic elution of 100% methanol (v/v in water, 0.1% formic acid) over 20 min with a flow rate of 0.4 mL/min. The injection volume is 10 μL and the UV-VIS absorption was monitored at 190-800 nm. With Separation Method I, the retention time of 9-cis-carotene is 7.78 min, and all-trans-β-carotene is 8.17 min (447&471 nm).

Separation Method I: C₁₈ column, poroshell 120 EC-C18 (100 mm×3.0 mm, 100 Å, particle size 2.7 μm; Aglient, Santa Clara, CA, USA); column temperature, 40° C.; gradient elution solvents system, (A) 0.1% formic acid in water and (B) 0.1% formic acid in methanol; injection volume, 20 μL; total run time, 45 min. The gradient was as follows: 0-18 min, 5%-100% B; 18-43 min, 100% B; 43-45 min, 100%-5% B. The flow rate was maintained at 0.5 mL min⁻¹. The chromatograms were monitored at 190-800 nm. Under this condition, the retention time of 9-cis-β-apo-10′-carotenol was 12.60 min (373&390 nm). Separation Method III: C18 column, Kinetex® C18 (100 mm×2.1 mm, 100 Å, particle size 2.6 μm; Phenomex, Torrance, CA, USA); column temperature, 40° C.; gradient elution solvents system, (A) 0.1% formic acid in water and (B) 0.1% formic acid in acetonitrile; injection volume, 10 μL; total run time, 40 min. The gradient was as follows: 0-28 min, 5%-100% B; 28-35 min, 100% B; 35-40 min, 5% B. The flow rate was maintained at 0.4 mL min-. The chromatograms were monitored at 190-800 nm. Under this condition, the retention time of β-ionone was 15.91 min (298 nm), CL was 20.58 mm (269 nm); Mass spectra were obtained over the mass range of m/z 50-800 Da in the positive and negative ion modes. The DL temperature was 250° C. The nebulizing gas and drying gas flow rates were 1.5 L/min and 15 L/min, respectively.

### Phylogenetic Analysis

The Phylogenetic tree was constructed by MEGA X using ClustalW module and Neighbor-joining trees. The parameters are set as follows, p-distance, 500 bootstrap replications, partial deletion (50%). The accession numbers of proteins are listed in Tables 4 and 9.

All accession numbers, publications, patents, and patent applications cited herein are hereby incorporated by reference with respect to the material for which they are expressly cited.

Although the foregoing disclosure has been described in some detail by way of illustration and example for purposes of clarity of understanding, it will be readily apparent to those of ordinary skill in the art in light of the teachings of this disclosure that certain changes and modifications can be made thereto without departing from the spirit or scope of the appended claims.

### REFERENCES CITED IN THE SPECIFICATION BY NUMBER

1. W. L. P. Cook C. E, Turner B, Wall M. E., Germination of witchweed (Striga lutea Lour.) isolation and properties of a potent stimulant.pdf. Science 154, 1189-1190 (1966).
2. K. Akiyama, K. Matsuzaki, H. Hayashi, Plant sesquiterpenes induce hyphal branching in arbuscular mycorrhizal fungi. Nature 435, 824-827 (2005); published online Epub June 9 (10.1038/nature03608).
3. S. Al-Babili, H. J. Bouwmeester, Strigolactones, a novel carotenoid-derived plant hormone. Annual review of plant biology 66, 161-186 (2015)10.1146/annurev-arplant-043014-114759).
4. M. Vurro, C. Prandi, F. Baroccio, Strigolactones: how far is their commercial use for agricultural purposes? Pest management science 72, 2026-2034 (2016); published online Epub November (10.1002/ps.4254).
5. S. M. Smith, Q&A: What are strigolactones and why are they important to plants and soil microbes?.pdf. BMC biology 12, (2014)10.1186/1741-7007-12-19).
6. E. B. Aliche, C. Screpanti, A. De Mesmaeker, T. Munnik, H. J. Bouwmeester, Science and application of strigolactones. The New phytologist, (2020); published online Epub February 17 (10.1111/nph.16489).
7. B. Zwanenburg, T. Pospisil, Structure and activity of strigolactones: new plant hormones with a rich future. Molecular plant 6, 38-62 (2013); published online Epub January (10.1093/mp/sss141).
8. G. C. Waters M. T., Bennett T, Nelson D. C., Strigolactone Signaling and Evolution. Annual review of plant biology 68, 291-322 (2017)10.1146/annurev-arplant-042916040925).
9. S. Abe, A. Sado, K. Tanaka, T. Kisugi, K. Asami, S. Ota, H. I. Kim, K. Yoneyama, X. Xie, T. Ohnishi, Y. Seto, S. Yamaguchi, K. Akiyama, K. Yoneyama, T. Nomura, Carlactone is converted to carlactonoic acid by MAX1 in Arabidopsis and its methyl ester can directly interact with AtD14 in vitro. Proceedings of the National Academy of Sciences of the United States of America 111, 18084-18089 (2014); published online Epub December 16 (10.1073/pnas.1410801111).
10. T. V. Charnikhova, K. Gaus, A. Lumbroso, M. Sanders, J. P. Vincken, A. De Mesmaeker, C. P. Ruyter-Spira, C. Screpanti, H. J. Bouwmeester, Zealactones. Novel natural strigolactones from maize. Phytochemistry 137, 123-131 (2017); published online Epub May (10.1016/j.phytochem.2017.02.010).
11. H. I. Kim, T. Kisugi, P. Khetkam, X. Xie, K. Yoneyama, K. Uchida, T. Yokota, T. Nomura, C. S. P. McErlean, K. Yoneyama, Avenaol, a germination stimulant for root parasitic plants from Avena strigosa. Phytochemistry 103, 85-88 (2014); published online Epub July (10.1016/j.phytochem.2014.03.030).
12. X. Xie, N. Mori, K. Yoneyama, T. Nomura, K. Uchida, K. Yoneyama, K. Akiyama, Lotuslactone, a non-canonical strigolactone from Lotus japonicus. Phytochemistry 157, 200-205 (2019); published online Epub January (10.1016/j.phytochem.2018.10.034).
13. K. Yoneyama, X. Xie, K. Yoneyama, Y. Takeuchi, Strigolactones: structures and biological activities. Pest management science 65, 467-470 (2009); published online Epub May (10.1002/ps.1726).
14. A. A. Sato D, Chae S H, Yokota T, Sugimoto Y, Takeuchi Y, Yoneyama K., Analysis of Strigolactones, Germination Stimulants for Striga and Orobanche, by High-Performance Liquid Chromatography/Tandem Mass Spectrometry.pdf. J. Agric. Food Chem. 51, 1162-1168 (2003).
15. A. J. Humphrey, M. H. Beale, Strigol: biogenesis and physiological activity. Phytochemistry 67, 636-640 (2006); published online Epub April (10.1016/j.phytochem.2005.12.026).
16. C. Ruyter-Spira, W. Kohlen, T. Charnikhova, A. van Zeijl, L. van Bezouwen, N. de Ruijter, C. Cardoso, J. A. Lopez-Raez, R. Matusova, R. Bours, F. Verstappen, H. Bouwmeester, Physiological effects of the synthetic strigolactone analog GR24 on root system architecture in Arabidopsis: another belowground role for strigolactones? Plant physiology 155, 721-734 (2011); published online Epub February (10.1104/pp. 110.166645).
17. A. Scaffidi, M. T. Waters, Y. K. Sun, B. W. Skelton, K. W. Dixon, E. L. Ghisalberti, G. R. Flematti, S. M. Smith, Strigolactone Hormones and Their Stereoisomers Signal through Two Related Receptor Proteins to Induce Different Physiological Responses in Arabidopsis. Plant physiology 165, 1221-1232 (2014); published online Epub July (10.1104/pp. 114.240036).
18. B. Zwanenburg, S. Cavar Zeljkovic, T. Pospisil, Synthesis of strigolactones, a strategic account. Pest management science 72, 15-29 (2016); published online Epub January (10.1002/ps.4105).
19. A. Alder, M. Jamil, M. Marzorati, M. Bruno, M. Vermathen, P. Bigler, S. Ghisla, H. Bouwmeester, P. Beyer, S. Al-Babili, The path from beta-carotene to carlactone, a strigolactone-like plant hormone. Science 335, 1348-1351 (2012); published online Epub March 16 (10.1126/science.1218094).
20. M. Bruno, M. Vermathen, A. Alder, F. Wust, P. Schaub, R. van der Steen, P. Beyer, S. Ghisla, S. Al-Babili, Insights into the formation of carlactone from in-depth analysis of the CCD8-catalyzed reactions. FEBS letters 591, 792-800 (2017); published online Epub March 50 (10.1002/1873-3468.12593).
21. M. Lopez-Obando, Y. Ligerot, S. Bonhomme, F. D. Boyer, C. Rameau, Strigolactone biosynthesis and signaling in plant development. Development 142, 3615-3619 (2015); published online Epub November 1 (10.1242/dev.120006).
22. K. Yoneyama, N. Mori, T. Sato, A. Yoda, X. Xie, M. Okamoto, M. Iwanaga, T. Ohnishi, H. Nishiwaki, T. Asami, T. Yokota, K. Akiyama, K. Yoneyama, T. Nomura, Conversion of carlactone to carlactonoic acid is a conserved function of MAX1 homologs in strigolactone biosynthesis. The New phytologist 218, 1522-1533 (2018); published online Epub June (10.1111/nph.15055).
23. K. Yoneyama, X. Xie, K. Yoneyama, T. Kisugi, T. Nomura, Y. Nakatani, K. Akiyama, C. S. P. McErlean, Which are the major players, canonical or non-canonical strigolactones? Journal of experimental botany 69, 2231-2239 (2018); published online Epub April 23 (10.1093/jxb/ery090).
24. X. Xie, Structural diversity of strigolactones and their distribution in the plant kingdom. Journal of pesticide science 41, 175-180 (2016); published online Epub November 20 (10.1584/jpestics.J16-02).
25. K. Y. X. Xie, T. Kisugi, K. Uchida, S. Ito, K. Akiyama, H., T. Y. Hayashi, T. Nomura and K. Yoneyama, Confirming Stereochemical Structures of Strigolactones Produced by Rice and Tobacco. Mol. Plant 6, 153-163 (2013)10.1093/mp/sss139).
26. Y. Zhang, A. D. van Dijk, A. Scaffidi, G. R. Flematti, M. Hofmann, T. Charnikhova, F. Verstappen, J. Hepworth, S. van der Krol, O. Leyser, S. M. Smith, B. Zwanenburg, S. Al-Babili, C. Ruyter-Spira, H. J. Bouwmeester, Rice cytochrome P450 MAX1 homologs catalyze distinct steps in strigolactone biosynthesis. Nature chemical biology 10, 1028-1033 (2014); published online Epub December (10.1038/nchembio.1660).
27. K. Ueno, H. Nakashima, M. Mizutani, H. Takikawa, Y. Sugimoto, Bioconversion of 5-deoxystrigol stereoisomers to monohydroxylated strigolactones by plants. Journal of pesticide science 43, 198-206 (2018); published online Epub August 20 (10.1584/jpestics.D18-021).
28. M. Iseki, K. Shida, K. Kuwabara, T. Wakabayashi, M. Mizutani, H. Takikawa, Y. Sugimoto, Evidence for species-dependent biosynthetic pathways for converting carlactone to strigolactones in plants. Journal of experimental botany 69, 2305-2318 (2018); published online Epub April 23 (10.1093/jxb/erx428).
29. T. Wakabayashi, M. Hamana, A. Mori, R. Akiyama, K. Ueno, K. Osakabe, Y. Osakabe, H. Suzuki, H. Takikawa, M. Mizutani, Y. Sugimoto, Direct conversion of carlactonoic acid to orobanchol by cytochrome P450 CYP722C in strigolactone biosynthesis. Science advances 5, eaax9067 (2019); published online Epub December (10.1126/sciadv.aax9067).
30. N. Mori, A. Sado, X. Xie, K. Yoneyama, K. Asami, Y. Seto, T. Nomura, S. Yamaguchi, K. Yoneyama, K. Akiyama, Chemical identification of 18-hydroxycarlactonoic acid as an LjMAX1 product and in planta conversion of its methyl ester to canonical and non-canonical strigolactones in Lotus japonicus. Phytochemistry 174, 112349 (2020); published online Epub June (10.1016/j.phytochem.2020.112349).
31. T. Wakabayashi, K. Shida, Y. Kitano, H. Takikawa, M. Mizutani, Y. Sugimoto, CYP722C from Gossypium arboreum catalyzes the conversion of carlactonoic acid to 5-deoxystrigol. Planta 251, 97 (2020); published online Epub April 18 (10.1007/s00425-020-03390-6).
32. D. Gobena, M. Shimels, P. J. Rich, C. Ruyter-Spira, H. Bouwmeester, S. Kanuganti, T. Mengiste, G. Ejeta, Mutation in sorghum LOW GERMINATION STIMULANT 1 alters strigolactones and causes Striga resistance. Proceedings of the National Academy of Sciences 114, 4471-4476 (2017)10.1073/pnas.1618965114).
33. S. Carlsen, P. K. Ajikumar, L. R. Formenti, K. Zhou, T. H. Phon, M. L. Nielsen, A. E. Lantz, M. C. Kielland-Brandt, G. Stephanopoulos, Heterologous expression and characterization of 50 bacterial 2-C-methyl-D-erythritol-4-phosphate pathway in Saccharomyces cerevisiae. Applied microbiology and biotechnology 97, 5753-5769 (2013); published online Epub July (10.1007/s00253-013-4877-y).
34. P. J. Harrison, S. A. Newgas, F. Descombes, S. A. Shepherd, A. J. Thompson, T. D. Bugg, Biochemical characterization and selective inhibition of beta-carotene cis-trans isomerase D27 and carotenoid cleavage dioxygenase CCD8 on the strigolactone biosynthetic pathway. The FEBS journal 282, 3986-4000 (2015); published online Epub October (10.1111/febs.13400).
35. B. W. Biggs, C. G. Lim, K. Sagliani, S. Shankar, G. Stephanopoulos, M. De Mey, P. K. Ajikumar, Overcoming heterologous protein interdependency to optimize P450-mediated Taxol precursor synthesis in Escherichia coli. Proceedings of the National Academy of Sciences of the United States of America 113, 3209-3214 (2016); published online Epub March 22 (10.1073/pnas.1515826113).
36. J. Guo, X. Ma, Y. Cai, Y. Ma, Z. Zhan, Y. J. Zhou, W. Liu, M. Guan, J. Yang, G. Cui, L. Kang, L. Yang, Y. Shen, J. Tang, H. Lin, X. Ma, B. Jin, Z. Liu, R. J. Peters, Z. K. Zhao, L. Huang, Cytochrome P450 promiscuity leads to a bifurcating biosynthetic pathway for tanshinones. The New phytologist 210, 525-534 (2016); published online Epub April (10.1111/nph.13790).
37. K. Zhou, K. Qiao, S. Edgar, G. Stephanopoulos, Distributing a metabolic pathway among a microbial consortium enhances production of natural products. Nature biotechnology 33, 377-383 (2015); published online Epub April (10.1038/nbt.3095).
38. R. Verwaal, J. Wang, J. P. Meijnen, H. Visser, G. Sandmann, J. A. van den Berg, A. J. van Ooyen, High-level production of beta-carotene in Saccharomyces cerevisiae by successive transformation with carotenogenic genes from Xanthophyllomyces dendrorhous. Applied and environmental microbiology 73, 4342-4350 (2007); published online Epub July (10.1128/AEM.02759-06).
39. V. F. Cataldo, Arenas, N., Salgado, V., Camilo, C., Ibáñez, F., and Agosin, E., Heterologous production of the epoxycarotenoid violaxanthin in Saccharomyces cerevisiae. Metab. Eng. 59, 53-63 (2020)10.1016/j.ymben.2020.01.006).
40. J. Yuan, C. B. Ching, Mitochondrial acetyl-CoA utilization pathway for terpenoid productions. Metabolic engineering 38, 303-309 (2016); published online Epub November (10.1016/j.ymben.2016.07.008).
41. N. Werner, C. A. Ramirez-Sarmiento, E. Agosin, Protein engineering of carotenoid cleavage dioxygenases to optimize beta-ionone biosynthesis in yeast cell factories. Food chemistry 299, 125089 (2019); published online Epub November 30 (10.1016/j.foodchem.2019.125089).
42. M. K. Dhar, S. Mishra, A. Bhat, S. Chib, S. Kaul, Plant carotenoid cleavage oxygenases: structure-function relationships and role in development and metabolism. Briefings in functional genomics 19, 1-9 (2020); published online Epub January 22 (10.1093/bfgp/elz037).
43. J. T. Vogel, M. H. Walter, P. Giavalisco, A. Lytovchenko, W. Kohlen, T. Charnikhova, A. J. Simkin, C. Goulet, D. Strack, H. J. Bouwmeester, A. R. Fernie, H. J. Klee, SICCD7 controls strigolactone biosynthesis, shoot branching and mycorrhiza-induced apocarotenoid formation in tomato. The Plant journal: for cell and molecular biology 61, 300-311 (2010); published online Epub January (10.1111/j.1365-313X.2009.04056.x).
44. S. H. Schwartz, X. Qin, M. C. Loewen, The biochemical characterization of two carotenoid cleavage enzymes from Arabidopsis indicates that a carotenoid-derived compound inhibits lateral branching. The Journal of biological chemistry 279, 46940-46945 (2004); published online Epub November 5 (10.1074/jbc.M409004200).
45. F. C. Huang, P. Molnar, W. Schwab, Cloning and functional characterization of carotenoid cleavage dioxygenase 4 genes. Journal of experimental botany 60, 3011-3022 (2009)10.1093/jxb/erp137).
46. R. Halouzka, P. Tarkowski, B. Zwanenburg, S. Cavar Zeljkovic, Stability of strigolactone analog GR24 toward nucleophiles. Pest management science 74, 896-904 (2018); published online Epub April (10.1002/ps.4782).
47. J. T. Quinlan R, Wurtzel E T, Escherichia coli as a platform for functional expression of plant P450 carotene hydroxylases. Arch Biochem Biophys 458, 146-157 (2007).
48. J. E. Kim, I. S. Jang, S. H. Son, Y. J. Ko, B. K. Cho, S. C. Kim, J. Y. Lee, Tailoring the Saccharomyces cerevisiae endoplasmic reticulum for functional assembly of terpene synthesis pathway. Metabolic engineering 56, 50-59 (2019); published online Epub December (10.1016/j.ymben.2019.08.013).
49. Y. Zhang, X. Cheng, Y. Wang, C. Diez-Simon, K. Flokova, A. Bimbo, H. J. Bouwmeester, C. Ruyter-Spira, The tomato MAX1 homolog, SIMAX1, is involved in the biosynthesis of tomato strigolactones from carlactone. The New phytologist 219, 297-309 (2018); published online Epub July (10.1111/nph.15131).
50. N. Mori, T. Nomura, K. Akiyama, Identification of two oxygenase genes involved in the respective biosynthetic pathways of canonical and non-canonical strigolactones in Lotus japonicus. Planta 251, 40 (2020); published online Epub January 6 (10.1007/s00425-019-03332-x).
51. K. Yoneyama, K. Akiyama, P. B. Brewer, N. Mori, M. Kawano-Kawada, S. Haruta, H. Nishiwaki, S. Yamauchi, X. Xie, M. Umehara, C. A. Beveridge, K. Yoneyama, T. Nomura, Hydroxyl carlactone derivatives are predominant strigolactones in Arabidopsis. Plant direct 4, e00219 (2020); published online Epub May (10.1002/pld3.219).
52. R. J. Chesterfield, C. E. Vickers, C. A. Beveridge, Translation of Strigolactones from Plant Hormone to Agriculture: Achievements, Future Perspectives, and Challenges. Trends in plant science 25, 1087-1106 (2020); published online Epub November (10.1016/j.tplants.2020.06.005).
53. M. N. Hasan, S. S. I. Razvi, A. Kuerban, K. S. Balamash, W. M. Al-Bishri, K. O. Abulnaja, H. Choudhry, J. A. Khan, S. S. Moselhy, Z. M, T. A. Kumosani, A. L. Al-Malki, M. Alhosin, T. Asami, Strigolactones-a novel class of phytohormones as anti-cancer agents. Journal of pesticide science 43, 168-172 (2018); published online Epub August 20 (10.1584/jpestics.D17-090).
54. K. Yoneyama, R. Arakawa, K. Ishimoto, H. I. Kim, T. Kisugi, X. Xie, T. Nomura, F. Kanampiu, T. Yokota, T. Ezawa, K. Yoneyama, Difference in Striga-susceptibility is reflected in strigolactone secretion profile, but not in compatibility and host preference in arbuscular mycorrhizal symbiosis in two maize cultivars. The New phytologist 206, 983-989 (2015); published online Epub May (10.1111/nph.13375).
55. P. B. Brewer, K. Yoneyama, F. Filardo, E. Meyers, A. Scaffidi, T. Frickey, K. Akiyama, Y. Seto, E. A. Dun, J. E. Cremer, S. C. Kerr, M. T. Waters, G. R. Flematti, M. G. Mason, G. Weiller, S. Yamaguchi, T. Nomura, S. M. Smith, K. Yoneyama, C. A. Beveridge, LATERAL BRANCHING OXIDOREDUCTASE acts in the final stages of strigolactone biosynthesis in Arabidopsis. Proceedings of the National Academy of Sciences of the United States of America 113, 6301-6306 (2016); published online Epub May 31 (10.1073/pnas.1601729113).
56. T. Wakabayashi, H. Shinde, N. Shiotani, S. Yamamoto, M. Mizutani, H. Takikawa, Y. Sugimoto, Conversion of methyl carlactonoate to heliolactone in sunflower. Natural product research, 1-8 (2020); published online Epub October 9 (10.1080/14786419.2020.1826477).
57. S. Alberti, Gitler, A. D., Lindquist, S., A suite of Gateway cloning vectors for high-throughput genetic analysis in Saccharomyces cerevisiae. Yeast 24, 913-919 (2007)10.1002/yea.1502).
58. D. G. Gibson, Young, L., Chuang, R. Y., Venter, J. C., Hutchison 3rd, C. A., Smith, H. O., Enzymatic assembly of DNA molecules up to several hundred kilobases. Nat. Methods 6, 343-345 (2009)10.1038/NMETH.1318).
59. Z. Shao, Zhao, H., Zhao, H., DNA assembler, an in vivo genetic method for rapid construction of biochemical pathways. Nucleic Acids Res. 37, e16 (2009)10.1093/nar/gkn991).

1. A co-culture system for the production of strigolactones (SLs), comprising a genetically modified bacterial strain comprising genes that encode enzymes to produce carlactone; and a genetically modified yeast strain comprising genes that encode enzymes to produce SLs; wherein
(a) the genetically modified bacterial strain comprises a polynucleotide comprising a nucleic acid sequence encoding a DWARF27 (D27) polypeptide; a polynucleotide comprising a nucleic acid sequence encoding a carotenoid cleavage dioxygenase CCD7 polypeptide; and a polynucleotide comprising a nucleic acid sequence encoding a carotenoid cleavage dioxygenase CCD8 polypeptide; and
(b) the genetically modified yeast strain comprises a polynucleotide encoding a cytochrome P450 reductase polypeptide, a cytochrome p450 polypeptide and an SL synthase and/or SL synthesetase gene that produces an SL.

2. The co-culture system of claim 1, wherein the D27 polypeptide, the CCD7 polypeptide, and the CCD8 polypeptide each comprise an amino acid sequence of the region of a naturally occurring plant D27, CCD7, and CCD8 polypeptide that lacks the transit polypeptide sequence; or comprises a variant of the naturally occurring D27, CCD7, or CCD8 polypeptide that has at least 90% identity to the naturally occurring plant 27, CCD7, or CCD8 polypeptide sequence.

3. The co-culture system of claim 2, wherein the naturally occurring plant D27 is from rice and the naturally occurring plant CCD7 and/or CCD8 polypeptide is from Arabidopsis thaliana.

4. The co-culture system of claim 2, wherein the D27 polypeptide, the CCD7 polypeptide, and the CCD8 polypeptide each comprise the amino acid sequence as encoded by a corresponding DNA sequence provided in Table 8, or is a variant of the D27, CCD7, and CCD8 polypeptide having at least 95% identity to the corresponding D27, CCD7, and CCD8 amino acid sequence as encoded by the DNA sequence provided in Table 8.

5. The co-culture system of claim 2, wherein the D27 polypeptide comprises an amino acid sequence having at least 95% identity to the D27 polypeptide as encoded by the tPpD27 DNA sequence or PpD27 DNA sequence shown in Table 8, or comprises the amino acid sequence as encoded by the tPpD27 DNA sequence or PpD27 DNA sequence shown in Table 8.

6. The co-culture system of claim 2, wherein the D27 polypeptide comprises an amino acid sequence comprising a 28aa tag sequence or SohB tag sequence as encoded by a DNA sequence as shown in Table 8; or the CCD8 polypeptide comprises an amino acid sequence comprising a 28aa tag sequence or SohB tag sequence as encoded by a DNA sequence as shown in Table 8.

7. The co-culture system of any one of the preceding claims, wherein the yeast strain comprises:
(i) a naturally occurring P450 reductase polypeptide ATR1 from A. thaliana or a naturally occurring homolog from a different plant; and a naturally occurring carlactonoic acid (CLA) synthetase polypeptide MAX1 from A. thaliana, or a naturally occurring homolog polypeptide from a different plant; or a variant of any one of the naturally occurring polypeptide sequences having at least 90% identity to the naturally occurring polypeptide sequence
(ii) a naturally occurring P450 reductase polypeptide ATR1 from A. thaliana or a naturally occurring homolog from a different plant; a naturally occurring CLA synthetase polypeptide MAX1 from A. thaliana, or Zea maize, or a naturally occurring homolog polypeptide from a different plant; and a naturally occurring 5DS synthase CYP722C polypeptide from Gossypium arboretum, or an RcCYP722C2 polypeptide; or a naturally occurring homolog of the 5DS synthase polypeptide; or a variant of any one of the naturally occurring polypeptide sequences having at least 90% identity to the naturally occurring polypeptide sequence;
(iii) a naturally occurring P450 reductase polypeptide ATR1 from A. thaliana or a naturally occurring homolog from a different plant, a naturally occurring carlactonoic acid CLA synthetase MAX1 polypeptide from A. thaliana, or a naturally occurring homolog polypeptide from a different plant; and a naturally occurring orobanchol synthase CYP722c from Capsicum annuum, or a naturally occurring homolog of the CYP722c orobanchol synthase from another plant; or a variant of any one of the naturally occurring polypeptide sequences having at least 90% identity to the naturally occurring polypeptide sequence;
(iv) a naturally occurring P450 reductase polypeptide ATR1 from A. thaliana or a naturally occurring homolog from a different plant; and a naturally occurring 4DO synthase CYP711A2 from Oryza sativa or a naturally occurring homolog from a different plant; or a variant of any one of the naturally occurring polypeptide sequences having at least 90% identity to the naturally occurring polypeptide sequence; or
(v) a naturally occurring P450 reductase polypeptide ATR1 from A. thaliana or a naturally occurring homolog from a different plant; a naturally occurring 4DO synthase CYP711A2 from Oryza sativa or a naturally occurring homolog from a different plant; and a naturally occurring CYP711A3 orobanchol synthase from Oryza sativa; or a variant of any one of the naturally occurring polypeptide sequences having at least 90% identity to the naturally occurring polypeptide sequence.

8. A method of producing CLA, the method comprising culturing the co-culture of claim 4(i) under conditions in which CLA is synthesized.

9. A method of producing 5DS, the method comprising culturing the co-culture of claim 4(ii) under conditions in which 5DS is synthesized.

10. The method of claim 9, wherein the yeast strain has the yeast endogenous NCP1 gene (NP_011908.1) replaced by an Arabidopsis thaliana gene (accession number NP_194183.1.

11. A method of producing orobanchol, the method comprising culturing the co-culture of claim 4(iii) or 4(v) under conditions in which orobanchol is synthesized.

12. A method of producing 4DO, the method comprising culturing the co-culture of claim 4(iv) under conditions in which 4DO is synthesized.
